(19) United States
(12) Patent Application Publication
(10) Pub. No.: US 2022/0285621 A1
(43) Pub. Date: Sep. 8, 2022

(54) ORGANIC MOLECULES FOR OPTOELECTRONIC DEVICES

(71) Applicant: CYNORA GMBH, Bruchsal (DE)

(72) Inventors: Damien Thirion, Karlsdorf-Neuthard (DE); Sebastian Dück, Heidelberg (DE); Daniel Zink, Graben-Neudorf (DE); Anastasia Resetnic, Bruchsal (DE)

(21) Appl. No.: 17/628,641

(22) PCT Filed: Jul. 24, 2020
(86) PCT No.: PCT/EP2020/070965

§ 371 Date: Jan. 20, 2022

(30) Foreign Application Priority Data
Jul. 25, 2019 (EP) 19188361.0
Oct. 11, 2019 (EP) 19202645.8
Dec. 20, 2019 (EP) 19218997.5
Dec. 23, 2019 (EP) 19219378.7
Mar. 11, 2020 (EP) 20162459.0
Mar. 16, 2020 (EP) 20163321.1

(51) Int. Cl. H01L 51/00; C07F 5/02; C09K 11/06

(52) U.S. Cl. CPC H01L 51/008; C07F 5/027; C09K 11/06; H01L 51/5016

(57) ABSTRACT

The invention relates to an organic molecule, in particular for the application in optoelectronic devices. According to the invention, the organic molecule has a structure of formula I:

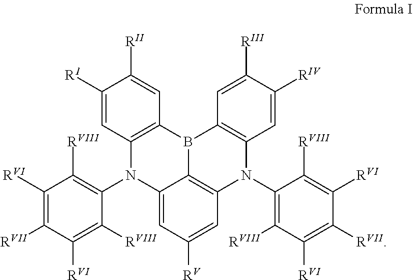

wherein
R^{I}, R^{II}, R^{III} and R^{IV} is independently from another selected from the group consisting of: hydrogen, deuterium, N(R⁵)₂, OR⁵, SR⁵, Si(R⁵)₃, B(OR⁵)₂, OSO₂R⁵, CF₃, CN, halogen, C₁-C₄₀-alkyl, C₁-C₄₀-alkoxy, C₁-C₄₀-thioalkoxy, C₂-C₄₀-alkenyl, C₂-C₄₀-alkynyl, C₆-C₆₀-aryl, and C₃-C₅₇-heteroaryl,
and
R^{V} is selected from the group of C₁-C₅ alkyl, C₆-C₁₈ aryl and C₃-C₁₅ heteroaryl.

The invention relates to organic light-emitting molecules and their use in organic light-emitting diodes (OLEDs) and in other optoelectronic devices.

### DESCRIPTION

The object of the present invention is to provide molecules which are suitable for use in optoelectronic devices.

This object is achieved by the invention which provides a new class of organic molecules.

According to the invention the organic molecules are purely organic molecules, i.e. they do not contain any metal ions in contrast to metal complexes known for the use in optoelectronic devices. The organic molecules of the invention, however, include metalloids, in particular B, Si, Sn, Se, and/or Ge.

According to the present invention, the organic molecules exhibit emission maxima in the sky-blue or green spectral range. As sky-blue or green emitters, the organic molecules emit light in particular between 485 nm and 560 nm. As green emitters, the organic molecules emit between 500 nm and 560 nm or 510 nm and 550 nm, even more preferably between 515 nm and 540 nm. The photoluminescence quantum yields of the organic molecules according to the invention are, in particular, 50% or more. The use of the molecules according to the invention in an optoelectronic device, for example an organic light-emitting diode (OLED), leads to higher efficiencies or higher color purity, expressed by the full width at half maximum (FWHM) of emission, of the device. Corresponding OLEDs have a higher stability than OLEDs with known emitter materials and comparable color.

The organic molecules described herein in particular comprise a severely decreased tendency to form intermolecular aggregates which are known to cause broadening of the photo luminescence (PL) spectra in doped films with increasing concentration.

A measure of this spectral broadening in doped films (e.g. spin coated thin films containing 1 wt % or more of the organic molecule in a PMMA matrix) with increasing concentration is the Concentration Dependent Spectral Purity (CDSP) value.

If two organic molecules have a comparable λₘₐₓ in doped films of the same concentration, the one with a lower CDSP value is preferred in terms of spectral purity. Especially the difference |ΔCDSP| between two concentrations gives evidence whether a material shows a high tendency to aggregate or not: the smaller ΔCDSP, the lower the aggregation tendency of the organic molecule.

The organic light-emitting molecules according to the invention comprise or consist of a structure of formula I:

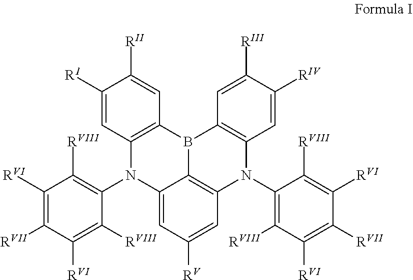

wherein
R^{I}, R^{II}, R^{III} and R^{IV} is independently from another selected from the group consisting of:
hydrogen,
deuterium,
N(R⁵)₂,

### OR⁵,

### SR⁵,

Si(R⁵)₃,
B(OR⁵)₂,
OSO₂R⁵,

### CF₃,

### CN,

halogen,
C₁-C₄₀-alkyl,
which is optionally substituted with one or more substituents R⁵ and
wherein one or more non-adjacent CH₂-groups are optionally substituted by R⁵C≡CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
   C₁-C₄₀-alkoxy,
which is optionally substituted with one or more substituents R⁵ and
wherein one or more non-adjacent CH₂-groups are optionally substituted by R⁵C≡CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-thioalkoxy,
which is optionally substituted with one or more substituents R⁵ and
wherein one or more non-adjacent CH₂-groups are optionally substituted by R⁵C≡CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵
   C₂-C₄₀-alkenyl,
which is optionally substituted with one or more substituents R⁵ and
wherein one or more non-adjacent CH₂-groups are optionally substituted by R⁵C≡CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵
   C₂-C₄₀-alkynyl,
which is optionally substituted with one or more substituents R⁵ and
wherein one or more non-adjacent CH₂-groups are optionally substituted by R⁵C≡CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵
   C₆-C₆₀-aryl,
which is optionally substituted with one or more substituents R⁵; and
   C₃-C₅₇-heteroaryl,
which is optionally substituted with one or more substituents R⁵.

R⁵ is at each occurrence independently from another selected from the group consisting of:

hydrogen, deuterium, OPh, SPh, CF₃, CN, F, Si(C₁-C₅-alkyl)₃, Si(Ph)₃,
C₁-C₅-alkyl,
wherein optionally one or more hydrogen atoms are independently from each other substituted by deuterium, CN, CF₃, or F;
   C₁-C₅-alkoxy,
wherein optionally one or more hydrogen atoms are independently from each other substituted by deuterium, CN, CF₃, or F;
   C₁-C₅-thioalkoxy,
wherein optionally one or more hydrogen atoms are independently from each other substituted by deuterium, CN, CF₃, or F;
   C₂-C₅-alkenyl,
wherein optionally one or more hydrogen atoms are independently from each other substituted by deuterium, CN, CF₃, or F;
   C₂-C₅-alkynyl,
wherein optionally one or more hydrogen atoms are independently from each other substituted by deuterium, CN, CF₃, or F;
   C₆-C₁₃-aryl,
which is optionally substituted with one or more C₁-C₅-alkyl substituents;
   C₃-C₁₇-heteroaryl,
which is optionally substituted with one or more C₁-C₅-alkyl substituents;
   N(C₆-C₁₆-aryl)₂,
   N(C₃-C₁₇-heteroaryl)₂; and
   N(C₃-C₁₇-heteroaryl)(C₆-C₁₈-aryl).

R^{V} is at each occurrence independently from another selected from the group consisting of:

C₁-C₅-alkyl,
wherein one or more hydrogen atoms are optionally substituted by deuterium;
   C₆-C₁₃-aryl,
wherein optionally one or more hydrogen atoms are independently from each other substituted by C₁-C₅-alkyl, C₆-C₁₈ aryl or C₃-C₁₇-heteroaryl;
   C₃-C₁₅-heteroaryl,
wherein optionally one or more hydrogen atoms are independently from each other substituted by C₁-C₅-alkyl, C₆-C₁₈ aryl or C₃-C₁₇-heteroaryl;
   a C₃-C₁₅-heteroaryl, in particular with electron withdrawing properties,
wherein optionally one or more hydrogen atoms are independently from each other substituted by deuterium, halogen, C₁-C₅-alkyl, CN, CF₃, SiMe₃, SiPh₃, C₃-C₁₅-heteroaryl, and
C₆-C₁₈-aryl, wherein optionally one or more hydrogen atoms are independently from each other substituted by C₁-C₅-alkyl, CN, CF₃ and Ph;

R^{VI}, R^{VII} and R^{VIII} are at each occurrence independently from another selected from the group consisting of: hydrogen, deuterium,

C₁-C₅-alkyl,
wherein one or more hydrogen atoms are optionally substituted by deuterium;
   C₆-C₁₃-aryl,
wherein optionally one or more hydrogen atoms are independently from each other substituted by C₁-C₅-alkyl, C₆-C₁₈ aryl or C₃-C₁₇-heteroaryl;
   C₃-C₁₅-heteroaryl,
wherein optionally one or more hydrogen atoms are independently from each other substituted by C₁-C₅-alkyl, C₆-C₁₈ aryl or C₃-C₁₇-heteroaryl.

According to this invention, R^{V} is a C₃-C₁₅-heteroaryl, in particular with electron withdrawing properties,
wherein optionally one or more hydrogen atoms are independently from each other substituted by deuterium, halogen, C₁-C₅-alkyl, CN, CF₃, SiMe₃, SiPh₃, C₃-C₁₅-heteroaryl, and
C₆-C₁₆-aryl, wherein optionally one or more hydrogen atoms are independently from each other substituted by C₁-C₅-alkyl, CN, CF₃ and Ph.

In one embodiment, R^{V} is a C₃-C₁₅-heteroaryl, in particular with electron withdrawing properties, wherein optionally one or more hydrogen atoms are independently from each other substituted by C₁-C₅-alkyl, C₆-C₁₈-aryl or C₃-C₁₇-heteroaryl;

In one embodiment, R^{V} is a C₃-C₁₅-heteroaryl, in particular with electron withdrawing properties, wherein optionally one or more hydrogen atoms are independently from each other substituted by C₁-C₅-alkyl, C₆-C₁₈-aryl or C₃-C₁₇-heteroaryl;

In one embodiment, R^{V} is a nitrogen containing C₃-C₁₅-heteroaryl, in particular with electron withdrawing properties, wherein optionally one or more hydrogen atoms are independently from each other substituted by C₁-C₅-alkyl, C₆-C₁₈ aryl or C₃-C₁₇-heteroaryl;

In a preferred embodiment, the organic molecules according to the invention comprise or consist a structure of formula 3

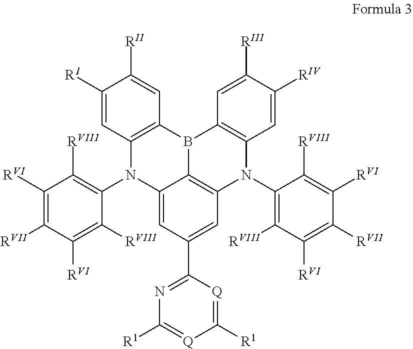

wherein
Q is at each occurrence independently from each other selected from the group consisting of N and C—R².
R¹ and R² are at each occurrence independently from another selected from the group consisting of:
hydrogen, deuterium, halogen, Me, ⁱPr, ⁱBu, CN, CF₃, SiMe₃, SiPh₃ (Ph=phenyl), and C₆-C₁₃-aryl,
wherein optionally one or more hydrogen atoms are independently from each other substituted by C₁-C₅-alkyl, CN, CF₃ and Ph.

R^{I}, R^{II}, R^{III} and R^{IV} is independently from another selected from the group consisting of:

hydrogen,
deuterium,
N(R⁵)₂,

### OR⁵,

### SR⁵,

Si(R⁵)₃,
B(OR⁵)₂,
OSO₂R⁵,

### CF₃,

### CN,

halogen,
C₁-C₄₀-alkyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵
   C₁-C₄₀-alkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵
   C₁-C₄₀-thioalkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵
   C₂-C₄₀-alkenyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵
   C₂-C₄₀-alkynyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
   C₆-C₆₀-aryl,
which is optionally substituted with one or more substituents R⁵; and
   C₃-C₅₇-heteroaryl,
which is optionally substituted with one or more substituents R⁵.

R⁵ is at each occurrence independently from another selected from the group consisting of:

hydrogen, deuterium, OPh, SPh, CF₃, CN, F, Si(C₁-C₅-alkyl)₃, Si(Ph)₃,
C₁-C₅-alkyl,
wherein optionally one or more hydrogen atoms are independently from each other substituted by deuterium, CN, CF₃, or F;
   C₁-C₅-alkoxy,
wherein optionally one or more hydrogen atoms are independently from each other substituted by deuterium, CN, CF₃, or F;
   C₁-C₅-thioalkoxy,
wherein optionally one or more hydrogen atoms are independently from each other substituted by deuterium, CN, CF₃, or F;
   C₂-C₅-alkenyl,
wherein optionally one or more hydrogen atoms are independently from each other substituted by deuterium, CN, CF₃, or F;
   C₂-C₅-alkynyl,
wherein optionally one or more hydrogen atoms are independently from each other substituted by deuterium, CN, CF₃, or F;
   C₆-C₁₈-aryl,
which is optionally substituted with one or more C₁-C₅-alkyl substituents;
   C₃-C₁₇-heteroaryl,
which is optionally substituted with one or more C₁-C₅-alkyl substituents;
   N(C₆-C₁₈-aryl)₂,
   N(C₃-C₁₇-heteroaryl)₂; and
   N(C₃-C₁₇-heteroaryl)(C₆-C₁₈-aryl).

R^{VI}, R^{VII} and R^{VIII} are at each occurrence independently from another selected from the group consisting of: hydrogen, deuterium,

C₁-C₅-alkyl,
wherein one or more hydrogen atoms are optionally substituted by deuterium;
   C₆-C₁₈-aryl,
wherein optionally one or more hydrogen atoms are independently from each other substituted by C₁-C₅-alkyl, C₆-C₁₈ aryl or C₃-C₁₇-heteroaryl;
   C₃-C₁₅-heteroaryl,
wherein optionally one or more hydrogen atoms are independently from each other substituted by C₁-C₅-alkyl, C₆-C₁₈ aryl or C₃-C₇-heteroaryl.

In a particularly preferred embodiment of this invention, R^{V} comprises or consists of a structure of formula I-0:

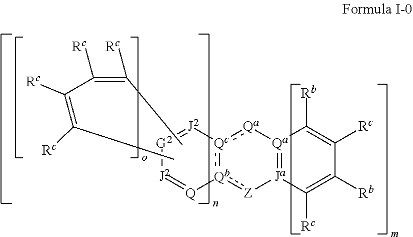

wherein
m is 0 or 1;
n is 0 or 1;
is 0 or 1;
if n=0, then o=0;
G^{a} is C if m=1; G^{a} is CR^{a} or N if m=0;
J^{a} is C if m=1; J^{a} is CR^{a} or N if m=0;
Q^{b} is C if n=1; Q^{b} is CR¹ or N if n=0;
Q^{c} is C if n=1; Q^{c} is CR² or N if n=0;
G² is C if o=1; G² is CR^{b} or N if o=0;
if o=1 exactly one J² is C and the other J² is N or CR^{b};
if o=0 J² is at each occurrence independently from another selected from the group consisting of N and CR^{b};
Q is at each occurrence independently from each other selected from the group consisting of N and C—R²
Z is Q or a direct (single) bond;
if Z is a direct bond:
   Q^{a} is selected from a group consisting of NR^{c}, O;
   Q^{b} and Q^{c} are connected via a double bond and Q^{c} and Q^{a} are connected via a single bond as shown in Formula I-0a:

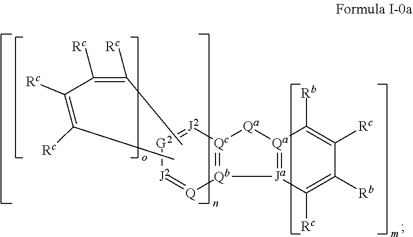

if Z is Q:
   Q^{a} is selected from a group consisting N, O, C—R^{a};
   Z and Q^{b} are connected via a double bond, while Q^{b} and Q^{c} are connected via a single bond and Q^{c} and Q^{a} are connected via a double bond as shown in Formula I-0b, wherein Z is already shown as Q:

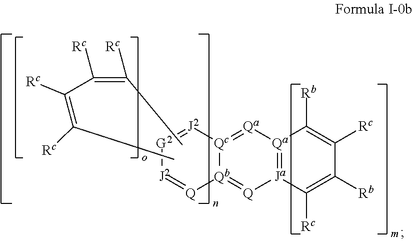

R^{a} is at each occurrence independently from another selected from the group consisting of the binding site of R^{V} to the rest of the organic molecule as shown in formula I or R²
R¹ and R² are at each occurrence independently from another selected from the group consisting of:
hydrogen, deuterium, halogen, C₁-C₅-alkyl, CN, CF₃, SiMe₃, SiPh₃ (Ph=phenyl), C₃-C₁₅-heteroaryl, and
C₆-C₁₆-aryl, wherein optionally one or more hydrogen atoms are independently from each other substituted by C₁-C₅-alkyl, CN, CF₃ and Ph;
R^{b} is at each occurrence independently from another selected from the group consisting of the binding site, hydrogen, deuterium, C₁-C₅-alkyl, C₆-C₁₈ aryl or C₃-C₇-heteroaryl;
R^{c} is at each occurrence independently from another selected from the group consisting of hydrogen, deuterium, C₁-C₅-alkyl, C₆-C₁₈ aryl or C₃-C₇-heteroaryl; exactly one of R^{a} and R^{b} is the binding site of R^{V} to the rest of the organic molecule as shown in formula I;

According to the invention, at least one atom of Formula I-0 is N or O.

Examples of the organic molecules according to the invention, wherein R^{V} comprises or consists of a structure of formula I-0 are listed below:

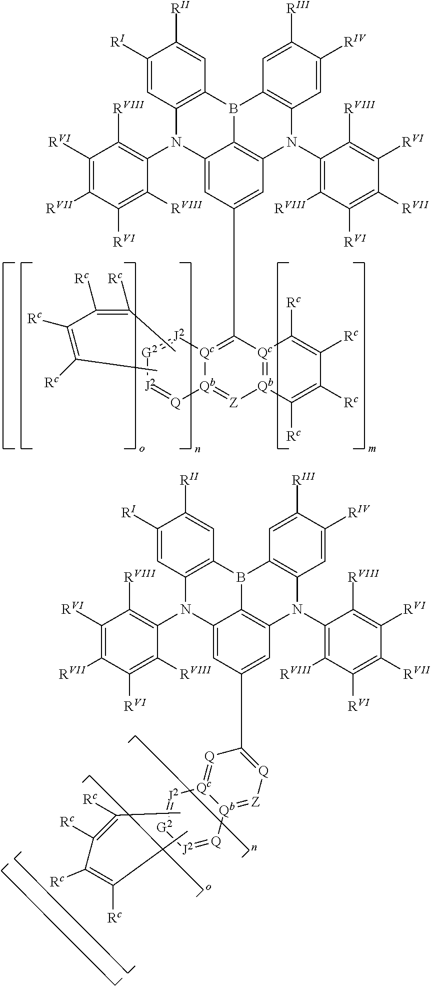

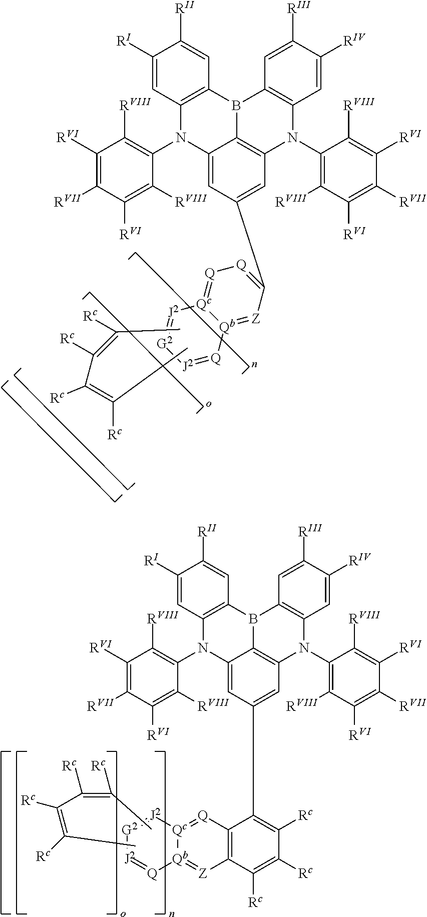

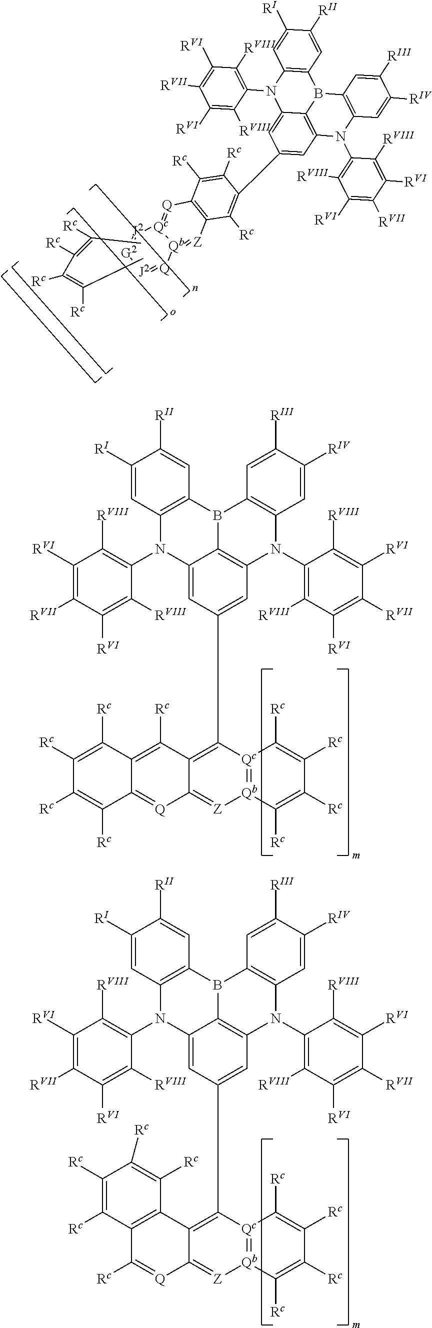

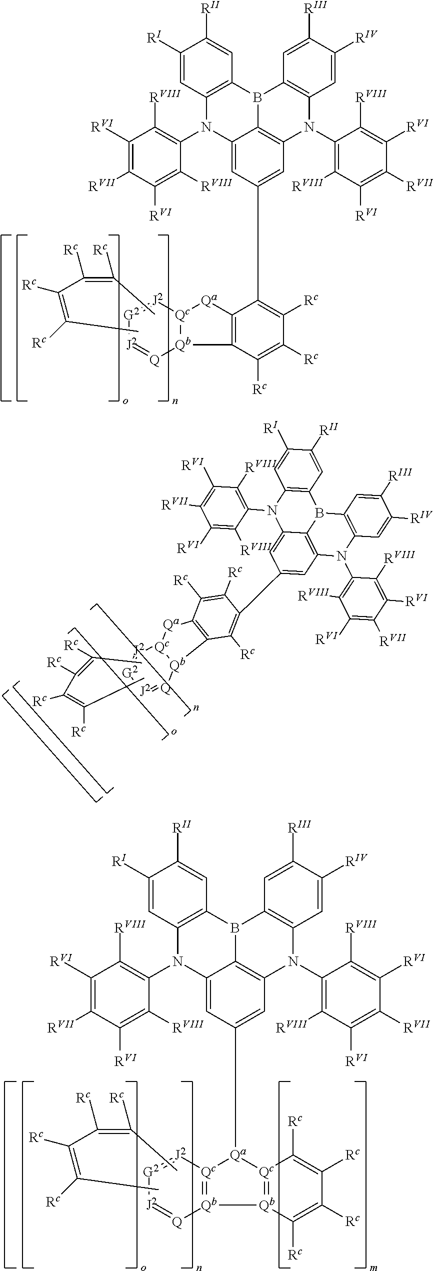

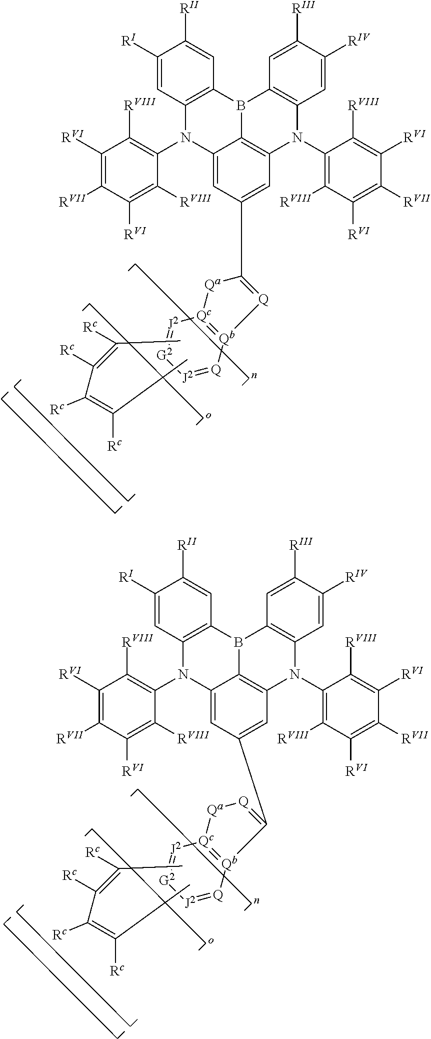

In embodiments with n=0, m=0, and o=0, formula I-0:

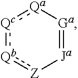

is represented by

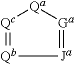

if Z is a direct bond, and by

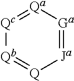

### if Z is Q.

In embodiments with n=1, m=0, and o=0, formula I-0:

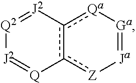

is represented by

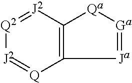

if Z is a direct bond, and by

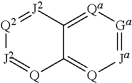

### if Z is Q.

In the embodiment n=1, m=1, and o=0, Formula I-0:

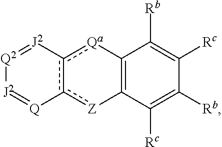

is represented by

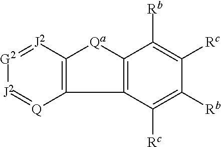

if Z is a direct bond, and by

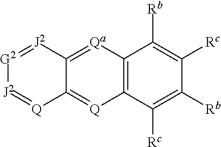

### if Z is Q.

In embodiments with n=1, m=0, and o=1, formula I-0:

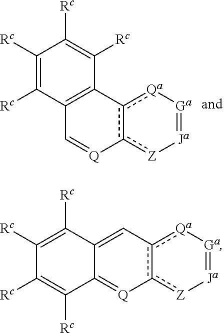

is represented by

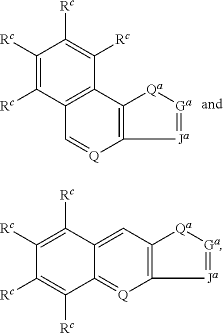

if Z is a direct bond, and by

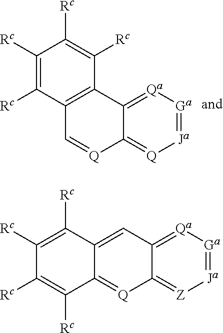

### if Z is Q.

In embodiments with n=1, m=1, and o=1, formula I-0:

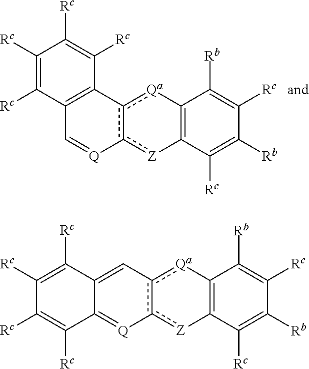

is represented by

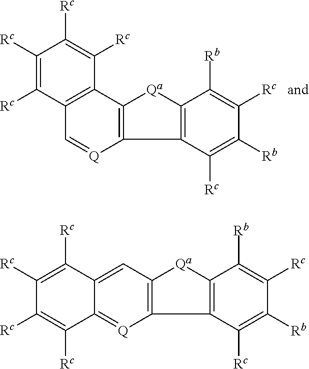

is a direct bond, and by

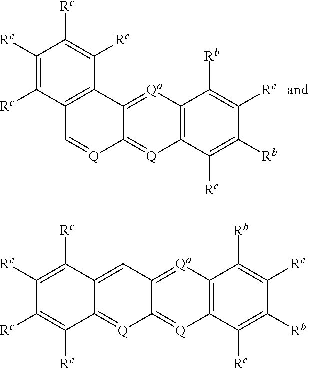

### if Z is Q.

In the embodiment n=0, m=1, and o=0, formula I-0:

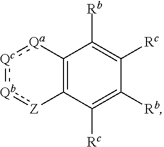

is represented by

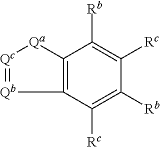

if Z is a direct bond, and by

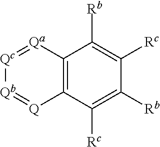

### if Z is Q.

In a particularly preferred embodiment, n+m+o<3, i.e. the sum of n, m, and o is smaller than three, namely 0, 1, or 2.

In one embodiment, R^{V} comprises or consists of a structure of formula I-0, wherein
R^{b} is at each occurrence independently from another selected from the group consisting of the binding site, hydrogen, deuterium, C₁-C₅-alkyl, or C₆-C₁₈ aryl;
R^{c} is at each occurrence independently from another selected from the group consisting of hydrogen, deuterium, C₁-C₅-alkyl, or C₆-C₁₈ aryl;
exactly one of R^{a} and R^{b} is the binding site;
at least one of Q, Q^{a}, Q^{b}, Q^{c}, G^{a}, J^{a}, J², G² or Z is N or Q^{a} is O.

In one embodiment, R^{V} comprises or consists of a structure of formula I-0, wherein
R^{b} is at each occurrence independently from another selected from the group consisting of the binding site, hydrogen, deuterium, Me, ⁱPr, ^{t}Bu, or C₆-C₁₈ aryl;
R^{c} is at each occurrence independently from another selected from the group consisting of hydrogen, deuterium, Me, ⁱPr, ^{t}Bu, or C₆-C₁₈ aryl;
exactly one of R^{a} and R^{b} is the binding site;
at least one of Q, Q^{a}, Q^{b}, Q^{c}, G^{a}, J^{a}, J², G² or Z is N or Q^{a} is O.

In one embodiment, R^{V} comprises or consists of a structure of formula I-0, wherein
R^{a} and R^{b} are at each occurrence independently from another selected from the group consisting of the binding site, hydrogen, deuterium, Me, ⁱPr, ^{t}Bu, or C₆-C₁₈ aryl;
R^{c} is at each occurrence independently from another selected from the group consisting of hydrogen, deuterium, Me, ⁱPr, ^{t}Bu, or C₆-C₁₈ aryl;
exactly one of R^{a} and R^{b} is the binding site;
at least one of Q, Q^{a}, Q^{b}, Q^{c}, G^{a}, J^{a}, J², G² or Z is N or Q^{a} is O.

In one embodiment, R^{V} is selected from the group consisting of formulas 2a, 2b, 2c, 2d, 2e, 2f, 2g, 2h, 2i, 2j, 2k and 2l:

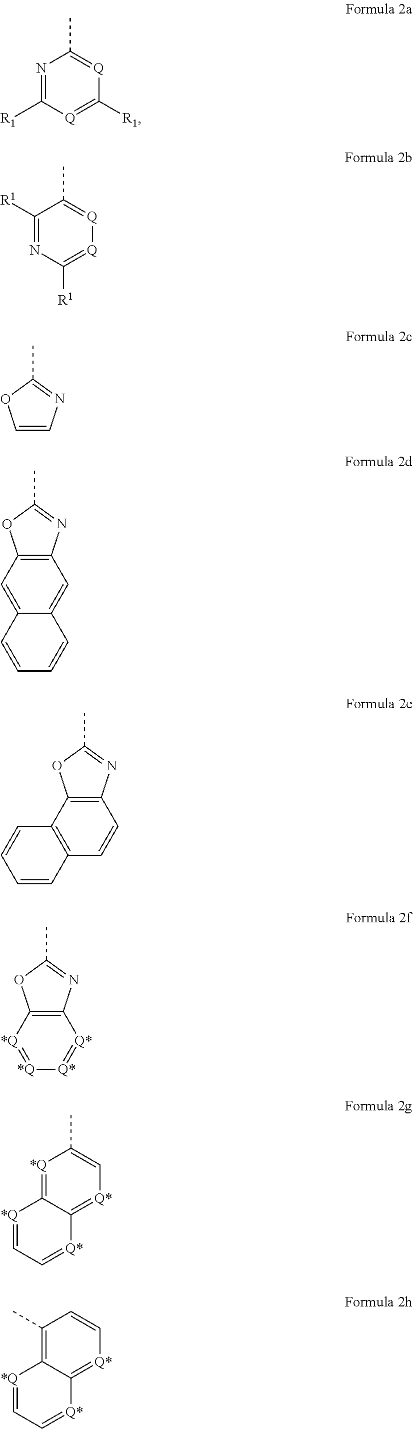

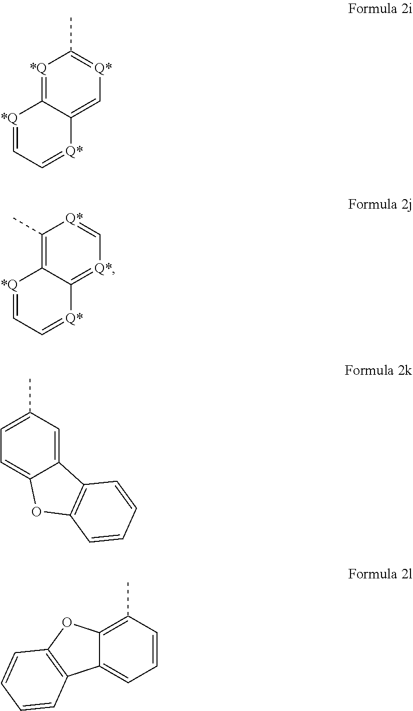

wherein Q* is at each occurrence independently from each other selected from the group consisting of N and C—H;
wherein for 2g, 2h, 2i, 2j, at least one Q*=N;
wherein optionally one or more hydrogen atoms are independently from each other substituted by R^{c}; and
which is bonded via the position marked by a dotted line.

In a preferred embodiment, R^{V} is selected from a group consisting of formula 2a, 2b, 2c, 2d, 2e, 2f, 2g, 2h, 2i, 2j, 2k or 2l,

wherein Q is at each occurrence independently from each other selected from the group consisting of N and C—R².
R¹ and R² are at each occurrence independently from another selected from the group consisting of:
hydrogen, deuterium, halogen, Me, ⁱPr, ^{t}Bu, CN, CF₃, SiMe₃, SiPh₃ (Ph=phenyl), and C₆-C₁₃-aryl,
wherein optionally one or more hydrogen atoms are independently from each other substituted by C₁-C₅-alkyl, CN, CF₃ and Ph.

In an even more preferred embodiment, R^{V} is comprising or consisting of formula 2a

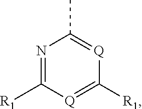

### Formula 2a

wherein
Q is at each occurrence independently from each other selected from the group consisting of N and C—R².
R¹ and R² are at each occurrence independently from another selected from the group consisting of:
hydrogen, deuterium, halogen, Me, ⁱPr, ^{t}Bu, CN, CF₃, SiMe₃, SiPh₃ (Ph=phenyl), and C₆-C₁₃-aryl,
   wherein optionally one or more hydrogen atoms are independently from each other substituted by C₁-C₅-alkyl, CN, CF₃ and Ph.

Examples of the organic molecules according to the invention are listed below:

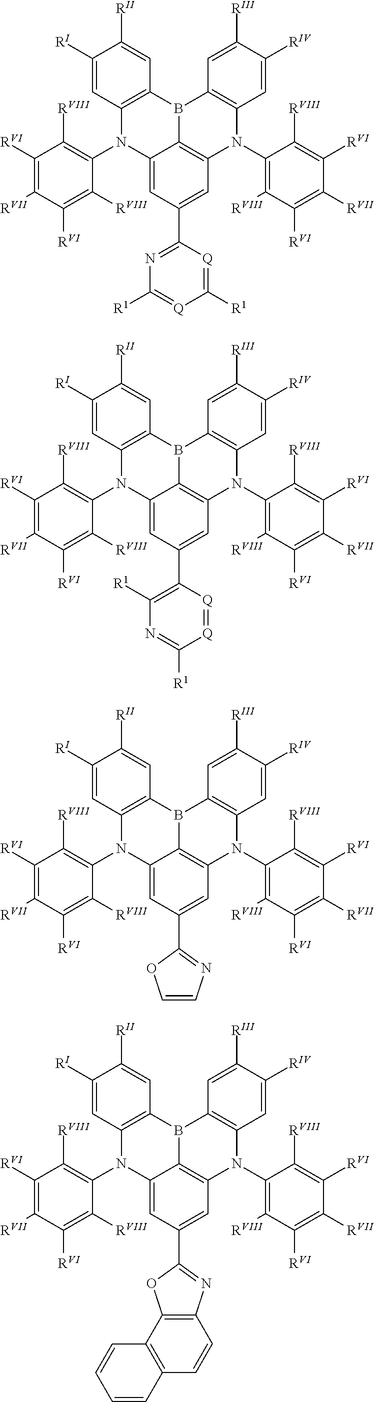

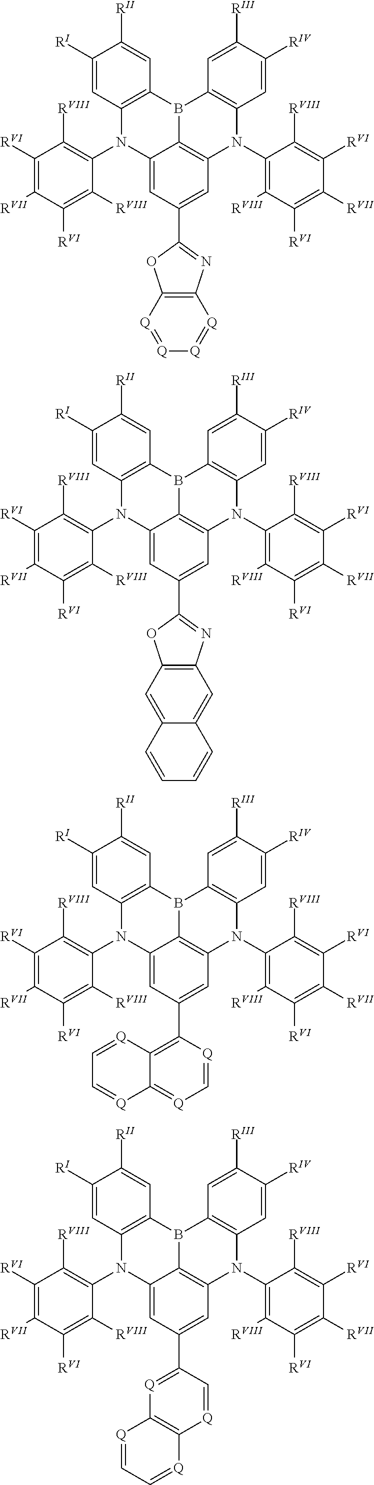

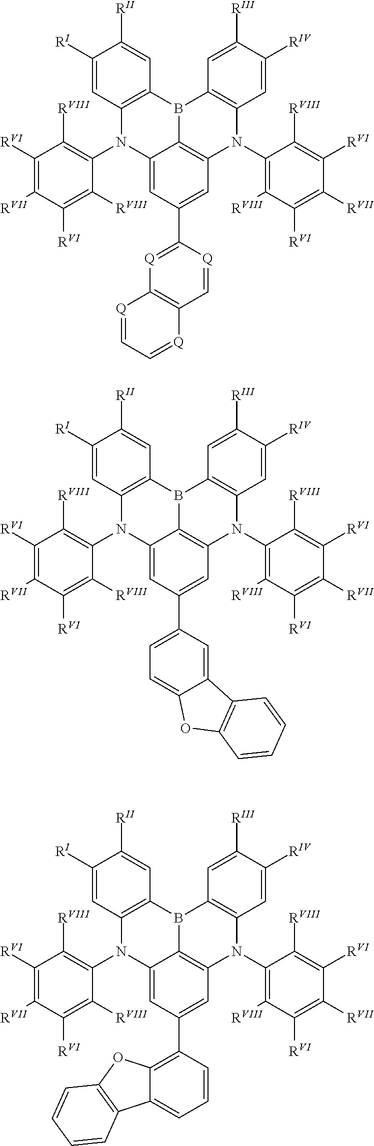

In one embodiment, R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, C₁-C₅-alkyl, and C₆-C₁₃ aryl.

In one embodiment, R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, Me, ⁱPr, ^{t}Bu, and C₆-C₁₈ aryl;

In one embodiment, R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, Me, ⁱPr, or ^{t}Bu.

In one embodiment, R^{V} is selected from the group consisting of formulas 2a, 2b, 2c, 2d, 2e, 2f, 2g, 2h, 2i, 2j, 2k and 2l, wherein R^{c} is hydrogen.

In one embodiment, R^{V} is selected from the group consisting of formulas 2a, 2b, 2c, 2d, 2e, 2f, 2g, 2h, 2i, 2j, 2k and 2l, wherein R¹ and R² are at each occurrence independently from another selected from the group consisting of hydrogen, deuterium, Me, ⁱPr, ^{t}Bu, CN, or C₆-C₁₈ aryl.

In one embodiment, R^{V} is selected from the group consisting of formulas 2a, 2b, 2c, 2d, 2e, 2f, 2g, 2h, 2i, 2j, 2k and 2l, wherein R¹, R² and R^{c} are at each occurrence independently from another selected from the group consisting of hydrogen, deuterium, Me, ⁱPr, ^{t}Bu, or C₆-C₁₈ aryl.

In one embodiment, R^{V} is selected from the group consisting of formulas 2a, 2b, 2c, 2d, 2e, 2f, 2g, 2h, 2i, and 2j.

In one embodiment, R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, C₁-C₅-alkyl, and C₆-C₁₈ aryl.

In one embodiment, R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, Me, ⁱPr, ^{t}Bu, and C₆-C₁₈ aryl.

In one embodiment, R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, Me, ⁱPr, and ^{t}Bu.

In one embodiment, R^{V} is selected from the group consisting of formulas 2a, 2b, 2c, 2d, 2e, 2f, 2g, 2h, 2i, and 2j, wherein R^{c} is hydrogen.

In one embodiment, R^{V} is selected from the group consisting of formulas 2a, 2b, 2c, 2d, 2e, 2f, 2g, 2h, 2i, and 2j, wherein R¹ and R² are at each occurrence independently from another selected from the group consisting of hydrogen, deuterium, Me, ⁱPr, ^{t}Bu. CN, an C₆-C₁₈ aryl.

In one embodiment, R^{V} is selected from the group consisting of formulas 2a, 2b, 2c, 2d, 2e, 2f, 2g, 2h, 2i, and 2j, wherein R¹, R² and R^{c} are at each occurrence independently from another selected from the group consisting of hydrogen, deuterium, Me, ⁱPr, ^{t}Bu, and C₆-C₁₈ aryl.

In one embodiment of the organic molecule, R^{I}, R^{II}, R^{III} and R^{IV} are independently from another selected from the group consisting of:
hydrogen, deuterium, halogen, Me, ⁱPr, ^{t}Bu, CN, CF₃, SiMe₃, SiPh₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph,
carbazolyl, which is optionally substituted with one or more substituents independently from each other selected from the group consisting of Me, ⁱPr, ^{t}Bu, CN, CF₃, and Ph,
triazinyl, which is optionally substituted with one or more substituents independently from each other selected from the group consisting of Me, ⁱPr, ^{t}Bu, CN, CF₃, and Ph,
and N(Ph)₂.

In one embodiment, R^{I}, R^{II}, R^{III} and R^{IV} are independently from another selected from the group consisting of:
hydrogen, deuterium, halogen, Me, ⁱPr, ^{t}Bu, CN, CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph,
carbazolyl, which is optionally substituted with one or more substituents independently from each other selected from the group consisting of Me, ⁱPr, ^{t}Bu, CN, CF₃, and Ph,
triazinyl, which is optionally substituted with one or more substituents independently from each other selected from the group consisting of Me, ⁱPr, ^{t}Bu, CN, CF₃, and Ph,
and N(Ph)₂.

In one embodiment, R^{I}, R^{II}, R^{III} and R^{IV} is independently from another selected from the group consisting of: hydrogen, deuterium, halogen, Me, ⁱPr, ^{t}Bu, CN, CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph, and
triazinyl, which is optionally substituted with one or more substituents independently from each other selected from the group consisting of Me, ⁱPr, ^{t}Bu, CN, CF₃, and Ph,
and N(Ph)₂.

In one embodiment, R^{I}, R^{II}, R^{III} and R^{IV} is independently from another selected from the group consisting of: hydrogen, deuterium, Me, ⁱPr, ^{t}Bu,
Ph, which is optionally substituted with one or more substituents independently from each other selected from the group consisting of Me, ⁱPr, ^{t}Bu, and Ph,
carbazolyl, which is optionally substituted with one or more substituents independently from each other selected from the group consisting of Me, ⁱPr, ^{t}Bu, CN, CF₃, and Ph,
and N(Ph)₂.

In one embodiment R^{I}, R^{II}, R^{III} and R^{IV} is independently from another selected from the group consisting of: hydrogen, deuterium, Me, ⁱPr, ^{t}Bu,
Ph, which is optionally substituted with one or more substituents independently from each other selected from the group consisting of Me, ⁱPr, ^{t}Bu, and Ph,
and N(Ph)₂.

In one embodiment, R^{I}, R^{II}, R^{III} and R^{IV} is independently from another selected from the group consisting of: hydrogen, deuterium, Me, ⁱPr, ^{t}Bu,
Ph, which is optionally substituted with one or more substituents independently from each other selected from the group consisting of Me, ⁱPr, ^{t}Bu, and Ph,
and N(Ph)₂.

In one embodiment, R^{I}, R^{II}, R^{III} and R^{IV} is independently from another selected from the group consisting of: hydrogen, ^{t}Bu, and Ph.

In one embodiment, R^{I} is hydrogen.

In one embodiment, R^{I} is ^{t}Bu.

In one embodiment, R^{I} is Ph.

In one embodiment, R^{II} is hydrogen.

In one embodiment, R^{II} is ^{t}Bu.

In one embodiment, R^{III} is Ph.

In one embodiment, R^{III} is hydrogen.

In one embodiment, R^{III} is ^{t}Bu.

In one embodiment, R^{III} is Ph.

In one embodiment, R^{IV} is hydrogen.

In one embodiment, R^{IV} is ^{t}Bu.

In one embodiment, R^{IV} is Ph.

In one embodiment, R^{I} and R^{IV} are hydrogen, and R^{II} is equal to R^{III}, in particular, R^{II} and R^{III} are identical.

In one embodiment, R^{I} and R^{IV} are hydrogen, and R^{II} and R^{III} are ^{t}Bu.

In one embodiment, R^{I} and R^{IV} are hydrogen, and R^{II} and R^{III} are Ph.

In one embodiment, R^{V} is mesityl (Mes) or 2-meta-ter-phenyl.

In one embodiment, R^{V} is 2-meta-ter-phenyl.

In one embodiment, R^{VI}, R^{VII} and R^{VIII} are at each occurrence independently from each other selected from the group consisting of hydrogen,

C₁-C₅-alkyl,
C₆-C₁₃-aryl,
wherein optionally one or more hydrogen atoms are independently from each other substituted by C₁-C₅-alkyl or C₆-C₁₈-aryl.

In one embodiment, R^{VI}, R^{VII} and R^{VIII} are at each occurrence independently from each other selected from the group consisting of hydrogen, Ph and ^{t}Bu.

In one embodiment, R^{VI} is Ph, and R^{VII} and R^{VIII} are hydrogen.

In one embodiment, R^{VII} is Ph, and R^{VI} and R^{VIII} are hydrogen.

In one embodiment, R^{VI} is ^{t}Bu, and R^{VII} and R^{VIII} are hydrogen.

In one embodiment, R^{VII} is ^{t}Bu, and R^{VI} and R^{VIII} are hydrogen.

In one embodiment, the organic molecule comprises or consists of a structure of formula Ia:

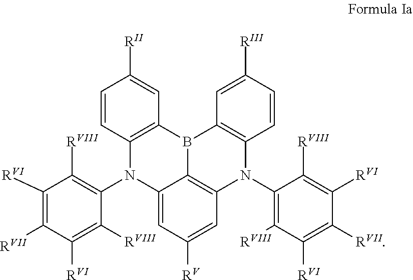

wherein R^{II} and R^{III} is independently from another selected from the group consisting of:
hydrogen, deuterium, halogen, Me, ⁱPr, ^{t}Bu, CN, CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph, and
triazinyl, which is optionally substituted with one or more substituents independently from each other selected from the group consisting of Me, ⁱPr, ^{t}Bu, CN, CF₃, and Ph,
and N(Ph)₂.

In one embodiment, the organic molecule comprises or consists of a structure of formula Ia, wherein R^{I} and R^{III} is independently from another selected from the group consisting of:
hydrogen, deuterium, Me, ⁱPr, ^{t}Bu,
Ph, which is optionally substituted with one or more substituents independently from each other selected from the group consisting of Me, ⁱPr, ^{t}Bu, and Ph,
carbazolyl, which is optionally substituted with one or more substituents independently from each other selected from the group consisting of Me, ⁱPr, ^{t}Bu, CN, CF₃, and Ph,
and N(Ph)₂.

In one embodiment, the organic molecule comprises or consists of a structure of formula Ia, wherein R^{I} and R^{III} is each independently from another selected from the group consisting of: hydrogen, deuterium, Me, ⁱPr, ^{t}Bu,
Ph, which is optionally substituted with one or more substituents independently from each other selected from the group consisting of Me, ⁱPr, ^{t}Bu, and Ph,
and N(Ph)₂.

In one embodiment, the organic molecule comprises or consists of a structure of formula Ia, wherein R^{II} and R^{III} is independently from another selected from the group consisting of:

hydrogen, deuterium, Me, ⁱPr, ^{t}Bu,
Ph, which is optionally substituted with one or more substituents independently from each other selected from the group consisting of Me, ⁱPr, ^{t}Bu, and Ph,

### and N(Ph)₂.

In one embodiment, the organic molecule comprises or consists of a structure of formula Ia, wherein R^{II} and R^{III} is independently from another selected from the group consisting of: hydrogen, ^{t}Bu, and Ph.

In one embodiment, the organic molecule comprises or consists of a structure of formula Ia, wherein R^{II} is hydrogen.

In one embodiment, the organic molecule comprises or consists of a structure of formula Ia, wherein R^{II} is ^{t}Bu.

In one embodiment, the organic molecule comprises or consists of a structure of formula Ia, wherein R^{II} is Ph.

In one embodiment, the organic molecule comprises or consists of a structure of formula Ia, wherein R^{III} is hydrogen.

In one embodiment, the organic molecule comprises or consists of a structure of formula Ia, wherein R^{III} is ^{t}Bu.

In one embodiment, the organic molecule comprises or consists of a structure of formula Ia, wherein R^{III} is Ph.

In one embodiment, the organic molecule comprises or consists of a structure of formula Ia, wherein R^{II} is equal to R^{III}, in particular, R^{II} and R^{III} are identical.

In one embodiment, the organic molecule comprises or consists of a structure of formula Ia, wherein R^{I} and R^{III} are ^{t}Bu.

In one embodiment, the organic molecule comprises or consists of a structure of formula Ia, wherein R^{II} and R^{III} are Ph.

In one embodiment, the organic molecule comprises or consists of a structure of formula Ia, wherein R^{II} and R^{III} are hydrogen.

In one embodiment, the organic molecule comprises or consists of a structure of formula Ia, wherein R^{V} is mesityl (Mes) or 2-meta-ter-phenyl.

In one embodiment, the organic molecule comprises or consists of a structure of formula Ia, wherein R^{V} is 2-meta-ter-phenyl.

In one embodiment, the organic molecule comprises or consists of a structure of formula Ia, wherein R^{V} is C₃-C₁₅-heteroaryl, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Ia, wherein R^{V} is a C₃-C₁₅-heteroaryl, in particular with electron withdrawing properties, wherein optionally one or more hydrogen atoms are independently from each other substituted by C₁-C₅-alkyl, C₆-C₁₈ aryl or C₃-C₁₇-heteroaryl;

In a preferred embodiment, the organic molecule comprises or consists of a structure of formula Ia, wherein R^{V} consists of a structure of formula I-0.

In one embodiment, the organic molecule comprises or consists of a structure of formula Ia, wherein R^{V} consists of a structure of formula I-0, wherein
R^{b} is at each occurrence independently from another selected from the group consisting of the binding site, hydrogen, deuterium, Me, ⁱPr, ^{t}Bu, or C₆-C₁₈ aryl;
R^{c} is at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula Ia, wherein R^{V} is selected from a group consisting of formula 2a, 2b, 2c, 2d, 2e, 2f, 2g, 2h, 2i, 2j, 2k or 2l;

In one embodiment, the organic molecule comprises or consists of a structure of formula Ia, wherein R^{V} is selected from the group consisting of formulas 2a, 2b, 2c, 2d, 2e, 2f, 2g, 2h, 2i, and 2j.

In one embodiment, the organic molecule comprises or consists of a structure of formula Ia, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, C₁-C₅-alkyl, and C₆-C₁₈ aryl.

In one embodiment, the organic molecule comprises or consists of a structure of formula Ia, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, Me, ⁱPr, ^{t}Bu, and C₆-C₁₈ aryl.

In one embodiment, the organic molecule comprises or consists of a structure of formula Ia, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, Me, ⁱPr, and ^{t}Bu.

In one embodiment, the organic molecule comprises or consists of a structure of formula Ia, wherein R^{V} is selected from the group consisting of formulas 2a, 2b, 2c, 2d, 2e, 2f, 2g, 2h, 2i, 2j, 2k and 2l, wherein R^{c} is hydrogen.

In one embodiment, the organic molecule comprises or consists of a structure of formula Ia, wherein R^{V} is selected from a group consisting of formula 2a, 2b, 2c, 2d, 2e, 2f, 2g, 2h, 2i, 2j, 2k or 2l, wherein R¹ and R² are at each occurrence independently from another selected from the group consisting of hydrogen, deuterium, Me, ⁱPr, ^{t}Bu, CN, or C₆-C₁₈ aryl;

In one embodiment, the organic molecule comprises or consists of a structure of formula Ia, wherein R^{V} is selected from a group consisting of formula 2a, 2b, 2c, 2d, 2e, 2f, 2g, 2h, 2i, 2j, 2k or 2l, wherein R¹, R² and R^{c} are at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ia, wherein R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, C₁-C₅-alkyl, and C₆-C₁₈ aryl.

In one embodiment, the organic molecule comprises or consists of a structure of formula Ia, wherein R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, Me, ⁱPr, ^{t}Bu, and C₆-C₁₈ aryl.

In one embodiment, the organic molecule comprises or consists of a structure of formula Ia, wherein R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, Me, ⁱPr, and ^{t}Bu.

In one embodiment, the organic molecule comprises or consists of a structure of formula Ia, wherein R^{V} is selected from the group consisting of formulas 2a, 2b, 2c, 2d, 2e, 2f, 2g, 2h, 2i, and 2j, wherein R^{c} is hydrogen.

In one embodiment, the organic molecule comprises or consists of a structure of formula Ia, wherein R^{V} is selected from the group consisting of formulas 2a, 2b, 2c, 2d, 2e, 2f, 2g, 2h, 2i, and 2j, wherein R¹ and R² are at each occurrence independently from another selected from the group consisting of hydrogen, deuterium, Me, ⁱPr, ^{t}Bu, CN, and C₆-C₁₈ aryl.

In one embodiment, the organic molecule comprises or consists of a structure of formula Ia, wherein R^{V} is selected from the group consisting of formulas 2a, 2b, 2c, 2d, 2e, 2f, 2g, 2h, 2i, and 2j, wherein R¹, R² and R^{c} are at each occurrence independently from another selected from the group consisting of hydrogen, deuterium, Me, ⁱPr, ^{t}Bu, and C₆-C₁₈ aryl.

In one embodiment, R^{VI}, R^{VII} and R^{VIII} are at each occurrence independently from each other selected from the group consisting of hydrogen,

C₁-C₅-alkyl,
C₆-C₁₃-aryl,
wherein optionally one or more hydrogen atoms are independently from each other substituted by C₁-C₅-alkyl or C₆-C₁₆-aryl.

In one embodiment, the organic molecule comprises or consists of a structure of formula Ia, wherein R^{VI}, R^{VII} and R^{VIII} are at each occurrence independently from each other selected from the group consisting of hydrogen, Ph and ^{t}Bu.

In one embodiment, the organic molecule comprises or consists of a structure of formula Ia, wherein R^{VI} is Ph, and R^{VII} and R^{VIII} are hydrogen.

In one embodiment, the organic molecule comprises or consists of a structure of formula Ia, wherein R^{VII} is Ph, and R^{VI} and R^{VIII} are hydrogen.

In one embodiment, the organic molecule comprises or consists of a structure of formula Ia, wherein R^{VI} is ^{t}Bu, and R^{VII} and R^{VIII} are hydrogen.

In one embodiment the organic molecule comprises or consists of a structure of formula Ia, wherein, R^{VII} is ^{t}Bu, and R^{VI} and R^{VIII} are hydrogen.

In one embodiment, the organic molecule comprises or consists of a structure of formula Ib:

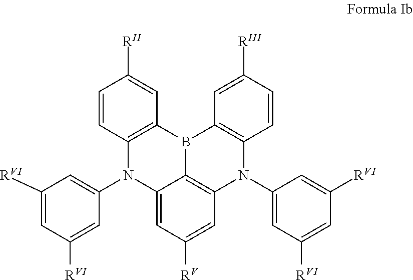

wherein R^{II} and R^{III} is independently from another selected from the group consisting of:
hydrogen, deuterium, halogen, Me, ⁱPr, ^{t}Bu, CN, CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph, and
triazinyl, which is optionally substituted with one or more substituents independently from each other selected from the group consisting of Me, ⁱPr, ^{t}Bu, CN, CF₃, and Ph,
and N(Ph)₂.

In one embodiment, the organic molecule comprises or consists of a structure of formula Ib, wherein R^{I} and R^{III} is independently from another selected from the group consisting of:
hydrogen, deuterium, Me, ⁱPr, ^{t}Bu,
Ph, which is optionally substituted with one or more substituents independently from each other selected from the group consisting of Me, ⁱPr, ^{t}Bu, and Ph,
carbazolyl, which is optionally substituted with one or more substituents independently from each other selected from the group consisting of Me, ⁱPr, ^{t}Bu, CN, CF₃, and Ph,
and N(Ph)₂.

In one embodiment, the organic molecule comprises or consists of a structure of formula Ib, wherein R^{II} and R^{III} is each independently from another selected from the group consisting of:
hydrogen, deuterium, Me, ⁱPr, ^{t}Bu,
Ph, which is optionally substituted with one or more substituents independently from each other selected from the group consisting of Me, ⁱPr, ^{t}Bu, and Ph,
and N(Ph)₂.

In one embodiment, the organic molecule comprises or consists of a structure of formula Ib, wherein R^{II} and R^{III} is independently from another selected from the group consisting of:
hydrogen, deuterium, Me, ⁱPr, ^{t}Bu,
Ph, which is optionally substituted with one or more substituents independently from each other selected from the group consisting of Me, ⁱPr, ^{t}Bu, and Ph,
and N(Ph)₂.

In one embodiment, the organic molecule comprises or consists of a structure of formula Ib, wherein R^{II} and R^{III} is independently from another selected from the group consisting of:
hydrogen, ^{t}Bu, and Ph.

In one embodiment, the organic molecule comprises or consists of a structure of formula Ib, wherein R^{II} is hydrogen.

In one embodiment, the organic molecule comprises or consists of a structure of formula Ib, wherein R^{II} is ^{t}Bu.

In one embodiment, the organic molecule comprises or consists of a structure of formula Ib, wherein R^{II} is Ph.

In one embodiment, the organic molecule comprises or consists of a structure of formula Ib, wherein R^{III} is hydrogen.

In one embodiment, the organic molecule comprises or consists of a structure of formula Ib, wherein R^{III} is ^{t}Bu.

In one embodiment, the organic molecule comprises or consists of a structure of formula Ib, wherein R^{III} is Ph.

In one embodiment, the organic molecule comprises or consists of a structure of formula Ib, wherein R^{II} is equal to R^{III}, in particular, R^{II} and R^{III} are identical.

In one embodiment, the organic molecule comprises or consists of a structure of formula Ib, wherein R^{II} and R^{III} are ^{t}Bu.

In one embodiment, the organic molecule comprises or consists of a structure of formula Ib, wherein R^{II} and R^{III} are Ph.

In one embodiment, the organic molecule comprises or consists of a structure of formula Ib, wherein R^{II} and R^{III} are hydrogen.

In one embodiment, the organic molecule comprises or consists of a structure of formula Ib, wherein R^{V} is mesityl (Mes) or 2-meta-ter-phenyl.

In one embodiment, the organic molecule comprises or consists of a structure of formula Ib, wherein R^{V} is 2-meta-ter-phenyl.

In one embodiment, the organic molecule comprises or consists of a structure of formula Ib, wherein R^{V} is C₃-C₁₅-heteroaryl, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Ib, wherein R^{V} is a C₃-C₁₅-heteroaryl, in particular with electron withdrawing properties, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Ib, wherein R^{V} consists of a structure of formula I-0.

In one embodiment, the organic molecule comprises or consists of a structure of formula Ib, wherein R^{V} consists of a structure of formula I-0, wherein
R^{b} is at each occurrence independently from another selected from the group consisting of the binding site, hydrogen, deuterium, Me, ⁱPr, ^{t}Bu, or C₆-C₁₈ aryl;
R^{c} is at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula Ib, wherein R^{V} is selected from a group consisting of formula 2a, 2b, 2c, 2d, 2e, 2f, 2g, 2h, 2i, 2j, 2k or 2l;

In one embodiment, the organic molecule comprises or consists of a structure of formula Ib, wherein R^{V} is selected from a group consisting of formula 2a, 2b, 2c, 2d, 2e, 2f, 2g, 2h, 2i, or 2j;

In one embodiment, the organic molecule comprises or consists of a structure of formula Ib, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, C₁-C₅-alkyl, and C₆-C₁₈ aryl.

In one embodiment, the organic molecule comprises or consists of a structure of formula Ib, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, Me, ⁱPr, ^{t}Bu, and C₆-C₁₈ aryl.

In one embodiment, the organic molecule comprises or consists of a structure of formula Ib, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, Me, ⁱPr, or ^{t}Bu.

In one embodiment, the organic molecule comprises or consists of a structure of formula Ib, wherein R^{V} is selected from the group consisting of formulas 2a, 2b, 2c, 2d, 2e, 2f, 2g, 2h, 2i, 2j, 2k and 2l, wherein R^{c} is hydrogen.

In one embodiment, the organic molecule comprises or consists of a structure of formula Ib, wherein R^{V} is selected from the group consisting of formulas 2a, 2b, 2c, 2d, 2e, 2f, 2g, 2h, 2i, 2j, 2k and 2l, wherein R¹ and R² are at each occurrence independently from another selected from the group consisting of hydrogen, deuterium, Me, ⁱPr, ^{t}Bu, CN, and C₆-C₁₈ aryl;

In one embodiment, the organic molecule comprises or consists of a structure of formula Ib, wherein R^{V} is selected from the group consisting of formulas 2a, 2b, 2c, 2d, 2e, 2f, 2g, 2h, 2i, 2j, 2k and 2l, wherein R¹, R² and R^{c} are at each occurrence independently from another selected from the group consisting of hydrogen, deuterium, Me, ⁱPr, ^{t}Bu, and C₆-C₁₈ aryl.

In one embodiment, the organic molecule comprises or consists of a structure of formula Ib, wherein R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, C₁-C₅-alkyl, and C₆-C₁₈ aryl.

In one embodiment, the organic molecule comprises or consists of a structure of formula Ib, wherein R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, Me, ⁱPr, ^{t}Bu, and C₆-C₁₈ aryl.

In one embodiment, the organic molecule comprises or consists of a structure of formula Ib, wherein R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, Me, ⁱPr, and ^{t}Bu.

In one embodiment, the organic molecule comprises or consists of a structure of formula Ib, wherein R^{V} is selected from the group consisting of formulas 2a, 2b, 2c, 2d, 2e, 2f, 2g, 2h, 2i, and 2j, wherein R^{c} is hydrogen.

In one embodiment, the organic molecule comprises or consists of a structure of formula Ib, wherein R^{V} is selected from the group consisting of formulas 2a, 2b, 2c, 2d, 2e, 2f, 2g, 2h, 2i, and 2j, wherein R¹ and R² are at each occurrence independently from another selected from the group consisting of hydrogen, deuterium, Me, ⁱPr, ^{t}Bu, CN, or C₆-C₁₈ aryl.

In one embodiment, the organic molecule comprises or consists of a structure of formula Ib, wherein R^{V} is selected from the group consisting of formulas 2a, 2b, 2c, 2d, 2e, 2f, 2g, 2h, 2i, and 2j, wherein R¹, R² and R^{c} are at each occurrence independently from another selected from the group consisting of hydrogen, deuterium, Me, ⁱPr, ^{t}Bu. and C₆-C₁₈ aryl.

In one embodiment, R^{VI}, R^{VII} and R^{VIII} are at each occurrence independently from each other selected from the group consisting of hydrogen,

C₁-C₅-alkyl,
C₆-C₁₃-aryl,
wherein optionally one or more hydrogen atoms are independently from each other substituted by C₁-C₅-alkyl or C₆-C₁₃-aryl.

In one embodiment, the organic molecule comprises or consists of a structure of formula Ib, wherein R^{VI} is at each occurrence independently from each other selected from the group consisting of hydrogen, Ph and ^{t}Bu.

In one embodiment, the organic molecule comprises or consists of a structure of formula Ib, wherein R^{VI} is Ph.

In one embodiment, the organic molecule comprises or consists of a structure of formula Ib, wherein R^{VI} is ^{t}Bu.

In one embodiment, the organic molecule comprises or consists of a structure of formula Ic:

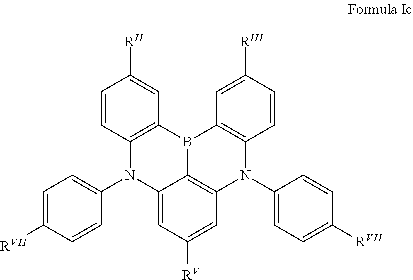

In one embodiment, the organic molecule comprises or consists of a structure of formula Ic, wherein R^{II} and R^{III} is independently from another selected from the group consisting of:
hydrogen, deuterium, Me, ⁱPr, ^{t}Bu,
Ph, which is optionally substituted with one or more substituents independently from each other selected from the group consisting of Me, ⁱPr, ^{t}Bu, and Ph,
carbazolyl, which is optionally substituted with one or more substituents independently from each other selected from the group consisting of Me, ⁱPr, ^{t}Bu, CN, CF₃, and Ph,
and N(Ph)₂.

In one embodiment, the organic molecule comprises or consists of a structure of formula Ic, wherein R^{II} and R^{III} is each independently from another selected from the group consisting of:
hydrogen, deuterium, Me, ⁱPr, ^{t}Bu,
Ph, which is optionally substituted with one or more substituents independently from each other selected from the group consisting of Me, ⁱPr, ^{t}Bu, and Ph,
and N(Ph)₂.

In one embodiment, the organic molecule comprises or consists of a structure of formula Ic, wherein R^{II} and R^{III} is independently from another selected from the group consisting of: hydrogen, deuterium, Me, ⁱPr, ^{t}Bu,
Ph, which is optionally substituted with one or more substituents independently from each other selected from the group consisting of Me, ⁱPr, ^{t}Bu, and Ph,
and N(Ph)₂.

In one embodiment, the organic molecule comprises or consists of a structure of formula Ic, wherein R^{II} and R^{III} is independently from another selected from the group consisting of: hydrogen, ^{t}Bu, and Ph.

In one embodiment, the organic molecule comprises or consists of a structure of formula Ic, wherein R^{II} is hydrogen.

In one embodiment, the organic molecule comprises or consists of a structure of formula Ic, wherein R^{II} is ^{t}Bu.

In one embodiment, the organic molecule comprises or consists of a structure of formula Ic, wherein R^{II} is Ph.

In one embodiment, the organic molecule comprises or consists of a structure of formula Ic, wherein R^{III} is hydrogen.

In one embodiment, the organic molecule comprises or consists of a structure of formula Ic, wherein R^{III} is ^{t}Bu.

In one embodiment, the organic molecule comprises or consists of a structure of formula Ic, wherein R^{III} is Ph.

In one embodiment, the organic molecule comprises or consists of a structure of formula Ic, wherein R^{II} is equal to R^{III}, in particular, R^{II} and R^{III} are identical.

In one embodiment, the organic molecule comprises or consists of a structure of formula Ic, wherein R^{II} and R^{III} are ^{t}Bu.

In one embodiment, the organic molecule comprises or consists of a structure of formula Ic, wherein R^{II} and R^{III} are Ph.

In one embodiment, the organic molecule comprises or consists of a structure of formula Ic, wherein R^{II} and R^{III} are hydrogen.

In one embodiment, the organic molecule comprises or consists of a structure of formula Ic, wherein R^{V} is mesityl (Mes) or 2-meta-ter-phenyl.

In one embodiment, the organic molecule comprises or consists of a structure of formula Ic, wherein R^{V} is 2-meta-ter-phenyl.

In one embodiment, the organic molecule comprises or consists of a structure of formula Ic, wherein R^{V} is C₃-C₁₅-heteroaryl, wherein optionally one or more hydrogen atoms are independently from each other substituted by C₁-C₅-alkyl, C₆-C₁₈ aryl or C₃-C₁₇-heteroaryl.

In one embodiment, the organic molecule comprises or consists of a structure of formula Ic, wherein R^{V} is a C₃-C₁₅-heteroaryl, in particular with electron withdrawing properties, wherein optionally one or more hydrogen atoms are independently from each other substituted by C₁-C₅-alkyl, C₆-C₁₈ aryl or C₃-C₁₇-heteroaryl.

In one embodiment, the organic molecule comprises or consists of a structure of formula Ic, wherein R^{V} consists of a structure of formula I-0.

In one embodiment, the organic molecule comprises or consists of a structure of formula Ic, wherein R^{V} consists of a structure of formula I-0, wherein
R^{b} is at each occurrence independently from another selected from the group consisting of the binding site, hydrogen, deuterium, Me, ⁱPr, ^{t}Bu, or C₆-C₁₈ aryl;
R^{c} is at each occurrence independently from another selected from the group consisting of hydrogen, deuterium, Me, ⁱPr, ^{t}Bu. or C₆-C₁₈ aryl.

In one embodiment, the organic molecule comprises or consists of a structure of formula Ic, wherein R^{V} is selected from a group consisting of formula 2a, 2b, 2c, 2d, 2e, 2f, 2g, 2h, 2i, 2j, 2k or 2l;

In one embodiment, the organic molecule comprises or consists of a structure of formula Ic, wherein R^{V} is selected from a group consisting of formula 2a, 2b, 2c, 2d, 2e, 2f, 2g, 2h, 2i, or 2j;

In one embodiment, the organic molecule comprises or consists of a structure of formula Ic, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, C₁-C₅-alkyl, or C₆-C₁₈ aryl;

In one embodiment, the organic molecule comprises or consists of a structure of formula Ic, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ic, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, Me, ⁱPr, or ^{t}Bu;

In one embodiment, the organic molecule comprises or consists of a structure of formula Ic, wherein R^{V} is selected from a group consisting of formula 2a, 2b, 2c, 2d, 2e, 2f, 2g, 2h, 2i, 2j, 2k or 2l, wherein R^{c} is hydrogen;

In one embodiment, the organic molecule comprises or consists of a structure of formula Ic, wherein R^{V} is selected from a group consisting of formula 2a, 2b, 2c, 2d, 2e, 2f, 2g, 2h, 2i, 2j, 2k or 2l, wherein R¹ and R² are at each occurrence independently from another selected from the group consisting of hydrogen, deuterium, Me, ⁱPr, ^{t}Bu, CN, or C₆-C₁₈ aryl;

In one embodiment, the organic molecule comprises or consists of a structure of formula Ic, wherein R^{V} is selected from a group consisting of formula 2a, 2b, 2c, 2d, 2e, 2f, 2g, 2h, 2i, 2j, 2k or 2l, wherein R¹, R² and R^{c} are at each occurrence independently from another selected from the group consisting of hydrogen, deuterium, Me, ⁱPr, ^{t}Bu. or C₆-C₁₈ aryl;

In one embodiment, the organic molecule comprises or consists of a structure of formula Ic, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, C₁-C₅-alkyl, or C₆-C₁₈ aryl;

In one embodiment, the organic molecule comprises or consists of a structure of formula Ic, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ic, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, or ^{t}Bu;

In one embodiment, the organic molecule comprises or consists of a structure of formula Ic, wherein R^{V} is selected from a group consisting of formula 2a, 2b, 2c, 2d, 2e, 2f, 2g, 2h, 2i, or 2j, wherein R^{c} is hydrogen;

In one embodiment, the organic molecule comprises or consists of a structure of formula Ic, wherein R^{V} is selected from a group consisting of formula 2a, 2b, 2c, 2d, 2e, 2f, 2g, 2h, 2i, or 2j, wherein R¹ and R² are at each occurrence independently from another selected from the group consisting of hydrogen, deuterium, Me, ⁱPr, ^{t}Bu, CN, or C₆-C₁₈ aryl;

In one embodiment, the organic molecule comprises or consists of a structure of formula Ic, wherein R^{V} is selected from a group consisting of formula 2a, 2b, 2c, 2d, 2e, 2f, 2g, 2h, 2i, or 2j, wherein R¹, R² and R^{c} are at each occurrence independently from another selected from the group consisting of hydrogen, deuterium, Me, ⁱPr, ^{t}Bu, or C₆-C₁₈ aryl;

In one embodiment, R^{VI}, R^{VII} and R^{VIII} are at each occurrence independently from each other selected from the group consisting of hydrogen,

C₁-C₅-alkyl,
C₆-C₁₃-aryl,
wherein optionally one or more hydrogen atoms are independently from each other substituted by C₁-C₅-alkyl or C₆-C₁₈-aryl.

In one embodiment, the organic molecule comprises or consists of a structure of formula Ic, wherein R^{VI}, R^{VII} and R^{VIII} are at each occurrence independently from each other selected from the group consisting of hydrogen, Ph and ^{t}Bu.

In one embodiment, the organic molecule comprises or consists of a structure of formula Ic, wherein R^{VI} is ^{t}Bu, and R^{VII} and R^{VIII} are hydrogen.

In one embodiment, the organic molecule comprises or consists of a structure of formula Ic, wherein R^{VII} is ^{t}Bu, and R^{VI} and R^{VIII} are hydrogen.

In one embodiment, the organic molecule comprises or consists of a structure of formula Ic, wherein R^{VI} is ^{t}Bu, and R^{VII} and R^{VIII} are hydrogen.

In one embodiment the organic molecule comprises or consists of a structure of formula Ic, wherein, R^{VII} is ^{t}Bu, and R^{VI} and R^{VIII} are hydrogen.

In one embodiment, the organic molecule comprises or consists of a structure of formula Id:

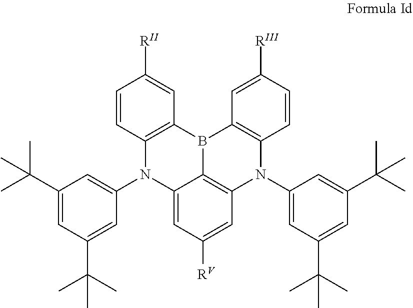

wherein R^{V} is selected from the group of mesityl (Mes) or 2-meta-ter-phenyl.

In one embodiment, the organic molecule comprises or consists of a structure of formula Id, wherein R^{V} is 2-meta-ter-phenyl.

In one embodiment, the organic molecule comprises or consists of a structure of formula Id, wherein R^{V} is C₃-C₁₅-heteroaryl, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Id, wherein R^{V} is a C₃-C₁₅-heteroaryl, in particular with electron withdrawing properties, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Id, wherein R^{V} consists of a structure of formula I-0;

In one embodiment, the organic molecule comprises or consists of a structure of formula Id, wherein R^{V} consists of a structure of formula I-0, wherein
R^{b} is at each occurrence independently from another selected from the group consisting of the binding site, hydrogen, deuterium, Me, ⁱPr, ^{t}Bu, or C₆-C₁₈ aryl;
R^{c} is at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula Id, wherein R^{V} is selected from the group consisting of formulas 2a, 2b, 2c, 2d, 2e, 2f, 2g, 2h, 2i, 2j, 2k and 2l.

In one embodiment, the organic molecule comprises or consists of a structure of formula Id, wherein R^{V} is selected from the group consisting of formulas 2a, 2b, 2c, 2d, 2e, 2f, 2g, 2h, 2i, and 2j.

In one embodiment, the organic molecule comprises or consists of a structure of formula Id, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, C₁-C₅-alkyl, and C₆-C₁₈ aryl;

In one embodiment, the organic molecule comprises or consists of a structure of formula Id, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, Me, ⁱPr, ^{t}Bu, and C₆-C₁₈ aryl.

In one embodiment, the organic molecule comprises or consists of a structure of formula Id, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, Me, ⁱPr, and ^{t}Bu.

In one embodiment, the organic molecule comprises or consists of a structure of formula Id, wherein R^{V} is selected from the group consisting of formulas 2a, 2b, 2c, 2d, 2e, 2f, 2g, 2h, 2i, 2j, 2k and 2l, wherein R^{c} is hydrogen.

In one embodiment, the organic molecule comprises or consists of a structure of formula Id, wherein R^{V} is selected from the group consisting of formulas 2a, 2b, 2c, 2d, 2e, 2f, 2g, 2h, 2i, 2j, 2k or 2l, wherein R¹ and R² are at each occurrence independently from another selected from the group consisting of hydrogen, deuterium, Me, ⁱPr, ^{t}Bu, CN, and C₆-C₁₈ aryl.

In one embodiment, the organic molecule comprises or consists of a structure of formula Id, wherein R^{V} is selected from the group consisting of formulas 2a, 2b, 2c, 2d, 2e, 2f, 2g, 2h, 2i, 2j, 2k and 2l, wherein R¹, R² and R^{c} are at each occurrence independently from another selected from the group consisting of hydrogen, deuterium, Me, ⁱPr, ^{t}Bu, and C₆-C₁₈ aryl.

In one embodiment, the organic molecule comprises or consists of a structure of formula Id, wherein R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, C₁-C₅-alkyl, and C₆-C₁₈ aryl.

In one embodiment, the organic molecule comprises or consists of a structure of formula Id, wherein R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, Me, ⁱPr, ^{t}Bu, and C₆-C₁₈ aryl.

In one embodiment, the organic molecule comprises or consists of a structure of formula Id, wherein R^{V} is selected from the group consisting of formulas 2a, 2b, 2c, 2d, 2e, 2f, 2g, 2h, 2i, and 2j, wherein R^{c} is at each occurrence independently from another selected from the group consisting of hydrogen, deuterium, Me, ⁱPr, and ^{t}Bu.

In one embodiment, the organic molecule comprises or consists of a structure of formula Id, wherein R^{V} is selected from the group consisting of formulas 2a, 2b, 2c, 2d, 2e, 2f, 2g, 2h, 2i, and 2j, wherein R^{c} is hydrogen.

In one embodiment, the organic molecule comprises or consists of a structure of formula Id, wherein R^{V} is selected from the group consisting of formulas 2a, 2b, 2c, 2d, 2e, 2f, 2g, 2h, 2i, and 2j, wherein R¹ and R² are at each occurrence independently from another selected from the group consisting of hydrogen, deuterium, Me, ⁱPr, ^{t}Bu. CN, and C₆-C₁₈ aryl.

In one embodiment, the organic molecule comprises or consists of a structure of formula Id, wherein R^{V} is selected from the group consisting of formulas 2a, 2b, 2c, 2d, 2e, 2f, 2g, 2h, 2i, and 2j, wherein R¹, R² and R^{c} are at each occurrence independently from another selected from the group consisting of hydrogen, deuterium, Me, ⁱPr, ^{t}Bu, and C₆-C₁₈ aryl.

In one embodiment, the organic molecule comprises or consists of a structure of formula Id, wherein R^{V} is 2-meta-ter-phenyl; R^{II} and R^{III} are hydrogen.

In one embodiment, the organic molecule comprises or consists of a structure of formula Id-1:

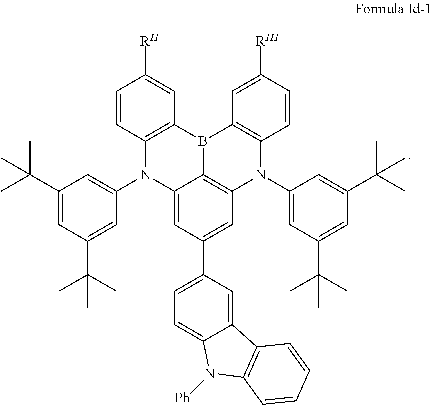

In one embodiment, the organic molecule comprises or consists of a structure of formula Id-2:

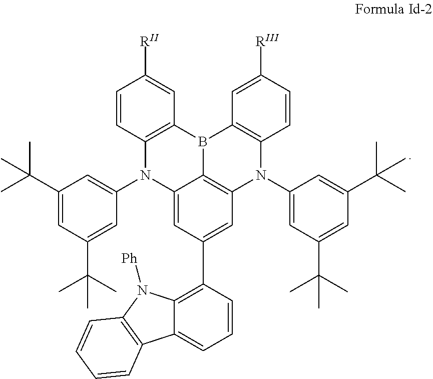

In one embodiment, the organic molecule comprises or consists of a structure of formula Id-3:

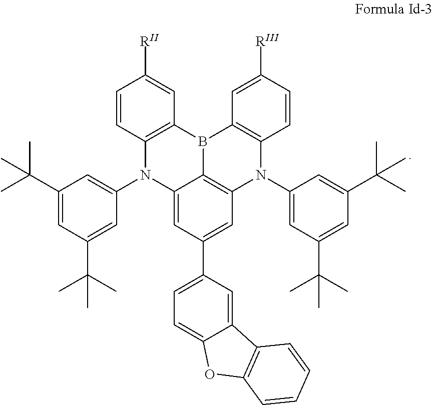

In one embodiment, the organic molecule comprises or consists of a structure of formula Id-4:

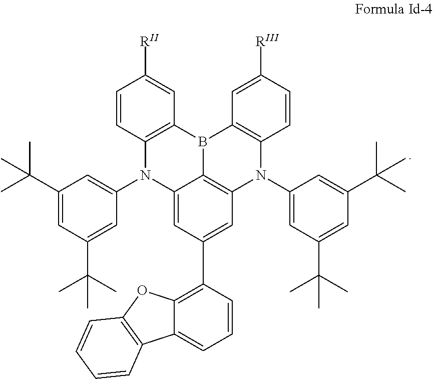

In one embodiment, the organic molecule comprises or consists of a structure of formula Ie:

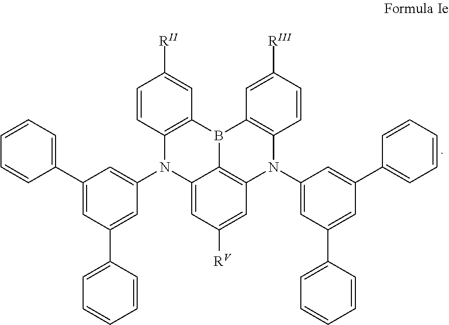

wherein R^{V} is selected from the group of mesityl (Mes) and 2-meta-ter-phenyl.

In one embodiment, the organic molecule comprises or consists of a structure of formula Ie, wherein R^{V} is 2-meta-ter-phenyl.

In one embodiment, the organic molecule comprises or consists of a structure of formula Ie, wherein R^{V} is C₃-C₁₅-heteroaryl, wherein optionally one or more hydrogen atoms are independently from each other substituted by C₁-C₅-alkyl, C₆-C₁₈ aryl or C₃-C₁₇-heteroaryl.

In one embodiment, the organic molecule comprises or consists of a structure of formula Ie, wherein R^{V} is a C₃-C₁₅-heteroaryl, in particular with electron withdrawing properties, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Ie, wherein R^{V} consists of a structure of formula I-0;

In one embodiment, the organic molecule comprises or consists of a structure of formula Ie, wherein R^{V} consists of a structure of formula I-0, wherein
R^{b} is at each occurrence independently from another selected from the group consisting of the binding site, hydrogen, deuterium, Me, ⁱPr, ^{t}Bu, or C₆-C₁₈ aryl;
R^{c} is at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula Ie, wherein R^{V} is selected from the group consisting of formulas 2a, 2b, 2c, 2d, 2e, 2f, 2g, 2h, 2i, 2j, 2k and 2l.

In one embodiment, the organic molecule comprises or consists of a structure of formula Ie, wherein R^{V} is selected from the group consisting of formulas 2a, 2b, 2c, 2d, 2e, 2f, 2g, 2h, 2i, and 2j.

In one embodiment, the organic molecule comprises or consists of a structure of formula Ie, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, C₁-C₅-alkyl, or C₆-C₁₈ aryl.

In one embodiment, the organic molecule comprises or consists of a structure of formula Ie, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula Ie, wherein R^{V} is selected from the group consisting of formulas 2a, 2b, 2c, 2d, 2e, 2f, 2g, 2h, 2i, 2j, 2k or 2l, wherein R^{c} is at each occurrence independently from another selected from the group consisting of hydrogen, deuterium, Me, ⁱPr, or ^{t}Bu.

In one embodiment, the organic molecule comprises or consists of a structure of formula Ie, wherein R^{V} is selected from the group consisting of formulas 2a, 2b, 2c, 2d, 2e, 2f, 2g, 2h, 2i, 2j, 2k or 2l, wherein R^{c} is hydrogen.

In one embodiment, the organic molecule comprises or consists of a structure of formula Ie, wherein R^{V} is selected from the group consisting of formulas 2a, 2b, 2c, 2d, 2e, 2f, 2g, 2h, 2i, 2j, 2k or 2l, wherein R¹ and R² are at each occurrence independently from another selected from the group consisting of hydrogen, deuterium, Me, ⁱPr, ^{t}Bu, CN, or C₆-C₁₈ aryl.

In one embodiment, the organic molecule comprises or consists of a structure of formula Ie, wherein R^{V} is selected from the group consisting of formulas 2a, 2b, 2c, 2d, 2e, 2f, 2g, 2h, 2i, 2j, 2k or 2l, wherein R¹, R² and R^{c} are at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula Ie, wherein R^{V} is selected from the group consisting of formulas 2a, 2b, 2c, 2d, 2e, 2f, 2g, 2h, 2i, or 2j, wherein R^{c} is at each occurrence independently from another selected from the group consisting of hydrogen, deuterium, C₁-C₅-alkyl, or C₆-C₁₈ aryl.

In one embodiment, the organic molecule comprises or consists of a structure of formula Ie, wherein R^{V} is selected from the group consisting of formulas 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula Ie, wherein R^{V} is selected from the group consisting of formulas 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, or ^{t}Bu.

In one embodiment, the organic molecule comprises or consists of a structure of formula Ie, wherein R^{V} is selected from the group consisting of formulas 2a, 2b, 2c, 2d, 2e, 2f, 2g, 2h, 2i, or 2j, wherein R^{c} is hydrogen.

In one embodiment, the organic molecule comprises or consists of a structure of formula Ie, wherein R^{V} is selected from the group consisting of formulas 2a, 2b, 2c, 2d, 2e, 2f, 2g, 2h, 2i, or 2j, wherein R¹ and R² are at each occurrence independently from another selected from the group consisting of hydrogen, deuterium, Me, ⁱPr, ^{t}Bu, CN, or C₆-C₁₈ aryl.

In one embodiment, the organic molecule comprises or consists of a structure of formula Ie, wherein R^{V} is selected from the group consisting of formulas 2a, 2b, 2c, 2d, 2e, 2f, 2g, 2h, 2i, or 2j, wherein R¹, R² and R^{c} are at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula Ie, wherein R^{V} is 2-meta-ter-phenyl; R^{II} and R^{III} are hydrogen.

In one embodiment, the organic molecule comprises or consists of a structure of formula Ie-1:

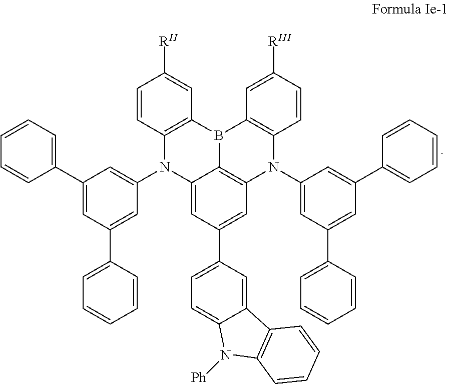

In one embodiment, the organic molecule comprises or consists of a structure of formula Ie-2:

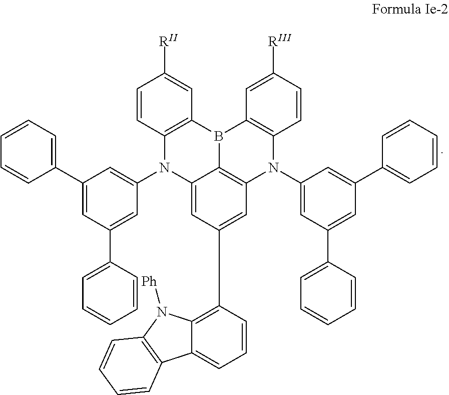

In one embodiment, the organic molecule comprises or consists of a structure of formula Ie-3:

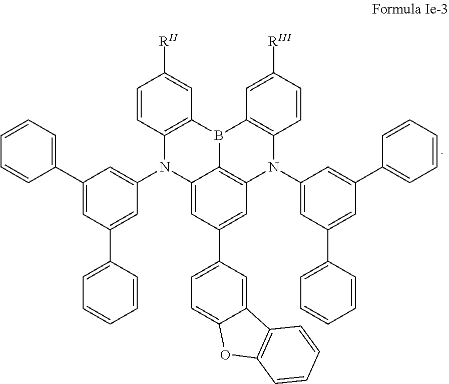

In one embodiment, the organic molecule comprises or consists of a structure of formula Ie-4:

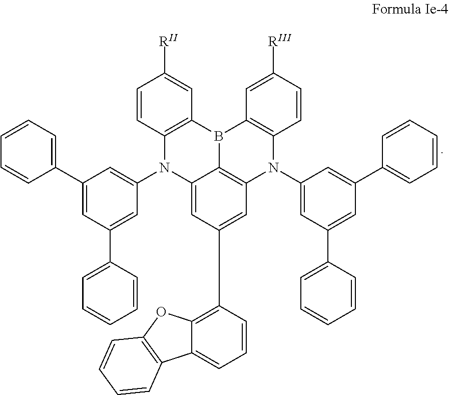

In one embodiment, the organic molecule comprises or consists of a structure of formula If:

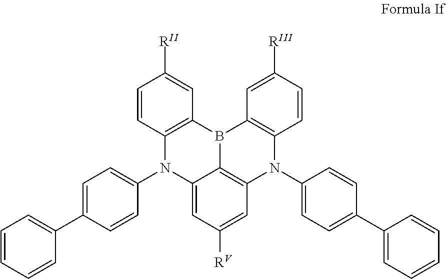

wherein R^{V} is selected from the group of mesityl (Mes) or 2-meta-ter-phenyl.

In one embodiment, the organic molecule comprises or consists of a structure of formula If, wherein R^{V} is 2-meta-ter-phenyl.

In one embodiment, the organic molecule comprises or consists of a structure of formula If, wherein R^{V} is C₃-C₁₅-heteroaryl, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula If, wherein R^{V} is a C₃-C₁₅-heteroaryl, in particular with electron withdrawing properties, wherein optionally one or more hydrogen atoms are independently from each other substituted by C₁-C₅-alkyl, C₆-C₁₈ aryl or C₅-C₁₇-heteroaryl;

In one embodiment, the organic molecule comprises or consists of a structure of formula If, wherein R^{V} consists of a structure of formula I-0;

In one embodiment, the organic molecule comprises or consists of a structure of formula If, wherein R^{V} consists of a structure of formula I-0, wherein
R^{b} is at each occurrence independently from another selected from the group consisting of the binding site, hydrogen, deuterium, Me, ⁱPr, ^{t}Bu, or C₆-C₁₈ aryl;
R^{c} is at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula If, wherein R^{V} is selected from the group consisting of formulas 2a, 2b, 2c, 2d, 2e, 2f, 2g, 2h, 2i, 2j, 2k and 2l;

In one embodiment, the organic molecule comprises or consists of a structure of formula If, wherein R^{V} is selected from the group consisting of formulas 2a, 2b, 2c, 2d, 2e, 2f, 2g, 2h, 2i, or 2j.

In one embodiment, the organic molecule comprises or consists of a structure of formula If, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, C₁-C₅-alkyl, or C₆-C₁₈ aryl.

In one embodiment, the organic molecule comprises or consists of a structure of formula If, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, Me, ⁱPr, ^{t}Bu, and C₆-C₁₈ aryl.

In one embodiment, the organic molecule comprises or consists of a structure of formula If, wherein R^{V} is selected from the group consisting of formulas 2a, 2b, 2c, 2d, 2e, 2f, 2g, 2h, 2i, 2j, 2k and 2l, wherein R^{c} is at each occurrence independently from another selected from the group consisting of hydrogen, deuterium, Me, ⁱPr, and ^{t}Bu.

In one embodiment, the organic molecule comprises or consists of a structure of formula If, wherein R^{V} is selected from the group consisting of formulas 2a, 2b, 2c, 2d, 2e, 2f, 2g, 2h, 2i, 2j, 2k and 2l, wherein R^{c} is hydrogen.

In one embodiment, the organic molecule comprises or consists of a structure of formula If, wherein R^{V} is selected from a group consisting of formula 2a, 2b, 2c, 2d, 2e, 2f, 2g, 2h, 2i, 2j, 2k or 2l, wherein R¹ and R² are at each occurrence independently from another selected from the group consisting of hydrogen, deuterium, Me, ⁱPr, ^{t}Bu, CN, or C₆-C₁₈ aryl;

In one embodiment, the organic molecule comprises or consists of a structure of formula If, wherein R^{V} is selected from a group consisting of formula 2a, 2b, 2c, 2d, 2e, 2f, 2g, 2h, 2i, 2j, 2k or 2l, wherein R¹, R² and R^{c} are at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula If, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, C₁-C₅-alkyl, or C₆-C₁₈ aryl;

In one embodiment, the organic molecule comprises or consists of a structure of formula If, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula If, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, or ^{t}Bu;

In one embodiment, the organic molecule comprises or consists of a structure of formula If, wherein R^{V} is selected from a group consisting of formula 2a, 2b, 2c, 2d, 2e, 2f, 2g, 2h, 2i, or 2j, wherein R^{c} is hydrogen;

In one embodiment, the organic molecule comprises or consists of a structure of formula If, wherein R^{V} is selected from a group consisting of formula 2a, 2b, 2c, 2d, 2e, 2f, 2g, 2h, 2i, or 2j, wherein R¹ and R² are at each occurrence independently from another selected from the group consisting of hydrogen, deuterium, Me, ⁱPr, ^{t}Bu, CN, or C₆-C₁₈ aryl;

In one embodiment, the organic molecule comprises or consists of a structure of formula If, wherein R^{V} is selected from a group consisting of formula 2a, 2b, 2c, 2d, 2e, 2f, 2g, 2h, 2i, or 2j, wherein R¹, R² and R^{c} are at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula If, wherein R^{V} is 2-meta-ter-phenyl; R^{II} and R^{III} are hydrogen.

In one embodiment, the organic molecule comprises or consists of a structure of formula If-1:

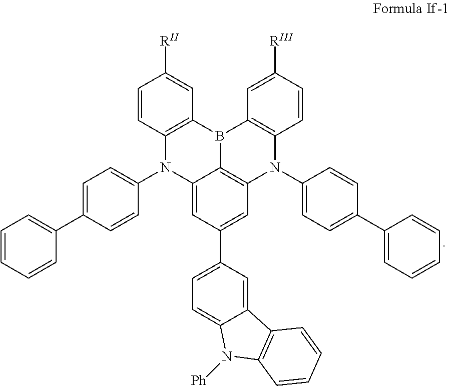

In one embodiment, the organic molecule comprises or consists of a structure of formula If-2:

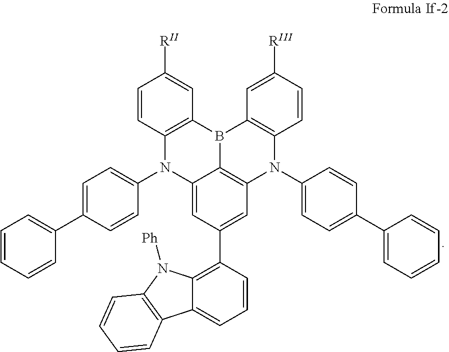

In one embodiment, the organic molecule comprises or consists of a structure of formula If-3:

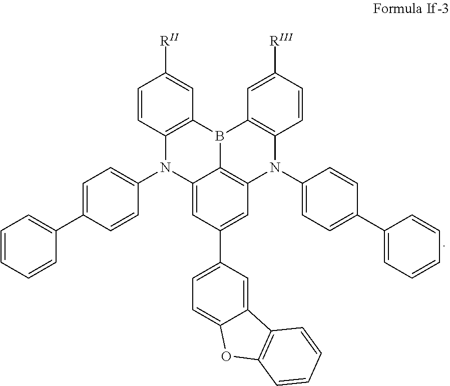

In one embodiment, the organic molecule comprises or consists of a structure of formula If-4:

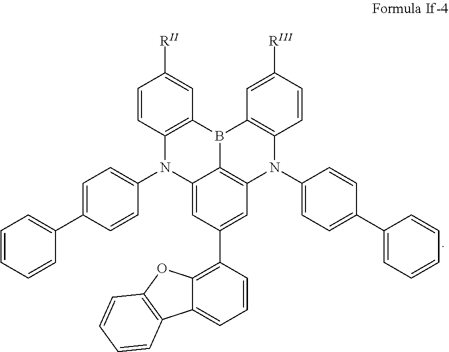

In some embodiments, at least one (i.e. 1, 2, 3 or 4) of R^{I}, R^{II}, R^{III}, and R^{IV} is not hydrogen.

In one embodiment, the organic molecule comprises or consists of a structure of formula Ig:

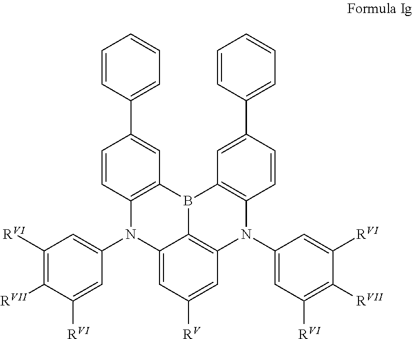

In one embodiment, the organic molecule comprises or consists of a structure of formula Ig:

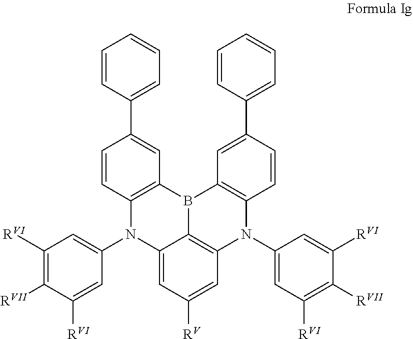

wherein R^{VI} and R^{VII} are at each occurrence independently from another selected from the group consisting of hydrogen, ^{t}Bu, or Ph.

In one embodiment, the organic molecule comprises or consists of a structure of formula Ig, wherein R^{V} is C₃-C₁₅-heteroaryl, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Ig, wherein R^{V} is a C₃-C₁₅-heteroaryl, in particular with electron withdrawing properties, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Ig, wherein R^{V} consists of a structure of formula I-0;

In one embodiment, the organic molecule comprises or consists of a structure of formula Ig, wherein R^{V} consists of a structure of formula I-0, wherein
R^{b} is at each occurrence independently from another selected from the group consisting of the binding site, hydrogen, deuterium, Me, ⁱPr, ^{t}Bu, or C₆-C₁₈ aryl;
R^{c} is at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula Ig, wherein R^{V} is selected from a group consisting of formula 2a, 2b, 2c, 2d, 2e, 2f, 2g, 2h, 2i, 2j, 2k or 2l;

In one embodiment, the organic molecule comprises or consists of a structure of formula Ig, wherein R^{V} is selected from a group consisting of formula 2a, 2b, 2c, 2d, 2e, 2f, 2g, 2h, 2i, or 2j;

In one embodiment, the organic molecule comprises or consists of a structure of formula Ig, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, C₁-C₅-alkyl, or C₆-C₁₈ aryl;

In one embodiment, the organic molecule comprises or consists of a structure of formula Ig, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ig, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, Me, ⁱPr, or ^{t}Bu;

In one embodiment, the organic molecule comprises or consists of a structure of formula Ig, wherein R^{V} is selected from a group consisting of formula 2a, 2b, 2c, 2d, 2e, 2f, 2g, 2h, 2i, 2j, 2k or 2l, wherein R^{c} is hydrogen;

In one embodiment, the organic molecule comprises or consists of a structure of formula Ig, wherein R^{V} is selected from a group consisting of formula 2a, 2b, 2c, 2d, 2e, 2f, 2g, 2h, 2i, 2j, 2k or 2l, wherein R¹ and R² are at each occurrence independently from another selected from the group consisting of hydrogen, deuterium, Me, ⁱPr, ^{t}Bu, CN, or C₆-C₁₈ aryl;

In one embodiment, the organic molecule comprises or consists of a structure of formula Ig, wherein R^{V} is selected from a group consisting of formula 2a, 2b, 2c, 2d, 2e, 2f, 2g, 2h, 2i, 2j, 2k or 2l, wherein R¹, R² and R^{c} are at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ig, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, C₁-C₅-alkyl, or C₆-C₁₈ aryl;

In one embodiment, the organic molecule comprises or consists of a structure of formula Ig, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ig, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, or ^{t}Bu;

In one embodiment, the organic molecule comprises or consists of a structure of formula Ig, wherein R^{V} is selected from a group consisting of formula 2a, 2b, 2c, 2d, 2e, 2f, 2g, 2h, 2i, or 2j, wherein R^{c} is hydrogen;

In one embodiment, the organic molecule comprises or consists of a structure of formula Ig, wherein R^{V} is selected from a group consisting of formula 2a, 2b, 2c, 2d, 2e, 2f, 2g, 2h, 2i, or 2j, wherein R¹ and R² are at each occurrence independently from another selected from the group consisting of hydrogen, deuterium, Me, ⁱPr, ^{t}Bu, CN, or C₆-C₁₈ aryl;

In one embodiment, the organic molecule comprises or consists of a structure of formula Ig, wherein R^{V} is selected from a group consisting of formula 2a, 2b, 2c, 2d, 2e, 2f, 2g, 2h, 2i, or 2j, wherein R¹, R² and R^{c} are at each occurrence independently from another selected from the group consisting of hydrogen, deuterium, Me, ⁱPr, ^{t}Bu. or C₆-C₁₈ aryl;

In one embodiment, the organic molecule comprises or consists of a structure of formula Ig-I:

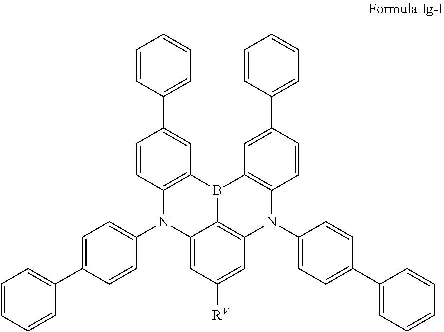

In one embodiment, the organic molecule comprises or consists of a structure of formula Ig-I, wherein R^{V} is C₃-C₁₅-heteroaryl, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Ig-I, wherein R^{V} is a C₃-C₁₅-heteroaryl, in particular with electron withdrawing properties, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Ig-I, wherein R^{V} consists of a structure of formula I-0;

In one embodiment, the organic molecule comprises or consists of a structure of formula Ig-I, wherein R^{V} consists of a structure of formula I-0, wherein
R^{b} is at each occurrence independently from another selected from the group consisting of the binding site, hydrogen, deuterium, Me, ⁱPr, ^{t}Bu, or C₆-C₁₈ aryl;
R^{c} is at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula Ig-I, wherein R^{V} is selected from a group consisting of formula 2a, 2b, 2c, 2d, 2e, 2f, 2g, 2h, 2i, 2j, 2k or 2l;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, or 2j;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, C₁-C₅-alkyl, or C₆-C₁₈ aryl;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, Me, ⁱPr, or ^{t}Bu;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, 2j, 2k or 2l, wherein R^{c} is hydrogen;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, 2j, 2k or 2l, wherein R¹ and R² are at each occurrence independently from another selected from the group consisting of hydrogen, deuterium, Me, ⁱPr, ^{t}Bu, CN, or C₆-C₁₈ aryl;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, 2j, 2k or 2l, wherein R¹, R² and R^{c} are at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, C₁-C₅-alkyl, or C₆-C₁₈ aryl;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, or ^{t}Bu;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, or 2j, wherein R^{c} is hydrogen;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, or 2j, wherein R¹ and R² are at each occurrence independently from another selected from the group consisting of hydrogen, deuterium, Me, ⁱPr, ^{t}Bu, CN, or C₆-C₁₈ aryl;

In one embodiment, the organic molecule comprises or consists of a structure of formula Ig-I, wherein R^{V} is selected from a group consisting of formula 2a, 2b, 2c, 2d, 2e, 2f, 2g, 2h, 2i, or 2j, wherein R¹, R² and R^{c} are at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ig-II:

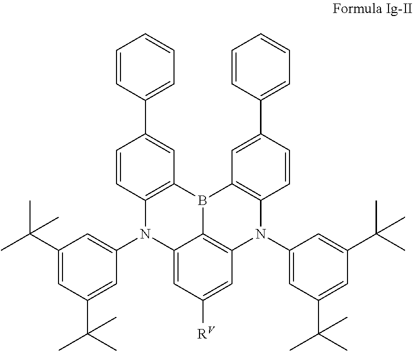

In one embodiment, the organic molecule comprises or consists of a structure of formula Ig-II, wherein R^{V} is C₃-C₁₅-heteroaryl, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Ig-II, wherein R^{V} is a C₃-C₁₅-heteroaryl, in particular with electron withdrawing properties, wherein optionally one or more hydrogen atoms are independently from each other substituted by C₁-C₅-alkyl, C₆-C₁₈ aryl or C₃-C₁₇-heteroaryl;

In one embodiment, the organic molecule comprises or consists of a structure of formula Ig-II, wherein R^{V} consists of a structure of formula I-0;

In one embodiment, the organic molecule comprises or consists of a structure of formula Ig-II, wherein R^{V} consists of a structure of formula I-0, wherein
R^{b} is at each occurrence independently from another selected from the group consisting of the binding site, hydrogen, deuterium, Me, ⁱPr, ^{t}Bu, or C₆-C₁₈ aryl;
R^{c} is at each occurrence independently from another selected from the group consisting of hydrogen, deuterium, Me, ⁱPr, ^{t}Bu, or C₆-C₁₈ aryl.

In one embodiment, the organic molecule comprises or consists of a structure of formula Ig-II, wherein R^{V} is selected from a group consisting of formula 2a, 2b, 2c, 2d, 2e, 2f, 2g, 2h, 2i, 2j, 2k or 2l;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, or 2j;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, C₁-C₅-alkyl, or C₆-C₁₈ aryl;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, 2j, 2k or 2l, wherein R^{c} is at each occurrence independently from another selected from the group consisting of hydrogen, deuterium, Me, ⁱPr, or ^{t}Bu;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, 2j, 2k or 2l, wherein R^{c} is hydrogen;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, 2j, 2k or 2l, wherein R¹ and R² are at each occurrence independently from another selected from the group consisting of hydrogen, deuterium, Me, ⁱPr, ^{t}Bu, CN, or C₆-C₁₈ aryl;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, 2j, 2k or 2l, wherein R¹, R² and R^{c} are at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, C₁-C₅-alkyl, or C₆-C₁₈ aryl;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, ^{t}Bu, or C₆-C₁₈ aryl;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, or 2j, wherein R^{c} is at each occurrence independently from another selected from the group consisting of hydrogen, deuterium, Me, ⁱPr, or ^{t}Bu;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, or 2j, wherein R^{c} is hydrogen;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, or 2j, wherein R¹ and R² are at each occurrence independently from another selected from the group consisting of hydrogen, deuterium, Me, ⁱPr, ^{t}Bu, CN, or C₆-C₁₈ aryl;

In one embodiment, the organic molecule comprises or consists of a structure of formula Ig-II, wherein R^{V} is selected from a group consisting of formula 2a, 2b, 2c, 2d, 2e, 2f, 2g, 2h, 2i, or 2j, wherein R¹, R² and R^{c} are at each occurrence independently from another selected from the group consisting of hydrogen, deuterium, Me, ⁱPr, ^{t}Bu, or C₆-C₁₈ aryl;

In one embodiment, the organic molecules comprise or consist a structure of formula 3

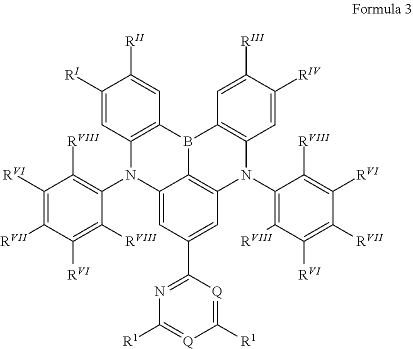

wherein

Q is at each occurrence independently from each other selected from the group consisting of N and C—R².

R¹ and R² are at each occurrence independently from another selected from the group consisting of:

hydrogen, deuterium, halogen, Me, ⁱPr, ^{t}Bu, CN, CF₃, SiMe₃, SiPh₃ (Ph=phenyl), and C₆-C₁₃-aryl,
wherein optionally one or more hydrogen atoms are independently from each other substituted by C₁-C₅-alkyl, CN, CF₃ and Ph.

In one embodiment of the invention, the organic molecule comprises or consists of a structure of formula 3-1:

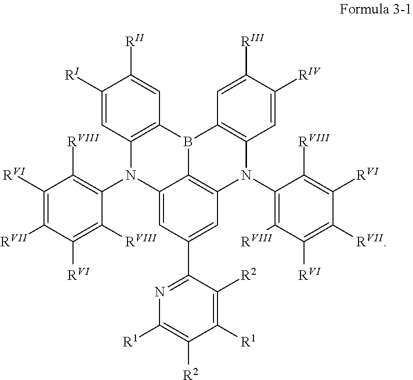

In one embodiment of the invention, the organic molecule comprises or consists of a structure of formula 3-2:

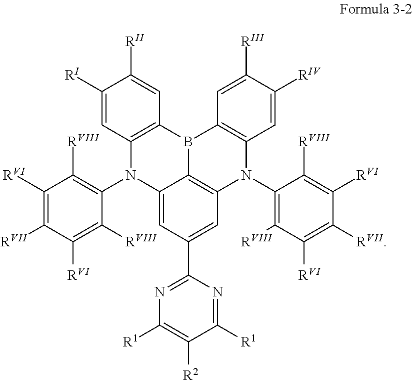

In one embodiment of the invention, the organic molecule comprises or consists of a structure of formula 3-3:

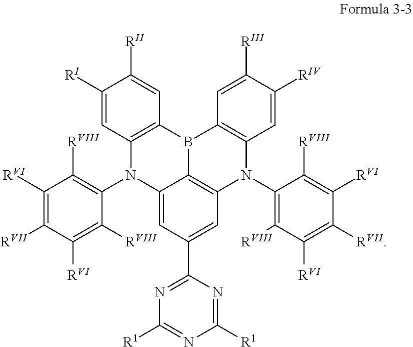

In a preferred embodiment of the invention, the organic molecule comprises or consists of a structure of formula 3-4:

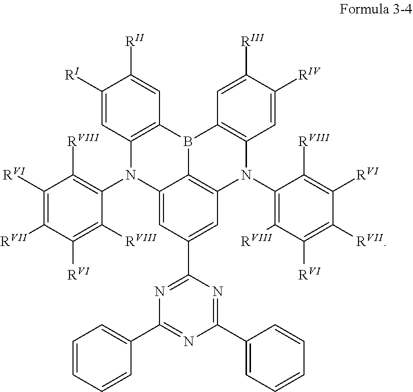

In a preferred embodiment of the invention, R^{I}, R^{II}, R^{III} and R^{IV} is independently from each other selected from the group consisting of:
hydrogen, deuterium, halogen, Me, ⁱPr, ^{t}Bu, CN, CF₃, SiMe₃, SiPh₃,
Ph, which is optionally substituted with one or more substituents independently from each other selected from the group consisting of Me, ⁱPr, ^{t}Bu, CN, CF₃, and Ph,
and N(Ph)₂.

In one embodiment of the invention, the organic molecule comprises or consists of a structure of formula 3a

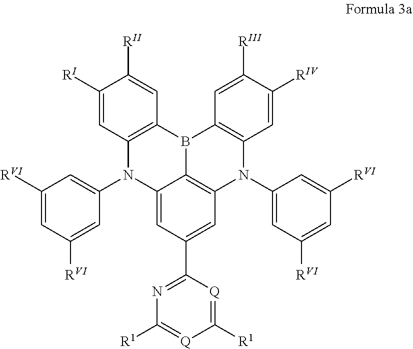

wherein
either R^{I}═R^{IV}═H and both R^{II} and R^{III} are independently from another selected from the group consisting of:
hydrogen, deuterium, halogen, Me, ⁱPr, ^{t}Bu, CN, CF₃, SiMe₃, SiPh₃,
Ph, which is optionally substituted with one or more substituents independently from each other selected from the group consisting of Me, ⁱPr, ^{t}Bu, CN, CF₃, and Ph, and N(Ph)₂;
or R^{II}═R^{III} ═H and both R^{I} and R^{IV} are independently from another selected from the group consisting of:
hydrogen, deuterium, halogen, Me, ⁱPr, ^{t}Bu, CN, CF₃, SiMe₃, SiPh₃,
Ph, which is optionally substituted with one or more substituents independently from each other selected from the group consisting of Me, ⁱPr, ^{t}Bu, CN, CF₃, and Ph,
and N(Ph)₂.

In one embodiment of the invention, the organic molecule comprises or consists of a structure of formula 3a-1

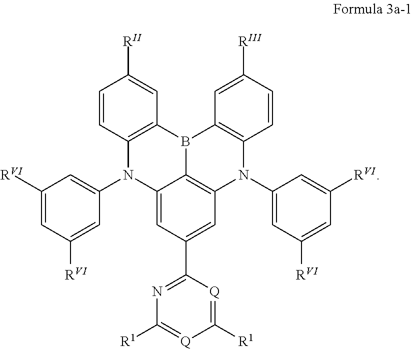

In one embodiment of the invention, the organic molecule comprises or consists of a structure of formula 3a-2

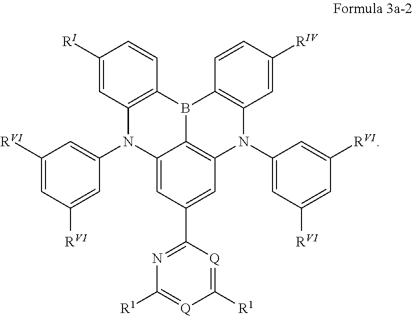

In another embodiment of the invention, R^{VI} is independently from each other selected from the group consisting of:
hydrogen, deuterium, halogen, Me, ⁱPr, ^{t}Bu, CN, CF₃, SiMe₃, SiPh₃,
Ph, which is optionally substituted with one or more substituents independently from each other selected from the group consisting of Me, ⁱPr, ^{t}Bu, CN, CF₃, and Ph,
and N(Ph)₂.

In another embodiment of the invention, the organic molecule comprises or consists of a structure of formula 3b

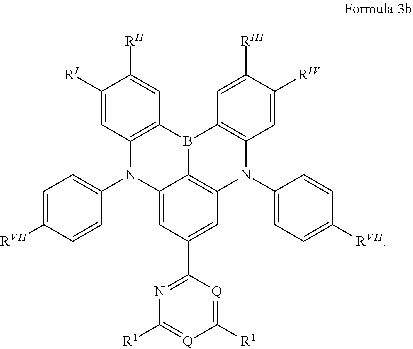

In one embodiment of the invention, the organic molecule comprises or consists of a structure of formula 3b-1

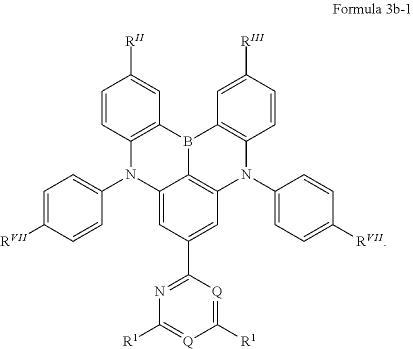

In one embodiment of the invention, the organic molecule comprises or consists of a structure of formula 3b-1a

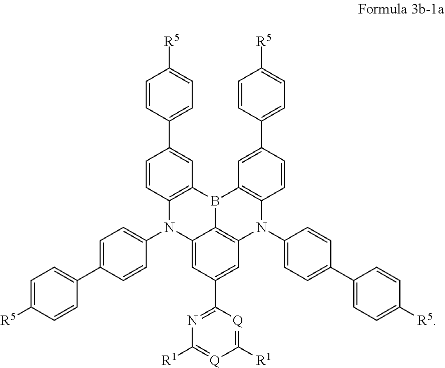

In one embodiment of the invention, the organic molecule comprises or consists of a structure of formula 3b-1a, wherein R¹ is phenyl at each occurrence.

In another embodiment of the invention, the organic molecule comprises or consists of a structure of formula 3b-1a, wherein R⁵ is hydrogen or a C₁-C₅-alkyl.

In another embodiment of the invention, the organic molecule comprises or consists of a structure of formula 3b-1a, wherein R⁵ is hydrogen or tert-butyl.

In another embodiment of the invention, the organic molecule comprises or consists of a structure of formula 3b-1a, wherein R⁵ is hydrogen.

In one embodiment of the invention, the organic molecule comprises or consists of a structure of formula 3b-2

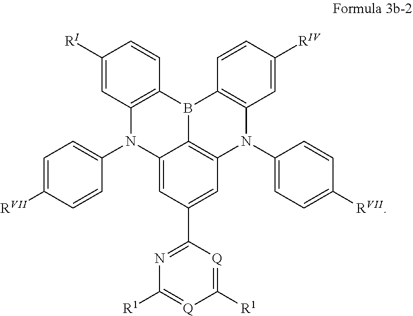

In another embodiment of the invention, R^{VII} is independently from each other selected from the group consisting of:
hydrogen, deuterium, halogen, Me, ⁱPr, ^{t}Bu, CN, CF₃, SiMe₃, SiPh₃,
Ph, which is optionally substituted with one or more substituents independently from each other selected from the group consisting of Me, ⁱPr, ^{t}Bu, CN, CF₃, and Ph;
and N(Ph)₂.

In certain embodiments of the invention, R^{I}═R^{IV} and/or R^{II}═R^{III}.

In certain embodiments of the invention, all Q=N.

In certain embodiments of the invention Q=N at any time and R¹ is independently from each other selected from the group consisting of:

hydrogen, and
C₆-C₁₃-aryl,
wherein optionally one or more hydrogen atoms are independently from each other substituted by C₁-C₅-alkyl, CN, CF₃ and Ph.

In certain embodiments of the invention, Q=N at any time and each of R¹ and R² is independently from each other selected from the group consisting of:

hydrogen,

### Ph,

wherein optionally one or more hydrogen atoms are independently from each other substituted by C₁-C₅-alkyl, CN, CF₃ and Ph.

In certain embodiments of the invention, each Q=N and

### R¹ is Ph,

wherein optionally one or more hydrogen atoms are independently from each other substituted by ^{t}Bu, and
R² is hydrogen.

In another embodiment of the invention, the organic molecules comprise or consist a structure of formula 4-1 4-2, 4-3 or 4-4:

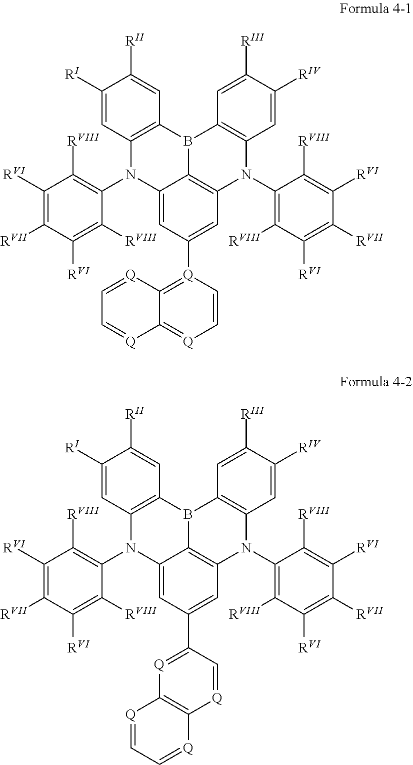

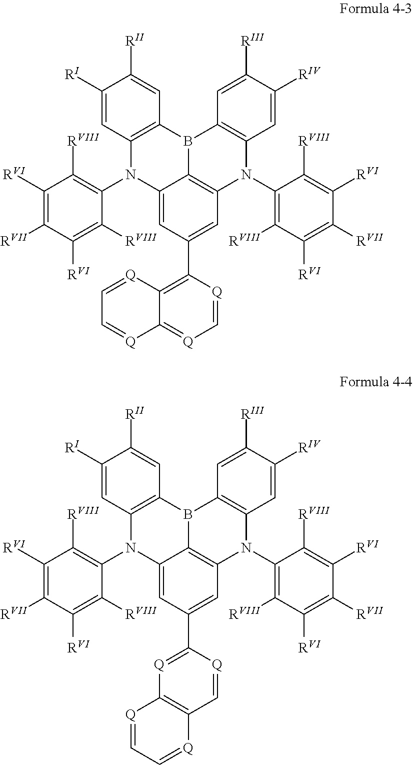

wherein
Q is at each occurrence independently from each other selected from the group consisting of N and C—R², wherein at least one Q is N.

In one embodiment of the invention, the organic molecule comprises or consists of a structure of formula 4-1:

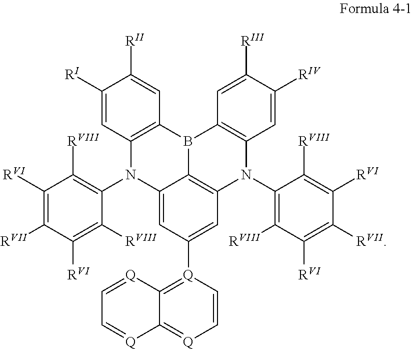

In one embodiment of the invention, the organic molecule comprises or consists of a structure of formula 4-2:

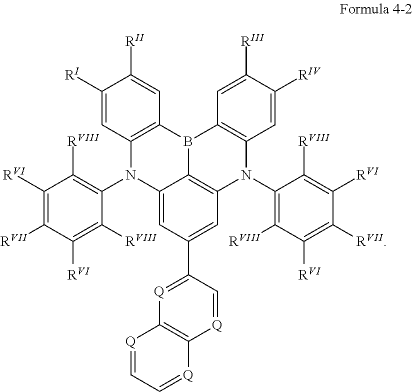

In a preferred embodiment of the invention, R^{I}, R^{II}, R^{III} and R^{IV} is independently from each other selected from the group consisting of:
hydrogen, deuterium, halogen, Me, ⁱPr, ^{t}Bu, CN, CF₃, SiMe₃, SiPh₃,
Ph, which is optionally substituted with one or more substituents independently from each other selected from the group consisting of Me, ⁱPr, ^{t}Bu, CN, CF₃, and Ph,
and N(Ph)₂.

In one embodiment of the invention, the organic molecule comprises or consists of a structure of formula 4a, 4b, 4c or 4d:

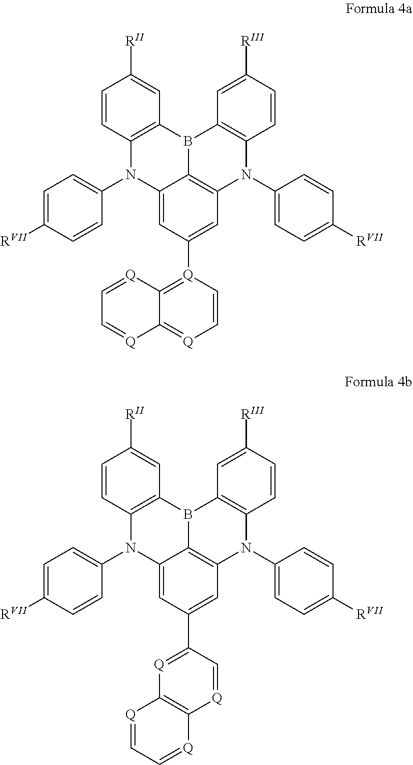

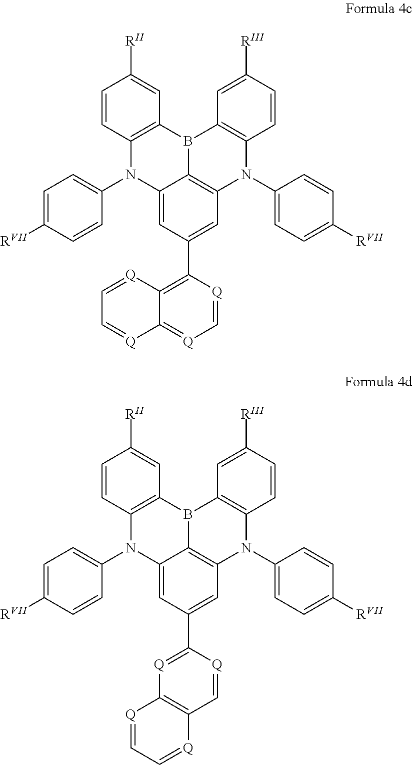

In one embodiment of the invention, the organic molecule comprises or consists of a structure of formula 4a:

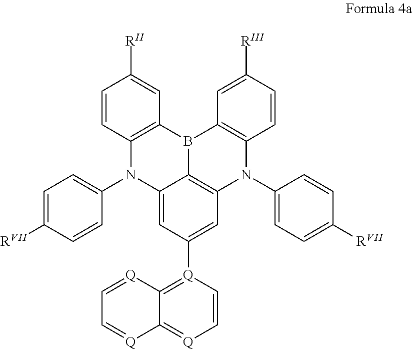

In one embodiment of the invention, the organic molecule comprises or consists of a structure of formula 4a-1a

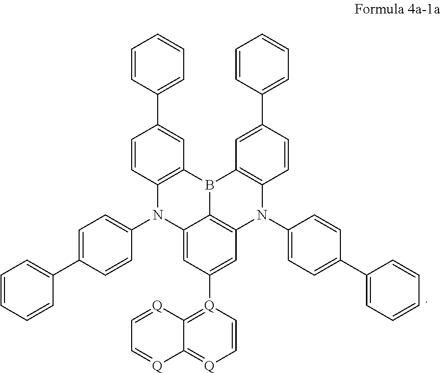

In one embodiment of the invention, the organic molecule comprises or consists of a structure of formula 4 b:

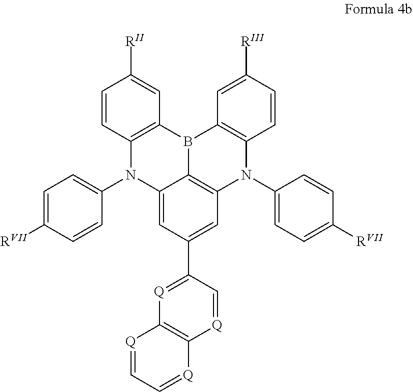

In one embodiment of the invention, the organic molecule comprises or consists of a structure of formula 4b-1a

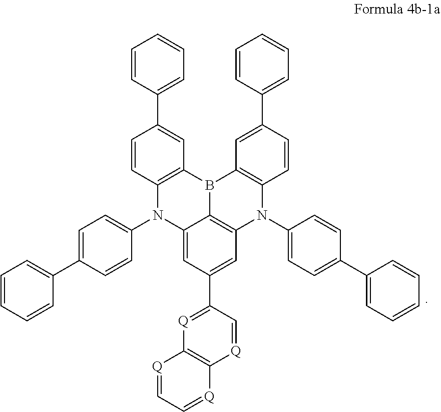

In one embodiment of the invention, the organic molecule comprises or consists of a structure of formula 5:

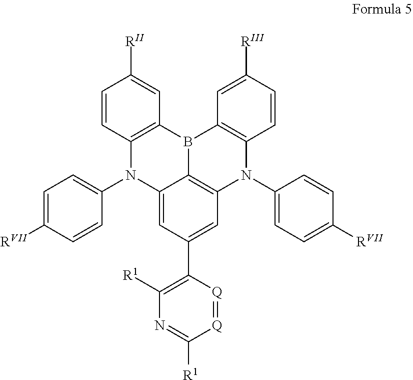

In one embodiment of the invention, the organic molecule comprises or consists of a structure of formula 5a

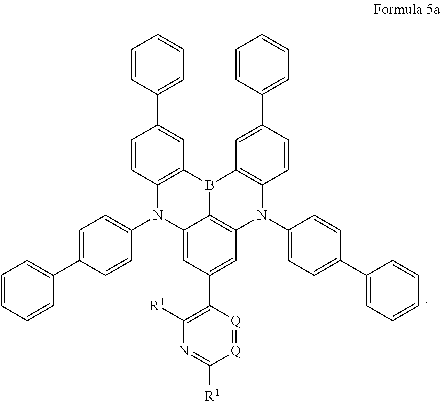

As used throughout the present application, the terms “aryl” and “aromatic” may be understood in the broadest sense as any mono-, bi- or polycyclic aromatic moieties. Accordingly, an aryl group contains 6 to 60 aromatic ring atoms, and a heteroaryl group contains 5 to 60 aromatic ring atoms, of which at least one is a heteroatom. Notwithstanding, throughout the application the number of aromatic ring atoms may be given as subscripted number in the definition of certain substituents. In particular, the heteroaromatic ring includes one to three heteroatoms. Again, the terms “heteroaryl” and “heteroaromatic” may be understood in the broadest sense as any mono-, bi- or polycyclic hetero-aromatic moieties that include at least one heteroatom. The heteroatoms may at each occurrence be the same or different and be individually selected from the group consisting of N, O and S. Accordingly, the term “arylene” refers to a divalent substituent that bears two binding sites to other molecular structures and thereby serving as a linker structure. In case, a group in the exemplary embodiments is defined differently from the definitions given here, for example, the number of aromatic ring atoms or number of heteroatoms differs from the given definition, the definition in the exemplary embodiments is to be applied. According to the invention, a condensed (annulated) aromatic or heteroaromatic polycycle is built of two or more single aromatic or heteroaromatic cycles, which formed the polycycle via a condensation reaction.

In particular, as used throughout, the term “aryl group or heteroaryl group” comprises groups which can be bound via any position of the aromatic or heteroaromatic group, derived from benzene, naphthaline, anthracene, phenanthrene, pyrene, dihydropyrene, chrysene, perylene, fluoranthene, benzanthracene, benzphenanthrene, tetracene, pentacene, benzpyrene, furan, benzofuran, isobenzofuran, dibenzofuran, thiophene, benzothiophene, isobenzothiophene, dibenzothiophene; pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthoimidazole, phenanthroimidazole, pyridoimidazole, pyrazinoimidazole, quinoxalinoimidazole, oxazole, benzoxazole, napthooxazole, anthroxazol, phenanthroxazol, isoxazole, 1,2-thiazole, 1,3-thiazole, benzothiazole, pyridazine, benzopyridazine, pyrimidine, benzopyrimidine, 1,3,5-triazine, quinoxaline, pyrazine, phenazine, naphthyridine, carboline, benzocarboline, phenanthroline, 1,2,3-triazole, 1,2,4-triazole, benzotriazole, 1,2,3-oxadiazole, 1,2,4-oxadiazole, 1,2,5-oxadiazole, 1,2,3,4-tetrazine, purine, pteridine, indolizine and benzothiadiazole or combinations of the abovementioned groups.

As used throughout the present application, the term “electron-withdrawing” with respect to heteroaryl groups or “electron-withdrawing heteroaryl group” may be understood as a C₃-C₁₅-heteroaromat group as part of the organic molecule that by a mesomeric and/or inductive effect causes a shift in the electron density from the remainder of the organic molecule towards itself and thereby reduces the electron density of the remainder of the organic molecule, namely to a greater extent than would be caused by a hydrogen atom located at the same position. The term “electron-withdrawing heteroaryl group” exemplarily comprises furan, benzofuran, isobenzofuran, dibenzofuran, thiophene, benzothiophene, isobenzothiophene, dibenzothiophene; pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthoimidazole, phenanthroimidazole, pyridoimidazole, pyrazinoimidazole, quinoxalinoimidazole, oxazole, benzoxazole, napthooxazole, anthroxazol, phenanthroxazol, isoxazole, 1,2-thiazole, 1,3-thiazole, benzothiazole, pyridazine, benzopyridazine, pyrimidine, benzopyrimidine, 1,3,5-triazine, quinoxaline, pyrazine, phenazine, naphthyridine, carboline, benzocarboline, phenanthroline, 1,2,3-triazole, 1,2,4-triazole, benzotriazole, 1,2,3-oxadiazole, 1,2,4-oxadiazole, 1,2,5-oxadiazole, 1,2,3,4-tetrazine, purine, pteridine, indolizine and benzothiadiazole. Examples of such groups are shown below:

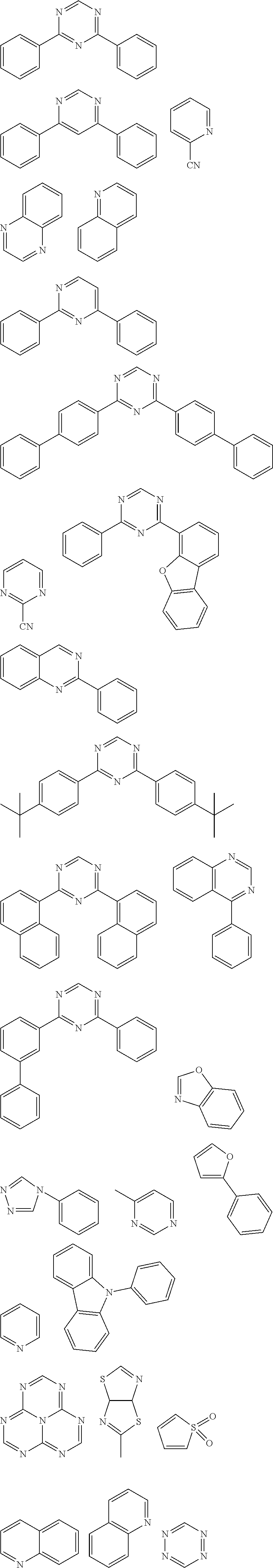

Specific examples for organic molecules according to formula I, wherein R^{V} is an electron-withdrawing heteroaryl group are shown below:

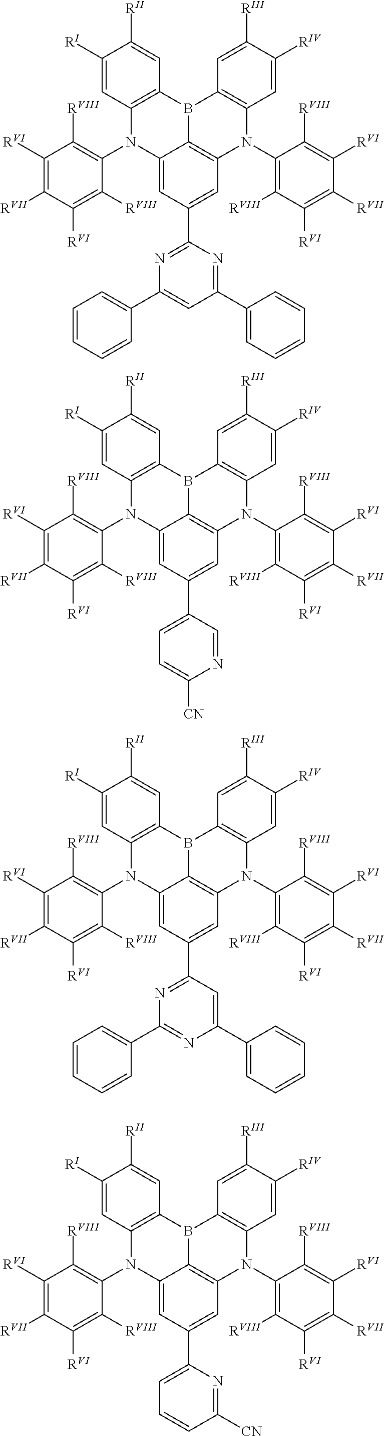

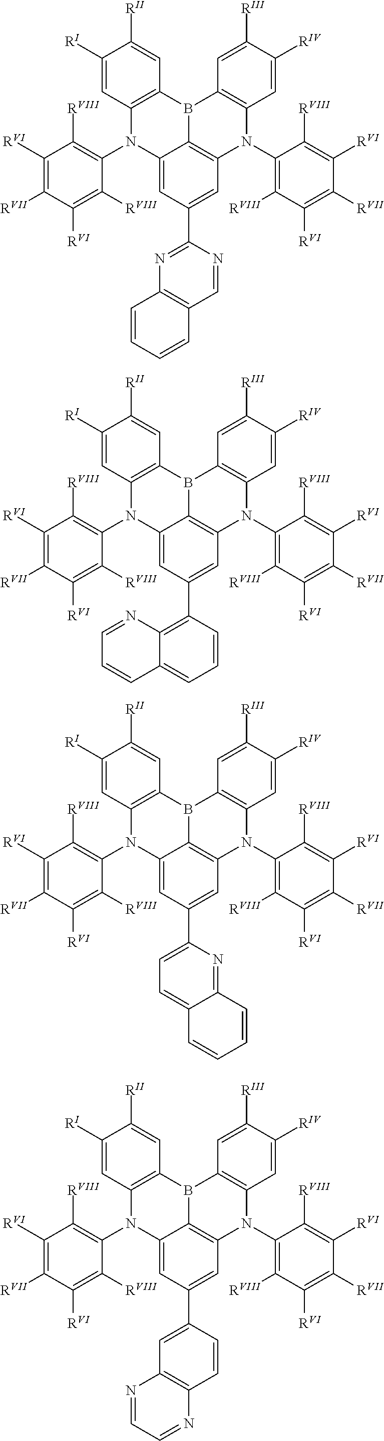

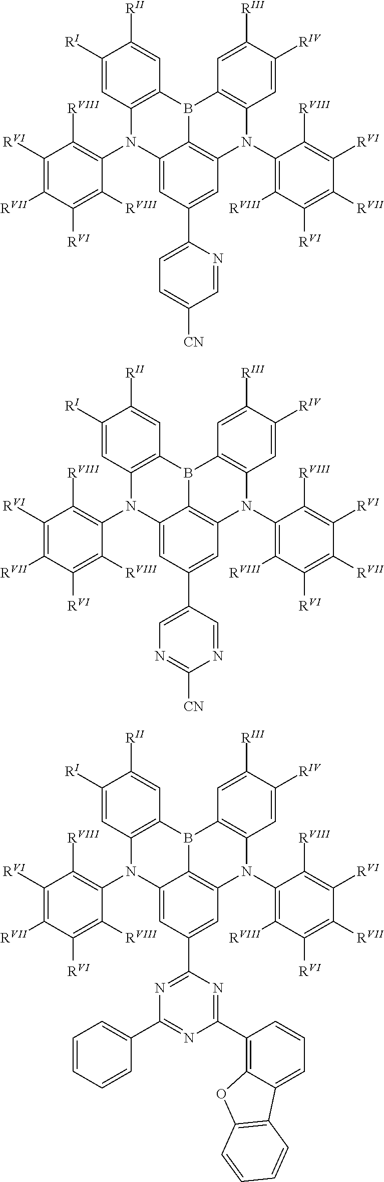

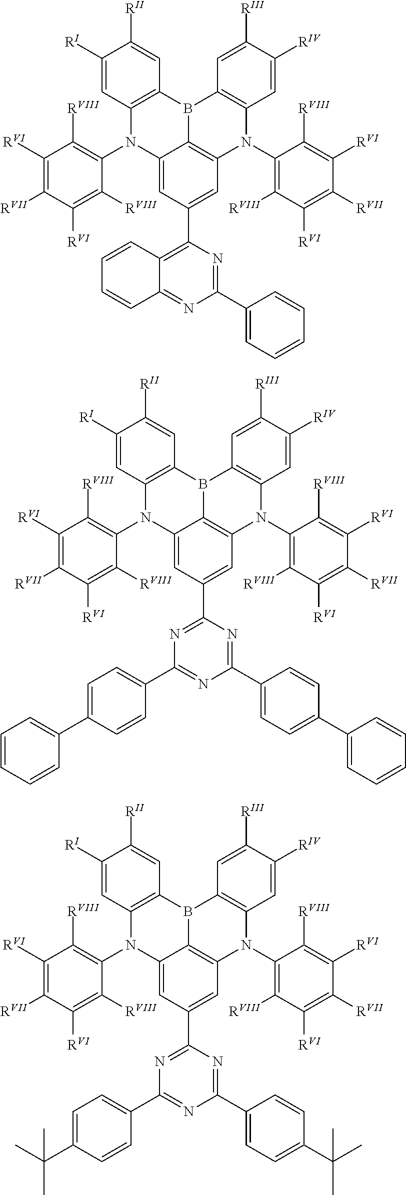

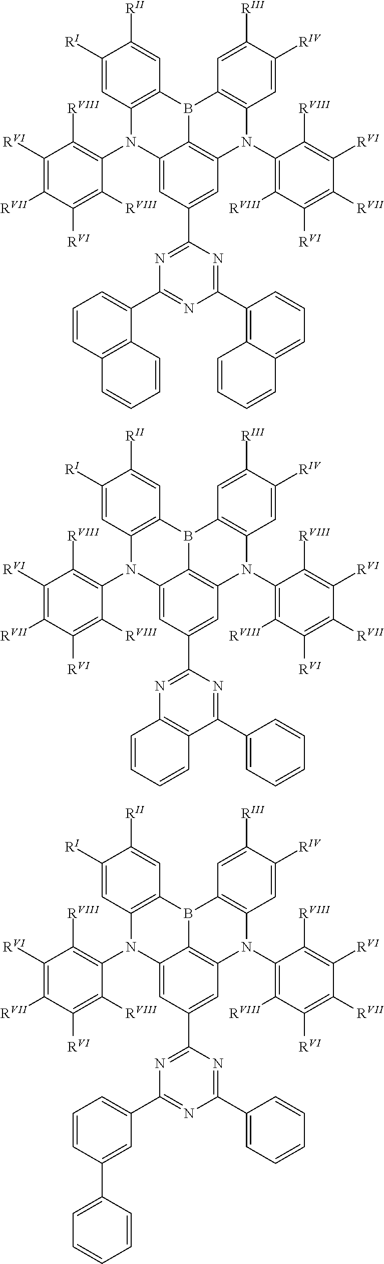

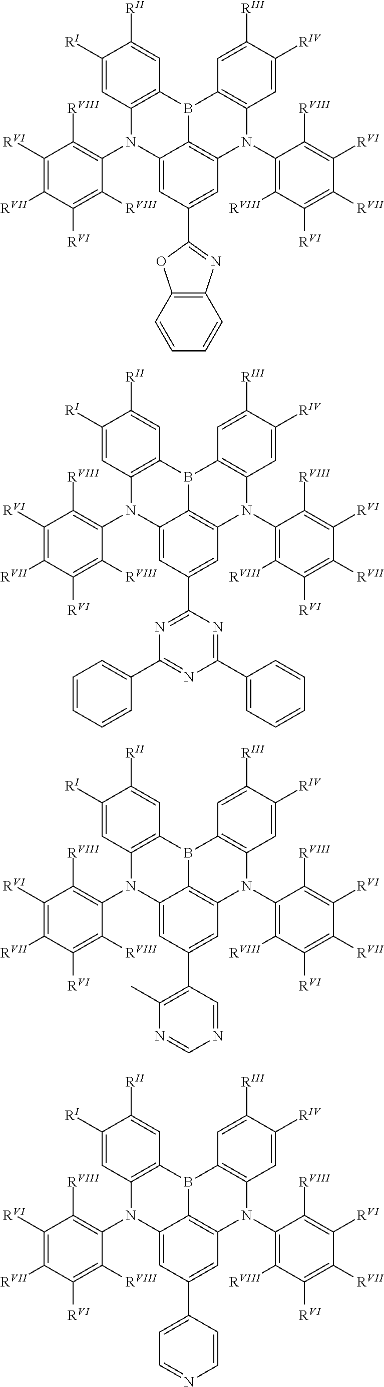

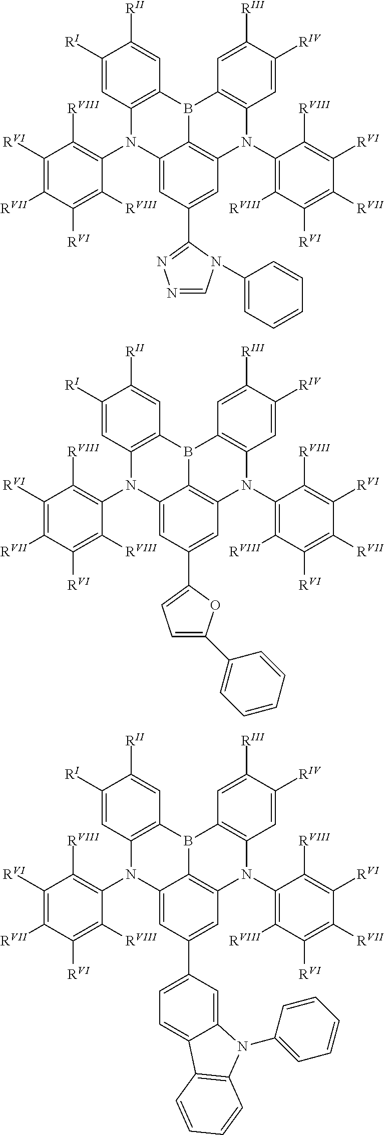

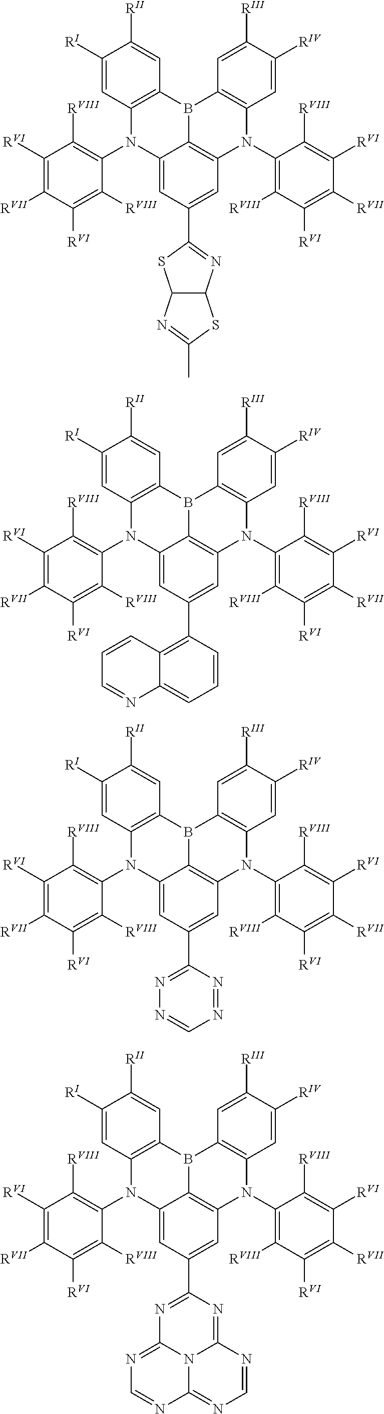

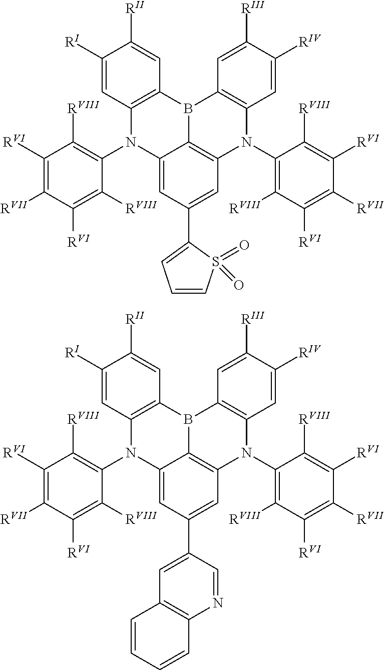

As used throughout, the term “cyclic group” may be understood in the broadest sense as any mono-, bi- or polycyclic moieties.

As used throughout, the term “biphenyl” as a substituent may be understood in the broadest sense as ortho-biphenyl, meta-biphenyl, or para-biphenyl, wherein ortho, meta and para is defined in regard to the binding site to another chemical moiety.

As used throughout, the term “alkyl group” may be understood in the broadest sense as any linear, branched, or cyclic alkyl substituent. In particular, the term alkyl comprises the substituents methyl (Me), ethyl (Et), n-propyl (ⁿPr), i-propyl (ⁱPr), cyclopropyl, n-butyl (ⁿBu), i-butyl (ⁱBu), s-butyl (^{s}Bu), t-butyl (^{t}Bu), cyclobutyl, 2-methylbutyl, n-pentyl, s-pentyl, t-pentyl, 2-pentyl, neo-pentyl, cyclopentyl, n-hexyl, s-hexyl, t-hexyl, 2-hexyl, 3-hexyl, neo-hexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2,2,2]octyl, 2-bicyclo[2,2,2]-octyl, 2-(2,6-dimethyl)octyl, 3-(3,7-dimethyl)octyl, adamantyl, 2,2,2-trifluorethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyln-n-hexadec-1-yl, 1,1-diethyl-n-octadec-1-yl, 1-(n-propyl)-cyclohex-1-yl, 1-(n-butyl)-cyclohex-1-yl, 1-(n-hexyl)-cyclohex-1-yl, 1-(n-octyl)-cyclohex-1-yl and 1-(n-decyl)-cyclohex-1-yl.

As used throughout, the term “alkenyl” comprises linear, branched, and cyclic alkenyl substituents. The term “alkenyl group”, for example, comprises the substituents ethenyl, propenyl, butenyl, pentenyl, cyclopentenyl, hexenyl, cyclohexenyl, heptenyl, cycloheptenyl, octenyl, cyclooctenyl or cyclooctadienyl.

As used throughout, the term “alkynyl” comprises linear, branched, and cyclic alkynyl substituents. The term “alkynyl group”, for example, comprises ethynyl, propynyl, butynyl, pentynyl, hexynyl, heptynyl or octynyl.

As used throughout, the term “alkoxy” comprises linear, branched, and cyclic alkoxy substituents. The term “alkoxy group” exemplarily comprises methoxy, ethoxy, n-propoxy, i-propoxy, n-butoxy, i-butoxy, s-butoxy, t-butoxy and 2-methylbutoxy.

As used throughout, the term “thioalkoxy” comprises linear, branched, and cyclic thioalkoxy substituents, in which the O of the exemplarily alkoxy groups is replaced by S.

As used throughout, the terms “halogen” and “halo” may be understood in the broadest sense as being preferably fluorine, chlorine, bromine or iodine.

Whenever hydrogen (H) is mentioned herein, it could also be replaced by deuterium at each occurrence.

It is understood that when a molecular fragment is described as being a substituent or otherwise attached to another moiety, its name may be written as if it were a fragment (e.g. naphtyl, dibenzofuryl) or as if it were the whole molecule (e.g. naphthalene, dibenzofuran). As used herein, these different ways of designating a substituent or attached fragment are considered to be equivalent.

In one embodiment the organic molecules exhibit emission maxima in the green spectral range.

In one embodiment, the organic molecules according to the invention have an excited state lifetime of not more than 150 μs, of not more than 100 μs, in particular of not more than 50 μs, more preferably of not more than 10 μs or not more than 7 μs in a film of poly(methyl methacrylate) (PMMA) with 1% to 5%, in particular with 2% by weight of organic molecule at room temperature.

In one embodiment, the organic molecules according to the invention have an excited state lifetime of not more than 25 μs, of not more than 15 μs, in particular of not more than 10 μs, more preferably of not more than 8 μs or not more than 6 μs, even more preferably of not more than 4 μs in a film of poly(methyl methacrylate) (PMMA) with 1% to 5%, in particular with 2% by weight of organic molecule at room temperature.

In a further embodiment of the invention, the organic molecules according to the invention have an emission peak in the visible or nearest ultraviolet range, i.e., in the range of a wavelength of from 380 nm to 800 nm, with a full width at half maximum of less than 0.23 eV, preferably less than 0.20 eV, more preferably less than 0.19 eV, even more preferably less than 0.18 eV or even less than 0.17 eV in a film of poly(methyl methacrylate) (PMMA) with 1% to 5%, in particular with 5% by weight of organic molecule at room temperature.

Orbital and excited state energies can be determined either by means of experimental methods. The energy of the highest occupied molecular orbital E^{HOMO} is determined by methods known to the person skilled in the art from cyclic voltammetry measurements with an accuracy of 0.1 eV. The energy of the lowest unoccupied molecular orbital E^{LUMO} is calculated as E^{HOMO}+E^{gap}, wherein E^{gap} is determined as follows: For host compounds, the onset of the emission spectrum of a film with 10% by weight of host in poly(methyl methacrylate) (PMMA) is used as E^{gap}, unless stated otherwise. For emitter molecules, E^{gap} is determined as the energy at which the excitation and emission spectra of a film with 10% by weight of emitter in PMMA cross. For the organic molecules according to the invention, E^{gap} is determined as the energy at which the excitation and emission spectra of a film with 1% to 5%, in particular with 2% by weight of emitter in PMMA cross.

The energy of the first excited triplet state T1 is determined from the onset of the emission spectrum at low temperature, typically at 77 K. For host compounds, where the first excited singlet state and the lowest triplet state are energetically separated by >0.4 eV, the phosphorescence is usually visible in a steady-state spectrum in 2-Me-THF. The triplet energy can thus be determined as the onset of the phosphorescence spectrum. For TADF emitter molecules, the energy of the first excited triplet state T1 is determined from the onset of the delayed emission spectrum at 77 K, if not otherwise stated, measured in a film of PMMA with 10% by weight of emitter and in case of the organic molecules according to the invention with 1% to 5%, in particular with 2% by weight of the organic molecules according to the invention. Both for host and emitter compounds, the energy of the first excited singlet state S1 is determined from the onset of the emission spectrum, if not otherwise stated, measured in a film of PMMA with 10% by weight of host or emitter compound and in case of the organic molecules according to the invention with 1% to 5%, in particular with 2% by weight of the organic molecules according to the invention.

The onset of an emission spectrum is determined by computing the intersection of the tangent to the emission spectrum with the x-axis. The tangent to the emission spectrum is set at the high-energy side of the emission band and at the point at half maximum of the maximum intensity of the emission spectrum.

In one embodiment, the organic molecules according to the invention have an onset of the emission spectrum, which is energetically close to the emission maximum, i.e. the energy difference between the onset of the emission spectrum and the energy of the emission maximum is below 0.14 eV, preferably below 0.13 eV, or even below 0.12 eV, while the full width at half maximum (FWHM) of the organic molecules is less than 0.23 eV, preferably less than 0.20 eV, more preferably less than 0.19 eV, even more preferably less than 0.18 eV or even less than 0.17 eV in a film of poly(methyl methacrylate) (PMMA) with 1% to 5%, in particular with 2% by weight of organic molecule at room temperature, resulting in a CIEy coordinate below 0.20, preferably below 0.18, more preferably below 0.16 or even more preferred below 0.14.

In another embodiment of the invention, the organic molecules according to the invention have an emission peak in the visible or nearest ultraviolet range, i.e., in the range of a wavelength of from 485 to 560 nm, with a full width at half maximum of less than 0.50 eV, preferably less than 0.48 eV, more preferably less than 0.45 eV, even more preferably less than 0.43 eV or even less than 0.40 eV in a film of poly(methyl methacrylate) (PMMA) with 1% to 5%, in particular with 2% by weight of organic molecule at room temperature.

In a further embodiment of the invention, the organic molecules according to the invention have an emission peak in the visible or nearest ultraviolet range, i.e., in the range of a wavelength of from 485 to 560 nm, with a full width at half maximum of less than 0.40 eV in a film of poly(methyl methacrylate) (PMMA) with 1% to 5%, in particular with 2% by weight of organic molecule at room temperature.

In a further embodiment of the invention, the organic molecules according to the invention have an emission peak in the visible or nearest ultraviolet range, i.e., in the range of a wavelength of from 485 to 560 nm, with a full width at half maximum of less than 0.40 eV, more preferably less than 0.30 eV, even, more preferably less than 0.28 eV, more preferably less than 0.25 eV or even less than 0.23 eV in a film of poly(methyl methacrylate) (PMMA) with 1% to 5%, in particular with 2% by weight of organic molecule at room temperature.

A further aspect of the invention relates to the use of an organic molecule of the invention as a luminescent emitter or as an absorber, and/or as a host material and/or as an electron transport material, and/or as a hole injection material, and/or as a hole blocking material in an optoelectronic device.

A preferred embodiment relates to the use of an organic molecule according to the invention as a luminescent emitter in an optoelectronic device.

The optoelectronic device may be understood in the broadest sense as any device based on organic materials that is suitable for emitting light in the visible or nearest ultraviolet (UV) range, i.e., in the range of a wavelength of from 380 to 800 nm. More preferably, the optoelectronic device may be able to emit light in the visible range, i.e., of from 400 nm to 800 nm.

In the context of such use, the optoelectronic device is more particularly selected from the group consisting of:
organic light-emitting diodes (OLEDs),
light-emitting electrochemical cells,
OLED sensors, especially in gas and vapor sensors that are not hermetically shielded to the surroundings,
organic diodes,
organic solar cells,
organic transistors,
organic field-effect transistors,
organic lasers, and
down-conversion elements.

In a preferred embodiment in the context of such use, the optoelectronic device is a device selected from the group consisting of an organic light emitting diode (OLED), a light emitting electrochemical cell (LEC), and a light-emitting transistor.

In the case of the use, the fraction of the organic molecule according to the invention in the emission layer in an optoelectronic device, more particularly in an OLED, is 0.1% to 99% by weight, more particularly 1% to 80% by weight. In an alternative embodiment, the proportion of the organic molecule in the emission layer is 100% by weight.

In one embodiment, the light-emitting layer comprises not only the organic molecules according to the invention, but also a host material whose triplet (T1) and singlet (S1) energy levels are energetically higher than the triplet (T1) and singlet (S1) energy levels of the organic molecule.

A further aspect of the invention relates to a composition comprising or consisting of:
(a) at least one organic molecule according to the invention, in particular in the form of an emitter and/or a host, and
(b) one or more emitter and/or host materials, which differ from the organic molecule according to the invention and
(c) optional one or more dyes and/or one or more solvents.

In one embodiment, the light-emitting layer comprises (or essentially consists of) a composition comprising or consisting of:
(a) at least one organic molecule according to the invention, in particular in the form of an emitter and/or a host, and
(b) one or more emitter and/or host materials, which differ from the organic molecule according to the invention and
(c) optional one or more dyes and/or one or more solvents.

In a further embodiment of the invention, the composition has a photoluminescence quantum yield (PLQY) of more than 10%, preferably more than 20%, more preferably more than 40%, even more preferably more than 60% or even more than 70% at room temperature.

Compositions with at Least One Further Emitter

One embodiment of the invention relates to a composition comprising or consisting of:
(i) 1-50% by weight, preferably 5-40% by weight, in particular 10-30% by weight, of the organic molecule according to the invention;
(ii) 5-98% by weight, preferably 30-93.9% by weight, in particular 40-88% by weight, of one host compound H;
(iii) 1-30% by weight, in particular 1-20% by weight, preferably 1-5% by weight, of at least one further emitter molecule F with a structure differing from the structure of the molecules according to the invention; and
(iv) optionally 0-94% by weight, preferably 0.1-65% by weight, in particular 1-50% by weight, of at least one further host compound D with a structure differing from the structure of the molecules according to the invention; and
(v) optionally 0-94% by weight, preferably 0-65% by weight, in particular 0-50% by weight, of a solvent.

The components or the compositions are chosen such that the sum of the weight of the components add up to 100%.

In a further embodiment of the invention, the composition has an emission peak in the visible or nearest ultraviolet range, i.e., in the range of a wavelength of from 380 to 800 nm.

In one embodiment of the invention, the at least one further emitter molecule F is a purely organic emitter.

In one embodiment of the invention, at least one further emitter molecule F is a purely organic TADF emitter. Purely organic TADF emitters are known from the state of the art, e.g. Wong and Zysman-Colman (“Purely Organic Thermally Activated Delayed Fluorescence Materials for Organic Light-Emitting Diodes.”, Adv. Mater. 2017 June; 29(22)).

In one embodiment of the invention, the at least one further emitter molecule F is a fluorescence emitter, in particular a red, a yellow or a green fluorescence emitter.

In a further embodiment of the invention, the composition, containing at least one further emitter molecule F shows an emission peak in the visible or nearest ultraviolet range, i.e., in the range of a wavelength of from 380 to 800 nm, with a full width at half maximum of less than 0.30 eV, in particular less than 0.25 eV, preferably less than 0.22 eV, more preferably less than 0.19 eV or even less than 0.17 eV at room temperature, with a lower limit of 0.05 eV.

### Composition Wherein the at Least One Further Emitter Molecule F is a Green Fluorescence Emitter

In a further embodiment of the invention, the at least one further emitter molecule F is a fluorescence emitter, in particular a green fluorescence emitter.

In one embodiment, the at least one further emitter molecule F is a fluorescence emitter selected from the following group:

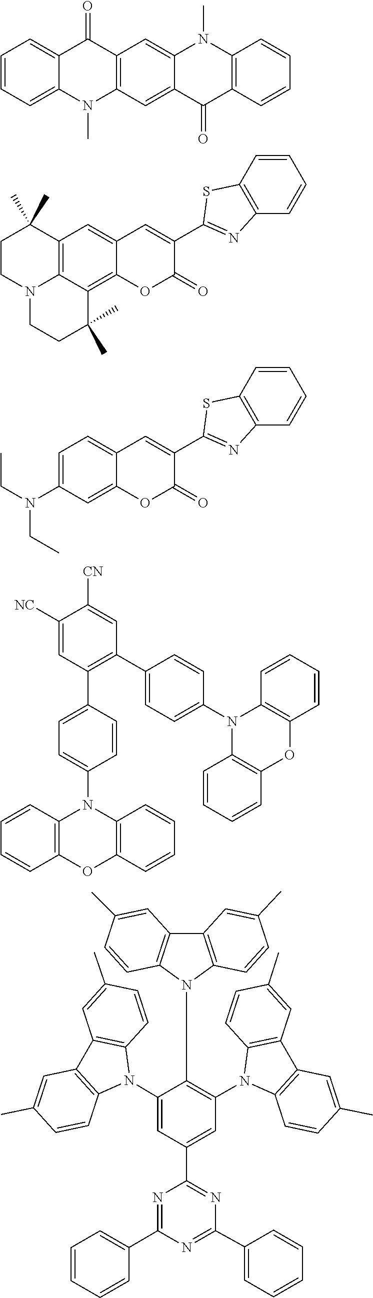

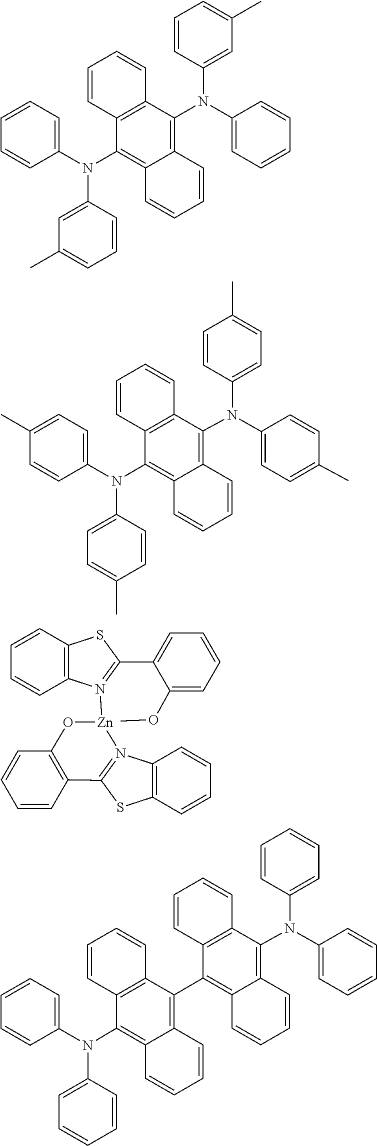

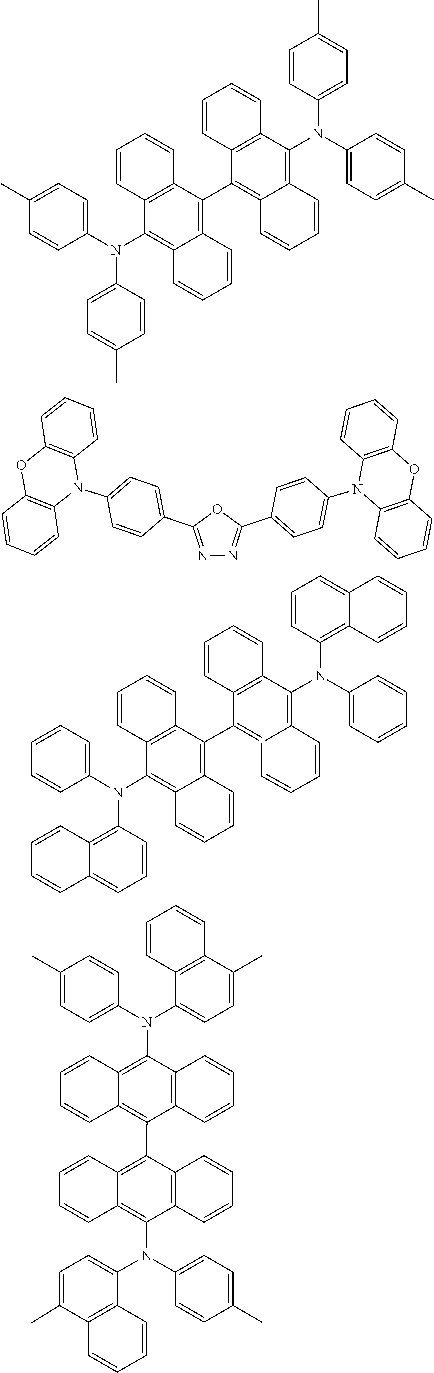

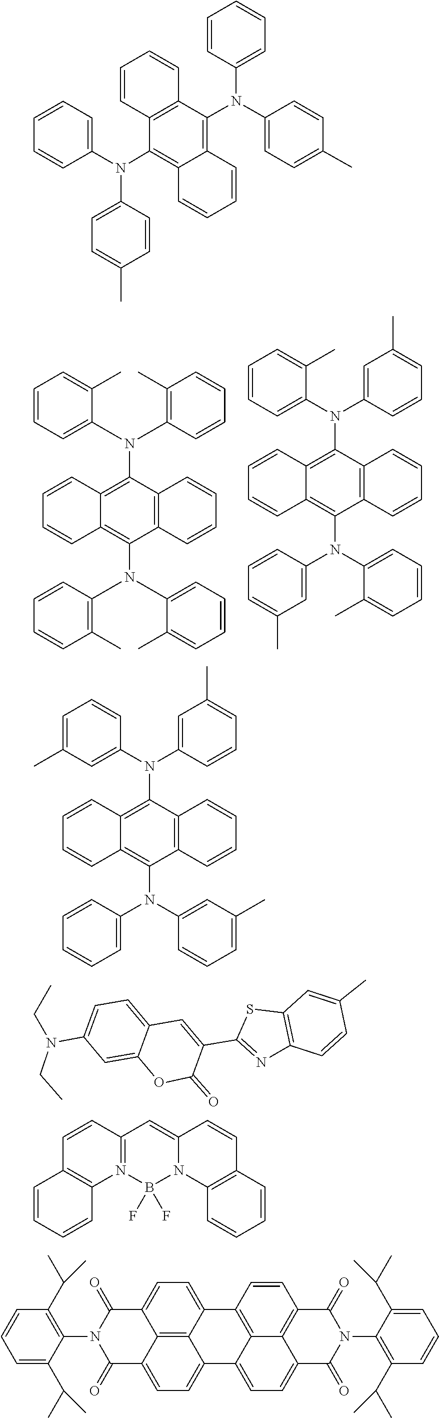

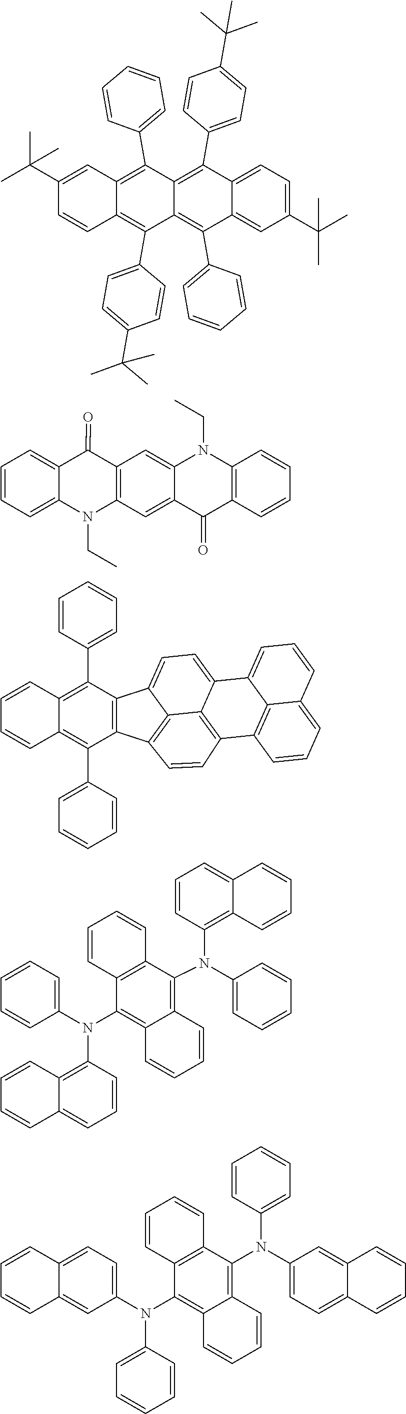

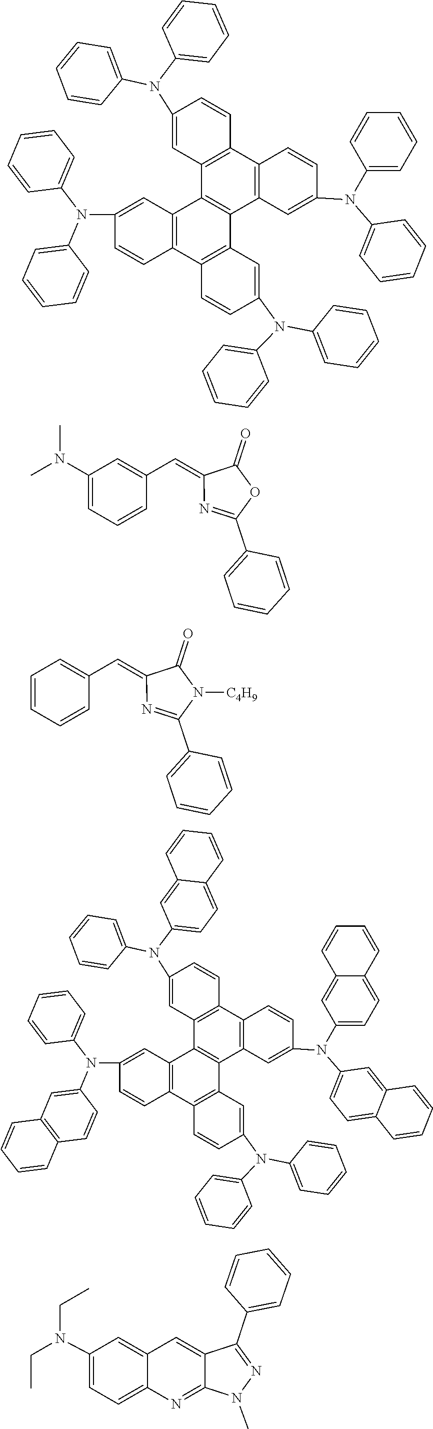

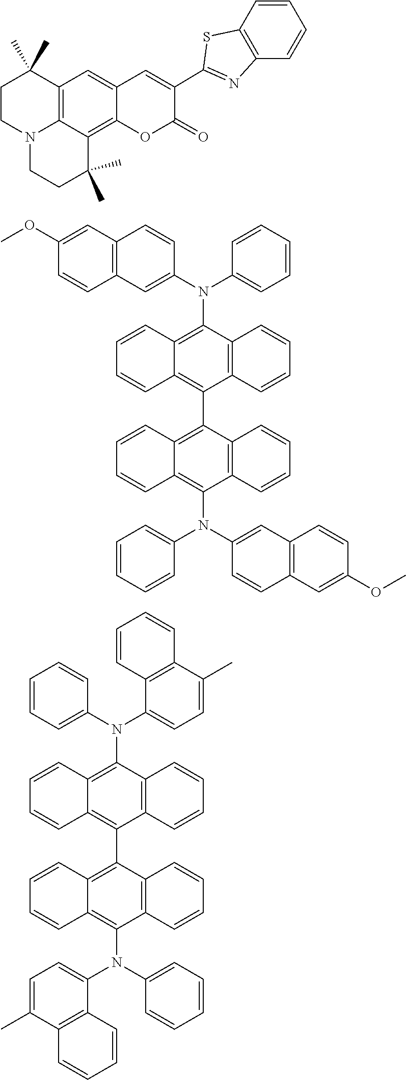

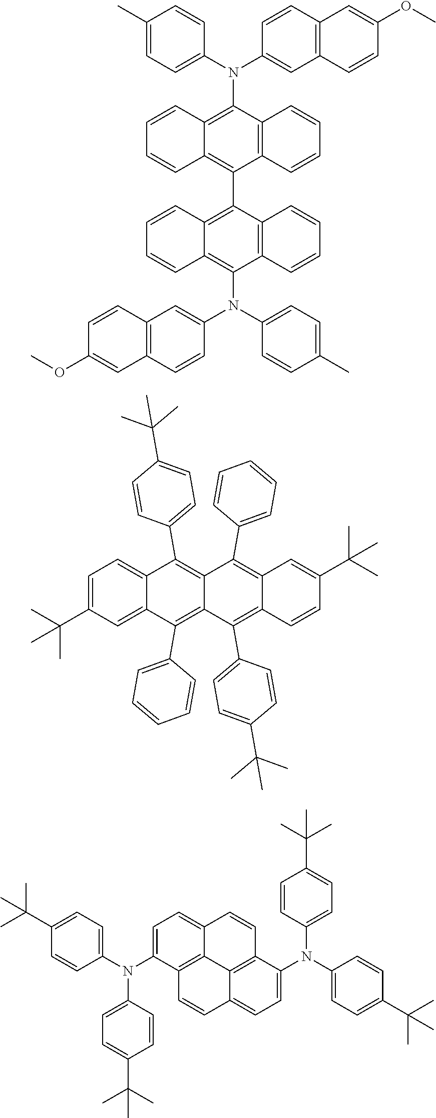

In a further embodiment of the invention, the composition has an emission peak in the visible or nearest ultraviolet range, i.e., in the range of a wavelength of from 380 to 800 nm, in particular between 485 nm and 590 nm, preferably between 505 nm and 565 nm, even more preferably between 515 nm and 545 nm.

### Composition Wherein the at Least One Further Emitter Molecule F is a Red Fluorescence Emitter

In a further embodiment of the invention, at least one further emitter molecule F is a fluorescence emitter, in particular a red fluorescence emitter.

In one embodiment, at least one further emitter molecule F is a fluorescence emitter selected from the following group:

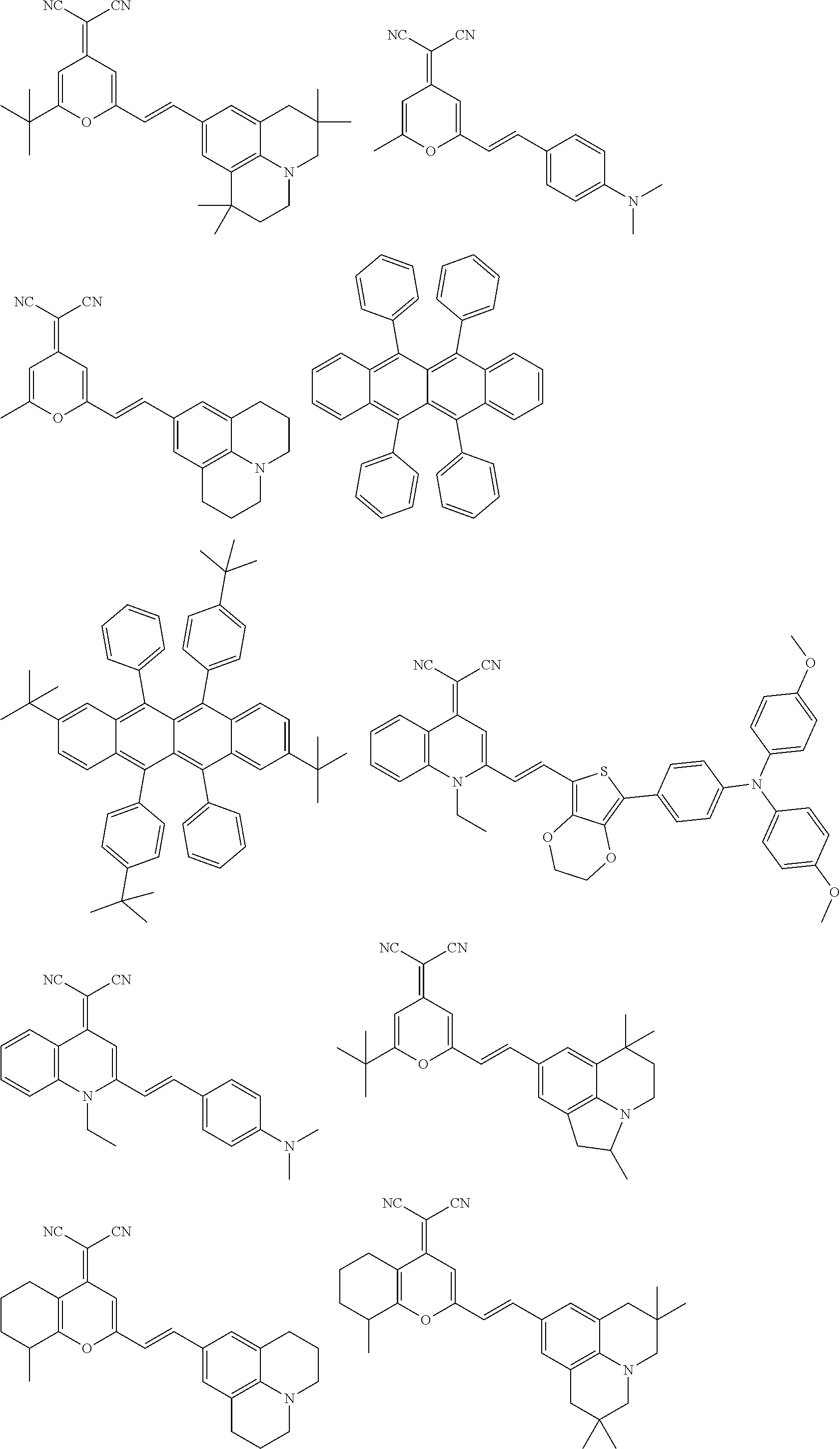

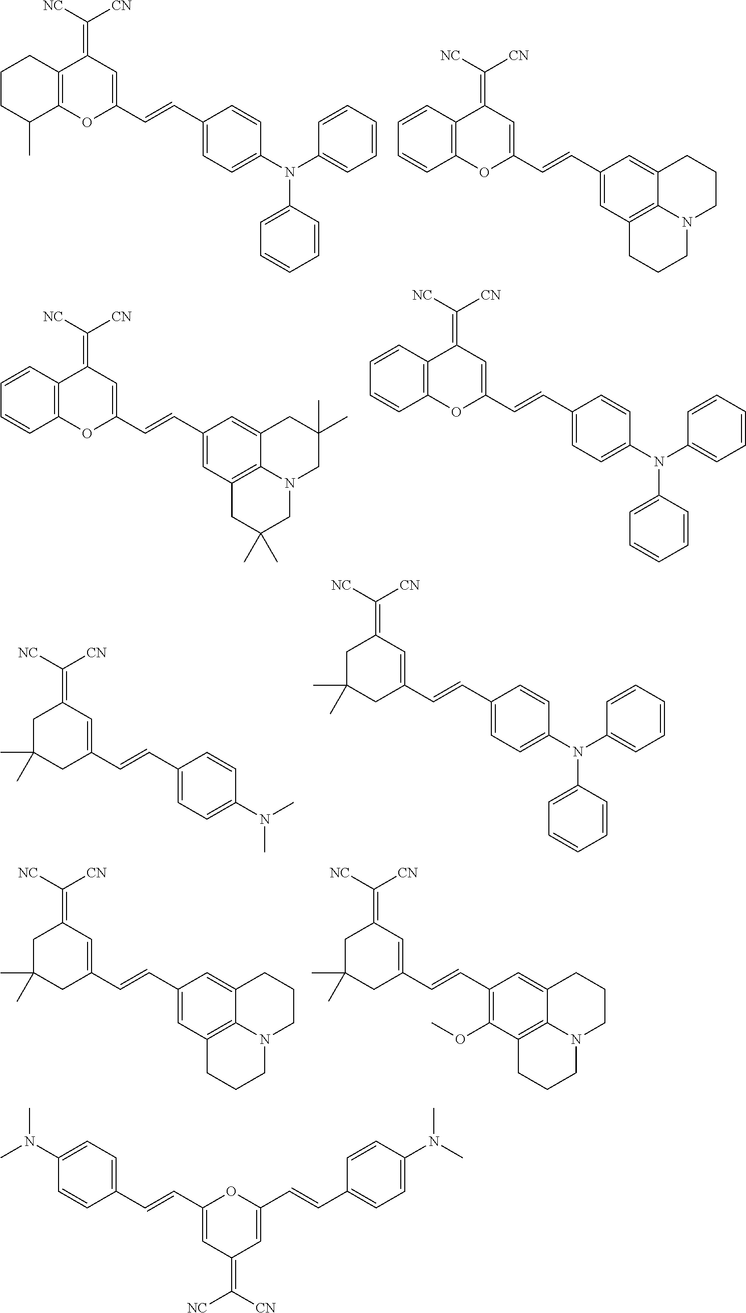

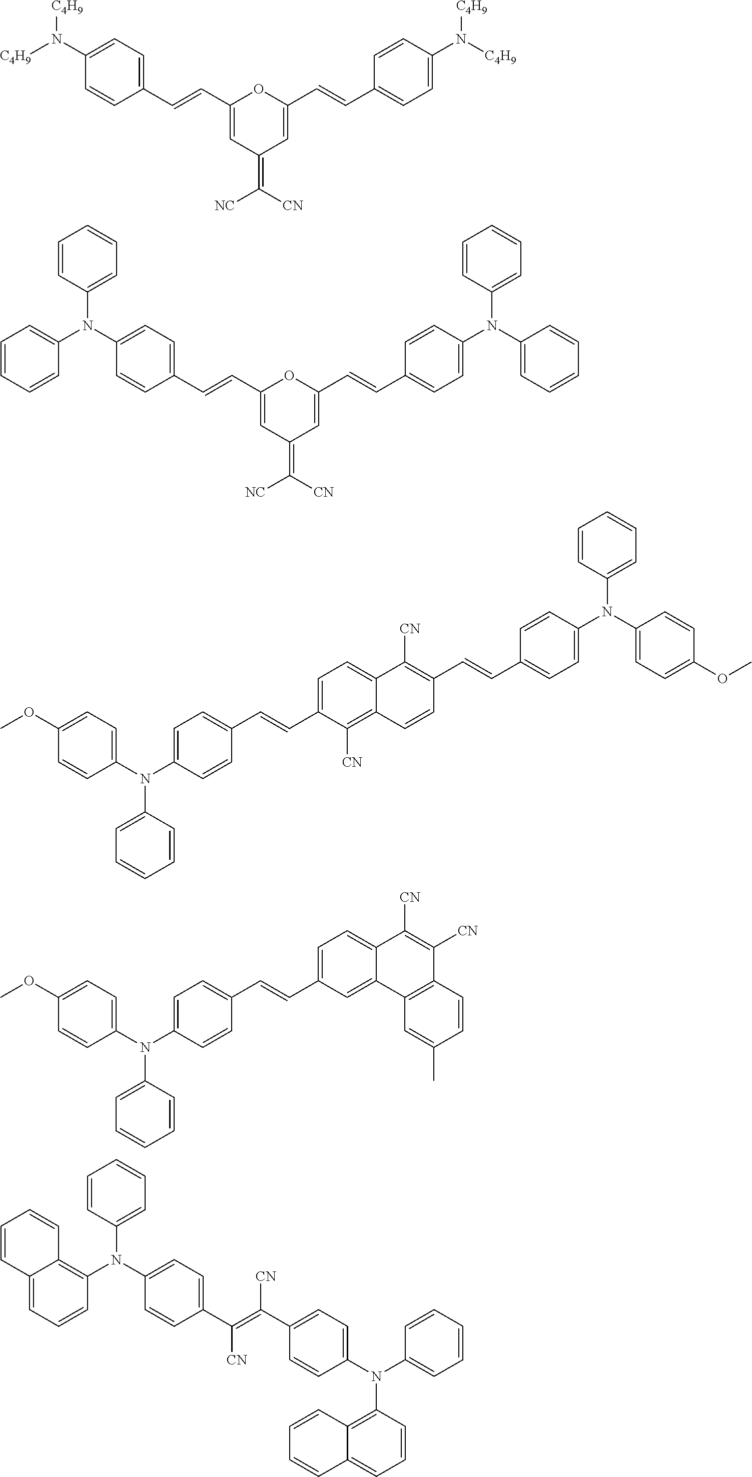

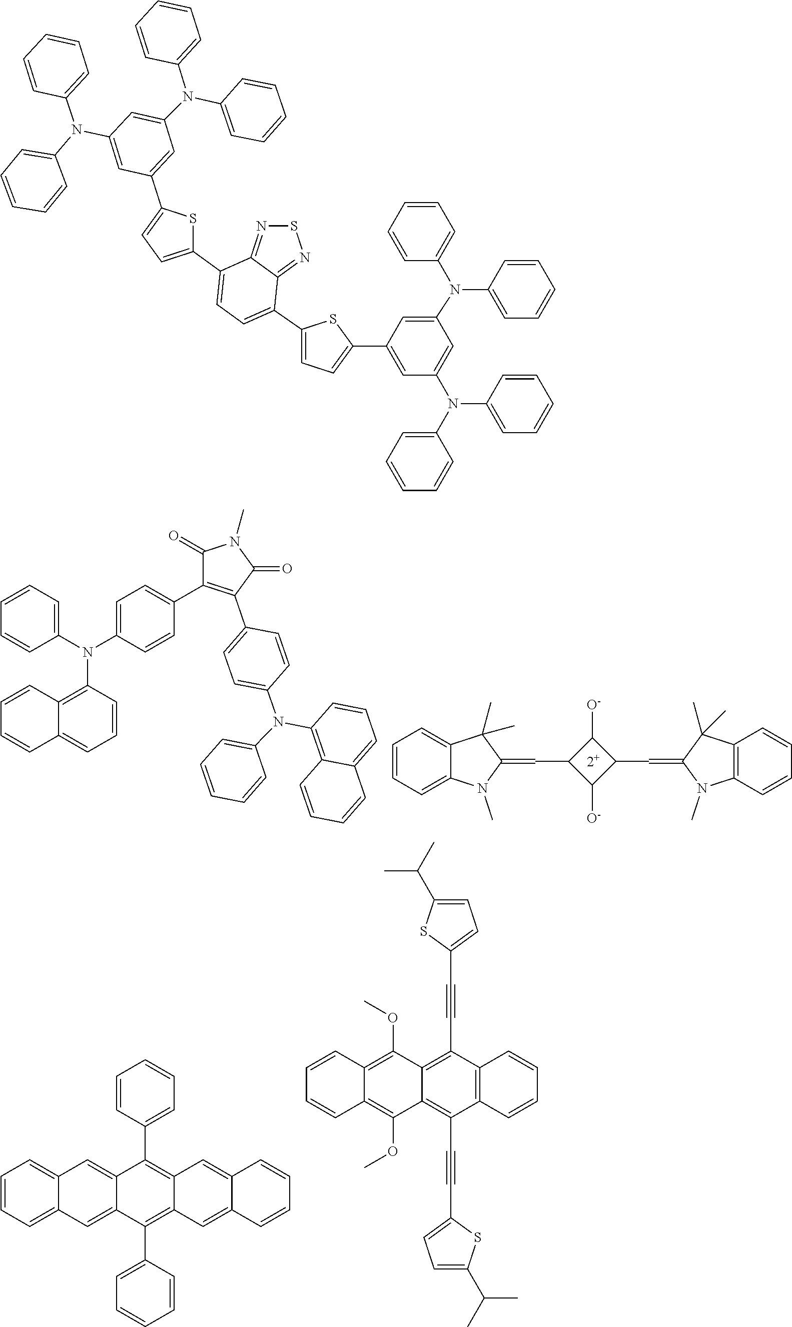

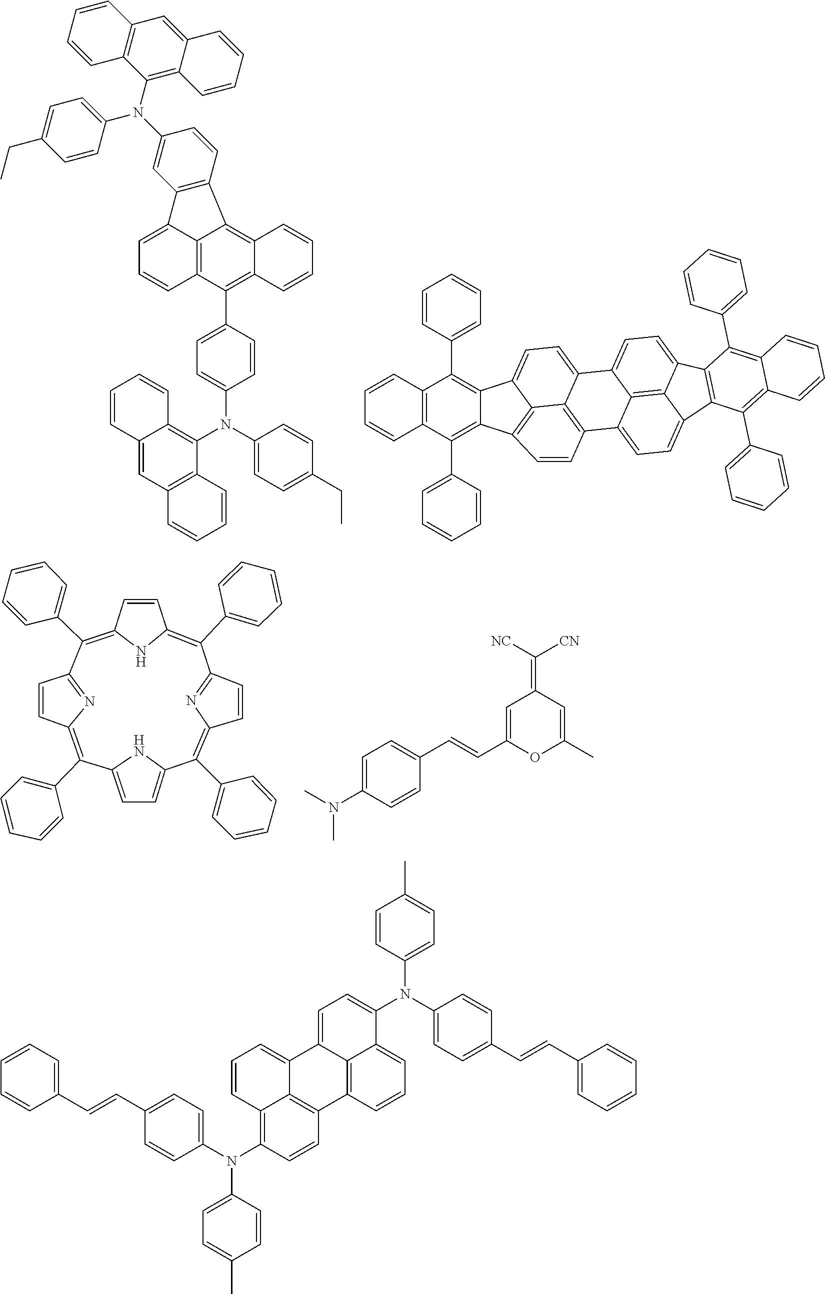

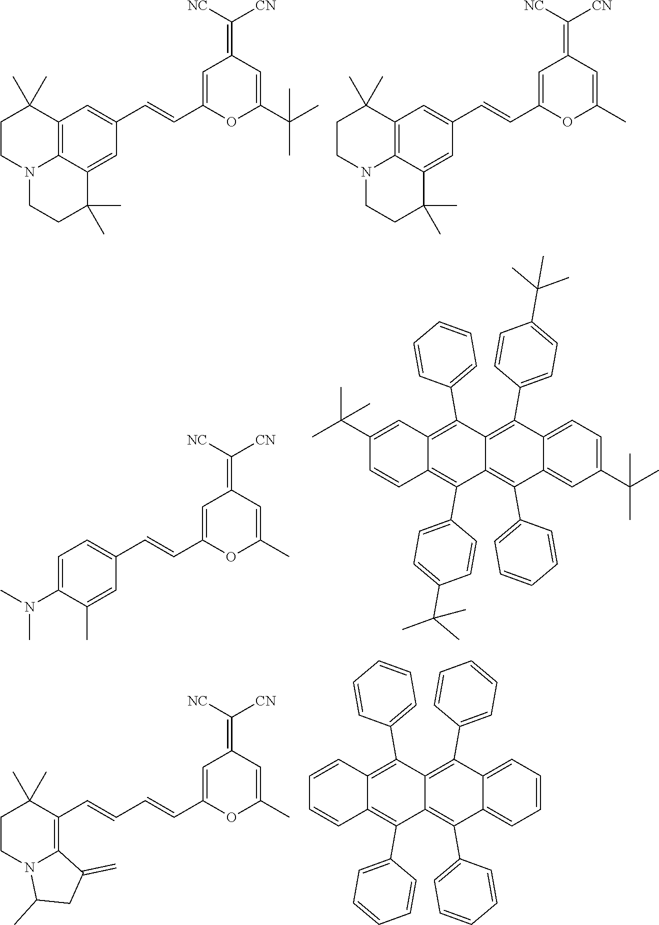

In a further embodiment of the invention, the composition has an emission peak in the visible or nearest ultraviolet range, i.e. in the range of a wavelength of from 380 to 800 nm, in particular between 590 nm and 690 nm, preferably between 610 nm and 665 nm, even more preferably between 620 nm and 640 nm.

### Light-Emitting Layer EML

In a particular embodiment, the light-emitting layer EML comprises (or essentially consists of) a composition comprising or consisting of:
(i) 0.1-10% by weight, preferably 0.5-5% by weight, in particular 1-3% by weight, of one or more organic molecules according to the invention;
(ii) 5-99% by weight, preferably 15-85% by weight, in particular 20-75% by weight, of at least one host compound H; and
(iii) 0.9-94.9% by weight, preferably 14.5-80% by weight, in particular 24-77% by weight, of at least one further host compound D with a structure differing from the structure of the molecules according to the invention; and
(iv) optionally 0-94% by weight, preferably 0-65% by weight, in particular 0-50% by weight, of a solvent; and
(v) optionally 0-30% by weight, in particular 0-20% by weight, preferably 0-5% by weight, of at least one further emitter molecule F with a structure differing from the structure of the molecules according to the invention.

In one embodiment, the light-emitting layer EML of an organic light-emitting diode of the invention comprises (or essentially consists of) a composition comprising or consisting of:
(i) 1-50% by weight, preferably 5-40% by weight, in particular 10-30% by weight, of one or more organic molecules according to the invention;
(ii) 5-99% by weight, preferably 30-94.9% by weight, in particular 40-89% by weight, of at least one host compound H; and
(iii) optionally 0-94% by weight, preferably 0.1-65% by weight, in particular 1-50% by weight, of at least one further host compound D with a structure differing from the structure of the molecules according to the invention; and
(iv) optionally 0-94% by weight, preferably 0-65% by weight, in particular 0-50% by weight, of a solvent; and
(v) optionally 0-30% by weight, in particular 0-20% by weight, preferably 0-5% by weight, of at least one further emitter molecule F with a structure differing from the structure of the molecules according to the invention.

Preferably, energy can be transferred from the host compound H to the one or more organic molecules according to the invention, in particular transferred from the first excited triplet state T1(H) of the host compound H to the first excited triplet state T1(E) of the one or more organic molecules according to the invention E and/or from the first excited singlet state S1(H) of the host compound H to the first excited singlet state S1(E) of the one or more organic molecules according to the invention E.

In one embodiment, the host compound H has a highest occupied molecular orbital HOMO(H) having an energy E^{HOMO}(H) in the range of from −5 to −6.5 eV and the at least one further host compound D has a highest occupied molecular orbital HOMO(D) having an energy E^{HOMO}(D), wherein E^{HOMO}(H)>E^{HOMO}(D).

In a further embodiment, the host compound H has a lowest unoccupied molecular orbital LUMO(H) having an energy E^{LUMO}(H) and the at least one further host compound D has a lowest unoccupied molecular orbital LUMO(D) having an energy E^{LUMO}(D), wherein E^{LUMO}(H) >E^{LUMO}(D).

### Light-Emitting Layer EML Comprising at Least One Further Host Compound D

In a further embodiment, the light-emitting layer EML of an organic light-emitting diode of the invention comprises (or essentially consists of) a composition comprising or consisting of:
(i) 1-50% by weight, preferably 5-40% by weight, in particular 10-30% by weight, of one organic molecule according to the invention;
(ii) 5-99% by weight, preferably 30-94.9% by weight, in particular 40-89% by weight, of one host compound H; and
(iii) 0-94% by weight, preferably 0.1-65% by weight, in particular 1-50% by weight, of at least one further host compound D with a structure differing from the structure of the molecules according to the invention; and
(iv) optionally 0-94% by weight, preferably 0-65% by weight, in particular 0-50% by weight, of a solvent; and
(v) optionally 0-30% by weight, in particular 0-20% by weight, preferably 0-5% by weight, of at least one further emitter molecule F with a structure differing from the structure of the molecules according to the invention.

In one embodiment of the organic light-emitting diode of the invention, the host compound H has a highest occupied molecular orbital HOMO(H) having an energy E^{HOMO}(H) in the range of from −5 eV to −6.5 eV and the at least one further host compound D has a highest occupied molecular orbital HOMO(D) having an energy E^{HOMO}(D), wherein E^{HOMO}(H) >E^{HOMO}(D). The relation E^{HOMO}(H) >E^{HOMO}(D) favors an efficient hole transport.

In a further embodiment, the host compound H has a lowest unoccupied molecular orbital LUMO(H) having an energy E^{LUMO}(H) and the at least one further host compound D has a lowest unoccupied molecular orbital LUMO(D) having an energy E^{LUMO}(D), wherein E^{LUMO}(H) >E^{LUMO}(D). The relation E^{LUMO}(H) >E^{LUMO}(D) favors an efficient electron transport.

In one embodiment, the host compound H has a highest occupied molecular orbital HOMO(H) having an energy E^{HOMO}(H) and a lowest unoccupied molecular orbital LUMO(H) having an energy E^{LUMO}(H), and
the at least one further host compound D has a highest occupied molecular orbital HOMO(D) having an energy E^{HOMO}(D) and a lowest unoccupied molecular orbital LUMO(D) having an energy E^{LUMO}(D)
the organic molecule according to the invention E has a highest occupied molecular orbital HOMO(E) having an energy E^{HOMO}(E) and a lowest unoccupied molecular orbital LUMO(E) having an energy E^{LUMO}(E),
   wherein

E^{HOMO}(H) >E^{HOMO}(D) and the difference between the energy level of the highest occupied molecular orbital HOMO(E) of the organic molecule according to the invention E (E^{HOMO}(E)) and the energy level of the highest occupied molecular orbital HOMO(H) of the host compound H (E^{HOMO}(H)) is between −0.5 eV and 0.5 eV, more preferably between −0.3 eV and 0.3 eV, even more preferably between −0.2 eV and 0.2 eV or even between −0.1 eV and 0.1 eV; and E^{LUMO}(H) >E^{LUMO}(D) and the difference between the energy level of the lowest unoccupied molecular orbital LUMO(E) of the organic molecule according to the invention E (E^{LUMO}(E)) and the lowest unoccupied molecular orbital LUMO(D) of the at least one further host compound D (E^{LUMO}(D)) is between −0.5 eV and 0.5 eV, more preferably between −0.3 eV and 0.3 eV, even more preferably between −0.2 eV and 0.2 eV or even between −0.1 eV and 0.1 eV.

In one embodiment of the invention the host compound D and/or the host compound H is a thermally-activated delayed fluorescence (TADF)-material. TADF materials exhibit a ΔE_{ST} value, which corresponds to the energy difference between the first excited singlet state (S1) and the first excited triplet state (T1), of less than 2500 cm⁻¹. Preferably the TADF material exhibits a ΔE_{ST} value of less than 3000 cm⁻¹, more preferably less than 1500 cm⁻¹, even more preferably less than 1000 cm⁻¹ or even less than 500 cm⁻¹.

In one embodiment, the host compound D is a TADF material and the host compound H exhibits a ΔE_{ST} value of more than 2500 cm⁻¹. In a particular embodiment, the host compound D is a TADF material and the host compound H is selected from group consisting of CBP, mCP, mCBP, 9-[3-(dibenzofuran-2-yl)phenyl]-9H-carbazole, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole and 9-[3,5-bis(2-dibenzothiophenyl)phenyl]-9H-carbazole.

In one embodiment, the host compound H is a TADF material and the host compound D exhibits a ΔE_{ST} value of more than 2500 cm⁻¹. In a particular embodiment, the host compound H is a TADF material and the host compound D is selected from group consisting of T2T (2,4,6-tris(biphenyl-3-yl)-1,3,5-triazine), T3T (2,4,6-tris(triphenyl-3-yl)-1,3,5-triazine) and/or TST (2,4,6-tris(9,9′-spirobifluorene-2-yl)-1,3,5-triazine).

### Light-Emitting Layer EML Comprising at Least One Further Emitter Molecule F

In a further embodiment, the light-emitting layer EML comprises (or (essentially) consists of) a composition comprising or consisting of:
(i) 1-50% by weight, preferably 5-40% by weight, in particular 10-30% by weight, of one organic molecule according to the invention;
(ii) 5-98% by weight, preferably 30-93.9% by weight, in particular 40-88% by weight, of one host compound H;
(iii) 1-30% by weight, in particular 1-20% by weight, preferably 1-5% by weight, of at least one further emitter molecule F with a structure differing from the structure of the molecules according to the invention; and
(iv) optionally 0-94% by weight, preferably 0.1-65% by weight, in particular 1-50% by weight, of at least one further host compound D with a structure differing from the structure of the molecules according to the invention; and
(v) optionally 0-94% by weight, preferably 0-65% by weight, in particular 0-50% by weight, of a solvent.

In a further embodiment, the light-emitting layer EML comprises (or (essentially) consists of) a composition as described in Compositions with at least one further emitter, with the at least one further emitter molecule F as defined in Composition wherein the at least one further emitter molecule F is a green fluorescence emitter.

In a further embodiment, the light-emitting layer EML comprises (or (essentially) consists of) a composition as described in Compositions with at least one further emitter, with the at least one further emitter molecule F as defined in Composition wherein the at least one further emitter molecule F is a red fluorescence emitter.

In one embodiment of the light-emitting layer EML comprising at least one further emitter molecule F, energy can be transferred from the one or more organic molecules of the invention E to the at least one further emitter molecule F, in particular transferred from the first excited singlet state S1(E) of one or more organic molecules of the invention E to the first excited singlet state S1(F) of the at least one further emitter molecule F.

In one embodiment, the first excited singlet state S1(H) of one host compound H of the light-emitting layer is higher in energy than the first excited singlet state S1(E) of the one or more organic molecules of the invention E: S1(H) >S1(E), and the first excited singlet state S1(H) of one host compound H is higher in energy than the first excited singlet state S1(F) of the at least one emitter molecule F: S1(H) >S1(F).

In one embodiment, the first excited triplet state T1(H) of one host compound H is higher in energy than the first excited triplet state T1(E) of the one or more organic molecules of the invention E: T1(H) >T1(E), and the first excited triplet state T1(H) of one host compound H is higher in energy than the first excited triplet state T1(F) of the at least one emitter molecule F: T1(H) >T1(F).

In one embodiment, the first excited singlet state S1(E) of the one or more organic molecules of the invention E is higher in energy than the first excited singlet state S1(F) of the at least one emitter molecule F: S1(E) >S1(F).

In one embodiment, the first excited triplet state T1(E) of the one or more organic molecules E of the invention is higher in energy than the first excited singlet state T1(F) of the at least one emitter molecule F: T1(E) >T1(F).

In one embodiment, the first excited triplet state T1(E) of the one or more organic molecules E of the invention is higher in energy than the first excited singlet state T1(F) of the at least one emitter molecule F: T1(E) >T1(F), wherein the absolute value of the energy difference between T1(E) and T1(F) is larger than 0.3 eV, preferably larger than 0.4 eV, or even larger than 0.5 eV.

In one embodiment, the host compound H has a highest occupied molecular orbital HOMO(H) having an energy E^{HOMO}(H) and a lowest unoccupied molecular orbital LUMO(H) having an energy E^{LUMO}(H), and
the one organic molecule according to the invention E has a highest occupied molecular orbital HOMO(E) having an energy E^{HOMO}(E) and a lowest unoccupied molecular orbital LUMO(E) having an energy E^{LUMO}(E),
the at least one further emitter molecule F has a highest occupied molecular orbital HOMO(F) having an energy E^{HOMO}(F) and a lowest unoccupied molecular orbital LUMO(E) having an energy E^{LUMO}(F),
   wherein
   E^{HOMO}(H) >E^{HOMO}(E) and the difference between the energy level of the highest occupied molecular orbital HOMO(F) of the at least one further emitter molecule (E^{HOMO}(F)) and the energy level of the highest occupied molecular orbital HOMO(H) of the host compound H (E^{HOMO}(H)) is between 0.5 eV and 0.5 eV, more preferably between −0.3 eV and 0.3 eV, even more preferably between −0.2 eV and 0.2 eV or even between −0.1 eV and 0.1 eV; and
   E^{LUMO}(H) >E^{LUMO}(E) and the difference between the energy level of the lowest unoccupied molecular orbital LUMO(F) of the at least one further emitter molecule (E^{LUMO}(F)) and the lowest unoccupied molecular orbital LUMO(E) of the one organic molecule according to the invention (E^{LUMO}(E)) is between −0.5 eV and 0.5 eV, more preferably between −0.3 eV and 0.3 eV, even more preferably between −0.2 eV and 0.2 eV or even between −0.1 eV and 0.1 eV.

### Optoelectronic Devices

In a further aspect, the invention relates to an optoelectronic device comprising an organic molecule or a composition of the type described here, more particularly in the form of a device selected from the group consisting of organic light-emitting diode (OLED), light-emitting electrochemical cell, OLED sensor, more particularly gas and vapour sensors not hermetically externally shielded, organic diode, organic solar cell, organic transistor, organic field-effect transistor, organic laser and down-conversion element.

In a preferred embodiment, the optoelectronic device is a device selected from the group consisting of an organic light emitting diode (OLED), a light emitting electrochemical cell (LEC), and a light-emitting transistor.

In one embodiment of the optoelectronic device of the invention, the organic molecule according to the invention E is used as emission material in a light-emitting layer EML.

In one embodiment of the optoelectronic device of the invention, the light-emitting layer EML consists of the composition according to the invention described here.

When the optoelectronic device is an OLED, it may, for example, have the following layer structure:

1. substrate
2. anode layer A
3. hole injection layer, HIL
4. hole transport layer, HTL
5. electron blocking layer, EBL
6. emitting layer, EML
7. hole blocking layer, HBL
8. electron transport layer, ETL
9. electron injection layer, EIL
10. cathode layer,
wherein the OLED comprises each layer selected from the group of HIL, HTL, EBL, HBL, ETL, and EIL only optionally, different layers may be merged and the OLED may comprise more than one layer of each layer type defined above.

Furthermore, the optoelectronic device may, in one embodiment, comprise one or more protective layers protecting the device from damaging exposure to harmful species in the environment including, for example, moisture, vapor and/or gases.

In one embodiment of the invention, the optoelectronic device is an OLED, with the following inverted layer structure:

1. substrate
2. cathode layer
3. electron injection layer, EIL
4. electron transport layer, ETL
5. hole blocking layer, HBL
6. emitting layer, B
7. electron blocking layer, EBL
8. hole transport layer, HTL
9. hole injection layer, HIL
10. anode layer A
wherein the OLED comprises each layer selected from the group of HIL, HTL, EBL, HBL, ETL, and EIL only optionally, different layers may be merged and the OLED may comprise more than one layer of each layer types defined above.

In one embodiment of the invention, the optoelectronic device is an OLED, which may have a stacked architecture. In this architecture, contrary to the typical arrangement in which the OLEDs are placed side by side, the individual units are stacked on top of each other. Blended light may be generated with OLEDs exhibiting a stacked architecture, in particular white light may be generated by stacking blue, green and red OLEDs. Furthermore, the OLED exhibiting a stacked architecture may comprise a charge generation layer (CGL), which is typically located between two OLED subunits and typically consists of a n-doped and p-doped layer with the n-doped layer of one CGL being typically located closer to the anode layer.

In one embodiment of the invention, the optoelectronic device is an OLED, which comprises two or more emission layers between anode and cathode. In particular, this so-called tandem OLED comprises three emission layers, wherein one emission layer emits red light, one emission layer emits green light and one emission layer emits blue light, and optionally may comprise further layers such as charge generation layers, blocking or transporting layers between the individual emission layers. In a further embodiment, the emission layers are adjacently stacked. In a further embodiment, the tandem OLED comprises a charge generation layer between each two emission layers. In addition, adjacent emission layers or emission layers separated by a charge generation layer may be merged.

The substrate may be formed by any material or composition of materials. Most frequently, glass slides are used as substrates. Alternatively, thin metal layers (e.g., copper, gold, silver or aluminum films) or plastic films or slides may be used. This may allow for a higher degree of flexibility. The anode layer A is mostly composed of materials allowing to obtain an (essentially) transparent film. As at least one of both electrodes should be (essentially) transparent in order to allow light emission from the OLED, either the anode layer A or the cathode layer C is transparent. Preferably, the anode layer A comprises a large content or even consists of transparent conductive oxides (TCOs). Such anode layer A may, for example, comprise indium tin oxide, aluminum zinc oxide, fluorine doped tin oxide, indium zinc oxide, PbO, SnO, zirconium oxide, molybdenum oxide, vanadium oxide, tungsten oxide, graphite, doped Si, doped Ge, doped GaAs, doped polyaniline, doped polypyrrol and/or doped polythiophene.

The anode layer A (essentially) may consist of indium tin oxide (ITO) (e.g., (InO₃)_{0.9}(SnO₂)_{0.1}). The roughness of the anode layer A caused by the transparent conductive oxides (TCOs) may be compensated by using a hole injection layer (HIL). Further, the HIL may facilitate the injection of quasi charge carriers (i.e., holes) in that the transport of the quasi charge carriers from the TCO to the hole transport layer (HTL) is facilitated. The hole injection layer (HIL) may comprise poly-3,4-ethylendioxy thiophene (PEDOT), polystyrene sulfonate (PSS), MoO₂, V₂O₅, CuPC or CuI, in particular a mixture of PEDOT and PSS. The hole injection layer (HIL) may also prevent the diffusion of metals from the anode layer A into the hole transport layer (HTL). The HIL may, for example, comprise PEDOT:PSS (poly-3,4-ethylendioxy thiophene: polystyrene sulfonate), PEDOT (poly-3,4-ethylendioxy thiophene), mMTDATA (4,4′,4″-tris[phenyl(m-tolyl)amino]triphenylamine), Spiro-TAD (2,2′,7,7′-tetrakis(n,n-diphenylamino)-9,9′-spirobifluorene), DNTPD (N1,N1′-(biphenyl-4,4′-diyl)bis(N1-phenyl-N4,N4-di-m-tolylbenzene-1,4-diamine), NPB (N,N′-nis-(1-naphthalenyl)-N,N′-bis-phenyl-(1,1′-biphenyl)-4,4′-diamine), NPNPB (N,N′-diphenyl-N,N′-di-[4-(N,N-diphenyl-amino)phenyl]benzidine), MeO-TPD (N,N,N′,N′-tetrakis(4-methoxyphenyl)benzidine), HAT-CN (1,4,5,8,9,11-hexaazatriphenylen-hexacarbonitrile) and/or Spiro-NPD (N,N′-diphenyl-N,N′-bis-(1-naphthyl)-9,9′-spirobifluorene-2,7-diamine).

Adjacent to the anode layer A or hole injection layer (HIL), a hole transport layer (HTL) is typically located. Herein, any hole transport compound may be used. For example, electron-rich heteroaromatic compounds such as triarylamines and/or carbazoles may be used as hole transport compound. The HTL may decrease the energy barrier between the anode layer A and the light-emitting layer EML. The hole transport layer (HTL) may also be an electron blocking layer (EBL). Preferably, hole transport compounds bear comparably high energy levels of their triplet states T1. For example, the hole transport layer (HTL) may comprise a star-shaped heterocycle such as tris(4-carbazoyl-9-ylphenyl)amine (TCTA), poly-TPD (poly(4-butylphenyl-diphenyl-amine)), [alpha]-NPD (poly(4-butylphenyl-diphenyl-amine)), TAPC (4,4′-cyclohexyliden-bis[N,N-bis(4-methylphenyl)benzenamine]), 2-TNATA (4,4′,4″-tris[2-naphthyl(phenyl)amino]triphenylamine), Spiro-TAD, DNTPD, NPB, NPNPB, MeO-TPD, HAT-CN and/or TrisPcz (9,9′-diphenyl-6-(9-phenyl-9H-carbazol-3-yl)-9H,9′H-3,3′-bicarbazole). In addition, the HTL may comprise a p-doped layer, which may be composed of an inorganic or organic dopant in an organic hole-transporting matrix. Transition metal oxides such as vanadium oxide, molybdenum oxide or tungsten oxide may, for example, be used as inorganic dopant. Tetrafluorotetracyanoquinodimethane (F₄-TCNQ), copper-pentafluorobenzoate (Cu(I)pFBz) or transition metal complexes may, for example, be used as organic dopant.

The EBL may, for example, comprise mCP (1,3-bis(carbazol-9-yl)benzene), TCTA, 2-TNATA, mCBP (3,3-di(9H-carbazol-9-yl)biphenyl), tris-Pcz, CzSi (9-(4-tert-Butylphenyl)-3,6-bis(triphenylsilyl)-9H-carbazole), and/or DCB (N,N′-dicarbazolyl-1,4-dimethylbenzene).

Adjacent to the hole transport layer (HTL), the light-emitting layer EML is typically located. The light-emitting layer EML comprises at least one light emitting molecule. Particularly, the EML comprises at least one light emitting molecule according to the invention E. In one embodiment, the light-emitting layer comprises only the organic molecules according to the invention. Typically, the EML additionally comprises one or more host materials H. For example, the host material H is selected from CBP (4,4′-Bis-(N-carbazolyl)-biphenyl), mCP, mCBP Sif87 (dibenzo[b,d]thiophen-2-yltriphenylsilane), CzSi, Sif88 (dibenzo[b,d]thiophen-2-yl)diphenylsilane), DPEPO (bis[2-(diphenylphosphino)phenyl] ether oxide), 9-[3-(dibenzofuran-2-yl)phenyl]-9H-carbazole, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole, 9-[3,5-bis(2-dibenzothiophenyl)phenyl]-9H-carbazole, T2T (2,4,6-tris(biphenyl-3-yl)-1,3,5-triazine), T3T (2,4,6-tris(triphenyl-3-yl)-1,3,5-triazine) and/or TST (2,4,6-tris(9,9′-spirobifluorene-2-yl)-1,3,5-triazine). The host material H typically should be selected to exhibit first triplet (T1) and first singlet (S1) energy levels, which are energetically higher than the first triplet (T1) and first singlet (S1) energy levels of the organic molecule.

In one embodiment of the invention, the EML comprises a so-called mixed-host system with at least one hole-dominant host and one electron-dominant host. In a particular embodiment, the EML comprises exactly one light emitting organic molecule according to the invention and a mixed-host system comprising T2T as electron-dominant host and a host selected from CBP, mCP, mCBP, 9-[3-(dibenzofuran-2-yl)phenyl]-9H-carbazole, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole and 9-[3,5-bis(2-dibenzothiophenyl)phenyl]-9H-carbazole as hole-dominant host. In a further embodiment the EML comprises 50-80% by weight, preferably 60-75% by weight of a host selected from CBP, mCP, mCBP, 9-[3-(dibenzofuran-2-yl)phenyl]-9H-carbazole, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole and 9-[3,5-bis(2-dibenzothiophenyl)phenyl]-9H-carbazole; 10-45% by weight, preferably 15-30% by weight of T2T and 5-40% by weight, preferably 10-30% by weight of light emitting molecule according to the invention.

Adjacent to the light-emitting layer EML, an electron transport layer (ETL) may be located. Herein, any electron transporter may be used. Exemplarily, electron-poor compounds such as, e.g., benzimidazoles, pyridines, triazoles, oxadiazoles (e.g., 1,3,4-oxadiazole), phosphinoxides and sulfone, may be used. An electron transporter may also be a star-shaped heterocycle such as 1,3,5-tri(1-phenyl-1H-benzo[d]imidazol-2-yl)phenyl (TPBi). The ETL may comprise NBphen (2,9-bis(naphthalen-2-yl)-4,7-diphenyl-1,10-phenanthroline), PYD2 (2,6-bis(9H-carbazol-9-yl)pyridine), Alq₃ (aluminum-tris(8-hydroxyquinoline)), TSPO1 (diphenyl-4-triphenylsilylphenyl-phosphinoxide), BPyTP2 (2,7-di(2,2′-bipyridin-5-yl)triphenyle), Sif87 (dibenzo[b,d]thiophen-2-yltriphenylsilane), Sif88 (dibenzo[b,d]thiophen-2-yl)diphenylsilane), BmPyPhB (1,3-bis[3,5-di(pyridin-3-yl)phenyl]benzene) and/or BTB (4,4′-bis-[2-(4,6-diphenyl-1,3,5-triazinyl)]-1,1′-biphenyl). Optionally, the ETL may be doped with materials such as Liq. The electron transport layer (ETL) may also block holes or a holeblocking layer (HBL) is introduced.

The HBL may, for example, comprise BCP (2,9-dimethyl-4,7-diphenyl-1,10-phenanthroline=Bathocuproine), BAlq (bis(8-hydroxy-2-methylquinoline)-(4-phenylphenoxy)aluminum), NBphen (2,9-bis(naphthalen-2-yl)-4,7-diphenyl-1,10-phenanthroline), Alq₃ (Aluminum-tris(8-hydroxyquinoline)), TSPO1 (diphenyl-4-triphenylsilylphenyl-phosphinoxide), T2T (2,4,6-tris(biphenyl-3-yl)-1,3,5-triazine), T3T (2,4,6-tris(triphenyl-3-yl)-1,3,5-triazine), TST (2,4,6-tris(9,9′-spirobifluorene-2-yl)-1,3,5-triazine), and/or TCB/TCP (1,3,5-tris(N-carbazolyl)benzol/1,3,5-tris(carbazol)-9-yl) benzene).

Adjacent to the electron transport layer (ETL), a cathode layer C may be located. The cathode layer C may, for example, comprise or may consist of a metal (e.g., Al, Au, Ag, Pt, Cu, Zn, Ni, Fe, Pb, LiF, Ca, Ba, Mg, In, W, or Pd) or a metal alloy. For practical reasons, the cathode layer may also consist of (essentially) intransparent metals such as Mg, Ca or Al. Alternatively or additionally, the cathode layer C may also comprise graphite and or carbon nanotubes (CNTs). Alternatively, the cathode layer C may also consist of nanoscalic silver wires.

An OLED may further, optionally, comprise a protection layer between the electron transport layer (ETL) and the cathode layer C (which may be designated as electron injection layer (EIL)). This layer may comprise lithium fluoride, cesium fluoride, silver, Liq (8-hydroxyquinolinolatolithium), Li₂O, BaF₂, MgO and/or NaF.

Optionally, the electron transport layer (ETL) and/or a hole blocking layer (HBL) may also comprise one or more host compounds H.

In order to modify the emission spectrum and/or the absorption spectrum of the light-emitting layer EML further, the light-emitting layer EML may further comprise one or more further emitter molecules F. Such an emitter molecule F may be any emitter molecule known in the art. Preferably such an emitter molecule F is a molecule with a structure differing from the structure of the molecules according to the invention E. The emitter molecule F may optionally be a TADF emitter. Alternatively, the emitter molecule F may optionally be a fluorescent and/or phosphorescent emitter molecule which is able to shift the emission spectrum and/or the absorption spectrum of the light-emitting layer EML. Exemplarily, the triplet and/or singlet excitons may be transferred from the organic emitter molecule according to the invention to the emitter molecule F before relaxing to the ground state S0 by emitting light typically red-shifted in comparison to the light emitted by an organic molecule. Optionally, the emitter molecule F may also provoke two-photon effects (i.e., the absorption of two photons of half the energy of the absorption maximum).

Optionally, an optoelectronic device (e.g., an OLED) may, for example, be an essentially white optoelectronic device. For example, such white optoelectronic device may comprise at least one (deep) blue emitter molecule and one or more emitter molecules emitting green and/or red light. Then, there may also optionally be energy transmittance between two or more molecules as described above.

As used herein, if not defined more specifically in the particular context, the designation of the colors of emitted and/or absorbed light is as follows:

violet: wavelength range of >380-420 nm;
deep blue: wavelength range of >420-480 nm;
sky blue: wavelength range of >480-500 nm;
green: wavelength range of >500-560 nm;
yellow: wavelength range of >560-580 nm;
orange: wavelength range of >580-620 nm;
red: wavelength range of >620-800 nm.

With respect to emitter molecules, such colors refer to the emission maximum. Therefore, for example, a deep blue emitter has an emission maximum in the range of from >420 to 480 nm, a sky blue emitter has an emission maximum in the range of from >480 to 500 nm, a green emitter has an emission maximum in a range of from >500 to 560 nm, a red emitter has an emission maximum in a range of from >620 to 800 nm.

A deep blue emitter may preferably have an emission maximum of below 480 nm, more preferably below 470 nm, even more preferably below 465 nm or even below 460 nm. It will typically be above 420 nm, preferably above 430 nm, more preferably above 440 nm or even above 450 nm.

A green emitter has an emission maximum of below 560 nm, more preferably below 550 nm, even more preferably below 545 nm or even below 540 nm. It will typically be above 500 nm, more preferably above 510 nm, even more preferably above 515 nm or even above 520 nm.

A further aspect of the present invention relates to an OLED, which emits light at a distinct color point. According to the present invention, the OLED emits light with a narrow emission band (small full width at half maximum (FWHM)). In one aspect, the OLED according to the invention emits light with a FWHM of the main emission peak of less than 0.30 eV, preferably less than 0.25 eV, more preferably less than 0.20 eV, even more preferably less than 0.19 eV or even less than 0.17 eV.

A further embodiment of the present invention relates to an OLED, which emits light with CIEx and CIEy color coordinates close to the CIEx (=0.170) and CIEy (=0.797) color coordinates of the primary color green (CIEx=0.170 and CIEy=0.797) as defined by ITU-R Recommendation BT. 2020 (Rec. 2020) and thus is suited for the use in Ultra High Definition (UHD) displays, e.g. UHD-TVs. In this context, the term “close to” refers to the ranges of CIEx and CIEy coordinates provided at the end of this paragraph. In commercial applications, typically top-emitting (top-electrode is transparent) devices are used, whereas test devices as used throughout the present application represent bottom-emitting devices (bottom-electrode and substrate are transparent). Accordingly, a further aspect of the present invention relates to an OLED, whose emission exhibits a CIEx color coordinate of between 0.15 and 0.45 preferably between 0.15 and 0.35, more preferably between 0.15 and 0.30 or even more preferably between 0.15 and 0.25 or even between 0.15 and 0.20 and/or a CIEy color coordinate of between 0.60 and 0.92, preferably between 0.65 and 0.90, more preferably between 0.70 and 0.88 or even more preferably between 0.75 and 0.86 or even between 0.79 and 0.84.

Accordingly, a further aspect of the present invention relates to an OLED, which exhibits an external quantum efficiency at 14500 cd/m² of more than 10%, more preferably of more than 13%, more preferably of more than 15%, even more preferably of more than 17% or even more than 20% and/or exhibits an emission maximum between 485 nm and 560 nm, preferably between 500 nm and 560 nm, more preferably between 510 nm and 550 nm, even more preferably between 515 nm and 540 nm and/or exhibits a LT97 value at 14500 cd/m² of more than 100 h, preferably more than 250 h, more preferably more than 50 h, even more preferably more than 750 h or even more than 1000 h.

In a further aspect, the invention relates to a method for producing an optoelectronic component. In this case an organic molecule of the invention is used.

The optoelectronic device, in particular the OLED according to the present invention can be fabricated by any means of vapor deposition and/or liquid processing. Accordingly, at least one layer is
prepared by means of a sublimation process,
prepared by means of an organic vapor phase deposition process,
prepared by means of a carrier gas sublimation process,
solution processed or printed.

The methods used to fabricate the optoelectronic device, in particular the OLED according to the present invention are known in the art. The different layers are individually and successively deposited on a suitable substrate by means of subsequent deposition processes. The individual layers may be deposited using the same or differing deposition methods.

Vapor deposition processes, for example, comprise thermal (co)evaporation, chemical vapor deposition and physical vapor deposition. For active matrix OLED display, an AMOLED backplane is used as substrate. The individual layer may be processed from solutions or dispersions employing adequate solvents. Solution deposition process, for example, comprise spin coating, dip coating and jet printing. Liquid processing may optionally be carried out in an inert atmosphere (e.g., in a nitrogen atmosphere) and the solvent may be completely or partially removed by means known in the state of the art.

### EXAMPLES

### General Synthesis Scheme I

General synthesis scheme I provides a synthesis scheme for organic molecules according to the invention wherein R^{I} ═R^{IV}, R^{II}═R^{III}:

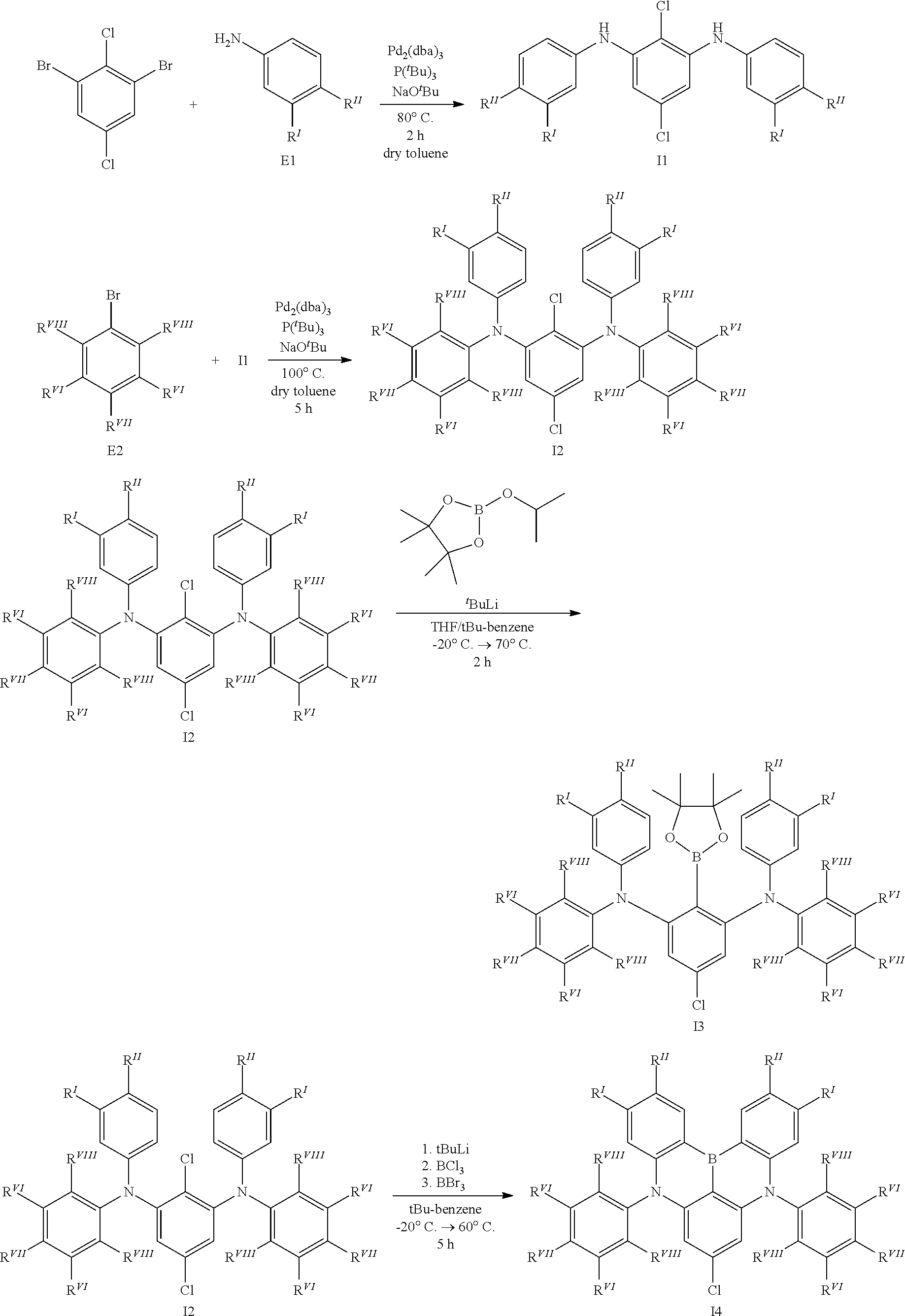

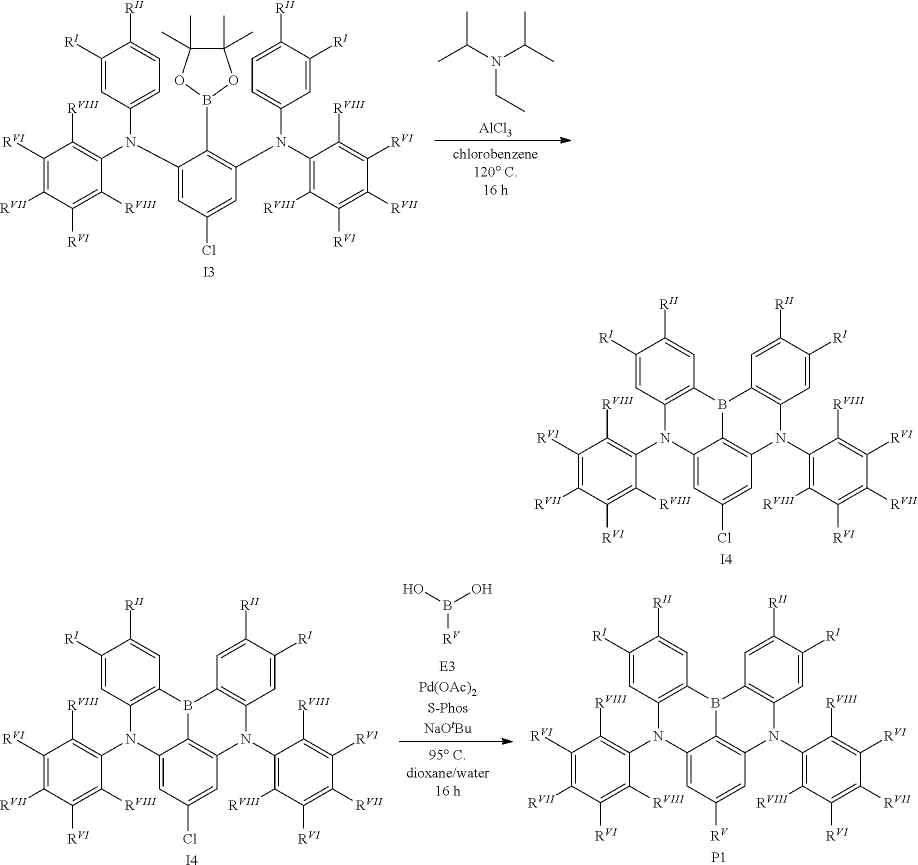

Alternative route to introduce R^{V}:

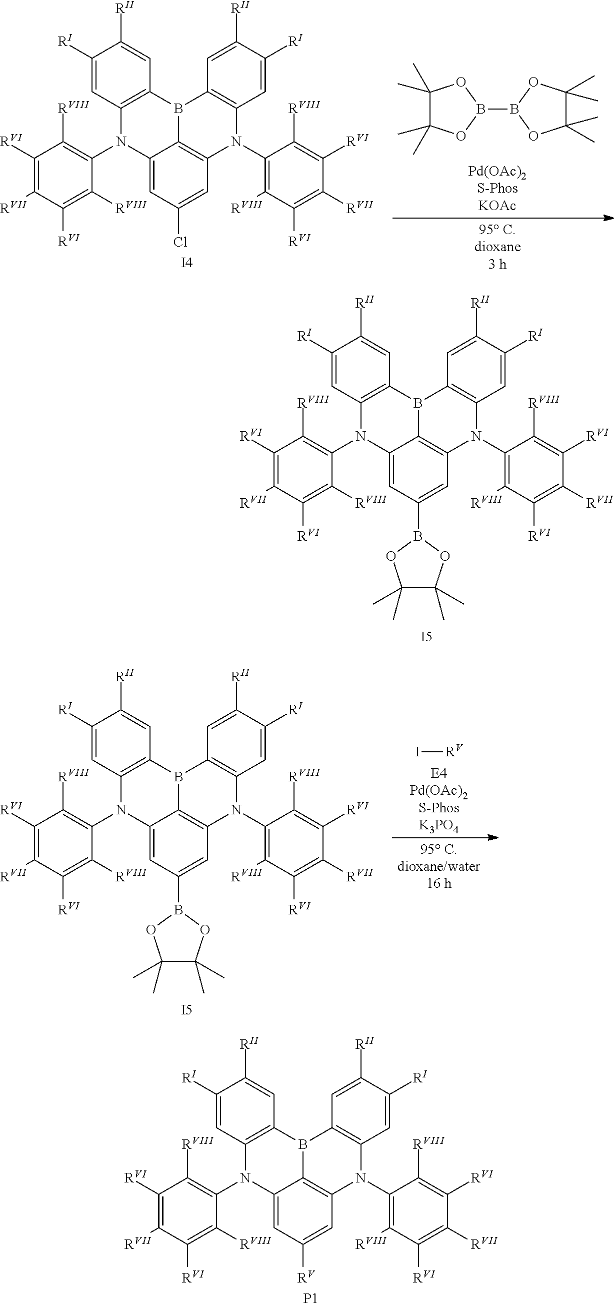

Alternative route to introduce R^{V}:

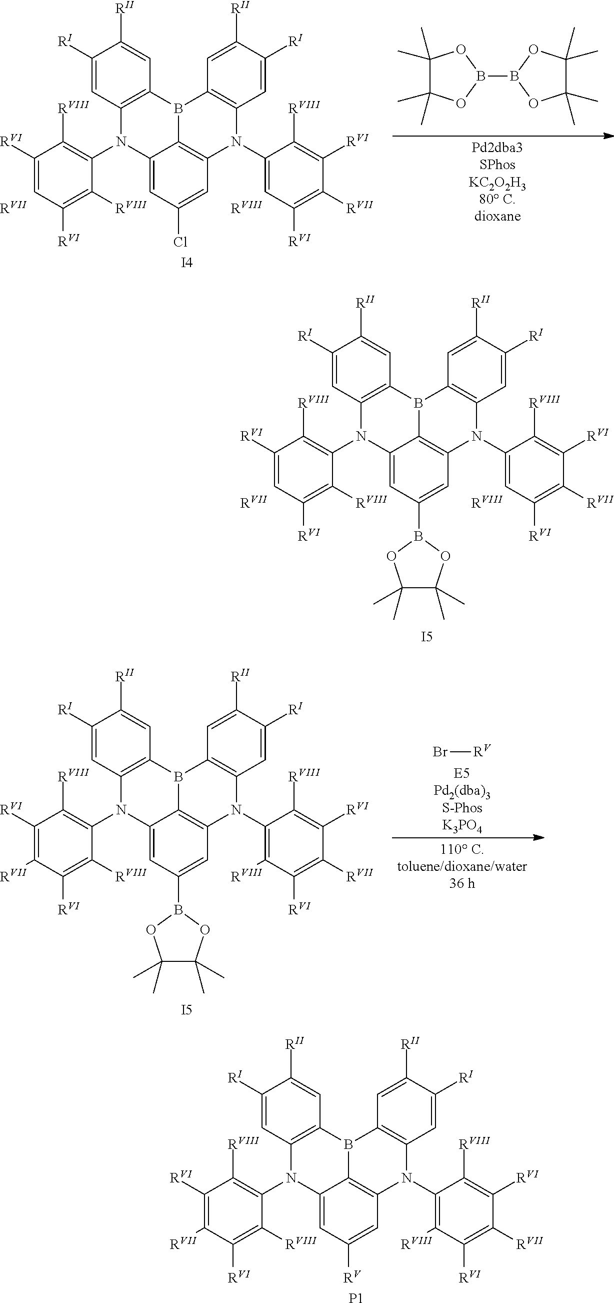

### General Procedure for Synthesis AAV1:

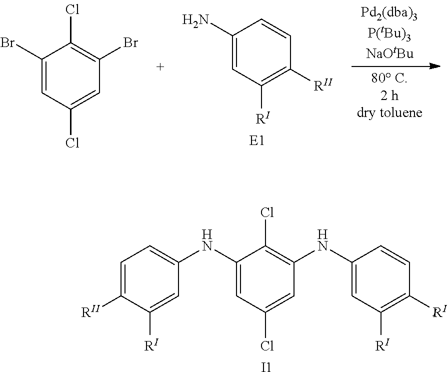

1,3-Dibromo-2,5-dichlorbenzene (CAS: 81067-41-6, 1.00 equivalents), E1 (2.20 equivalents), tris(dibenzylideneacetone)dipalladium Pd₂(dba)₃ (0.02 equivalents; CAS: 51364-51-3), tri-tert-butyl-phosphine (P(^{t}Bu)₃, CAS: 13716-12-6, 0.08 equivalents) and sodium tert-butoxide (NaO^{t}Bu; 6.00 equivalents) are stirred under nitrogen atmosphere in toluene at 80° C. for 2 h. After cooling down to room temperature (rt) the reaction mixture is extracted with toluene and brine and the phases are separated. The combined organic layers are dried over MgSO₄ and then the solvent is removed under reduced pressure. The crude product obtained is purified by recrystallization or column chromatography and I1 is obtained as solid.

### General Procedure for Synthesis AAV2:

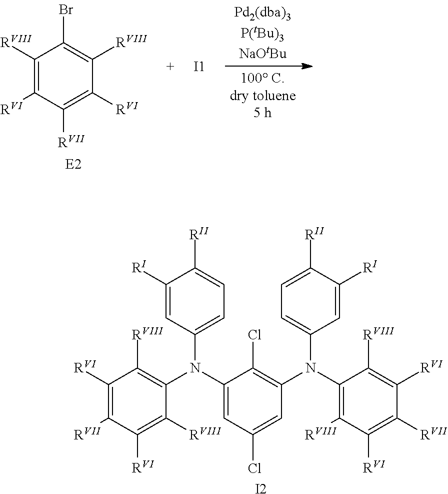

I1 (1.00 equivalents), E2 (2.20 equivalents, tris(dibenzylideneacetone)dipalladium Pd₂(dba)₃ (0.02 equivalents; CAS: 51364-51-3), tri-tert-butyl-phosphine (0.08 equivalents, P(^{t}Bu)₃, CAS: 13716-12-6) and sodium tert-butoxide (NaO^{t}Bu; 5.00 equivalents) are stirred under nitrogen atmosphere in toluene at 100° C. for 5 h. After cooling down to room temperature (rt) the reaction mixture is extracted with toluene and brine and the phases are separated. The combined organic layers are dried over MgSO₄ and then the solvent is removed under reduced pressure. The crude product obtained is purified by recrystallization or column chromatography and I2 is obtained as solid.

### General Procedure for Synthesis AAV3:

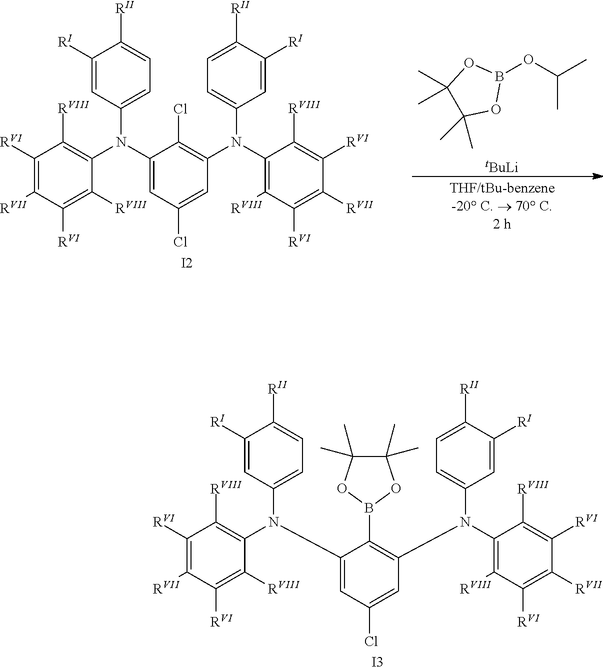

After dissolving I2 (1 equivalent) under nitrogen atmosphere in THF and cooling to −20° C. or in tert-butylbenzene and cooling to −10° C., ^{t}BuLi (2 equivalents, CAS: 594-19-4) is added and the reaction mixture is stirred at 0° C. After complete lithiation, the reaction is quenched and 1,3,2-dioxaborolane (2 equivalents, CAS: 61676-62-8) is added and the reaction mixture is stirred under reflux at 70° C. for 2 h. After cooling down to room temperature (rt), the reaction mixture is extracted between toluene and brine and the phases are separated. The combined organic layers are dried over MgSO₄ and then the solvent is removed under reduced pressure. The crude product obtained is purified by recrystallization or column chromatography and I3 is obtained as solid.

### General Procedure for Synthesis AAV4:

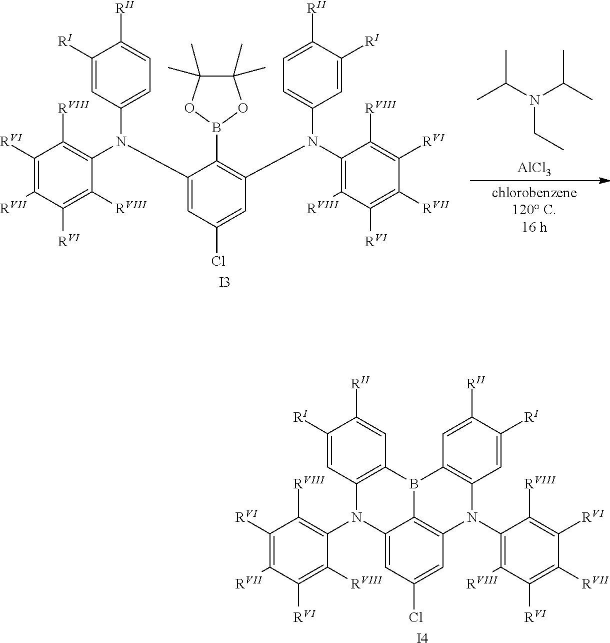

I3 (1 equivalent), N,N-diisopropylethylamine (10 equivalents, CAS: 7087-68-5) and AlCl₃ (10 equivalents, CAS: 7446-70-0) are stirred under nitrogen atmosphere in chlorobenzene at 120° C. for 16 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and I4 is obtained as solid.

### General Procedure for Synthesis AAV5:

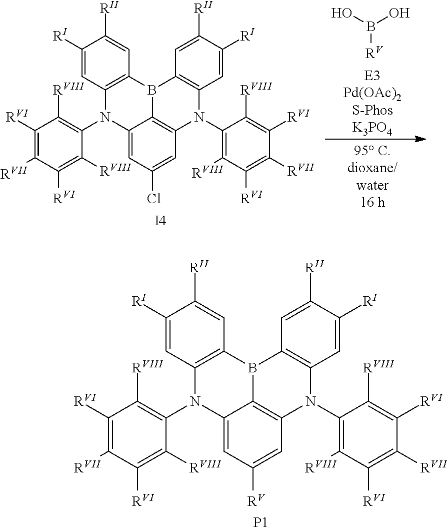

I4 (1 equivalent), E3 (1.1 equivalents), palladium(II) acetate (CAS: 3375-31-3, 0.1 equivalents), S-Phos (CAS: 657408-07-6, 0.24 equivalents) and potassium phosphate tribasic (5 equivalents) are stirred under nitrogen atmosphere in dioxane/water 5:1 at 100° C. for 16 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and P1 is obtained as solid.

### General Procedure for Synthesis AAV6:

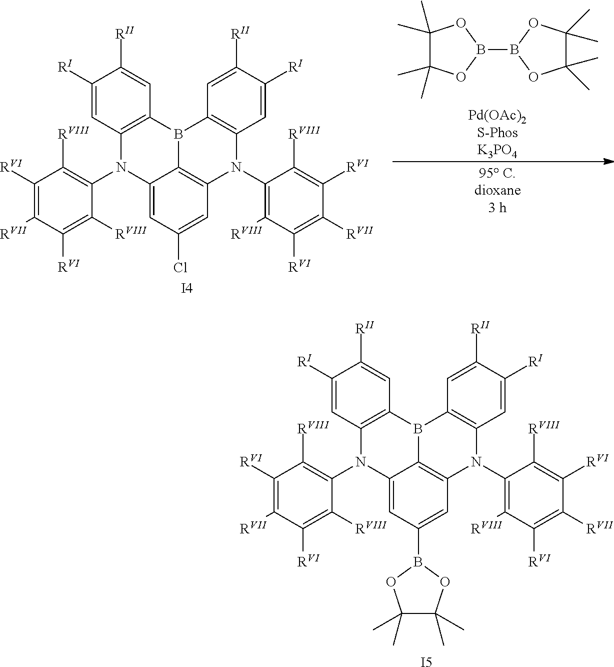

I4 (1 equivalent), bis(pinacolato)diboron (CAS: 73183-34-3, 1.5 equivalents), palladium(II) acetate (CAS: 3375-31-3, 0.1 equivalents), S-Phos (CAS: 657408-07-6, 0.2 equivalents) and potassium phosphate tribasic (K₃PO₄; 5 equivalents) are stirred under nitrogen atmosphere in dioxane at 100° C. for 3 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and I5 is obtained as solid.

### General Procedure for Synthesis AAV7:

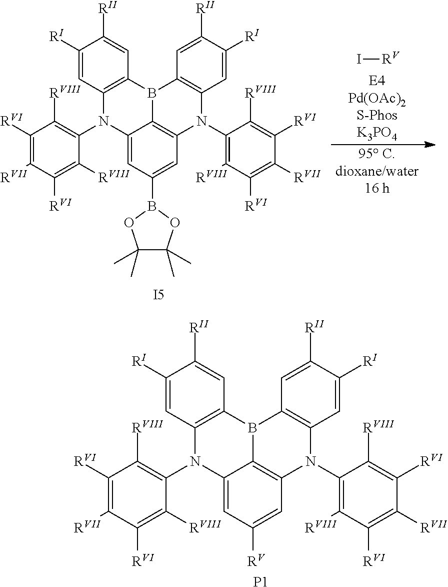

I5 (1 equivalent), E4 (1.2 equivalents), palladium(II) acetate (CAS: 3375-31-3, 0.1 equivalents), S-Phos (CAS: 657408-07-6, 0.24 equivalents) and potassium phosphate tribasic (K₃PO₄; 5 equivalents) are stirred under nitrogen atmosphere in dioxane/water 10:1 at 100° C. for 16 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and P1 is obtained as solid.

### General Procedure for Synthesis AAV8:

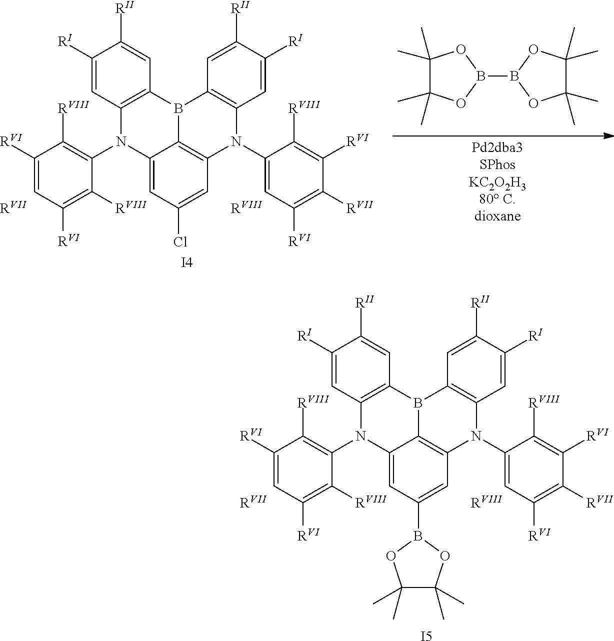

Under N₂ atmosphere, a two-necked flask is charged with I4 (1.0 equiv.), Bis(pinacolato)diboron [73183-34-3] (1.2 equiv.), Pd₂(dba)₃ [51364-51-3] (0.06 equiv.), S-Phos [657408-07-6] (0.12 equiv.) and potassium acetate [127-08-2] (3.0 equiv.). Dry Dioxane (10 mL/mmol of I4) is added and the resulting mixture degassed for 10 min. Subsequently, the mixture is heated at 80° C. overnight. After cooling down to room temperature (rt), dichloromethane and water are added, the phases separated, the aqueous layer extracted with dichloromethane and the combined organic layers dried over MgSO₄, filtered and concentrated. The crude product is purified with MPLC or recrystallization to obtain the corresponding product I5 as a solid

### General Procedure for Synthesis AAV9:

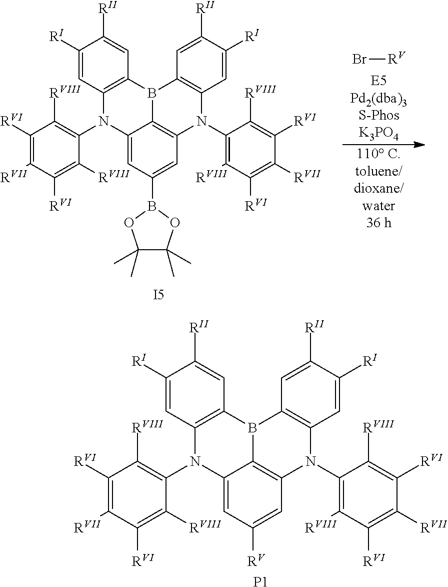

I5 (1 equivalent), E5 (1.25 equivalents), Tris(dibenzylideneacetone)dipalladium(0) (CAS: 51364-51-3, 0.04 equivalents), S-Phos (CAS: 657408-07-6, 0.08 equivalents) and potassium phosphate tribasic (K₃PO₄; 2.5 equivalents) are stirred under nitrogen atmosphere in toluene/dioxane/water 3:3:1 at 110° C. for 36 h. After cooling down to room temperature (rt), a solid precipitated, which was filtered and washed with CHCl₃. The filtrate was evaporated in vacuo to yield P1 as solid.

### General Synthesis Scheme II

General synthesis scheme II provides another synthesis scheme for organic molecules according to the invention, wherein R^{I}═R^{IV} and R^{II}═R^{III}.

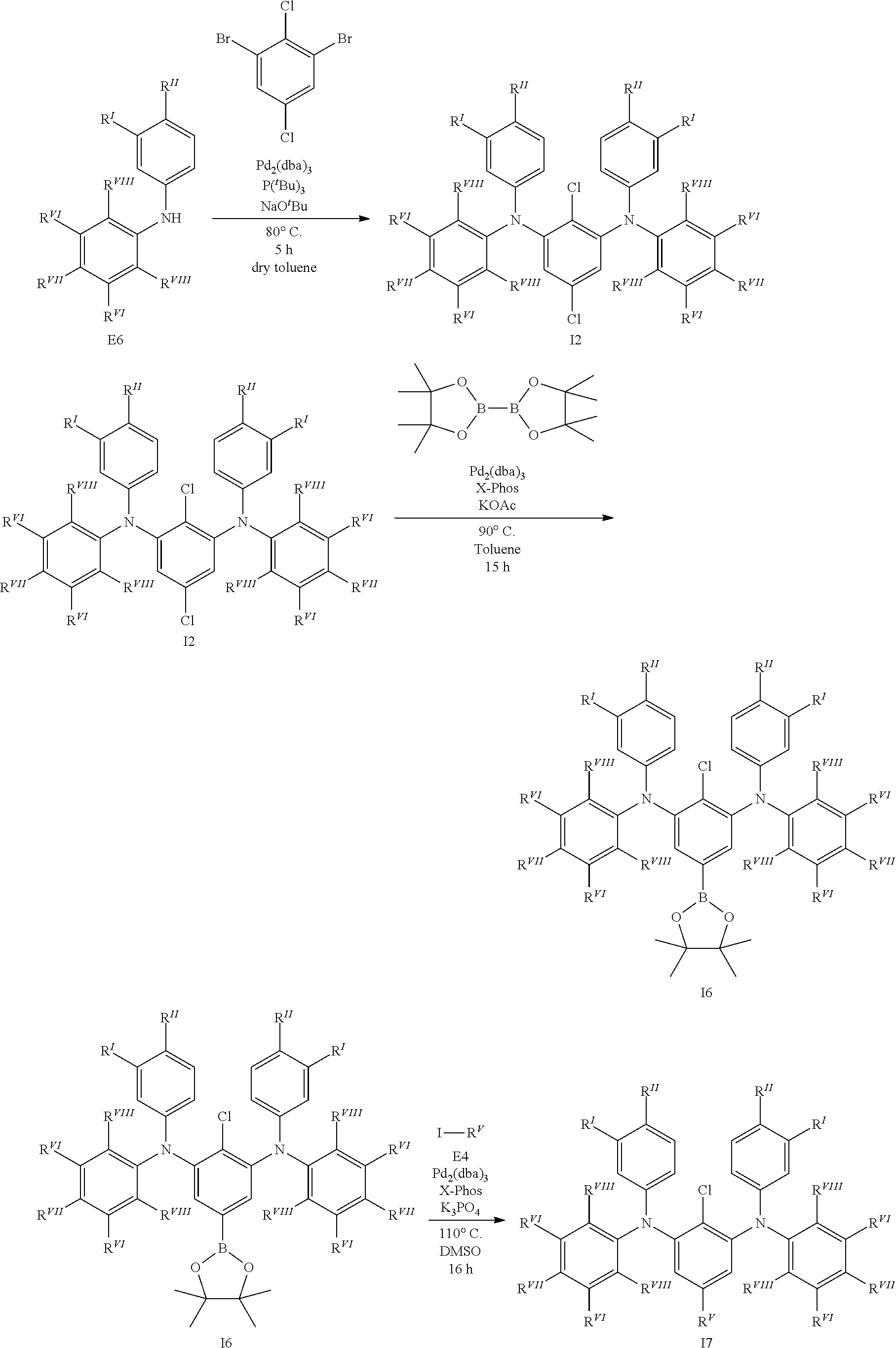

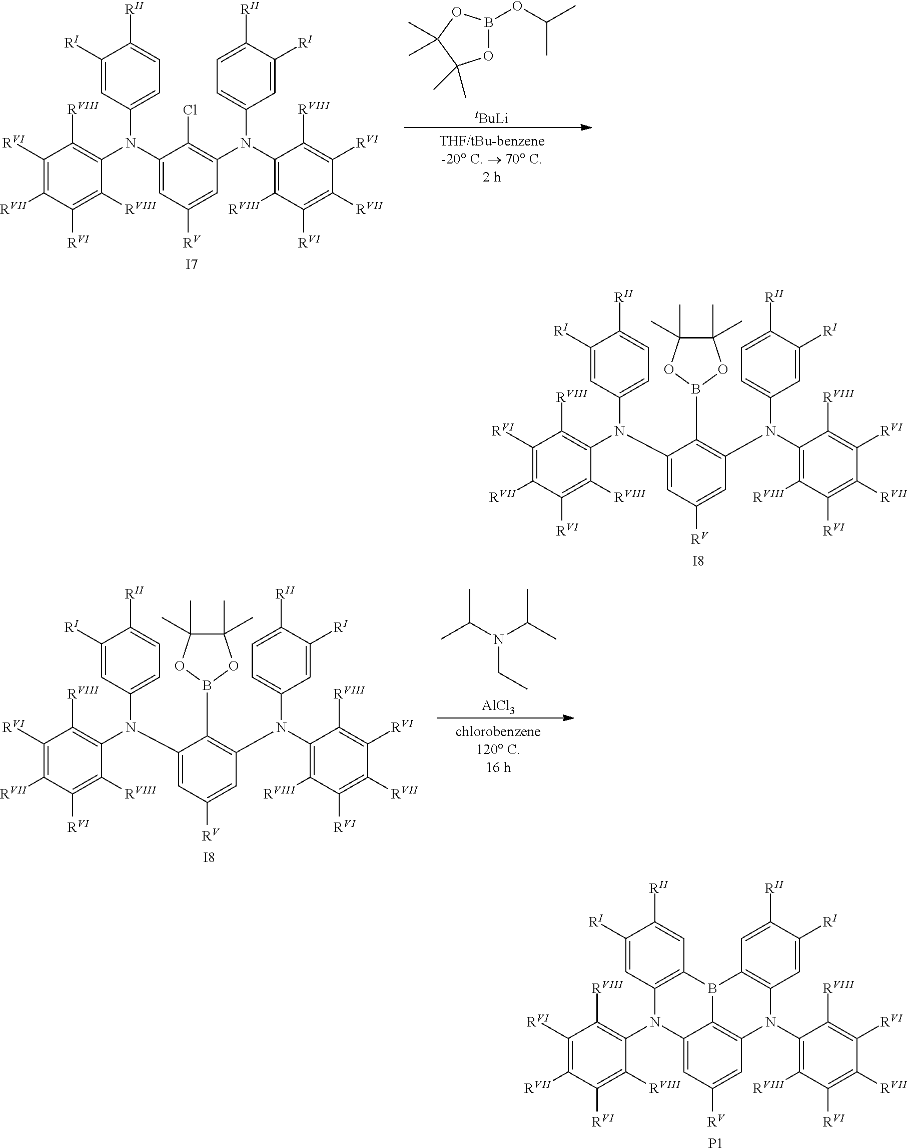

### General Procedure for Synthesis AAV10

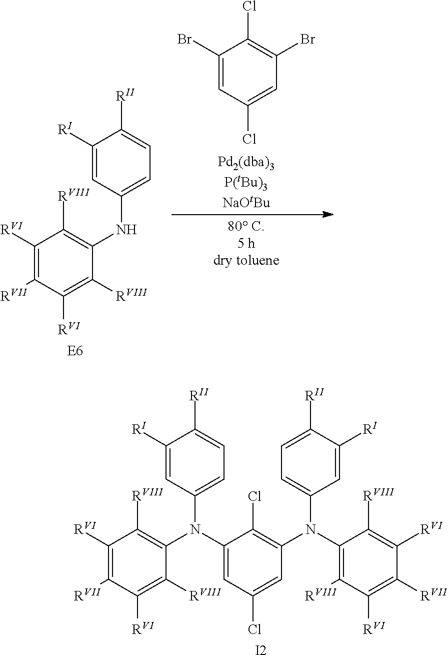

1,3-Dibromo-2,5-dichlorbenzene (CAS: 81067-41-6, 1.00 equivalents), E6 (2.10 equivalents), tris(dibenzylideneacetone)dipalladium Pd₂(dba)₃ (0.01 equivalents; CAS: 51364-51-3), tri-tert-butyl-phosphine (P(^{t}Bu)₃, CAS: 13716-12-6, 0.02 equivalents) and sodium tert-butoxide (NaO^{t}Bu; 3.00 equivalents) are stirred under nitrogen atmosphere in toluene at 80° C. for 8 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and I2 is obtained as solid.

### General Procedure for Synthesis AAV11:

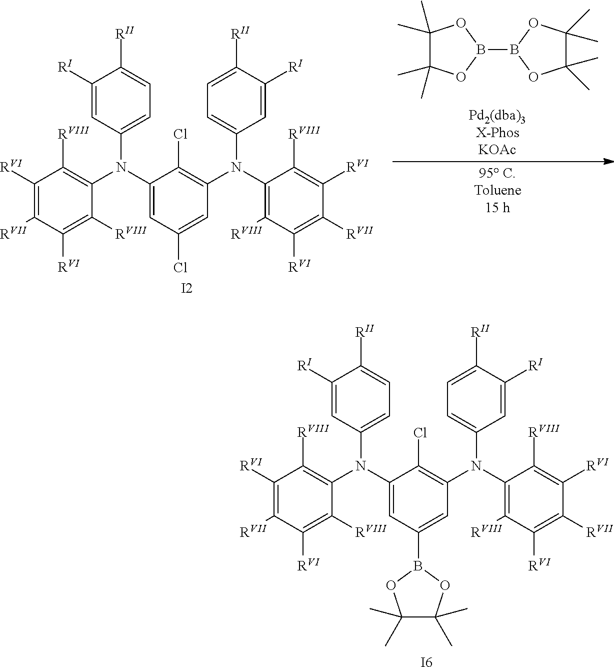

I2 (1 equivalent), bis(pinacolato)diboron (CAS: 73183-34-3, 1.5 equivalents), tris(dibenzylideneacetone)dipalladium Pd₂(dba)₃ (0.1 equivalents; CAS: 51364-51-3), X-Phos (CAS: 564483-18-7, 0.2 equivalents) and potassium acetate (4.00 equivalents) are stirred under nitrogen atmosphere in toluene at 95° C. for 16 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and I6 is obtained as solid.

### General Procedure for Synthesis AAV12:

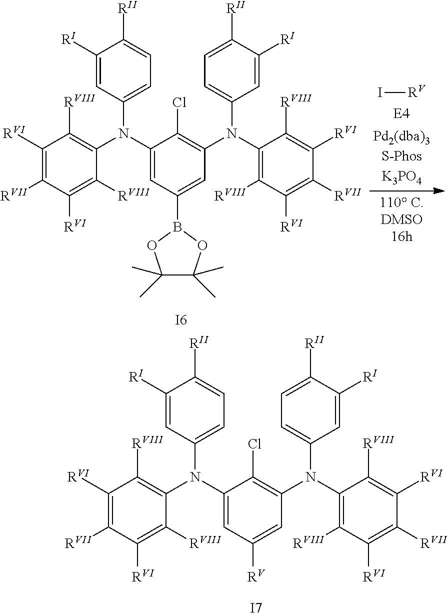

I6 (1.00 equivalents), E4 (1.50 equivalents), tris(dibenzylideneacetone)dipalladium Pd₂(dba)₃ (0.04 equivalents; CAS: 51364-51-3), S-Phos (CAS: 657408-07-6, 0.08 equivalents) and potassium phosphate tribasic (K₃PO₄; 4.00 equivalents) are stirred under nitrogen atmosphere in DMSO at 110° C. for 16 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and I7 is obtained as solid.

### General Procedure for the Synthesis AAV13:

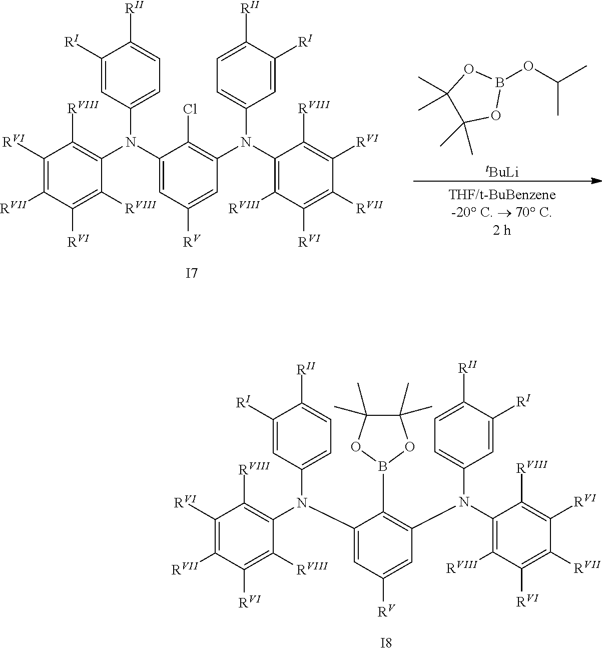

After dissolving I7 (1 equivalent) under nitrogen atmosphere in THF and cooling to −20° C. or in tert-butylbenzene and cooling to −10° C., ^{t}BuLi (2 equivalents, CAS: 594-19-4) is added and the reaction mixture is stirred at 0° C. After complete lithiation, the reaction is quenched and 1,3,2-dioxaborolane (2 equivalents, CAS: 61676-62-8) is added and the reaction mixture is stirred under reflux at 70° C. for 2 h. After cooling down to room temperature (rt), the reaction mixture is extracted between toluene and brine and the phases are separated. The combined organic layers are dried over MgSO₄ and then the solvent is removed under reduced pressure. The crude product obtained is purified by recrystallization or column chromatography and I8 is obtained as solid.

### General Procedure for the Synthesis AAV14:

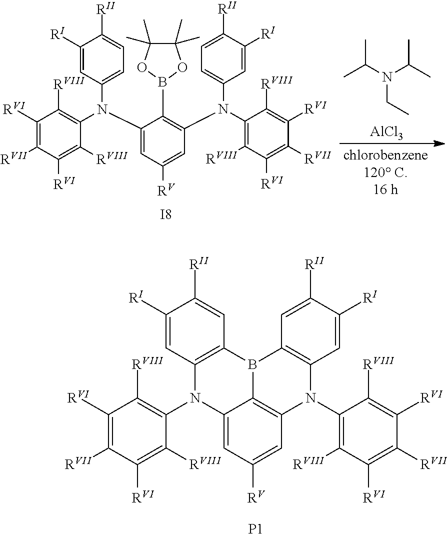

I8 (1 equivalent), N,N-diisopropylethylamine (10 equivalents, CAS: 7087-68-5) and AlCl₃ (10 equivalents, CAS: 7446-70-0) are stirred under nitrogen atmosphere in chlorobenzene at 120° C. for 16 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and P1 is obtained as solid.

### General Synthesis Scheme III

General synthesis scheme III provides a synthesis scheme for organic molecules according to the invention, where the limitations of scheme I and II (i.e. R^{I} ═R^{IV} and R^{II}═R^{III}) do not exist

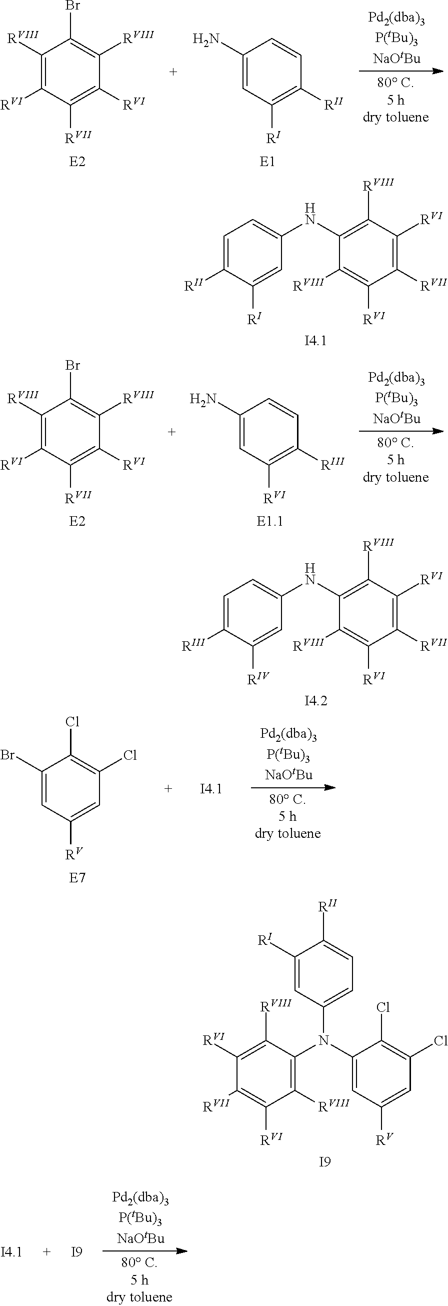

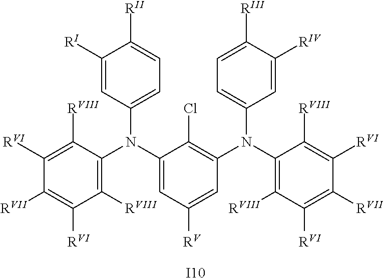

### General Procedure for Synthesis AAV15:

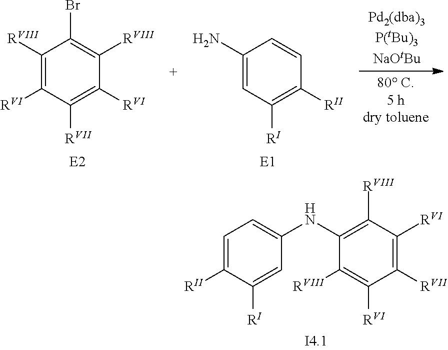

1-Bromo-3,5-diphenylbenzene (1 equivalents, CAS: 103068-20-8), E1 (2.20 equivalents), tris(dibenzylideneacetone)dipalladium Pd₂(dba)₃ (0.01 equivalents; CAS: 51364-51-3), tri-tert-butyl-phosphine (P(^{t}Bu)₃, CAS: 13716-12-6, 0.02 equivalents) and sodium tert-butoxide (NaO^{t}Bu; 3.00 equivalents) are stirred under nitrogen atmosphere in toluene at 80° C. for 5 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and I4.1 is obtained as solid.

### General Procedure for Synthesis AAV16:

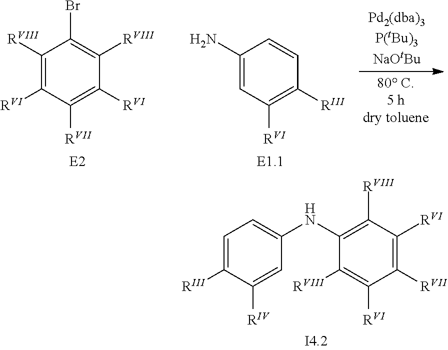

1-Bromo-3,5-diphenylbenzene (1 equivalents, CAS: 103068-20-8), E1.1 (2.20 equivalents), tris(dibenzylideneacetone)dipalladium Pd₂(dba)₃ (0.02 equivalents; CAS: 51364-51-3), tri-tert-butyl-phosphine (P(^{t}Bu)₃, CAS: 13716-12-6, 0.02 equivalents) and sodium tert-butoxide (NaO^{t}Bu; 3.00 equivalents) are stirred under nitrogen atmosphere in toluene at 80° C. for 5 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and I4.2 is obtained as solid.

### General Procedure for Synthesis AAV17:

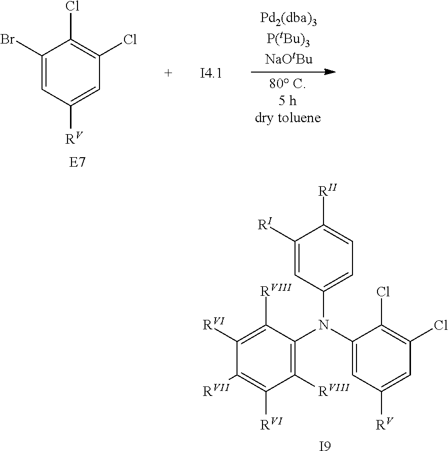

E7 (1.00 equivalent), I4.1 (1.10 equivalents), tris(dibenzylideneacetone)dipalladium Pd₂(dba)₃ (0.01 equivalents, CAS: 51364-51-3), tri-tert-butyl-phosphine P(^{t}Bu)₃ (0.02 equivalents, CAS: 13716-12-6) and sodium tert-butoxide NaO^{t}Bu (3.00 equivalents, CAS: 865-48-5) are stirred under nitrogen atmosphere in toluene at 80° C. for 5 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and I9 is obtained as solid.

### General Procedure for Synthesis AAV18:

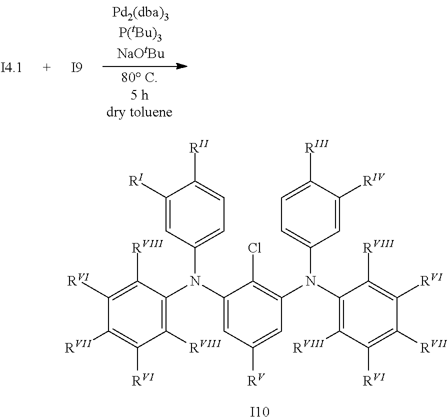

I4.2 (1.00 equivalent), I9 (1.10 equivalents), tris(dibenzylideneacetone)dipalladium Pd₂(dba)₃ (0.01 equivalents; CAS: 51364-51-3), tri-tert-butyl-phosphine P(^{t}Bu)₃ (0.02 equivalents, CAS: 13716-12-6) and sodium tert-butoxide NaO^{t}Bu (3.00 equivalents, CAS: 865-48-5) are stirred under nitrogen atmosphere in toluene at 110° C. for 16 h. After cooling down to room temperature (rt) the reaction mixture is extracted between toluene and brine and the phases are separated. The combined organic layers are dried over MgSO₄ and then the solvent is removed under reduced pressure. The crude product obtained is purified by recrystallization or column chromatography and I10 is obtained as solid.

The last synthesis steps of the general scheme III from I10 to P1 is carried out under similar conditions as described in AAV13 and AAV14.

The general synthesis scheme IV:

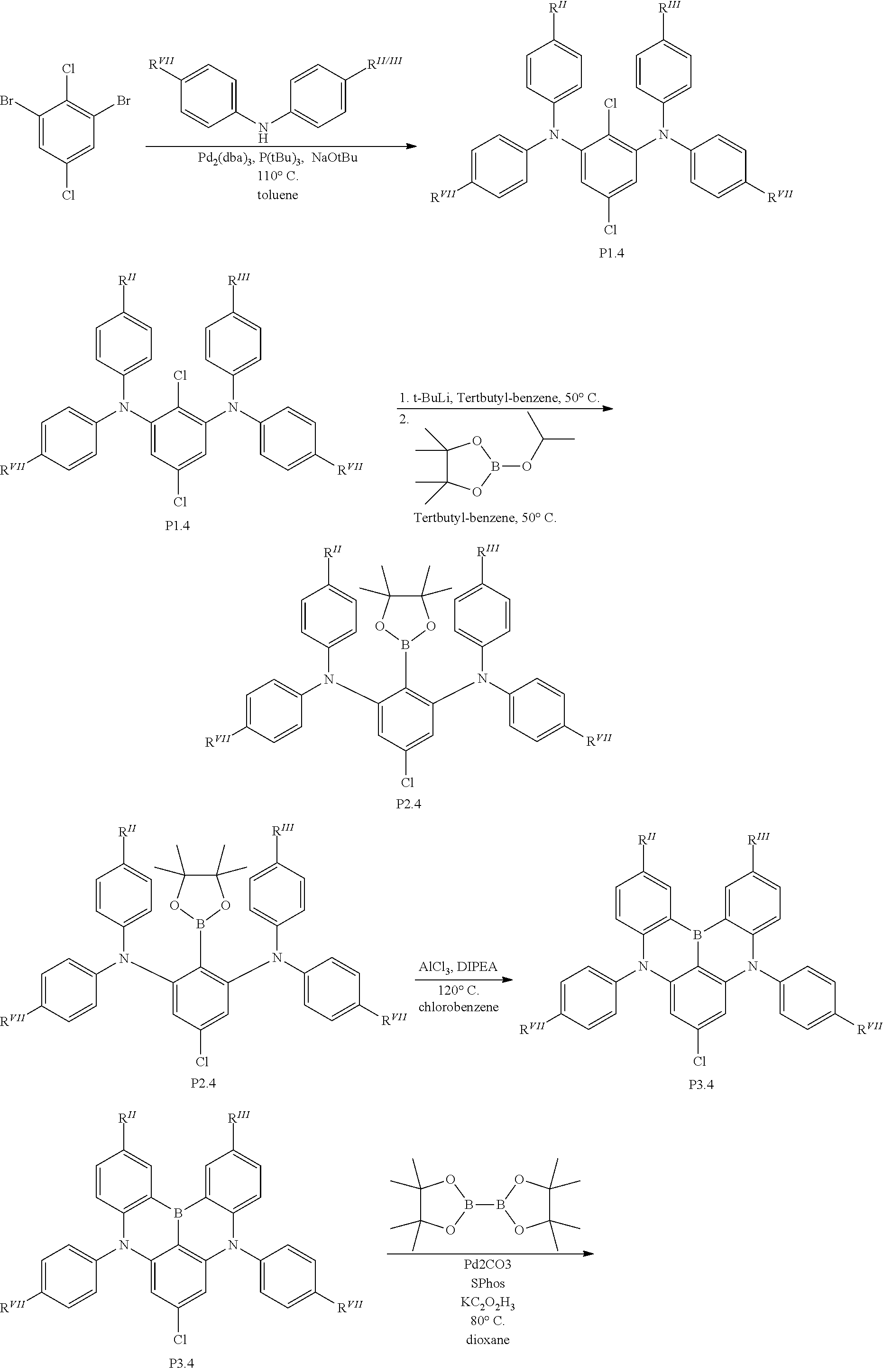

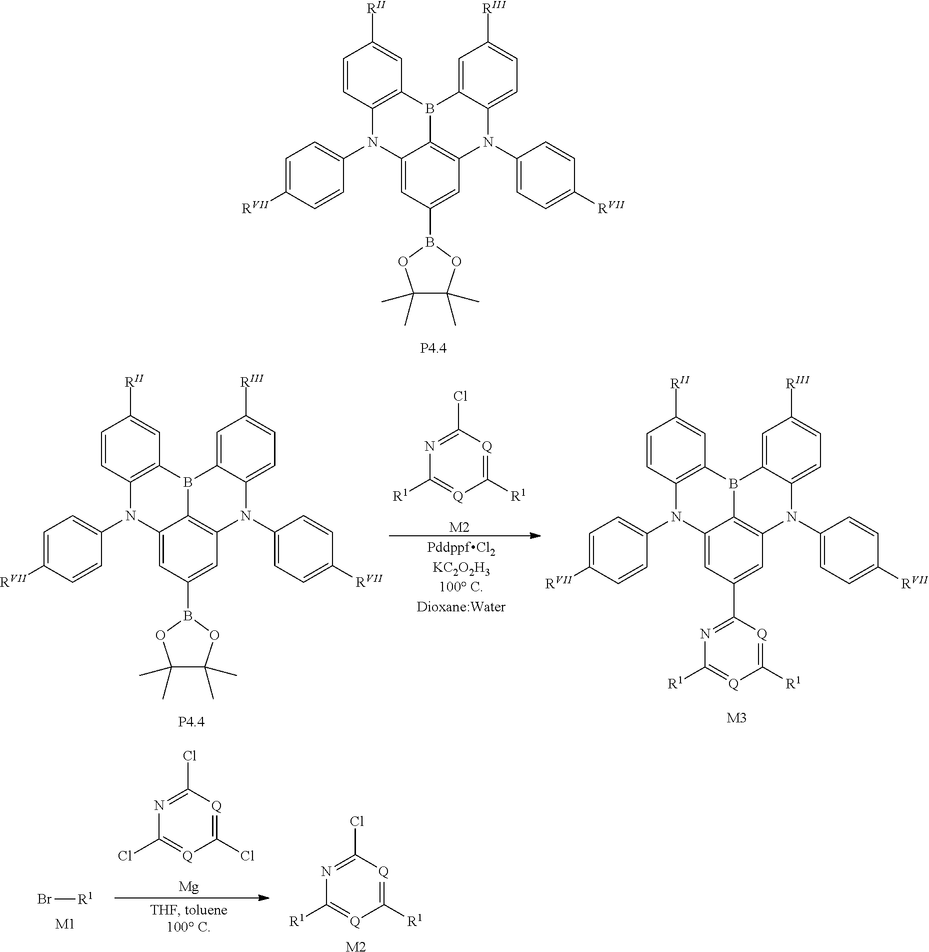

### General Procedure for Synthesis AAV19:

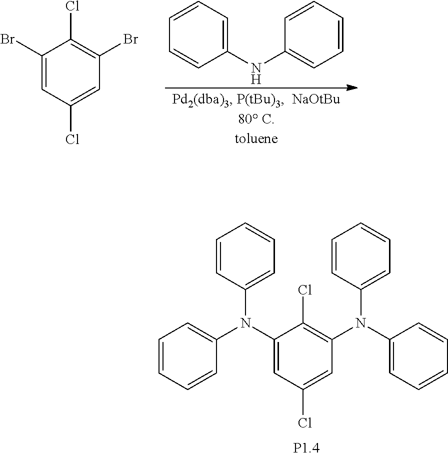

Under N₂ atmosphere, a two-necked flask is charged with 1,3-dibromo-2-chlorobenzene [81067-41-6] (1.0 equiv.), a diarylamine derivative (2.2 equiv.), Pd₂(dba)₃ [51364-51-3] (0.02 equiv.) and sodium tert-butoxide [865-48-5] (3.3 equiv.). Dry toluene (4 mL/mmol of 1,3-dibromo-2-chlorobenzene) and tri-tert-butylphosphine [13716-12-6] (0.06 equiv.) are added and the resulting suspension degassed for 10 min. Subsequently, the mixture is heated at 80° C. overnight. After cooling down to room temperature (rt), dichloromethane and water are added, the phases separated, the aqueous layer extracted with dichloromethane and the combined organic layers dried over MgSO₄, filtered and concentrated. The crude product is purified with MPLC or recrystallization to obtain the corresponding product P1.4 as a solid.

### General Procedure for Synthesis AAV20:

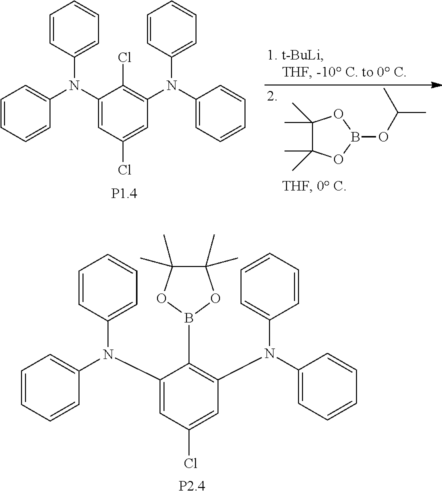

A two-necked flask is flame-dried under vacuum, allowed to cool down to rt under vacuum, backfilled with N₂ and subsequently charged with the aryl chloride P1.4 (1.0 equiv.). At 0° C., a 1.7 M solution of tert-butyllithium in pentane [594-19-4] (2.0 equiv.) is added with a syringe and the resulting mixture stirred at 50° C. until completion of the lithiation (usually 2 h, monitored by quenching with DMF and subsequent TLC). At 50° C., 2-isopropoxy-4,4,5,5-tetramethyl-1,3,2-dioxaborolane [61676-62-8] (2.0 equiv.) is added with a syringe and the mixture is stirred at 50° C. until completion of the reaction (usually 1 h, monitored by quenching with DMF and subsequent TLC). Water and ethyl acetate are added, the phases separated, and the organic layer dried over MgSO₄, filtered and concentrated. The crude product is purified by recrystallization or MPLC to obtain the corresponding boronic ester P2.4 as a solid.

### General Procedure for Synthesis AAV21:

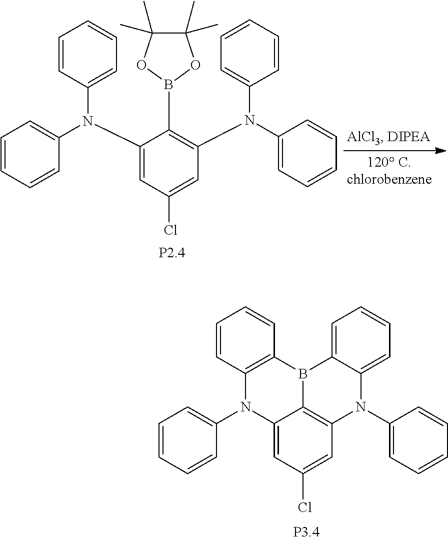

Under N₂ atmosphere, a two-necked flask is charged with the boronic ester P2.4 (1.0 equiv.). Dry chlorobenzene is added, followed by aluminum chloride [7446-70-0] (10 equiv.) and N,N-diisopropylethylamine (DIPEA) [7087-68-5] (15 equiv.). The resulting mixture is heated at 120° C. until completion of the reaction (usually 2 h, monitored with TLC). After cooling down to rt, the reaction is carefully quenched with water. Subsequently, dichloromethane is added, the phases are separated, and the aqueous layer extracted with dichloromethane. The combined organic layers are dried over MgSO₄, filtered and concentrated. The residue is purified by recrystallization or MPLC, yielding the desired material P3.4, as a solid.

### General Procedure for Synthesis AAV22:

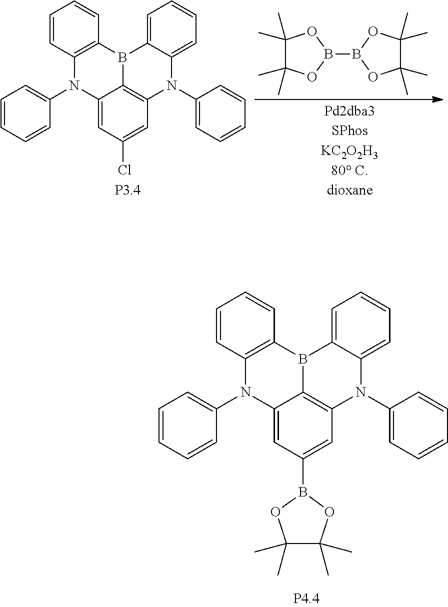

Under N₂ atmosphere, a two-necked flask is charged with P3.4 (1.0 equiv.), Bis(pinacolato)diboron [73183-34-3] (1.2 equiv.), Pd₂(dba)₃ [51364-51-3] (0.06 equiv.), S-Phos [657408-07-6] (0.12 equiv.) and potassium acetate [127-08-2] (3.0 equiv.). Dry Dioxane (10 mL/mmol of P3.4) is added and the resulting mixture degassed for 10 min. Subsequently, the mixture is heated at 80° C. overnight. After cooling down to room temperature (rt), dichloromethane and water are added, the phases separated, the aqueous layer extracted with dichloromethane and the combined organic layers dried over MgSO₄, filtered and concentrated. The crude product is purified with MPLC or recrystallization to obtain the corresponding product P4.4 as a solid.

### General Procedure for Synthesis AAV23:

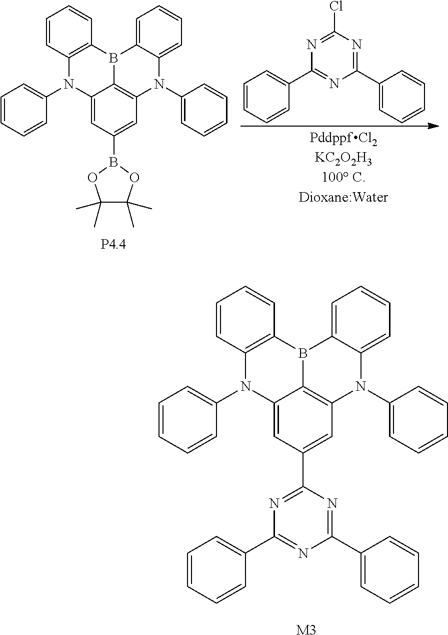

Under N₂ atmosphere, a two-necked flask is charged with P4.4 (1.0 equiv.), 2-chloro-4,6-diphenyl-1,3,5-triazine [3842-55-5] (1.2 equiv.), Pddppf.Cl₂ [72287-26-4] (0.05 equiv.) and potassium acetate [127-08-2] (3.0 equiv.). A 10:1 dioxane:water mixture (22 mL/mmol of P4.4) is added and the resulting mixture degassed for 10 min. Subsequently, the mixture is heated at 100° C. overnight. After cooling down to room temperature (rt), the mixture was poured into water. The crushed-out solid was filtered off and rinsed with ethanol. The crude product is purified with MPLC or recrystallization to obtain the corresponding product M3 as a solid.

### General Procedure for Synthesis AAV24:

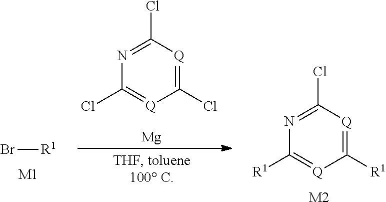

Under N₂ atmosphere, a two-necked flask is charged with Mg [7439-95-4] (2.0 equiv.) and the Mg was activated via iodine [7553-56-2] (0.02 equiv.). Dry THF (1 mL/mmol M1) was added and a solution of M1 (2.5 equiv.) in dry THF (1 mL/mmol M1) was added dropwise at room temperature and stirred at 80° C. for 3 h. After cooling down, a solution of cyanuric chloride [108-77-0] (1.0 equiv.) in toluene (1 mL/mmol M1) was added to the reaction mixture at room temperature and after addition the mixture was stirred at 100° C. for 16 h. After cooling down to room temperature (rt), the mixture was poured into water. The crushed-out solid was filtered off and extracted with DCM. The crude product is purified with column chromatography M2 as a solid.

### Cyclic Voltammetry

Cyclic voltammograms are measured from solutions having concentration of 10⁻³ mol/L of the organic molecules in dichloromethane or a suitable solvent and a suitable supporting electrolyte (e.g. 0.1 mol/L of tetrabutylammonium hexafluorophosphate). The measurements are conducted at room temperature under nitrogen atmosphere with a three-electrode assembly (Working and counter electrodes: Pt wire, reference electrode: Pt wire) and calibrated using FeCp₂/FeCp₂+ as internal standard. The HOMO data was corrected using ferrocene as internal standard against a saturated calomel electrode (SCE).

### Density Functional Theory Calculation

Molecular structures are optimized employing the BP86 functional and the resolution of identity approach (R^{I}). Excitation energies are calculated using the (BP86) optimized structures employing Time-Dependent DFT (TD-DFT) methods. Orbital and excited state energies are calculated with the B3LYP functional. Def2-SVP basis sets (and a m4-grid for numerical integration are used. The Turbomole program package is used for all calculations.

### Photophysical Measurements

Sample pretreatment: Spin-coating

Apparatus: Spin 150, SPS euro.

The sample concentration is 10 mg/ml, dissolved in a suitable solvent.

Program: 1) 3 s at 400 U/min; 20 s at 1000 U/min at 1000 Upm/s. 3) 10 s at 4000 U/min at 1000 Upm/s. After coating, the films are dried at 70° C. for 1 min.

Photoluminescence spectroscopy and Time-Correlated Single-Photon Counting (TCSPC)

Steady-state emission spectroscopy is measured by a Horiba Scientific, Modell FluoroMax-4 equipped with a 150 W Xenon-Arc lamp, excitation- and emissions monochromators and a Hamamatsu R928 photomultiplier and a time-correlated single-photon counting option. Emissions and excitation spectra are corrected using standard correction fits.

Excited state lifetimes are determined employing the same system using the TCSPC method with FM-2013 equipment and a Horiba Yvon TCSPC hub.

Excitation sources:
NanoLED 370 (wavelength: 371 nm, puls duration: 1.1 ns)
NanoLED 290 (wavelength: 294 nm, puls duration: <1 ns)
SpectraLED 310 (wavelength: 314 nm)
SpectraLED 355 (wavelength: 355 nm).

Data analysis (exponential fit) is done using the software suite DataStation and DAS6 analysis software. The fit is specified using the chi-squared-test.

### Photoluminescence Quantum Yield Measurements

For photoluminescence quantum yield (PLQY) measurements an Absolute PL Quantum Yield Measurement C9920-03G system (Hamamatsu Photonics) is used. Quantum yields and CIE coordinates are determined using the software U6039-05 version 3.6.0.

Emission maxima are given in nm, quantum yields Φ in % and CIE coordinates as x,y values. PLQY is determined using the following protocol:
1) Quality assurance: Anthracene in ethanol (known concentration) is used as reference
2) Excitation wavelength: the absorption maximum of the organic molecule is determined and the molecule is excited using this wavelength
3) Measurement
   Quantum yields are measured, for sample, of solutions or films under nitrogen atmosphere. The yield is calculated using the equation:

$${Φ}_{PL} = \frac{{n}_{photon} , emited}{{n}_{photon} , absorbed} = \frac{\int \frac{λ}{hc} [ {Int}_{emitted}^{sample} ( λ ) - {Int}_{absorbed}^{sample} ( λ ) ] d λ}{\int \frac{λ}{hc} [ {Int}_{emitted}^{reference} ( λ ) - {Int}_{absorbed}^{reference} ( λ ) ] d λ}$$

wherein nₚₕₒₜₒₙ denotes the photon count and Int. the intensity.

### Production and Characterization of Optoelectronic Devices

Optoelectronic devices, in particular OLED devices, comprising organic molecules according to the invention can be produced via vacuum-deposition methods. If a layer contains more than one compound, the weight-percentage of one or more compounds is given in %. The total weight-percentage values amount to 100%, thus if a value is not given, the fraction of this compound equals to the difference between the given values and 100%.

The not fully optimized OLEDs are characterized using standard methods and measuring electroluminescence spectra, the external quantum efficiency (in %) in dependency on the intensity, calculated using the light detected by the photodiode, and the current. The OLED device lifetime is extracted from the change of the luminance during operation at constant current density. The LT50 value corresponds to the time, where the measured luminance decreased to 50% of the initial luminance, analogously LT80 corresponds to the time point, at which the measured luminance decreased to 80% of the initial luminance, LT 95 to the time point, at which the measured luminance decreased to 95% of the initial luminance etc. Accelerated lifetime measurements are performed (e.g. applying increased current densities). For example, LT80 values at 500 cd/m² are determined using the following equation:

$$LT 80 ( 500 \frac{{cd}^{2}}{{m}^{2}} ) = LT 80 ( {L}_{0} ) {( \frac{{L}_{0}}{500 \frac{{cd}^{2}}{{m}^{2}}} )}^{1.6}$$

wherein L₀ denotes the initial luminance at the applied current density.

The values correspond to the average of several pixels (typically two to eight), the standard deviation between these pixels is given.

### HPLC-MS

HPLC-MS analysis is performed on an HPLC by Agilent (1100 series) with MS-detector (Thermo LTQ XL).

Exemplarily a typical HPLC method is as follows: a reverse phase column 4.6 mm×150 mm, particle size 3.5 μm from Agilent (ZORBAX Eclipse Plus 95 Å C18, 4.6×150 mm, 3.5 μm HPLC column) is used in the HPLC. The HPLC-MS measurements are performed at room temperature (rt) following gradients

| Flow rate [ml/min] | Time [min] | A[%] | B[%] | C[%] |
|---|---|---|---|---|
| 2.5 | 0 | 40 | 50 | 10 |
| 2.5 | 5 | 40 | 50 | 10 |
| 2.5 | 25 | 10 | 20 | 70 |
| 2.5 | 35 | 10 | 20 | 70 |
| 2.5 | 35.01 | 40 | 50 | 10 |
| 2.5 | 40.01 | 40 | 50 | 10 |
| 2.5 | 41.01 | 40 | 50 | 10 |

using the following solvent mixtures:

| | | |
|---|---|---|
| Solvent A: | H₂O (90%) | MeCN (10%) |
| Solvent B: | H₂O (10%) | MeCN (90%) |
| Solvent C: | THF (50%) | MeCN (50%) |

An injection volume of 5 μL from a solution with a concentration of 0.5 mg/mL of the analyte is taken for the measurements.

Ionization of the probe is performed using an atmospheric pressure chemical ionization (APCI) source either in positive (APCI+) or negative (APCI−) ionization mode.

### Concentration Dependent Spectral Purity

The organic molecules described herein in particular comprise severely decreased tendency to form intermolecular aggregates which are known to cause broadening of the photo luminescence (PL) spectra in doped films with increasing concentration. A measure of this spectral broadening in doped films (e.g. spin coated thin films containing 1 wt % or more of the organic molecule in PMMA matrix) with increasing concentration is the Concentration Dependent Spectral Purity (CDSP) value.

The CDSP is represented by the following relation:

CDSP=(λₘₐₓ*CIEy)/c

wherein λₘₐₓ is the maximum of the PL spectrum of a given organic molecule in nm, CIEy is the CIEy coordinate (Comission Internationale de l'Eclairage) derived from the PL spectrum of the organic molecule and c is the concentration of the organic molecule by weight in % in doped film.

The CDSP can be interpreted as follows:

If two different organic molecules have a comparable λₘₐₓ in doped films of the same concentration, the one with a lower CDSP is preferred in terms of spectral purity. Especially the difference |ΔCDSP| between two concentrations provides an indication whether a material shows a high tendency to aggregate or not: the smaller the ΔCDSP value, the lower the aggregation tendency of a material.

Comparable values are especially obtained for concentrations c≥2 wt %.

### Example 1

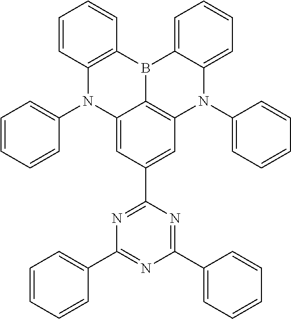 [SMILES: c1ccc(-c2nc(-c3ccccc3)nc(-c3cc4c5c(c3)N(c3ccccc3)c3ccccc3B5c3ccccc3N4c3ccccc3)n2)cc1]

Example 1 was synthesized according to

AAV19 (85% yield),
AAV20 (70% yield);
AAV21 (91% yield);
AAV22 (68% yield) and
AAV23 (62% yield).

MS (HPLC-MS), m/z (retention time): 652.50 (6.21 min).

The emission maximum of example 1 (5% by weight in PMMA) is at 518 nm, the full width at half maximum (FWHM) is 0.22 eV, the CIEy coordinate is 0.66. The photoluminescence quantum yield (PLQY) is 76%.

### Example 2

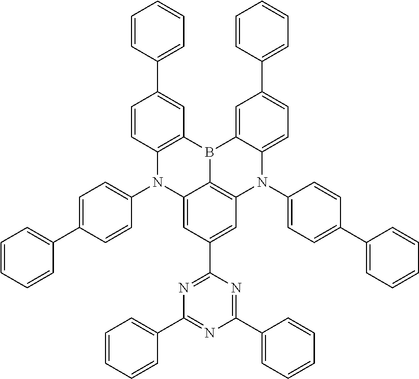 [SMILES: c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)nc(-c7ccccc7)n6)cc3c54)cc2)cc1]

Example 2 was synthesized according to

AAV19 (77% yield) using N-(biphenyl-4-yl)biphenyl-4-amine [102113-98-4] as the diarylamine reagent,

AAV20 (65% yield);

AAV21 (79% yield);

AAV22 (74% yield) and

AAV23 (60% yield).

MS (HPLC-MS), m/z (retention time): 956.70 (8.01 min).

The emission maximum of example 2 (1% by weight in PMMA) is at 534 nm, the full width at half maximum (FWHM) is 0.22 eV, the CIEy coordinate is 0.61. The photoluminescence quantum yield (PLQY) is 81%.

### Example 3

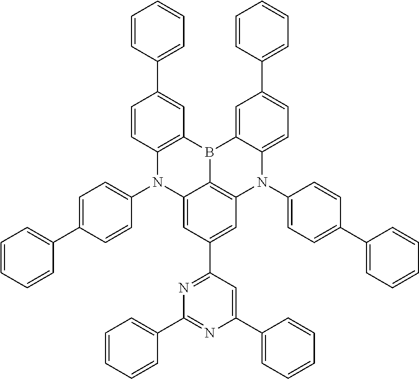 [SMILES: c1ccc(-c2ccc(N3c4ccc(-c5ccccc5)cc4B4c5cc(-c6ccccc6)ccc5N(c5ccc(-c6ccccc6)cc5)c5cc(-c6cc(-c7ccccc7)nc(-c7ccccc7)n6)cc3c54)cc2)cc1]

Example 3 was synthesized according to

AAV19 (77% yield) using N-(biphenyl-4-yl)biphenyl-4-amine [102113-98-4] as the diarylamine reagent,

AAV20 (65% yield);

AAV21 (79% yield);

AAV22 (74% yield) and

AAV23 (17% yield) using 4-chloro-2,6-diphenylpyrimidine [29509-91-9] as the halogenated reagent.

MS (HPLC-MS), m/z (retention time): 955.7 (7.98 min).

The emission maximum of example 3 (2% by weight in PMMA) is at 520 nm, the full width at half maximum (FWHM) is 0.18 eV, the CIEy coordinate is 0.67. The photoluminescence quantum yield (PLQY) is 79%.

### Example 4

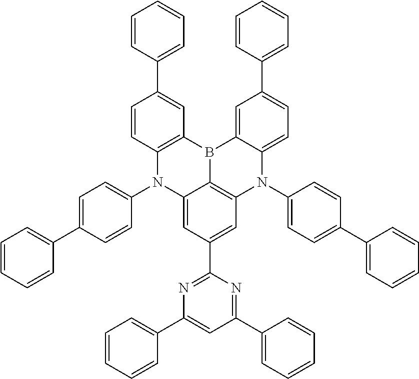 [SMILES: c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)cc(-c7ccccc7)n6)cc3c54)cc2)cc1]

Example 4 was synthesized according to

AAV19 (77% yield) using N-(biphenyl-4-yl)biphenyl-4-amine [102113-98-4] as the diarylamine reagent,

AAV20 (65% yield);

AAV21 (79% yield);

AAV22 (74% yield) and

AAV23 (22% yield) using 2-chloro-4,6-diphenylpyrimidine [2915-16-4] as the halogenated reagent.

MS (HPLC-MS), m/z (retention time): 955.70 (7.62 min).

The emission maximum of example 4 (1% by weight in PMMA) is at 507 nm, the full width at half maximum (FWHM) is 0.15 eV, the CIEy coordinate is 0.65.

### Example 5

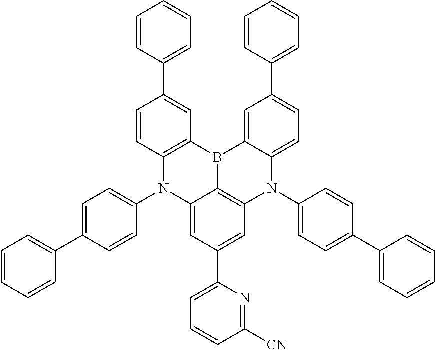 [SMILES: N#Cc1cccc(-c2cc3c4c(c2)N(c2ccc(-c5ccccc5)cc2)c2ccc(-c5ccccc5)cc2B4c2cc(-c4ccccc4)ccc2N3c2ccc(-c3ccccc3)cc2)n1]

Example 5 was synthesized according to

AAV19 (77% yield) using N-(biphenyl-4-yl)biphenyl-4-amine [102113-98-4] as the diarylamine reagent,

AAV20 (65% yield);

AAV21 (79% yield);

AAV22 (74% yield) and

AAV23 (35% yield) using 2-Bromo-6-cyanopyridine [122918-25-6] as the halogenated reagent.

MS (HPLC-MS), m/z (retention time): 827 (6.19 min).

The emission maximum of example 5 (2% by weight in PMMA) is at 505 nm, the full width at half maximum (FWHM) is 0.16 eV, the CIEy coordinate is 0.63. The photoluminescence quantum yield (PLQY) is 74%.

### Example 6

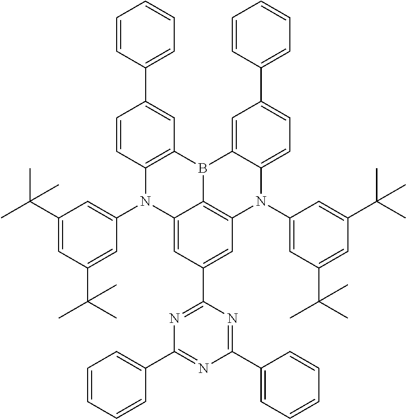 [SMILES: CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5nc(-c6ccccc6)nc(-c6ccccc6)n5)cc2c43)cc(C(C)(C)C)c1]

MS (HPLC-MS), m/z (retention time): 1028 (9.3 min).

The emission maximum of example 6 (2% by weight in PMMA) is at 537 nm, the full width at half maximum (FWHM) is 0.17 eV, the CIEy coordinate is 0.62. The photoluminescence quantum yield (PLQY) is 75%.

### Example 7

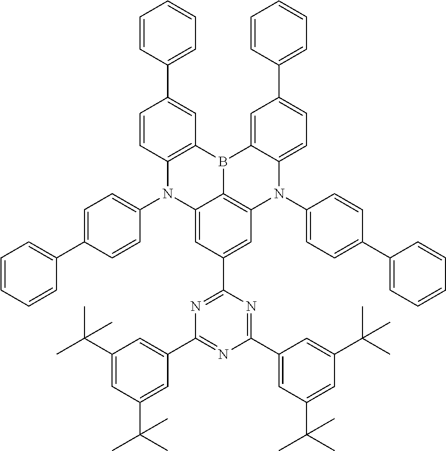 [SMILES: CC(C)(C)c1cc(-c2nc(-c3cc(C(C)(C)C)cc(C(C)(C)C)c3)nc(-c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)n2)cc(C(C)(C)C)c1]

Example 7 was synthesized according to

AAV19 (77% yield) using N-(biphenyl-4-yl)biphenyl-4-amine [102113-98-4] as the diarylamine reagent,

AAV20 (65% yield);

AAV21 (79% yield);

AAV22 (74% yield) and

AAV23 (28% yield) using 2,4-Bis[3,5-bis(1,1-dimethyl)phenyl]-6-chloro-1,3,5-triazine as the halogenated reagent.

2,4-Bis[3,5-bis(1,1-dimethyl)phenyl]-6-chloro-1,3,5-triazine was synthesized according to

AAV24 (52%).

MS (HPLC-MS), m/z (retention time): 1180 (9.30 min).

The emission maximum of example 7 (2% by weight in PMMA) is at 525 nm, the full width at half maximum (FWHM) is 0.18 eV, the CIEy coordinate is 0.66. The photoluminescence quantum yield (PLQY) is 80%.

### Example 8

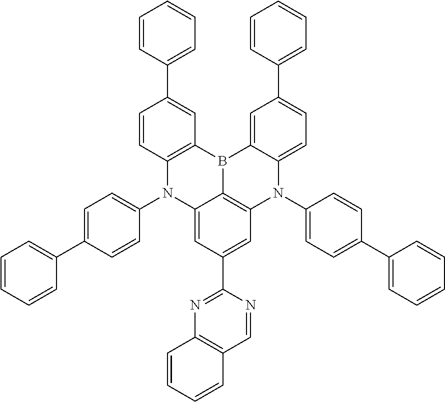 [SMILES: c1ccc(-c2ccc(N3c4ccc(-c5ccccc5)cc4B4c5cc(-c6ccccc6)ccc5N(c5ccc(-c6ccccc6)cc5)c5cc(-c6ncc7ccccc7n6)cc3c54)cc2)cc1]

Example 8 was synthesized according to

AAV19 (77% yield) using N-(biphenyl-4-yl)biphenyl-4-amine [102113-98-4] as the diarylamine reagent,

AAV20 (65% yield);

AAV21 (79% yield);

AAV22 (74% yield) and

AAV9 (39% yield) using 2-bromoquinoxaline [36856-91-4] as halogenated reagent.

MS (HPLC-MS), m/z (retention time): 853.6 (9.45 min)

The emission maximum of example 8 (2% by weight in PMMA) is at 541 nm, the full width at half maximum (FWHM) is 0.28 eV, the CIEy coordinate is 0.60. The photoluminescence quantum yield (PLQY) is 70%.

### Example 9

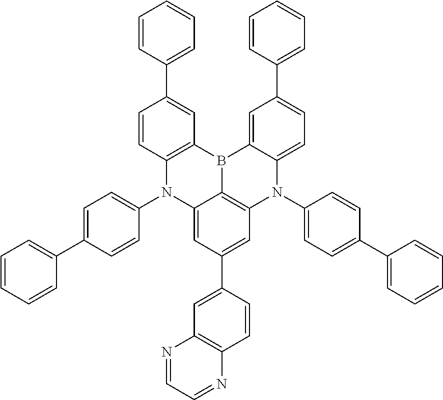 [SMILES: c1ccc(-c2ccc(N3c4ccc(-c5ccccc5)cc4B4c5cc(-c6ccccc6)ccc5N(c5ccc(-c6ccccc6)cc5)c5cc(-c6ccc7nccnc7c6)cc3c54)cc2)cc1]

Example 9 was synthesized according to

AAV19 (77% yield) using N-(biphenyl-4-yl)biphenyl-4-amine [102113-98-4] as the diarylamine reagent,

AAV20 (65% yield);

AAV21 (79% yield);

AAV22 (74% yield) and

AAV9 (43% yield) using 6-bromoquinoxaline [50998-17-9] as the halogenated reagent.

MS (HPLC-MS), m/z (retention time): 853.6 (6.45 min)

The emission maximum of example 9 (2% by weight in PMMA) is at 502 nm, the full width at half maximum (FWHM) is 0.26 eV, the CIEy coordinate is 0.58. The photoluminescence quantum yield (PLQY) is 68%.

### Example 10

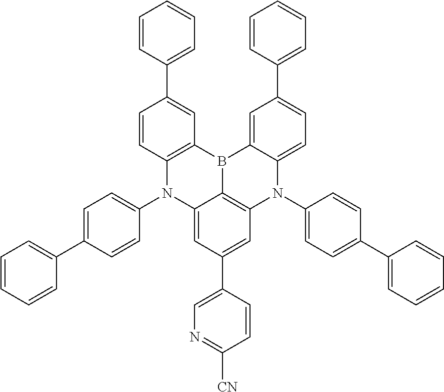 [SMILES: N#Cc1ccc(-c2cc3c4c(c2)N(c2ccc(-c5ccccc5)cc2)c2ccc(-c5ccccc5)cc2B4c2cc(-c4ccccc4)ccc2N3c2ccc(-c3ccccc3)cc2)cn1]

Example 10 was synthesized according to

AAV19 (77% yield) using N-(biphenyl-4-yl)biphenyl-4-amine [102113-98-4] as the diarylamine reagent,

AAV20 (65% yield);

AAV21 (79% yield);

AAV9 (14% yield) using P3 as the halogenated reagent and replacing P4 with 2-cyano-5-(4,4,5,5-tetramethyl-1,3,2-dioxaborolan-2-yl)pyridine [741709-63-7].

MS (HPLC-MS), m/z (retention time): 759.5 (6.36 min).

The emission maximum of example 10 (2% by weight in PMMA) is at 513 nm, the full width at half maximum (FWHM) is 0.23 eV, the CIEy coordinate is 0.63. The photoluminescence quantum yield (PLQY) is 78%.

### Example 11

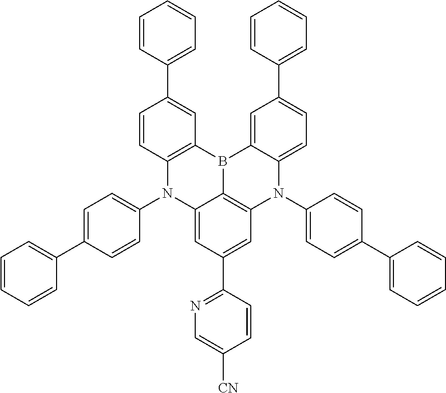 [SMILES: N#Cc1ccc(-c2cc3c4c(c2)N(c2ccc(-c5ccccc5)cc2)c2ccc(-c5ccccc5)cc2B4c2cc(-c4ccccc4)ccc2N3c2ccc(-c3ccccc3)cc2)nc1]

The emission maximum of example 11 (2% by weight in PMMA) is at 528 nm, the full width at half maximum (FWHM) is 0.23 eV, the CIEy coordinate is 0.64. The photoluminescence quantum yield (PLQY) is 88%.

### Example 12

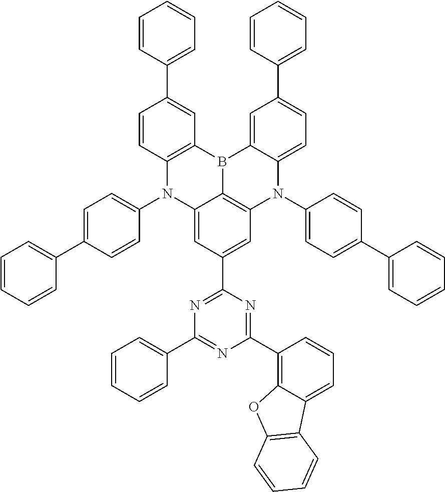 [SMILES: c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)nc(-c7cccc8c7oc7ccccc78)n6)cc3c54)cc2)cc1]

The emission maximum of example 12 (2% by weight in PMMA) is at 541 nm, the full width at half maximum (FWHM) is 0.21 eV, the CIEy coordinate is 0.60. The photoluminescence quantum yield (PLQY) is 75%.

### Example 13

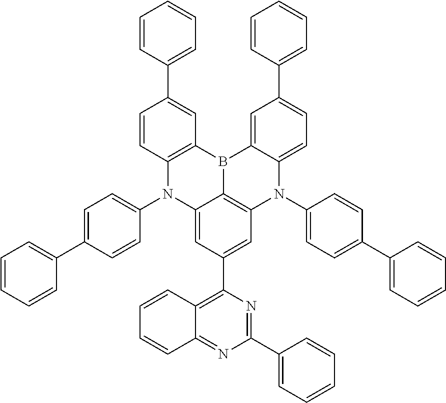 [SMILES: c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)nc7ccccc67)cc3c54)cc2)cc1]

The emission maximum of example 13 (2% by weight in PMMA) is at 542 nm, the full width at half maximum (FWHM) is 0.28 eV, the CIEy coordinate is 0.60. The photoluminescence quantum yield (PLQY) is 84%.

### Example 14

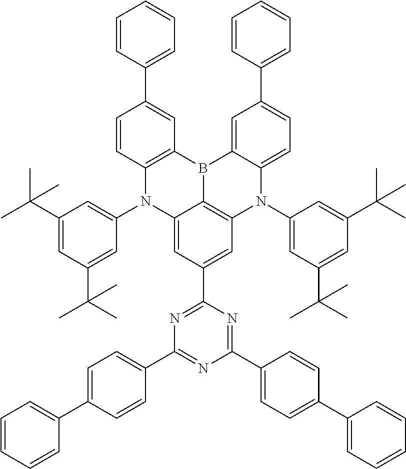 [SMILES: CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5nc(-c6ccc(-c7ccccc7)cc6)nc(-c6ccc(-c7ccccc7)cc6)n5)cc2c43)cc(C(C)(C)C)c1]

The emission maximum of example 14 (2% by weight in PMMA) is at 537 nm, the full width at half maximum (FWHM) is 0.17 eV, the CIEy coordinate is 0.62. The photoluminescence quantum yield (PLQY) is 82%.

### Example 15

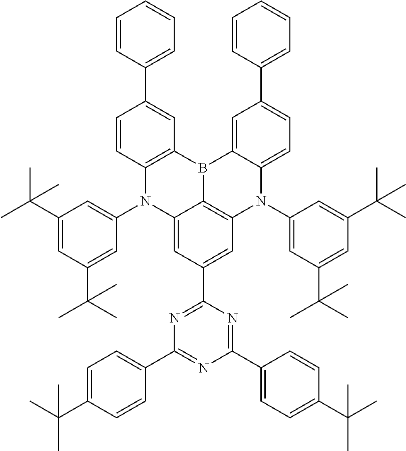 [SMILES: CC(C)(C)c1ccc(-c2nc(-c3ccc(C(C)(C)C)cc3)nc(-c3cc4c5c(c3)N(c3cc(C(C)(C)C)cc(C(C)(C)C)c3)c3ccc(-c6ccccc6)cc3B5c3cc(-c5ccccc5)ccc3N4c3cc(C(C)(C)C)cc(C(C)(C)C)c3)n2)cc1]

The emission maximum of example 15 (2% by weight in PMMA) is at 527 nm, the full width at half maximum (FWHM) is 0.15 eV, the CIEy coordinate is 0.66. The photoluminescence quantum yield (PLQY) is 86%.

### Example 16

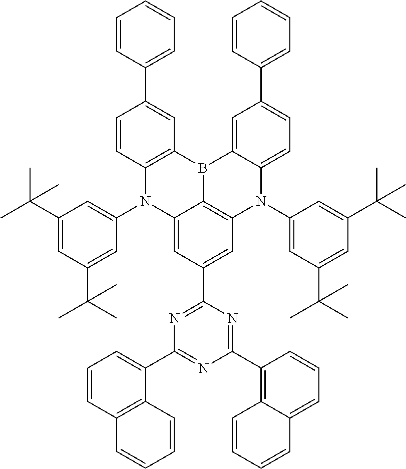 [SMILES: CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5nc(-c6cccc7ccccc67)nc(-c6cccc7ccccc67)n5)cc2c43)cc(C(C)(C)C)c1]

The emission maximum of example 16 (2% by weight in PMMA) is at 537 nm, the full width at half maximum (FWHM) is 0.18 eV, the CIEy coordinate is 0.62. The photoluminescence quantum yield (PLQY) is 87%.

### Example 17

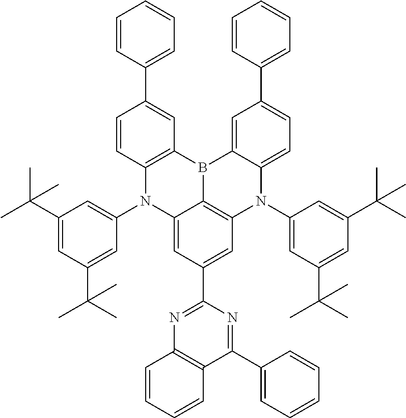 [SMILES: CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5nc(-c6ccccc6)c6ccccc6n5)cc2c43)cc(C(C)(C)C)c1]

The emission maximum of example 17 (2% by weight in PMMA) is at 517 nm, the full width at half maximum (FWHM) is 0.19 eV, the CIEy coordinate is 0.66. The photoluminescence quantum yield (PLQY) is 87%.

### Example 18

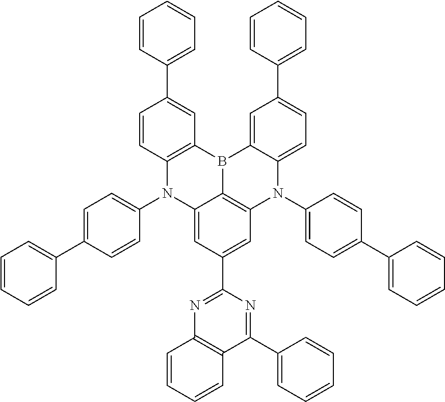 [SMILES: c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)c7ccccc7n6)cc3c54)cc2)cc1]

The emission maximum of example 18 (2% by weight in PMMA) is at 527 nm, the full width at half maximum (FWHM) is 0.26 eV, the CIEy coordinate is 0.64. The photoluminescence quantum yield (PLQY) is 74%.

### Example 19

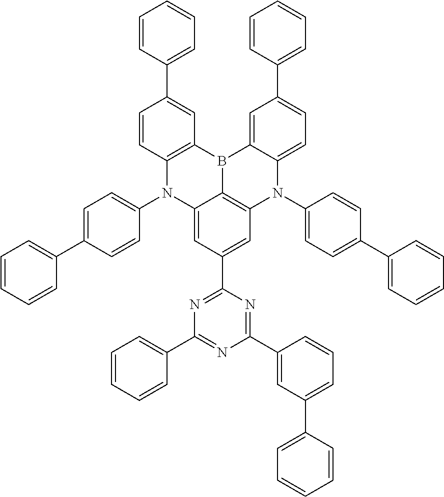 [SMILES: c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)nc(-c7cccc(-c8ccccc8)c7)n6)cc3c54)cc2)cc1]

The emission maximum of example 19 (2% by weight in PMMA) is at 544 nm, the full width at half maximum (FWHM) is 0.22 eV, the CIEy coordinate is 0.59. The photoluminescence quantum yield (PLQY) is 82%.

### Example 20

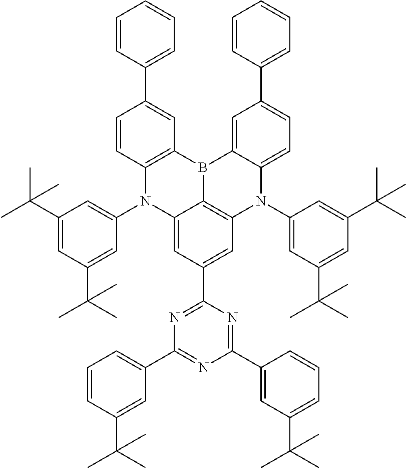 [SMILES: CC(C)(C)c1cccc(-c2nc(-c3cccc(C(C)(C)C)c3)nc(-c3cc4c5c(c3)N(c3cc(C(C)(C)C)cc(C(C)(C)C)c3)c3ccc(-c6ccccc6)cc3B5c3cc(-c5ccccc5)ccc3N4c3cc(C(C)(C)C)cc(C(C)(C)C)c3)n2)c1]

The emission maximum of example 20 (2% by weight in PMMA) is at 532 nm, the full width at half maximum (FWHM) is 0.16 eV, the CIEy coordinate is 0.64. The photoluminescence quantum yield (PLQY) is 86%.

### Example 21

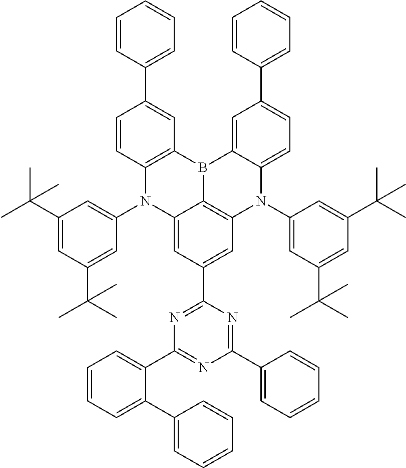 [SMILES: CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5nc(-c6ccccc6)nc(-c6ccccc6-c6ccccc6)n5)cc2c43)cc(C(C)(C)C)c1]

The emission maximum of example 21 (2% by weight in PMMA) is at 532 nm, the full width at half maximum (FWHM) is 0.14 eV, the CIEy coordinate is 0.66. The photoluminescence quantum yield (PLQY) is 81%.

### Example 22

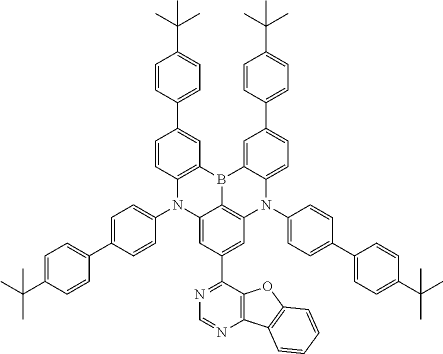 [SMILES: CC(C)(C)c1ccc(-c2ccc(N3c4ccc(-c5ccc(C(C)(C)C)cc5)cc4B4c5cc(-c6ccc(C(C)(C)C)cc6)ccc5N(c5ccc(-c6ccc(C(C)(C)C)cc6)cc5)c5cc(-c6ncnc7c6oc6ccccc67)cc3c54)cc2)cc1]

The emission maximum of example 22 (2% by weight in PMMA) is at 545 nm, the full width at half maximum (FWHM) is 0.21 eV, the CIEy coordinate is 0.61. The photoluminescence quantum yield (PLQY) is 74%.

### Example D1

Example 2 was tested in the OLED D1, which was fabricated with the following layer structure:

| Layer # | Thickness | D1 |
|---|---|---|
| 10 | 100 nm | Al |
| 9 | 2 nm | Liq |
| 8 | 20 nm | NBPhen |
| 7 | 10 nm | MAT1 |
| 6 | 50 nm | mCBP (79%) |
| | | MAT2 (20%) |
| | | Example 2 (1%) |
| 5 | 10 nm | mCBP |
| 4 | 10 nm | TCTA |
| 3 | 50 nm | NPB |
| 2 | 5 nm | HAT-CN |
| 1 | 50 nm | ITO |
| Substrate | | Glass |

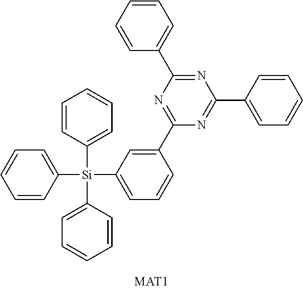 [SMILES: c1ccc(-c2nc(-c3ccccc3)nc(-c3cccc([Si](c4ccccc4)(c4ccccc4)c4ccccc4)c3)n2)cc1]

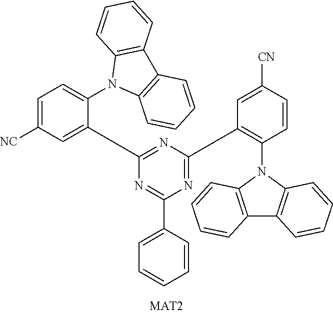 [SMILES: N#Cc1ccc(-n2c3ccccc3c3ccccc32)c(-c2nc(-c3ccccc3)nc(-c3cc(C#N)ccc3-n3c4ccccc4c4ccccc43)n2)c1]

OLED D1 yielded an external quantum efficiency (EQE) at 1000 cd/m² of 16.1%. The emission maximum is at 532 nm with a FWHM of 38 nm at 7.6 V. The corresponding CIEx value is 0.32 and the CIEy value is 0.65. A LT95-value at 1200 cd/m² of 1522 h was determined.

### Example D2

Example 2 was tested in the OLED D2, which was fabricated with the following layer structure:

| Layer # | Thickness | D2 |
|---|---|---|
| 10 | 100 nm | Al |
| 9 | 2 nm | Liq |
| 8 | 20 nm | NBPhen |
| 7 | 10 nm | MAT1 |
| 6 | 50 nm | mCBP (84%) |
| | | MAT2 (15%) |
| | | Example 2 (1%) |
| 5 | 10 nm | MAT3 |
| 4 | 10 nm | TCTA |
| 3 | 50 nm | NPB |
| 2 | 5 nm | HAT-CN |
| 1 | 50 nm | ITO |
| Substrate | | Glass |

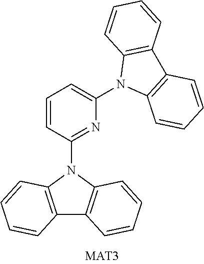 [SMILES: c1cc(-n2c3ccccc3c3ccccc32)nc(-n2c3ccccc3c3ccccc32)c1]

OLED D2 yielded an external quantum efficiency (EQE) at 1000 cd/m² of 17.7%. The emission maximum is at 532 nm with a FWHM of 36 nm at 7.6 V. The corresponding CIEx value is 0.31 and the CIEy value is 0.65. A LT95-value at 1200 cd/m² of 2006 h was determined.

### Example D3

Example 2 was tested in the OLED D3, which was fabricated with the following layer structure:

| Layer # | Thickness | D3 |
|---|---|---|
| 10 | 100 nm | Al |
| 9 | 2 nm | Liq |
| 8 | 20 nm | NBPhen |
| 7 | 10 nm | MAT1 |
| 6 | 50 nm | mCBP (75%) |
| | | MAT4 (5%) |
| | | MAT2 (15%) |
| | | Example 2 (1%) |
| 5 | 10 nm | MAT3 |
| 4 | 10 nm | TCTA |
| 3 | 50 nm | NPB |
| 2 | 5 nm | HAT-CN |
| 1 | 50 nm | ITO |
| Substrate | | Glass |

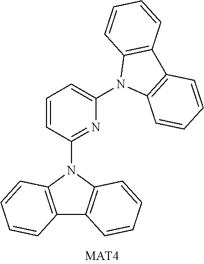 [SMILES: c1cc(-n2c3ccccc3c3ccccc32)nc(-n2c3ccccc3c3ccccc32)c1]

OLED D3 yielded an external quantum efficiency (EQE) at 1000 cd/m² of 20.2%. The emission maximum is at 532 nm with a FWHM of 38 nm at 7.6 V. The corresponding CIEx value is 0.32 and the CIEy value is 0.65. A LT95-value at 1200 cd/m² of 1866 h was determined.

### Example D4

Example 3 was tested in the OLED D4, which was fabricated with the following layer structure:

| Layer # | Thickness | D4 |
|---|---|---|
| 10 | 100 nm | Al |
| 9 | 2 nm | Liq |
| 8 | 20 nm | NBPhen |
| 7 | 10 nm | MAT1 |
| 6 | 50 nm | mCBP (84%): |
| | | MAT2 (15%): |
| | | Example 3 (1%) |
| 5 | 10 nm | mCBP |
| 4 | 10 nm | TCTA |
| 3 | 50 nm | NPB |
| 2 | 5 nm | HAT-CN |
| 1 | 50 nm | ITO |
| Substrate | | Glass |

OLED D4 yielded an external quantum efficiency (EQE) at 1000 cd/m² of 16.3%. The emission maximum is at 516 nm with a FWHM of 40 nm at 7.0 V. The corresponding CIEx value is 0.27 and the CIEy value is 0.65. A LT95-value at 1200 cd/m² of 1162 h was determined.

### Example D5

Example 6 was tested in the OLED D5, which was fabricated with the following layer structure:

| Layer # | Thickness | D5 |
|---|---|---|
| 10 | 100 nm | Al |
| 9 | 2 nm | Liq |
| 8 | 20 nm | NBPhen |
| 7 | 10 nm | MAT1 |
| 6 | 50 nm | mCBP (84%): |
| | | MAT5 (15%): |
| | | Example 6 (1%) |
| 5 | 10 nm | mCBP |
| 4 | 10 nm | TCTA |
| 3 | 50 nm | NPB |
| 2 | 5 nm | HAT-CN |
| 1 | 50 nm | ITO |
| Substrate | | Glass |

OLED D5 yielded an external quantum efficiency (EQE) at 1000 cd/m² of 14.9%. The emission maximum is at 535 nm with a FWHM of 36 nm at 7.3 V. The corresponding CIEx value is 0.32 and the CIEy value is 0.64. A LT95-value at 1200 cd/m² of 178 h was determined.

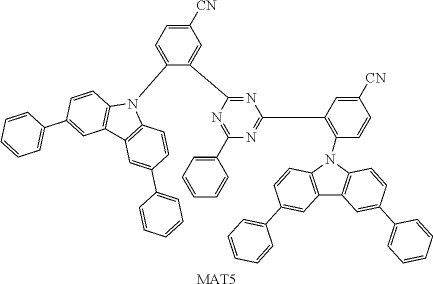

### Example D6

Example 7 was tested in the OLED D6, which was fabricated with the following layer structure:

| Layer # | Thickness | D6 |
|---|---|---|
| 10 | 100 nm | Al |
| 9 | 2 nm | Liq |
| 8 | 10 nm | NBPhen |
| 7 | 10 nm | MAT1 |
| 6 | 50 nm | mCBP (84%): |
| | | MAT5 (15%): |
| | | Example 7 (1%) |
| 5 | 10 nm | mCBP |
| 4 | 10 nm | TCTA |
| 3 | 50 nm | NPB |
| 2 | 5 nm | HAT-CN |
| 1 | 50 nm | ITO |
| Substrate | | Glass |

OLED D6 yielded an external quantum efficiency (EQE) at 1000 cd/m² of 18.5%. The emission maximum is at 524 nm with a FWHM of 36 nm at 7.9 V. The corresponding CIEx value is 0.28 and the CIEy value is 0.66. A LT95-value at 1200 cd/m² of 1144 h was determined.

### Example D7

Example 2 was tested in the OLED D7, which was fabricated with the following layer structure:

| Layer # | Thickness | D7 |
|---|---|---|
| 10 | 100 nm | Al |
| 9 | 2 nm | Liq |
| 8 | 10 nm | NBPhen |
| 7 | 10 nm | MAT1 |
| 6 | 50 nm | mCBP (84%): |
| | | MAT5 (15%): |
| | | Example 2 (1%) |
| 5 | 10 nm | mCBP |
| 4 | 10 nm | TCTA |
| 3 | 50 nm | NPB |
| 2 | 5 nm | HAT-CN |
| 1 | 50 nm | ITO |
| Substrate | | Glass |

OLED D7 yielded an external quantum efficiency (EQE) at 1000 cd/m² of 17.7%. The emission maximum is at 532 nm with a FWHM of 36 nm at 7.5 V. The corresponding CIEx value is 0.31 and the CIEy value is 0.65. A LT95-value at 1200 cd/m² of 1883 h was determined.

### Example D8

Example 2 was tested in the OLED D8, which was fabricated with the following layer structure:

| Layer # | Thickness | D8 |
|---|---|---|
| 10 | 100 nm | Al |
| 9 | 2 nm | Liq |
| 8 | 10 nm | NBPhen |
| 7 | 10 nm | MAT1 |
| 6 | 50 nm | MAT3 (84%): |
| | | MAT5 (15%): |
| | | Example 2 (1%) |
| 5 | 10 nm | MAT3 |
| 4 | 10 nm | TCTA |
| 3 | 50 nm | NPB |
| 2 | 5 nm | HAT-CN |
| 1 | 50 nm | ITO |
| Substrate | | Glass |

OLED D8 yielded an external quantum efficiency (EQE) at 1000 cd/m² of 13.6%. The emission maximum is at 534 nm with a FWHM of 42 nm at 7.5 V. The corresponding CIEx value is 0.32 and the CIEy value is 0.64. A LT95-value at 1200 cd/m² of 1624 h was determined.

### Additional Examples of Organic Molecules of the Invention

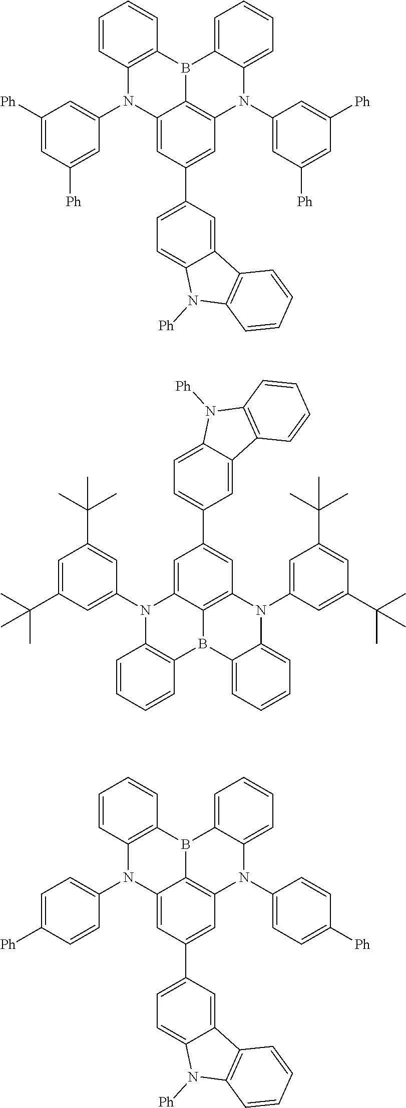 [SMILES: c1ccc(-c2cc(-c3ccccc3)cc(N3c4ccccc4B4c5ccccc5N(c5cc(-c6ccccc6)cc(-c6ccccc6)c5)c5cc(-c6ccc7c(c6)c6ccccc6n7-c6ccccc6)cc3c54)c2)cc1 ; CC(C)(C)c1cc(N2c3ccccc3B3c4ccccc4N(c4cc(C(C)(C)C)cc(C(C)(C)C)c4)c4cc(-c5ccc6c(c5)c5ccccc5n6-c5ccccc5)cc2c43)cc(C(C)(C)C)c1 ; c1ccc(-c2ccc(N3c4ccccc4B4c5ccccc5N(c5ccc(-c6ccccc6)cc5)c5cc(-c6ccc7c(c6)c6ccccc6n7-c6ccccc6)cc3c54)cc2)cc1]

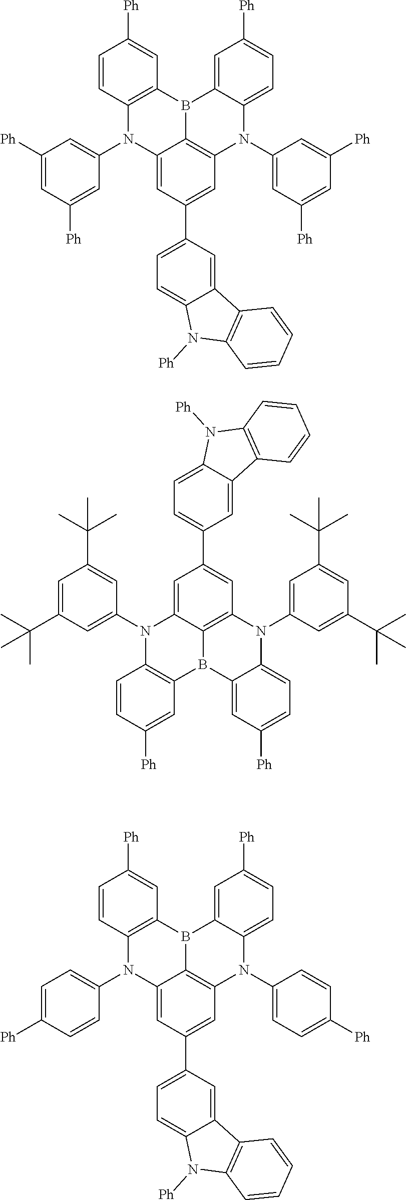

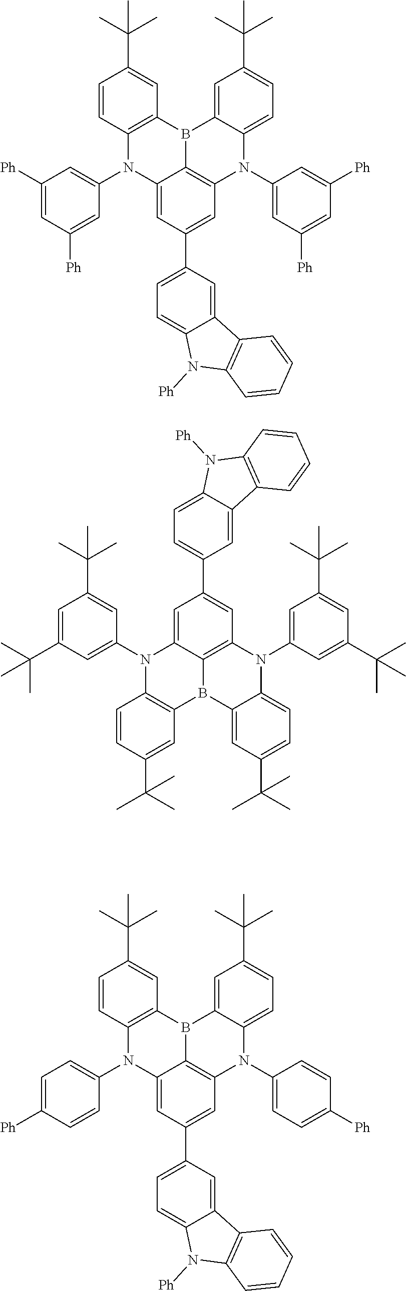

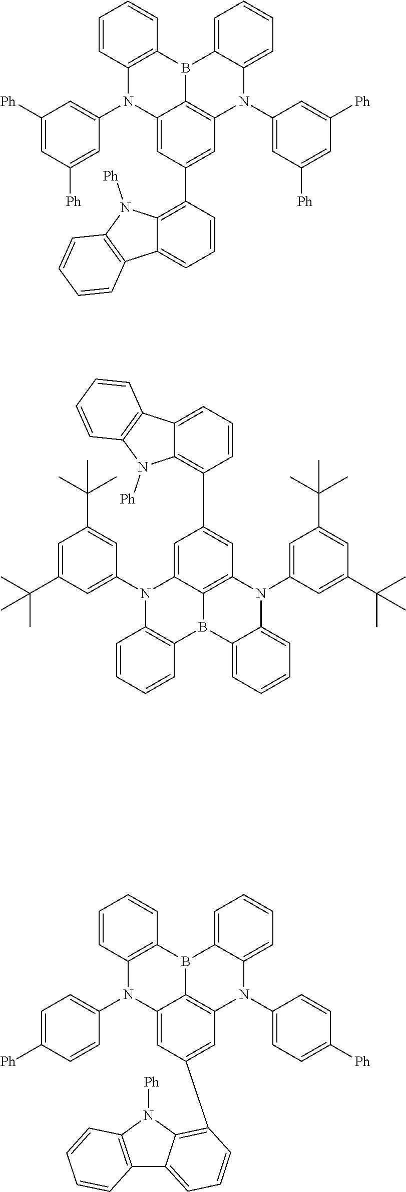 [SMILES: c1ccc(-c2cc(-c3ccccc3)cc(N3c4ccccc4B4c5ccccc5N(c5cc(-c6ccccc6)cc(-c6ccccc6)c5)c5cc(-c6cccc7c8ccccc8n(-c8ccccc8)c67)cc3c54)c2)cc1 ; CC(C)(C)c1cc(N2c3ccccc3B3c4ccccc4N(c4cc(C(C)(C)C)cc(C(C)(C)C)c4)c4cc(-c5cccc6c7ccccc7n(-c7ccccc7)c56)cc2c43)cc(C(C)(C)C)c1 ; c1ccc(-c2ccc(N3c4ccccc4B4c5ccccc5N(c5ccc(-c6ccccc6)cc5)c5cc(-c6cccc7c8ccccc8n(-c8ccccc8)c67)cc3c54)cc2)cc1]

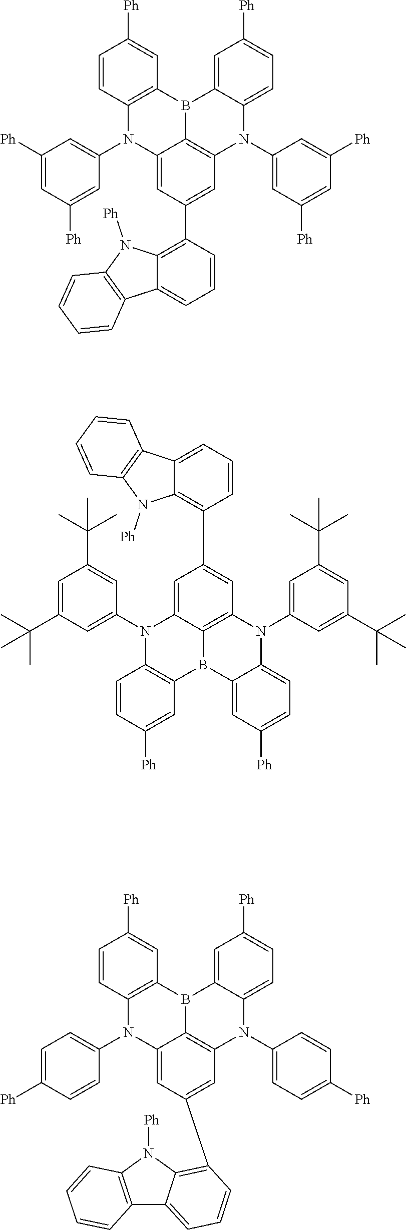 [SMILES: c1ccc(-c2cc(-c3ccccc3)cc(N3c4ccc(-c5ccccc5)cc4B4c5cc(-c6ccccc6)ccc5N(c5cc(-c6ccccc6)cc(-c6ccccc6)c5)c5cc(-c6cccc7c8ccccc8n(-c8ccccc8)c67)cc3c54)c2)cc1 ; CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5cccc6c7ccccc7n(-c7ccccc7)c56)cc2c43)cc(C(C)(C)C)c1 ; c1ccc(-c2ccc(N3c4ccc(-c5ccccc5)cc4B4c5cc(-c6ccccc6)ccc5N(c5ccc(-c6ccccc6)cc5)c5cc(-c6cccc7c8ccccc8n(-c8ccccc8)c67)cc3c54)cc2)cc1]

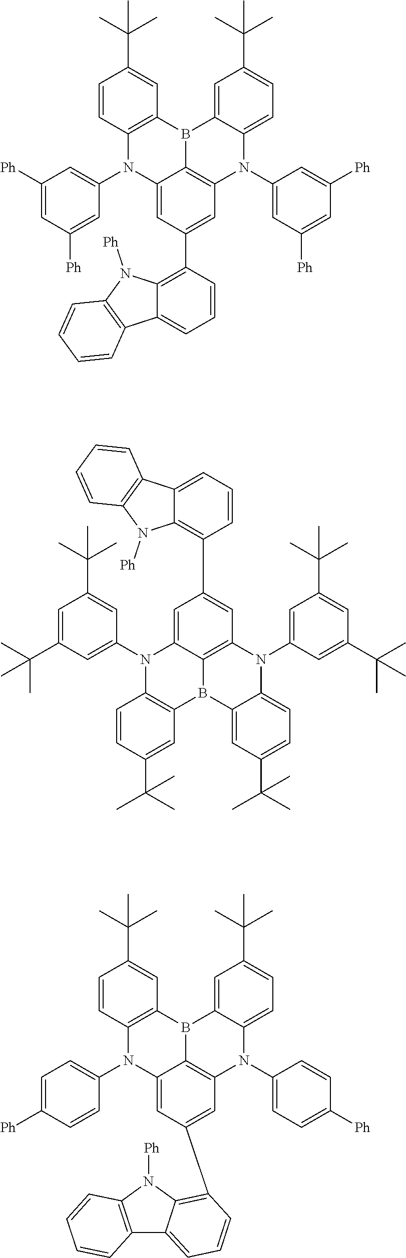 [SMILES: CC(C)(C)c1ccc2c(c1)B1c3cc(C(C)(C)C)ccc3N(c3cc(-c4ccccc4)cc(-c4ccccc4)c3)c3cc(-c4cccc5c6ccccc6n(-c6ccccc6)c45)cc(c31)N2c1cc(-c2ccccc2)cc(-c2ccccc2)c1 ; CC(C)(C)c1cc(N2c3ccc(C(C)(C)C)cc3B3c4cc(C(C)(C)C)ccc4N(c4cc(C(C)(C)C)cc(C(C)(C)C)c4)c4cc(-c5cccc6c7ccccc7n(-c7ccccc7)c56)cc2c43)cc(C(C)(C)C)c1 ; CC(C)(C)c1ccc2c(c1)B1c3cc(C(C)(C)C)ccc3N(c3ccc(-c4ccccc4)cc3)c3cc(-c4cccc5c6ccccc6n(-c6ccccc6)c45)cc(c31)N2c1ccc(-c2ccccc2)cc1]

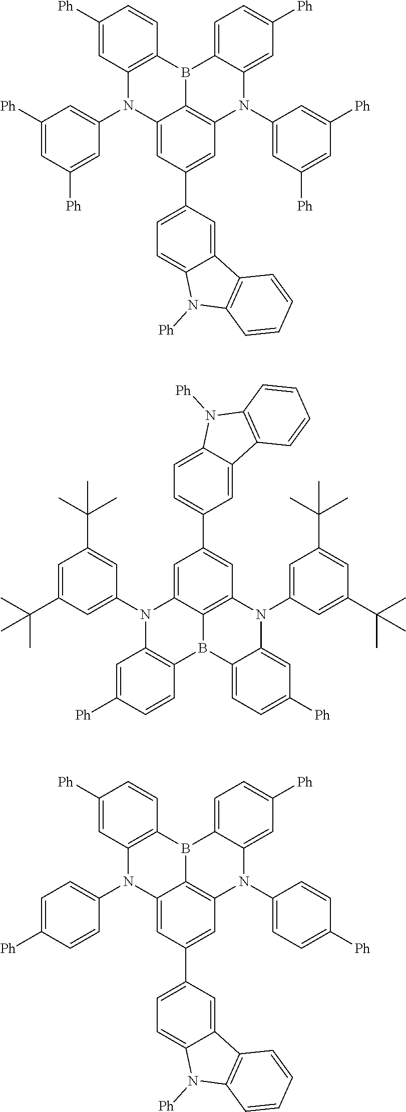 [SMILES: c1ccc(-c2cc(-c3ccccc3)cc(N3c4cc(-c5ccccc5)ccc4B4c5ccc(-c6ccccc6)cc5N(c5cc(-c6ccccc6)cc(-c6ccccc6)c5)c5cc(-c6ccc7c(c6)c6ccccc6n7-c6ccccc6)cc3c54)c2)cc1 ; CC(C)(C)c1cc(N2c3cc(-c4ccccc4)ccc3B3c4ccc(-c5ccccc5)cc4N(c4cc(C(C)(C)C)cc(C(C)(C)C)c4)c4cc(-c5ccc6c(c5)c5ccccc5n6-c5ccccc5)cc2c43)cc(C(C)(C)C)c1 ; c1ccc(-c2ccc(N3c4cc(-c5ccccc5)ccc4B4c5ccc(-c6ccccc6)cc5N(c5ccc(-c6ccccc6)cc5)c5cc(-c6ccc7c(c6)c6ccccc6n7-c6ccccc6)cc3c54)cc2)cc1]

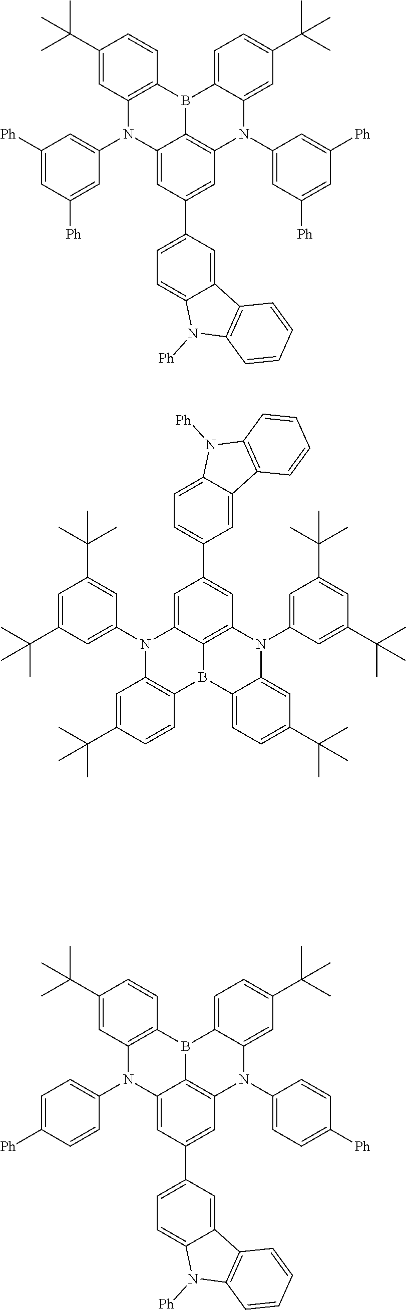 [SMILES: CC(C)(C)c1ccc2c(c1)N(c1cc(-c3ccccc3)cc(-c3ccccc3)c1)c1cc(-c3ccc4c(c3)c3ccccc3n4-c3ccccc3)cc3c1B2c1ccc(C(C)(C)C)cc1N3c1cc(-c2ccccc2)cc(-c2ccccc2)c1 ; CC(C)(C)c1cc(N2c3cc(C(C)(C)C)ccc3B3c4ccc(C(C)(C)C)cc4N(c4cc(C(C)(C)C)cc(C(C)(C)C)c4)c4cc(-c5ccc6c(c5)c5ccccc5n6-c5ccccc5)cc2c43)cc(C(C)(C)C)c1 ; CC(C)(C)c1ccc2c(c1)N(c1ccc(-c3ccccc3)cc1)c1cc(-c3ccc4c(c3)c3ccccc3n4-c3ccccc3)cc3c1B2c1ccc(C(C)(C)C)cc1N3c1ccc(-c2ccccc2)cc1]

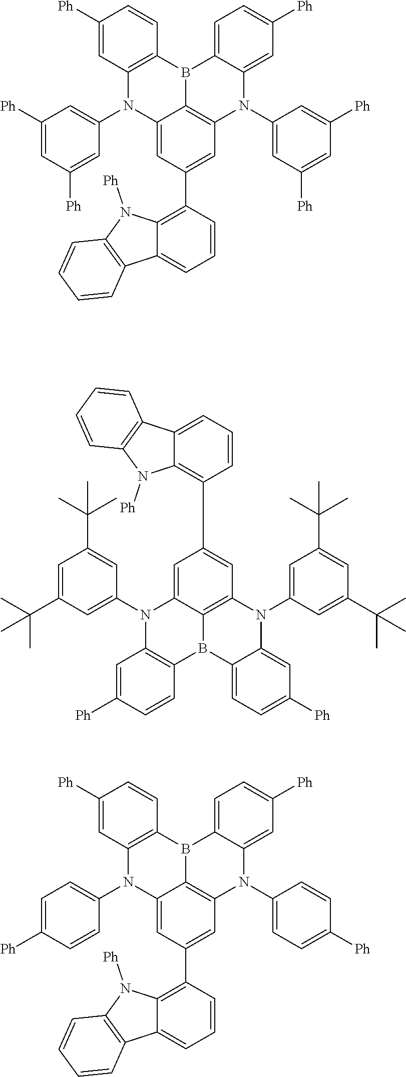 [SMILES: c1ccc(-c2cc(-c3ccccc3)cc(N3c4cc(-c5ccccc5)ccc4B4c5ccc(-c6ccccc6)cc5N(c5cc(-c6ccccc6)cc(-c6ccccc6)c5)c5cc(-c6cccc7c8ccccc8n(-c8ccccc8)c67)cc3c54)c2)cc1 ; CC(C)(C)c1cc(N2c3cc(-c4ccccc4)ccc3B3c4ccc(-c5ccccc5)cc4N(c4cc(C(C)(C)C)cc(C(C)(C)C)c4)c4cc(-c5cccc6c7ccccc7n(-c7ccccc7)c56)cc2c43)cc(C(C)(C)C)c1 ; c1ccc(-c2ccc(N3c4cc(-c5ccccc5)ccc4B4c5ccc(-c6ccccc6)cc5N(c5ccc(-c6ccccc6)cc5)c5cc(-c6cccc7c8ccccc8n(-c8ccccc8)c67)cc3c54)cc2)cc1]

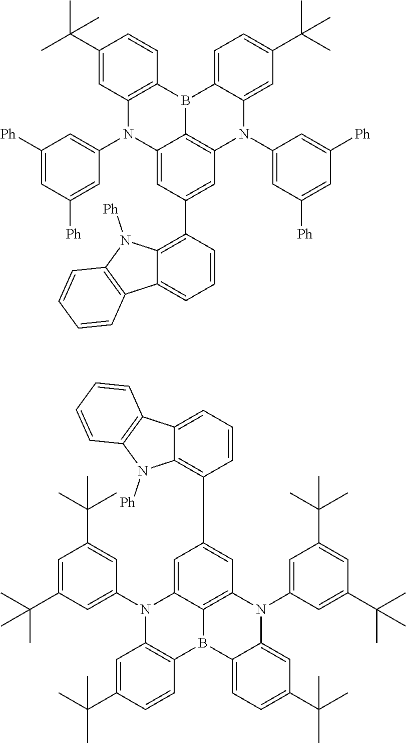 [SMILES: CC(C)(C)c1ccc2c(c1)N(c1cc(-c3ccccc3)cc(-c3ccccc3)c1)c1cc(-c3cccc4c5ccccc5n(-c5ccccc5)c34)cc3c1B2c1ccc(C(C)(C)C)cc1N3c1cc(-c2ccccc2)cc(-c2ccccc2)c1 ; CC(C)(C)c1cc(N2c3cc(C(C)(C)C)ccc3B3c4ccc(C(C)(C)C)cc4N(c4cc(C(C)(C)C)cc(C(C)(C)C)c4)c4cc(-c5cccc6c7ccccc7n(-c7ccccc7)c56)cc2c43)cc(C(C)(C)C)c1]

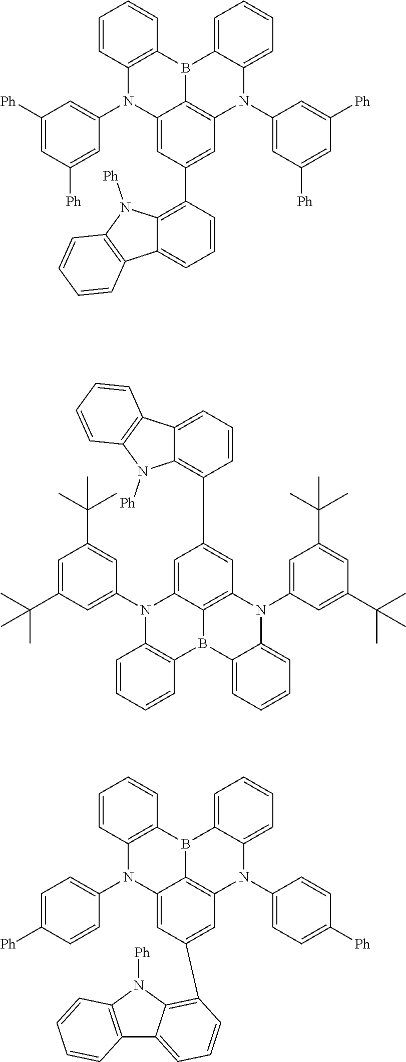 [SMILES: c1ccc(-c2cc(-c3ccccc3)cc(N3c4ccccc4B4c5ccccc5N(c5cc(-c6ccccc6)cc(-c6ccccc6)c5)c5cc(-c6cccc7c8ccccc8n(-c8ccccc8)c67)cc3c54)c2)cc1 ; CC(C)(C)c1cc(N2c3ccccc3B3c4ccccc4N(c4cc(C(C)(C)C)cc(C(C)(C)C)c4)c4cc(-c5cccc6c7ccccc7n(-c7ccccc7)c56)cc2c43)cc(C(C)(C)C)c1 ; c1ccc(-c2ccc(N3c4ccccc4B4c5ccccc5N(c5ccc(-c6ccccc6)cc5)c5cc(-c6cccc7c8ccccc8n(-c8ccccc8)c67)cc3c54)cc2)cc1]

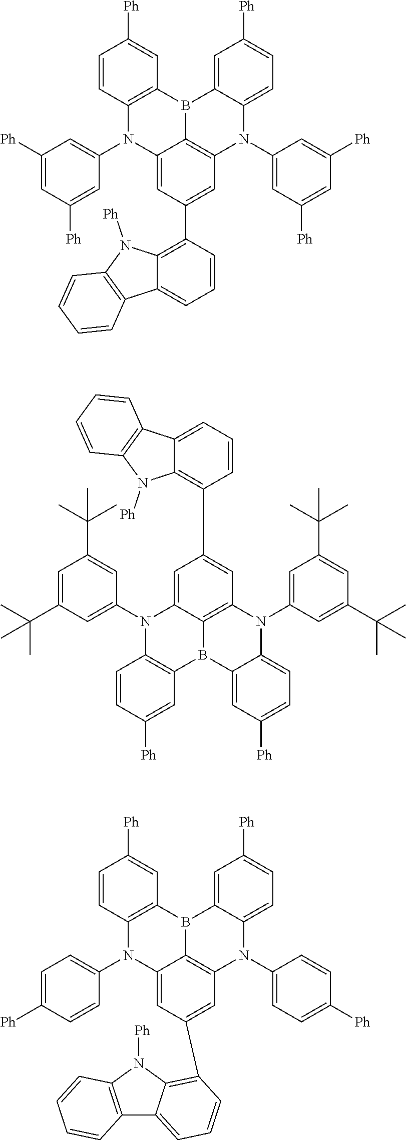 [SMILES: c1ccc(-c2cc(-c3ccccc3)cc(N3c4ccc(-c5ccccc5)cc4B4c5cc(-c6ccccc6)ccc5N(c5cc(-c6ccccc6)cc(-c6ccccc6)c5)c5cc(-c6cccc7c8ccccc8n(-c8ccccc8)c67)cc3c54)c2)cc1 ; CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5cccc6c7ccccc7n(-c7ccccc7)c56)cc2c43)cc(C(C)(C)C)c1 ; c1ccc(-c2ccc(N3c4ccc(-c5ccccc5)cc4B4c5cc(-c6ccccc6)ccc5N(c5ccc(-c6ccccc6)cc5)c5cc(-c6cccc7c8ccccc8n(-c8ccccc8)c67)cc3c54)cc2)cc1]

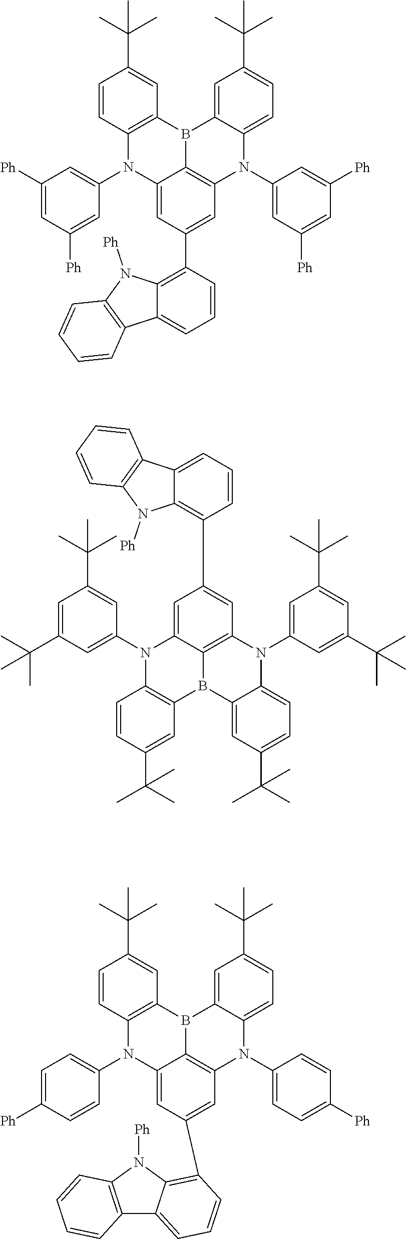 [SMILES: CC(C)(C)c1ccc2c(c1)B1c3cc(C(C)(C)C)ccc3N(c3cc(-c4ccccc4)cc(-c4ccccc4)c3)c3cc(-c4cccc5c6ccccc6n(-c6ccccc6)c45)cc(c31)N2c1cc(-c2ccccc2)cc(-c2ccccc2)c1 ; CC(C)(C)c1cc(N2c3ccc(C(C)(C)C)cc3B3c4cc(C(C)(C)C)ccc4N(c4cc(C(C)(C)C)cc(C(C)(C)C)c4)c4cc(-c5cccc6c7ccccc7n(-c7ccccc7)c56)cc2c43)cc(C(C)(C)C)c1 ; CC(C)(C)c1ccc2c(c1)B1c3cc(C(C)(C)C)ccc3N(c3ccc(-c4ccccc4)cc3)c3cc(-c4cccc5c6ccccc6n(-c6ccccc6)c45)cc(c31)N2c1ccc(-c2ccccc2)cc1]

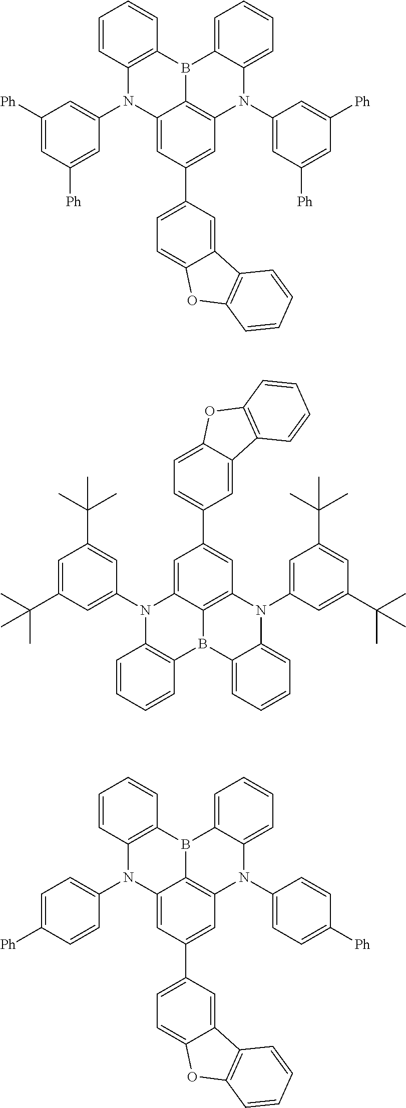 [SMILES: c1ccc(-c2cc(-c3ccccc3)cc(N3c4ccccc4B4c5ccccc5N(c5cc(-c6ccccc6)cc(-c6ccccc6)c5)c5cc(-c6ccc7oc8ccccc8c7c6)cc3c54)c2)cc1 ; CC(C)(C)c1cc(N2c3ccccc3B3c4ccccc4N(c4cc(C(C)(C)C)cc(C(C)(C)C)c4)c4cc(-c5ccc6oc7ccccc7c6c5)cc2c43)cc(C(C)(C)C)c1 ; c1ccc(-c2ccc(N3c4ccccc4B4c5ccccc5N(c5ccc(-c6ccccc6)cc5)c5cc(-c6ccc7oc8ccccc8c7c6)cc3c54)cc2)cc1]

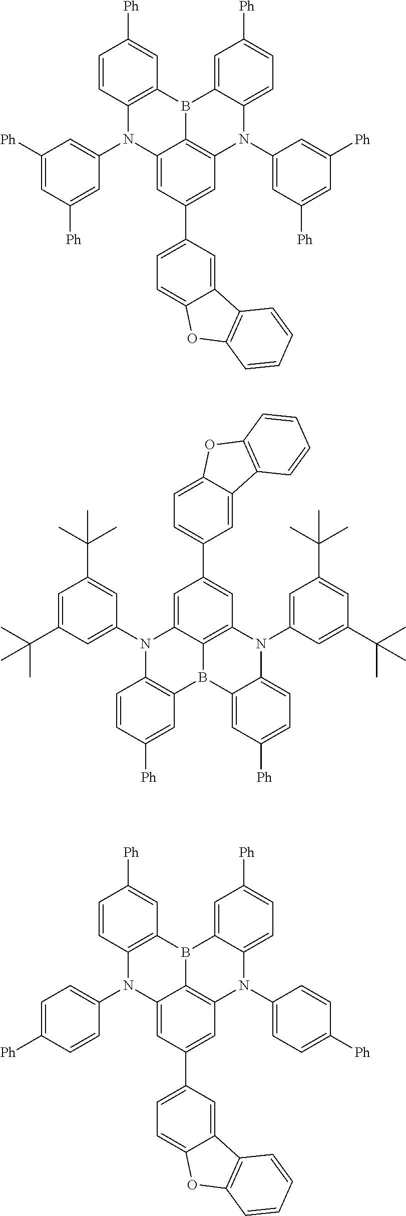 [SMILES: c1ccc(-c2cc(-c3ccccc3)cc(N3c4ccc(-c5ccccc5)cc4B4c5cc(-c6ccccc6)ccc5N(c5cc(-c6ccccc6)cc(-c6ccccc6)c5)c5cc(-c6ccc7oc8ccccc8c7c6)cc3c54)c2)cc1 ; CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5ccc6oc7ccccc7c6c5)cc2c43)cc(C(C)(C)C)c1 ; c1ccc(-c2ccc(N3c4ccc(-c5ccccc5)cc4B4c5cc(-c6ccccc6)ccc5N(c5ccc(-c6ccccc6)cc5)c5cc(-c6ccc7oc8ccccc8c7c6)cc3c54)cc2)cc1]

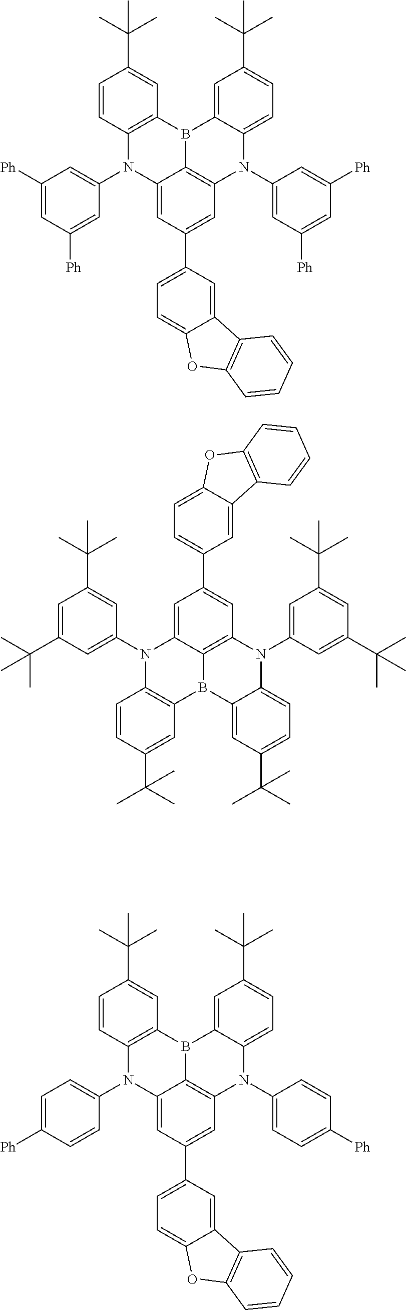

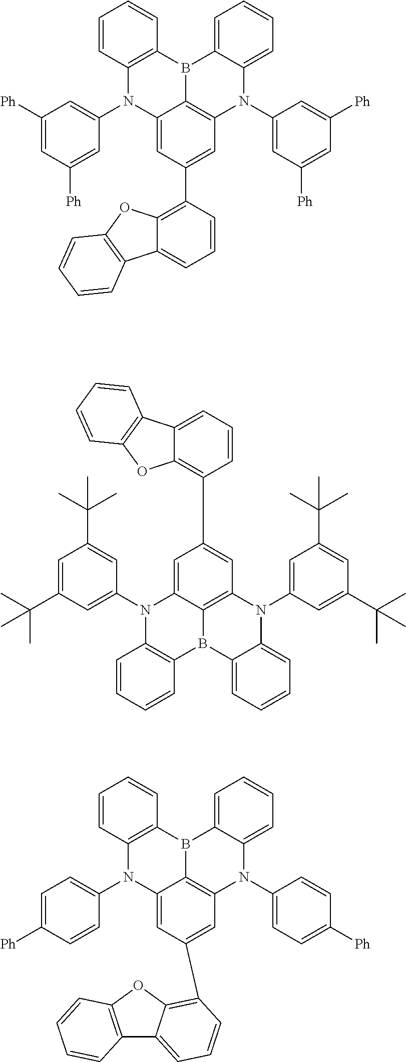 [SMILES: c1ccc(-c2cc(-c3ccccc3)cc(N3c4ccccc4B4c5ccccc5N(c5cc(-c6ccccc6)cc(-c6ccccc6)c5)c5cc(-c6cccc7c6oc6ccccc67)cc3c54)c2)cc1 ; CC(C)(C)c1cc(N2c3ccccc3B3c4ccccc4N(c4cc(C(C)(C)C)cc(C(C)(C)C)c4)c4cc(-c5cccc6c5oc5ccccc56)cc2c43)cc(C(C)(C)C)c1 ; c1ccc(-c2ccc(N3c4ccccc4B4c5ccccc5N(c5ccc(-c6ccccc6)cc5)c5cc(-c6cccc7c6oc6ccccc67)cc3c54)cc2)cc1]

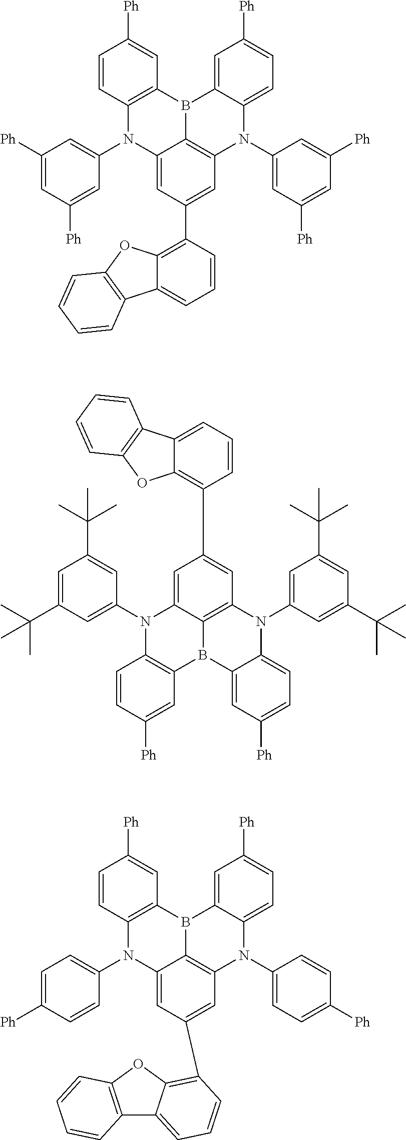 [SMILES: c1ccc(-c2cc(-c3ccccc3)cc(N3c4ccc(-c5ccccc5)cc4B4c5cc(-c6ccccc6)ccc5N(c5cc(-c6ccccc6)cc(-c6ccccc6)c5)c5cc(-c6cccc7c6oc6ccccc67)cc3c54)c2)cc1 ; CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5cccc6c5oc5ccccc56)cc2c43)cc(C(C)(C)C)c1 ; c1ccc(-c2ccc(N3c4ccc(-c5ccccc5)cc4B4c5cc(-c6ccccc6)ccc5N(c5ccc(-c6ccccc6)cc5)c5cc(-c6cccc7c6oc6ccccc67)cc3c54)cc2)cc1]

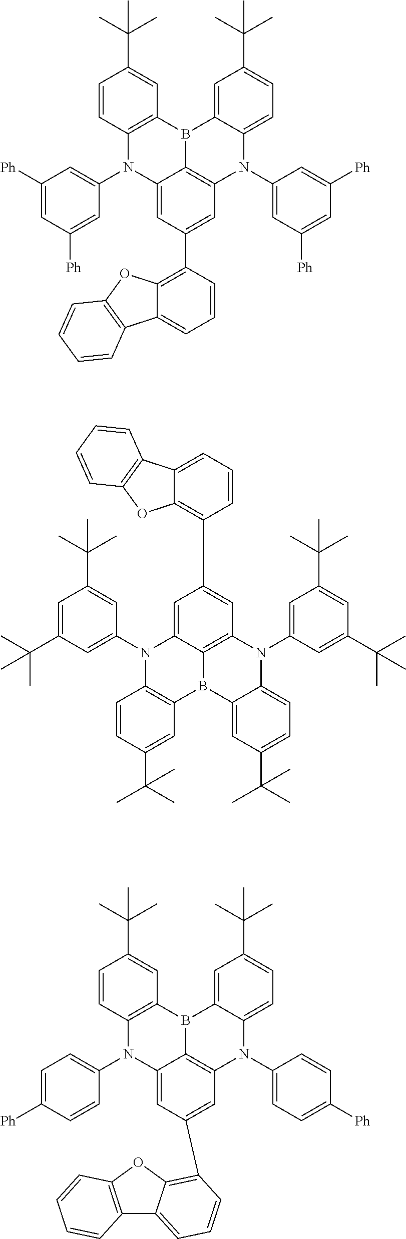 [SMILES: CC(C)(C)c1ccc2c(c1)B1c3cc(C(C)(C)C)ccc3N(c3cc(-c4ccccc4)cc(-c4ccccc4)c3)c3cc(-c4cccc5c4oc4ccccc45)cc(c31)N2c1cc(-c2ccccc2)cc(-c2ccccc2)c1 ; CC(C)(C)c1cc(N2c3ccc(C(C)(C)C)cc3B3c4cc(C(C)(C)C)ccc4N(c4cc(C(C)(C)C)cc(C(C)(C)C)c4)c4cc(-c5cccc6c5oc5ccccc56)cc2c43)cc(C(C)(C)C)c1 ; CC(C)(C)c1ccc2c(c1)B1c3cc(C(C)(C)C)ccc3N(c3ccc(-c4ccccc4)cc3)c3cc(-c4cccc5c4oc4ccccc45)cc(c31)N2c1ccc(-c2ccccc2)cc1]

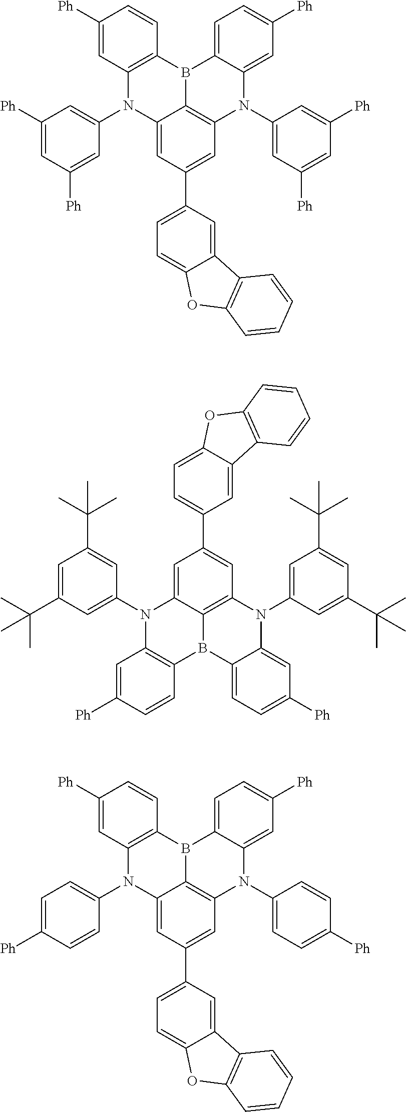 [SMILES: c1ccc(-c2cc(-c3ccccc3)cc(N3c4cc(-c5ccccc5)ccc4B4c5ccc(-c6ccccc6)cc5N(c5cc(-c6ccccc6)cc(-c6ccccc6)c5)c5cc(-c6ccc7oc8ccccc8c7c6)cc3c54)c2)cc1 ; CC(C)(C)c1cc(N2c3cc(-c4ccccc4)ccc3B3c4ccc(-c5ccccc5)cc4N(c4cc(C(C)(C)C)cc(C(C)(C)C)c4)c4cc(-c5ccc6oc7ccccc7c6c5)cc2c43)cc(C(C)(C)C)c1 ; c1ccc(-c2ccc(N3c4cc(-c5ccccc5)ccc4B4c5ccc(-c6ccccc6)cc5N(c5ccc(-c6ccccc6)cc5)c5cc(-c6ccc7oc8ccccc8c7c6)cc3c54)cc2)cc1]

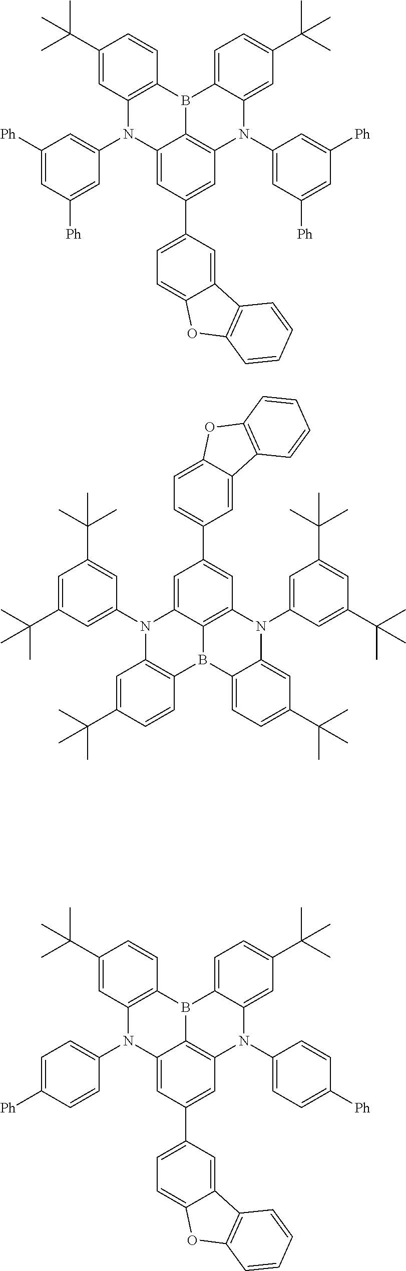

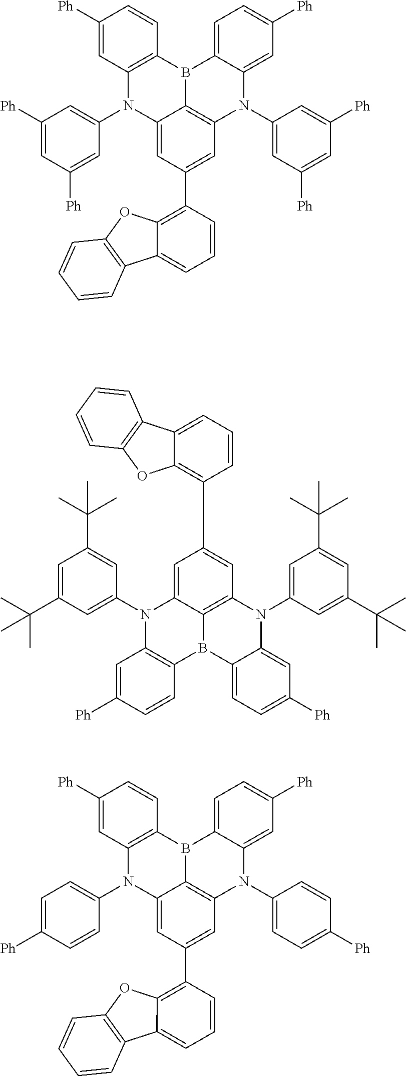 [SMILES: c1ccc(-c2cc(-c3ccccc3)cc(N3c4cc(-c5ccccc5)ccc4B4c5ccc(-c6ccccc6)cc5N(c5cc(-c6ccccc6)cc(-c6ccccc6)c5)c5cc(-c6cccc7c6oc6ccccc67)cc3c54)c2)cc1 ; CC(C)(C)c1cc(N2c3cc(-c4ccccc4)ccc3B3c4ccc(-c5ccccc5)cc4N(c4cc(C(C)(C)C)cc(C(C)(C)C)c4)c4cc(-c5cccc6c5oc5ccccc56)cc2c43)cc(C(C)(C)C)c1 ; c1ccc(-c2ccc(N3c4cc(-c5ccccc5)ccc4B4c5ccc(-c6ccccc6)cc5N(c5ccc(-c6ccccc6)cc5)c5cc(-c6cccc7c6oc6ccccc67)cc3c54)cc2)cc1]

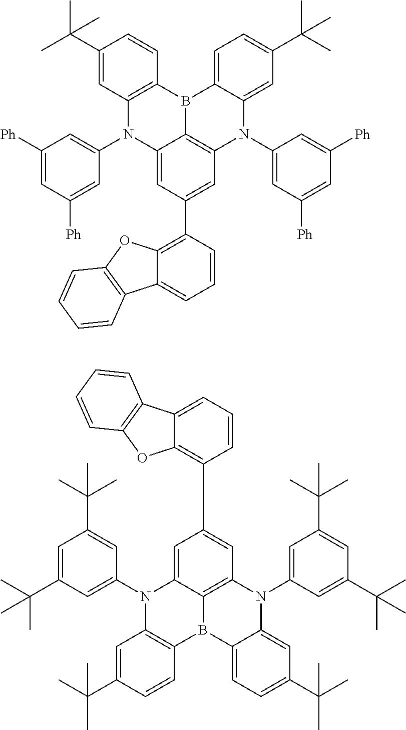

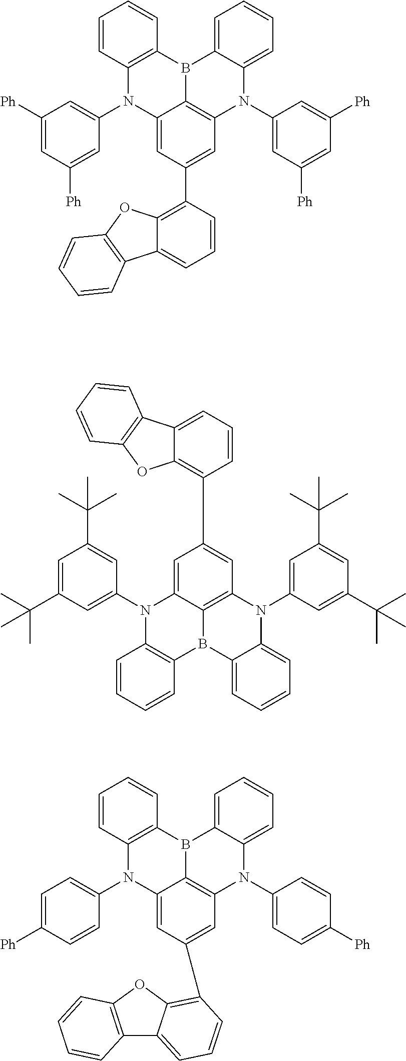 [SMILES: c1ccc(-c2cc(-c3ccccc3)cc(N3c4ccccc4B4c5ccccc5N(c5cc(-c6ccccc6)cc(-c6ccccc6)c5)c5cc(-c6cccc7c6oc6ccccc67)cc3c54)c2)cc1 ; CC(C)(C)c1cc(N2c3ccccc3B3c4ccccc4N(c4cc(C(C)(C)C)cc(C(C)(C)C)c4)c4cc(-c5cccc6c5oc5ccccc56)cc2c43)cc(C(C)(C)C)c1 ; c1ccc(-c2ccc(N3c4ccccc4B4c5ccccc5N(c5ccc(-c6ccccc6)cc5)c5cc(-c6cccc7c6oc6ccccc67)cc3c54)cc2)cc1]

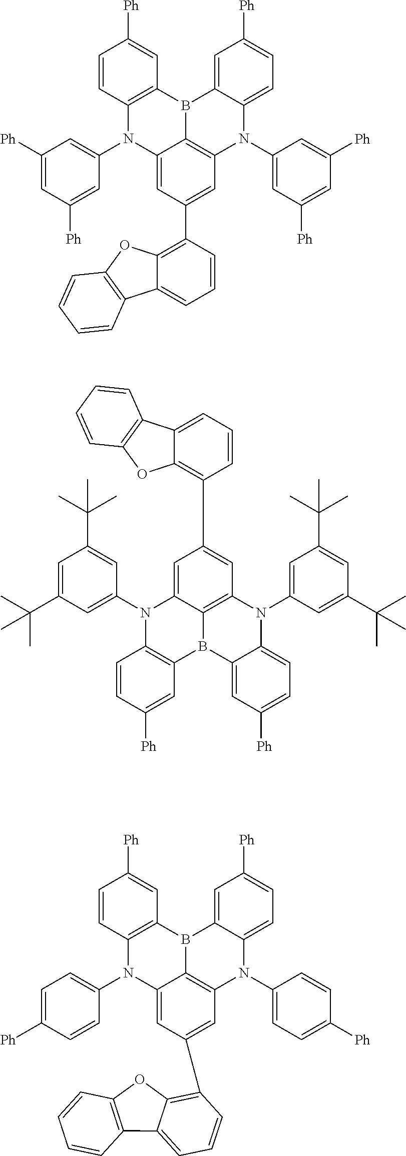 [SMILES: c1ccc(-c2cc(-c3ccccc3)cc(N3c4ccc(-c5ccccc5)cc4B4c5cc(-c6ccccc6)ccc5N(c5cc(-c6ccccc6)cc(-c6ccccc6)c5)c5cc(-c6cccc7c6oc6ccccc67)cc3c54)c2)cc1 ; CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5cccc6c5oc5ccccc56)cc2c43)cc(C(C)(C)C)c1 ; c1ccc(-c2ccc(N3c4ccc(-c5ccccc5)cc4B4c5cc(-c6ccccc6)ccc5N(c5ccc(-c6ccccc6)cc5)c5cc(-c6cccc7c6oc6ccccc67)cc3c54)cc2)cc1]

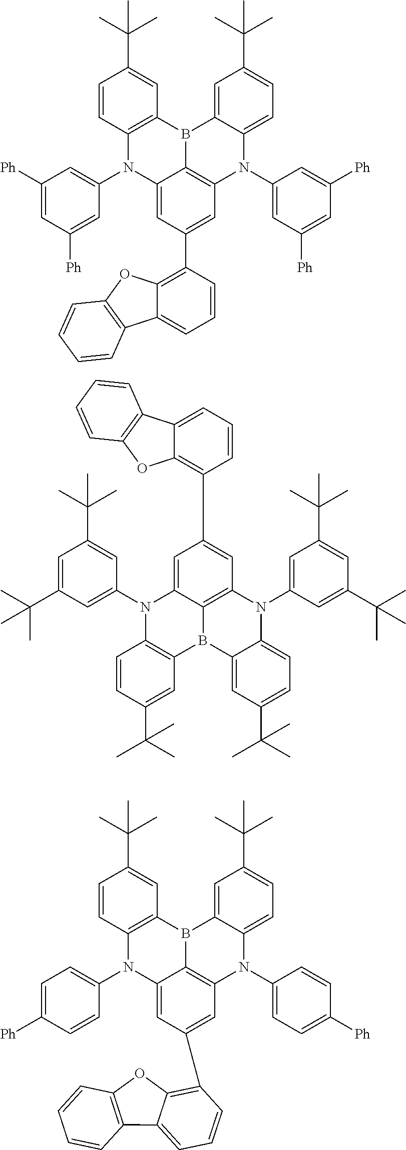

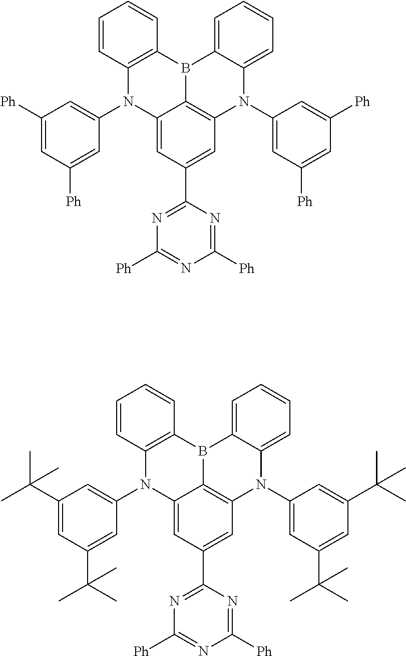 [SMILES: c1ccc(-c2cc(-c3ccccc3)cc(N3c4ccccc4B4c5ccccc5N(c5cc(-c6ccccc6)cc(-c6ccccc6)c5)c5cc(-c6nc(-c7ccccc7)nc(-c7ccccc7)n6)cc3c54)c2)cc1 ; CC(C)(C)c1cc(N2c3ccccc3B3c4ccccc4N(c4cc(C(C)(C)C)cc(C(C)(C)C)c4)c4cc(-c5nc(-c6ccccc6)nc(-c6ccccc6)n5)cc2c43)cc(C(C)(C)C)c1]

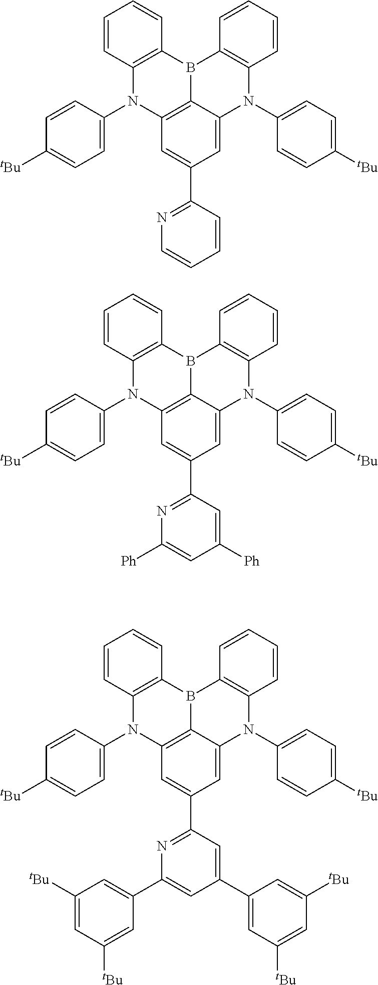 [SMILES: CC(C)(C)c1ccc(N2c3ccccc3B3c4ccccc4N(c4ccc(C(C)(C)C)cc4)c4cc(-c5ccccn5)cc2c43)cc1 ; CC(C)(C)c1ccc(N2c3ccccc3B3c4ccccc4N(c4ccc(C(C)(C)C)cc4)c4cc(-c5cc(-c6ccccc6)cc(-c6ccccc6)n5)cc2c43)cc1 ; CC(C)(C)c1ccc(N2c3ccccc3B3c4ccccc4N(c4ccc(C(C)(C)C)cc4)c4cc(-c5cc(-c6cc(C(C)(C)C)cc(C(C)(C)C)c6)cc(-c6cc(C(C)(C)C)cc(C(C)(C)C)c6)n5)cc2c43)cc1]

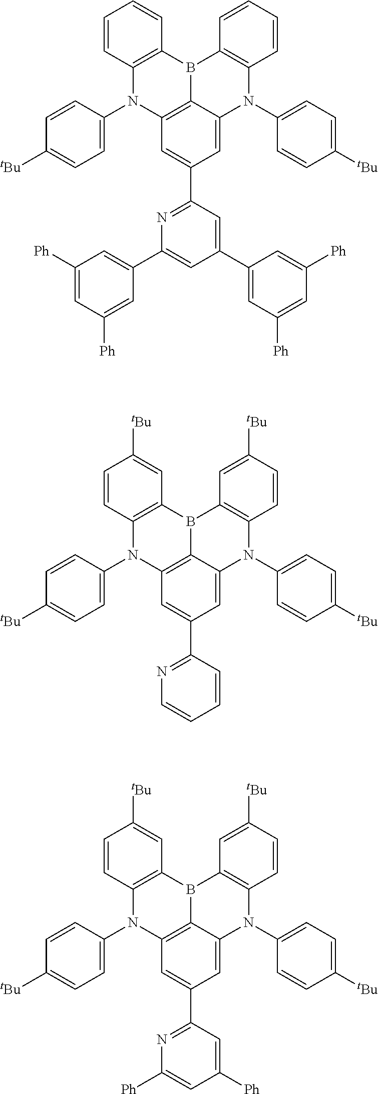 [SMILES: CC(C)(C)c1ccc(N2c3ccccc3B3c4ccccc4N(c4ccc(C(C)(C)C)cc4)c4cc(-c5cc(-c6cc(-c7ccccc7)cc(-c7ccccc7)c6)cc(-c6cc(-c7ccccc7)cc(-c7ccccc7)c6)n5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccccn5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5cc(-c6ccccc6)cc(-c6ccccc6)n5)cc2c43)cc1]

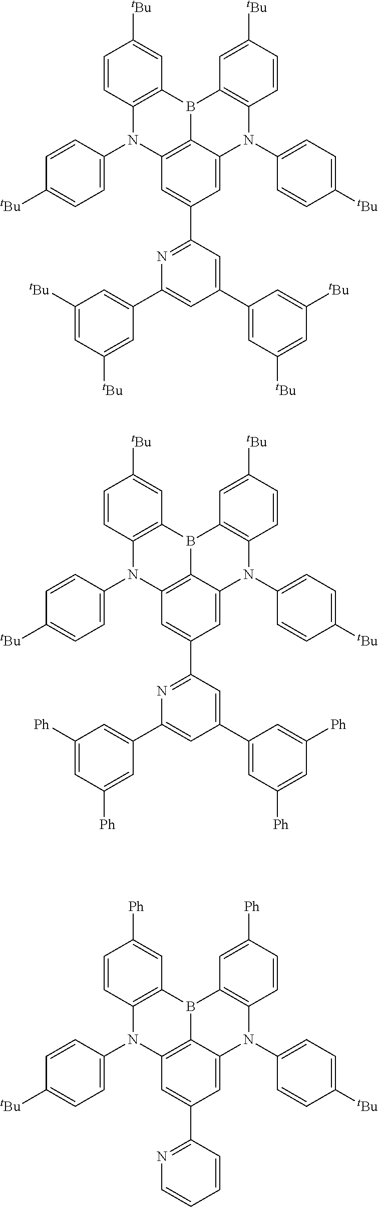 [SMILES: CC(C)(C)c1ccc(N2c3ccc(C(C)(C)C)cc3B3c4cc(C(C)(C)C)ccc4N(c4ccc(C(C)(C)C)cc4)c4cc(-c5cc(-c6cc(C(C)(C)C)cc(C(C)(C)C)c6)cc(-c6cc(C(C)(C)C)cc(C(C)(C)C)c6)n5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5cc(-c6cc(-c7ccccc7)cc(-c7ccccc7)c6)cc(-c6cc(-c7ccccc7)cc(-c7ccccc7)c6)n5)cc2c43)cc1 ; CC(C)(C)c1ccc(N2c3ccc(-c4ccccc4)cc3B3c4cc(-c5ccccc5)ccc4N(c4ccc(C(C)(C)C)cc4)c4cc(-c5ccccn5)cc2c43)cc1]

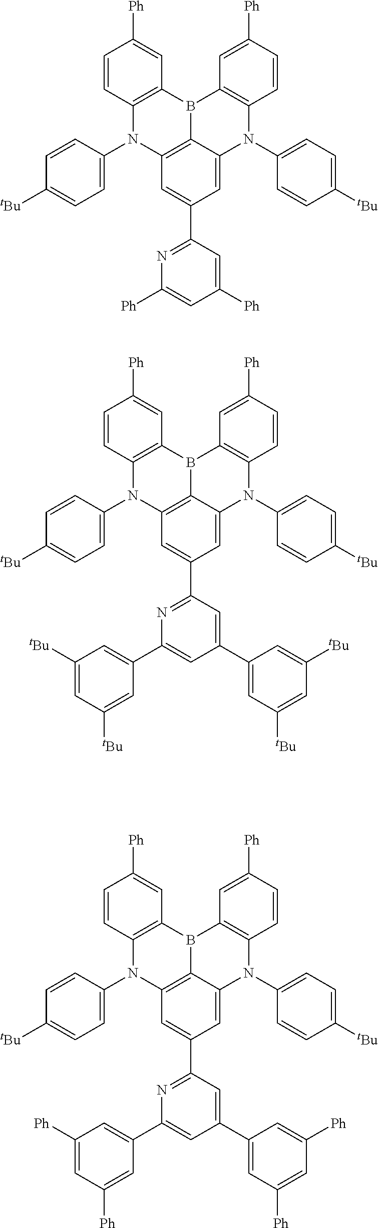 [SMILES: CC(C)(C)c1ccc(N2c3ccc(-c4ccccc4)cc3B3c4cc(-c5ccccc5)ccc4N(c4ccc(C(C)(C)C)cc4)c4cc(-c5cc(-c6ccccc6)cc(-c6ccccc6)n5)cc2c43)cc1 ; CC(C)(C)c1ccc(N2c3ccc(-c4ccccc4)cc3B3c4cc(-c5ccccc5)ccc4N(c4ccc(C(C)(C)C)cc4)c4cc(-c5cc(-c6cc(C(C)(C)C)cc(C(C)(C)C)c6)cc(-c6cc(C(C)(C)C)cc(C(C)(C)C)c6)n5)cc2c43)cc1 ; CC(C)(C)c1ccc(N2c3ccc(-c4ccccc4)cc3B3c4cc(-c5ccccc5)ccc4N(c4ccc(C(C)(C)C)cc4)c4cc(-c5cc(-c6cc(-c7ccccc7)cc(-c7ccccc7)c6)cc(-c6cc(-c7ccccc7)cc(-c7ccccc7)c6)n5)cc2c43)cc1]

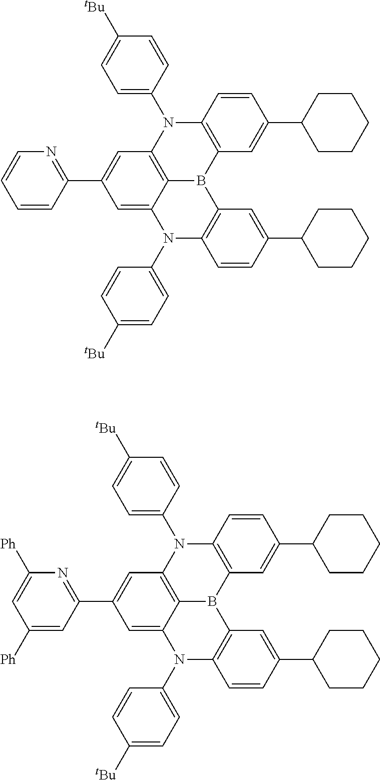 [SMILES: CC(C)(C)c1ccc(N2c3ccc(C4CCCCC4)cc3B3c4cc(C5CCCCC5)ccc4N(c4ccc(C(C)(C)C)cc4)c4cc(-c5ccccn5)cc2c43)cc1 ; CC(C)(C)c1ccc(N2c3ccc(C4CCCCC4)cc3B3c4cc(C5CCCCC5)ccc4N(c4ccc(C(C)(C)C)cc4)c4cc(-c5cc(-c6ccccc6)cc(-c6ccccc6)n5)cc2c43)cc1]

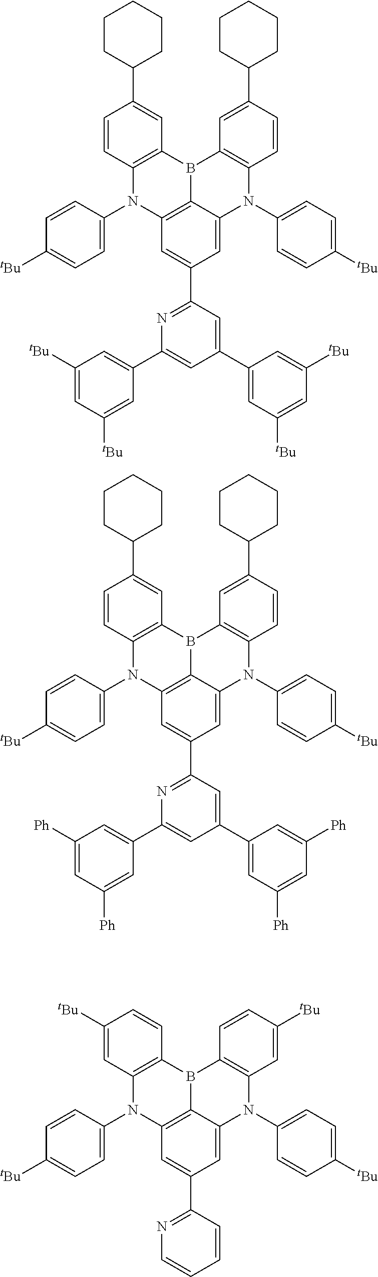

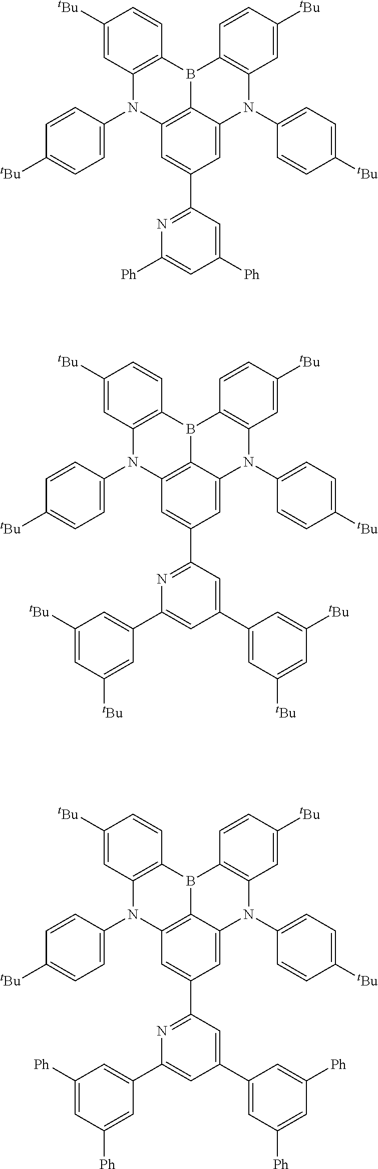 [SMILES: CC(C)(C)c1ccc(N2c3cc(C(C)(C)C)ccc3B3c4ccc(C(C)(C)C)cc4N(c4ccc(C(C)(C)C)cc4)c4cc(-c5cc(-c6ccccc6)cc(-c6ccccc6)n5)cc2c43)cc1 ; CC(C)(C)c1ccc(N2c3cc(C(C)(C)C)ccc3B3c4ccc(C(C)(C)C)cc4N(c4ccc(C(C)(C)C)cc4)c4cc(-c5cc(-c6cc(C(C)(C)C)cc(C(C)(C)C)c6)cc(-c6cc(C(C)(C)C)cc(C(C)(C)C)c6)n5)cc2c43)cc1 ; CC(C)(C)c1ccc(N2c3cc(C(C)(C)C)ccc3B3c4ccc(C(C)(C)C)cc4N(c4ccc(C(C)(C)C)cc4)c4cc(-c5cc(-c6cc(-c7ccccc7)cc(-c7ccccc7)c6)cc(-c6cc(-c7ccccc7)cc(-c7ccccc7)c6)n5)cc2c43)cc1]

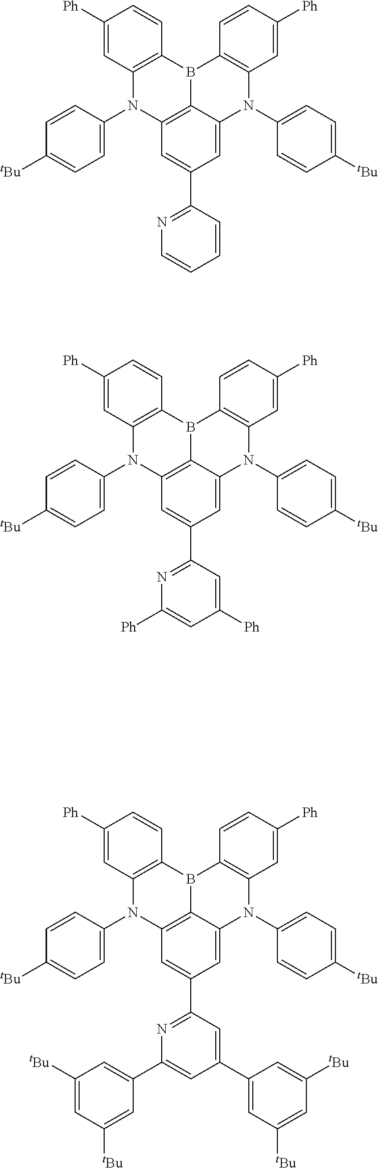 [SMILES: CC(C)(C)c1ccc(N2c3cc(-c4ccccc4)ccc3B3c4ccc(-c5ccccc5)cc4N(c4ccc(C(C)(C)C)cc4)c4cc(-c5ccccn5)cc2c43)cc1 ; CC(C)(C)c1ccc(N2c3cc(-c4ccccc4)ccc3B3c4ccc(-c5ccccc5)cc4N(c4ccc(C(C)(C)C)cc4)c4cc(-c5cc(-c6ccccc6)cc(-c6ccccc6)n5)cc2c43)cc1 ; CC(C)(C)c1ccc(N2c3cc(-c4ccccc4)ccc3B3c4ccc(-c5ccccc5)cc4N(c4ccc(C(C)(C)C)cc4)c4cc(-c5cc(-c6cc(C(C)(C)C)cc(C(C)(C)C)c6)cc(-c6cc(C(C)(C)C)cc(C(C)(C)C)c6)n5)cc2c43)cc1]

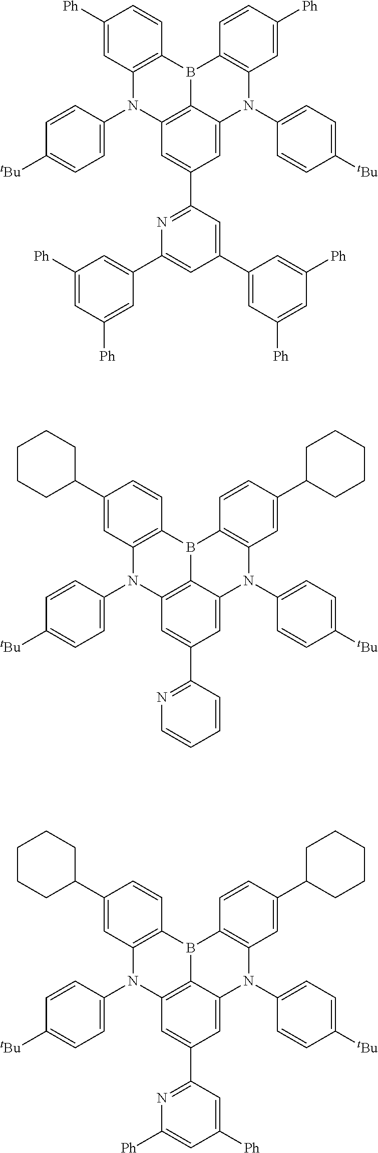 [SMILES: CC(C)(C)c1ccc(N2c3cc(-c4ccccc4)ccc3B3c4ccc(-c5ccccc5)cc4N(c4ccc(C(C)(C)C)cc4)c4cc(-c5cc(-c6cc(-c7ccccc7)cc(-c7ccccc7)c6)cc(-c6cc(-c7ccccc7)cc(-c7ccccc7)c6)n5)cc2c43)cc1 ; CC(C)(C)c1ccc(N2c3cc(C4CCCCC4)ccc3B3c4ccc(C5CCCCC5)cc4N(c4ccc(C(C)(C)C)cc4)c4cc(-c5ccccn5)cc2c43)cc1 ; CC(C)(C)c1ccc(N2c3cc(C4CCCCC4)ccc3B3c4ccc(C5CCCCC5)cc4N(c4ccc(C(C)(C)C)cc4)c4cc(-c5cc(-c6ccccc6)cc(-c6ccccc6)n5)cc2c43)cc1]

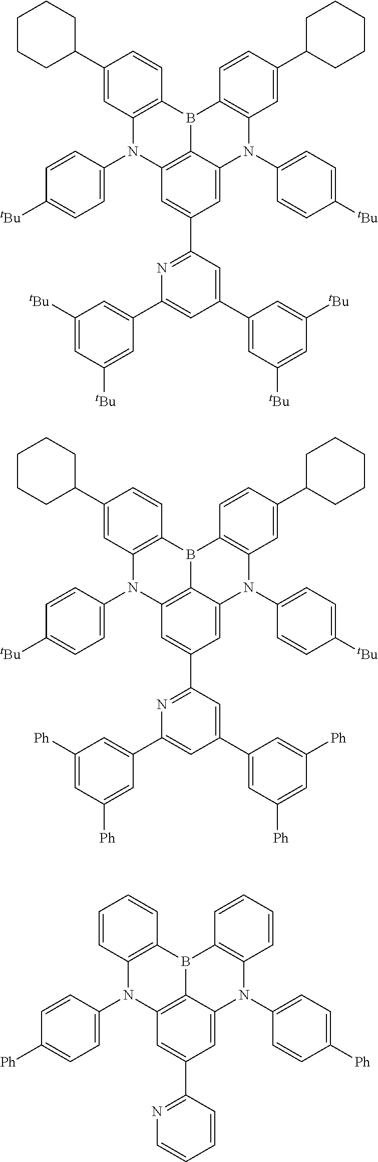 [SMILES: CC(C)(C)c1ccc(N2c3cc(C4CCCCC4)ccc3B3c4ccc(C5CCCCC5)cc4N(c4ccc(C(C)(C)C)cc4)c4cc(-c5cc(-c6cc(C(C)(C)C)cc(C(C)(C)C)c6)cc(-c6cc(C(C)(C)C)cc(C(C)(C)C)c6)n5)cc2c43)cc1 ; CC(C)(C)c1ccc(N2c3cc(C4CCCCC4)ccc3B3c4ccc(C5CCCCC5)cc4N(c4ccc(C(C)(C)C)cc4)c4cc(-c5cc(-c6cc(-c7ccccc7)cc(-c7ccccc7)c6)cc(-c6cc(-c7ccccc7)cc(-c7ccccc7)c6)n5)cc2c43)cc1 ; c1ccc(-c2ccc(N3c4ccccc4B4c5ccccc5N(c5ccc(-c6ccccc6)cc5)c5cc(-c6ccccn6)cc3c54)cc2)cc1]

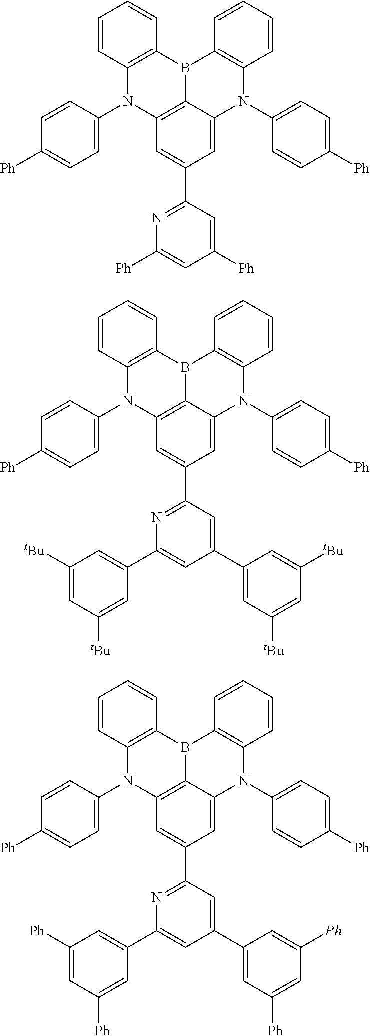

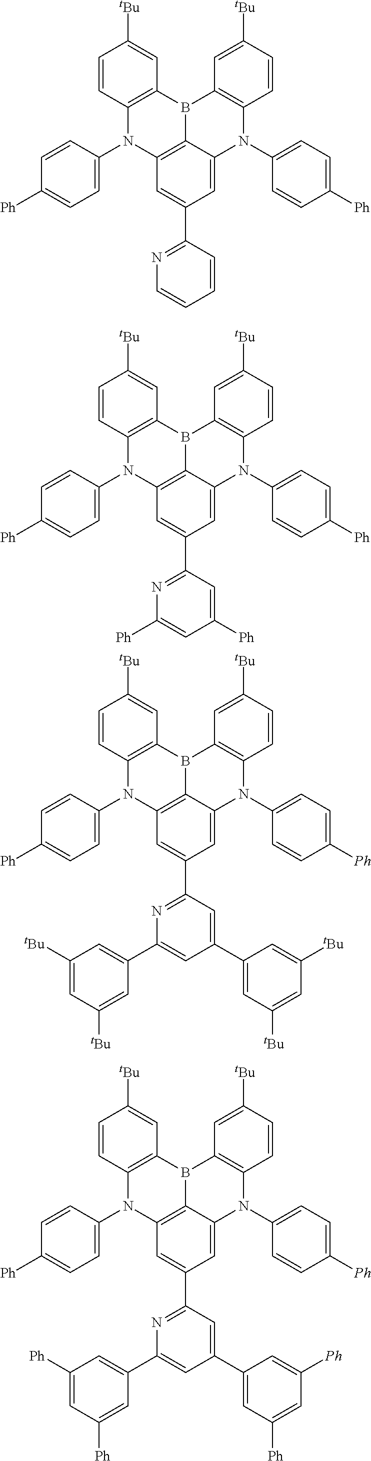

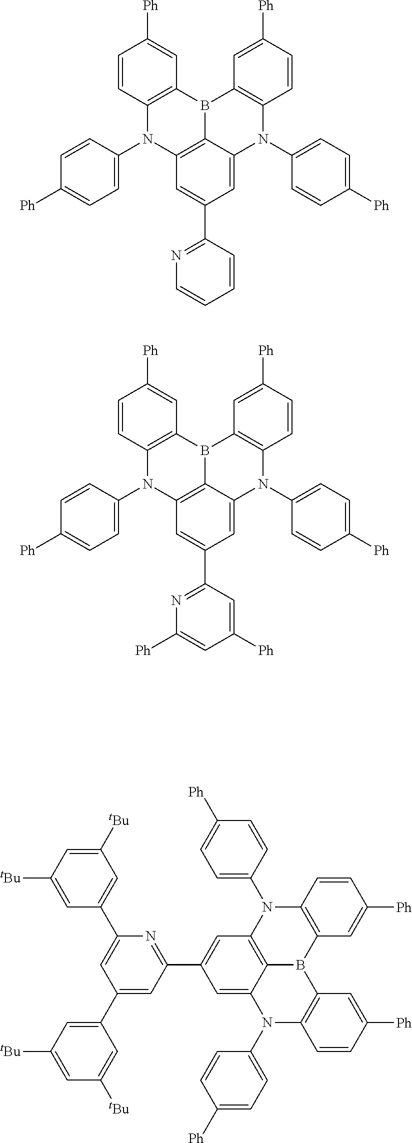 [SMILES: c1ccc(-c2ccc(N3c4ccc(-c5ccccc5)cc4B4c5cc(-c6ccccc6)ccc5N(c5ccc(-c6ccccc6)cc5)c5cc(-c6ccccn6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6cc(-c7ccccc7)cc(-c7ccccc7)n6)cc3c54)cc2)cc1 ; CC(C)(C)c1cc(-c2cc(-c3cc(C(C)(C)C)cc(C(C)(C)C)c3)nc(-c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)c2)cc(C(C)(C)C)c1]

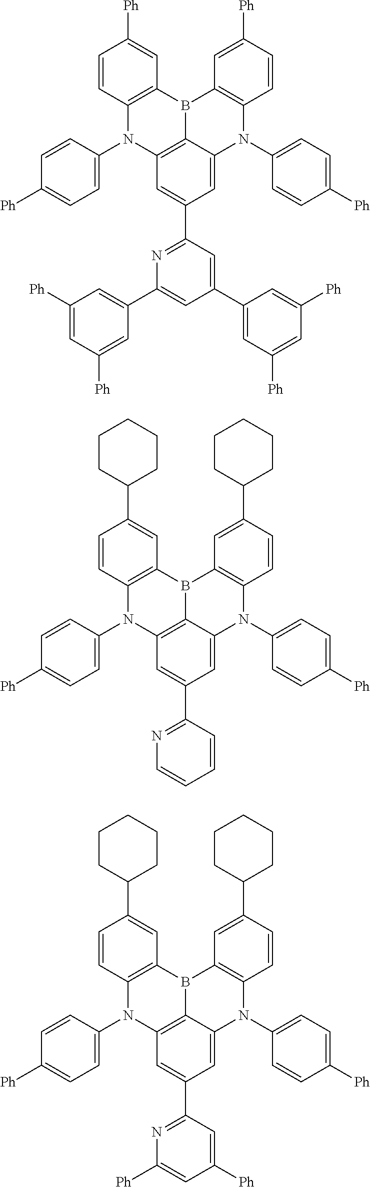

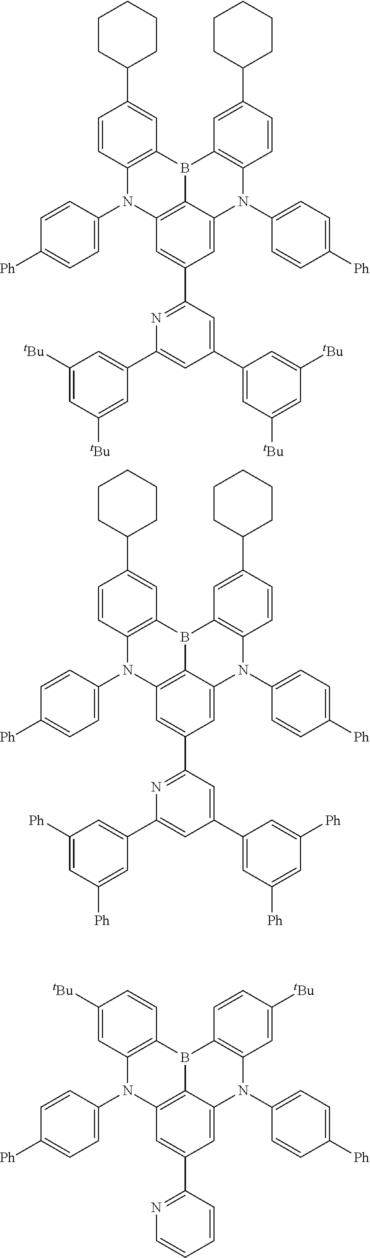

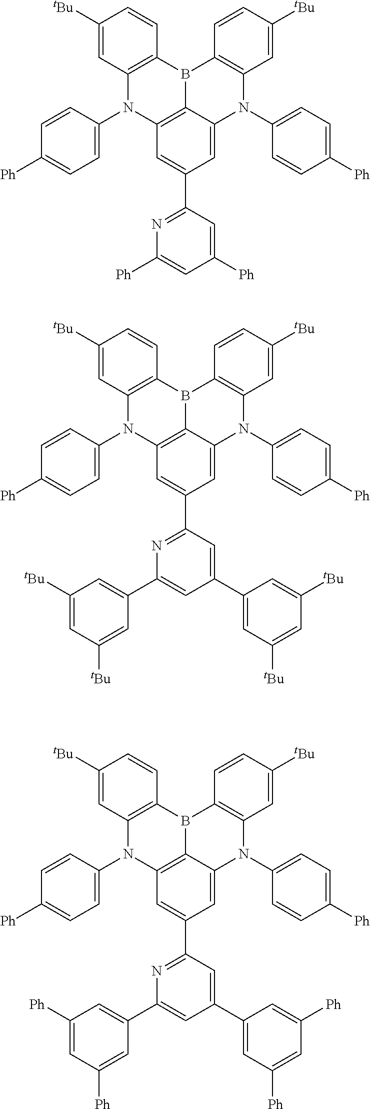 [SMILES: CC(C)(C)c1ccc2c(c1)N(c1ccc(-c3ccccc3)cc1)c1cc(-c3cc(-c4ccccc4)cc(-c4ccccc4)n3)cc3c1B2c1ccc(C(C)(C)C)cc1N3c1ccc(-c2ccccc2)cc1 ; CC(C)(C)c1cc(-c2cc(-c3cc(C(C)(C)C)cc(C(C)(C)C)c3)nc(-c3cc4c5c(c3)N(c3ccc(-c6ccccc6)cc3)c3cc(C(C)(C)C)ccc3B5c3ccc(C(C)(C)C)cc3N4c3ccc(-c4ccccc4)cc3)c2)cc(C(C)(C)C)c1 ; CC(C)(C)c1ccc2c(c1)N(c1ccc(-c3ccccc3)cc1)c1cc(-c3cc(-c4cc(-c5ccccc5)cc(-c5ccccc5)c4)cc(-c4cc(-c5ccccc5)cc(-c5ccccc5)c4)n3)cc3c1B2c1ccc(C(C)(C)C)cc1N3c1ccc(-c2ccccc2)cc1]

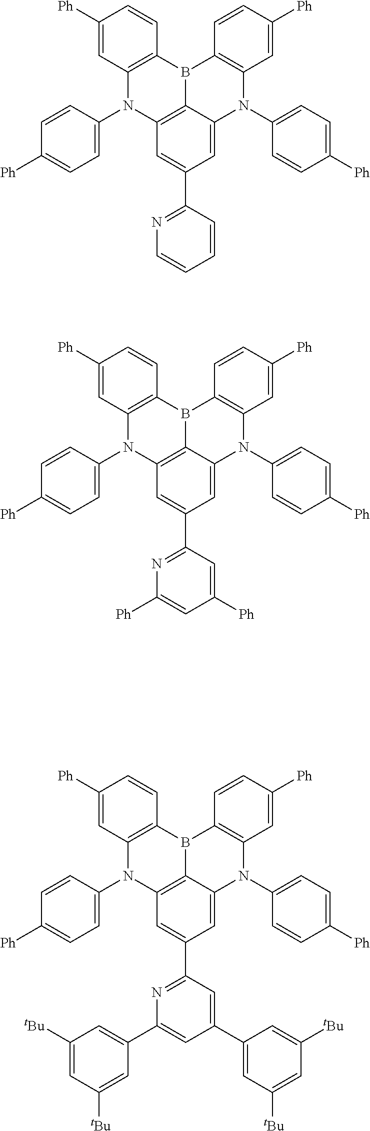 [SMILES: c1ccc(-c2ccc(N3c4cc(-c5ccccc5)ccc4B4c5ccc(-c6ccccc6)cc5N(c5ccc(-c6ccccc6)cc5)c5cc(-c6ccccn6)cc3c54)cc2)cc1 ; c1ccc(-c2ccc(N3c4cc(-c5ccccc5)ccc4B4c5ccc(-c6ccccc6)cc5N(c5ccc(-c6ccccc6)cc5)c5cc(-c6cc(-c7ccccc7)cc(-c7ccccc7)n6)cc3c54)cc2)cc1 ; CC(C)(C)c1cc(-c2cc(-c3cc(C(C)(C)C)cc(C(C)(C)C)c3)nc(-c3cc4c5c(c3)N(c3ccc(-c6ccccc6)cc3)c3cc(-c6ccccc6)ccc3B5c3ccc(-c5ccccc5)cc3N4c3ccc(-c4ccccc4)cc3)c2)cc(C(C)(C)C)c1]

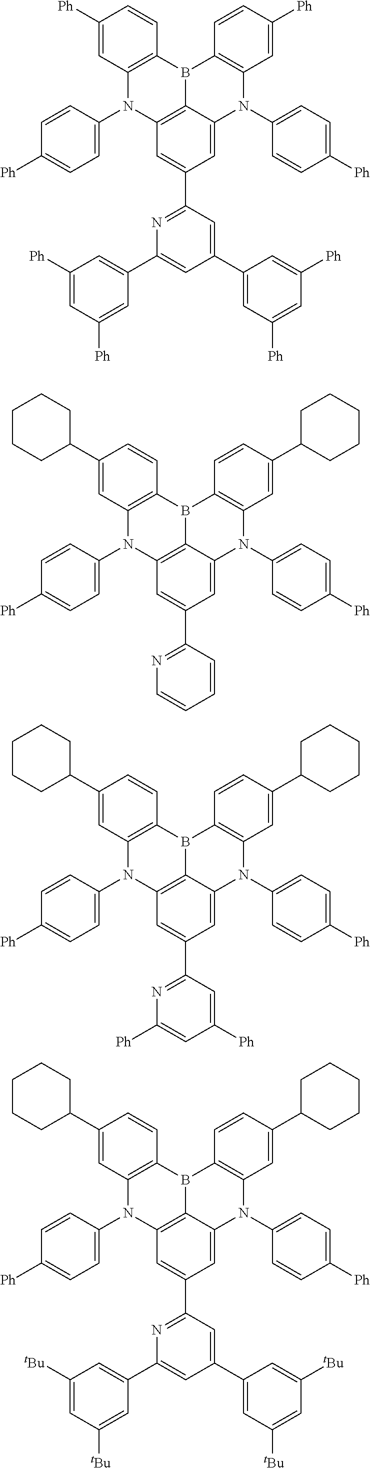

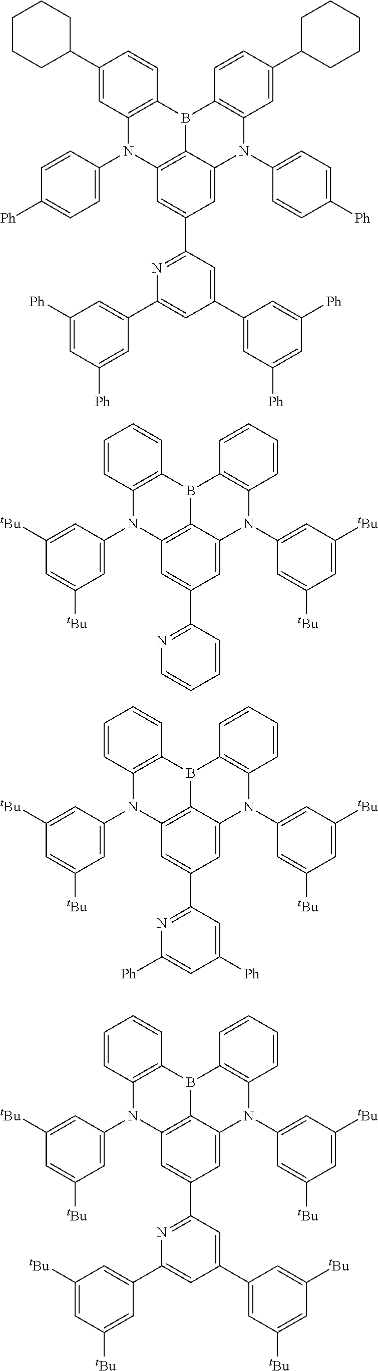

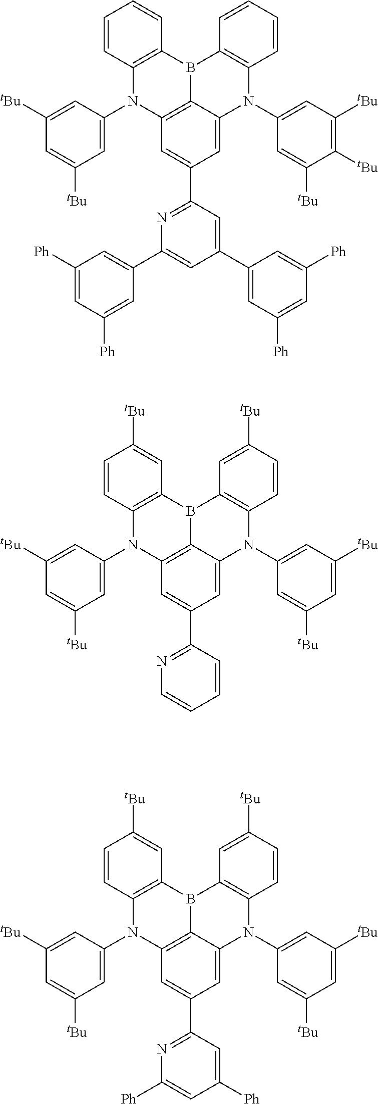 [SMILES: CC(C)(C)c1cc(N2c3ccccc3B3c4ccccc4N(c4cc(C(C)(C)C)c(C(C)(C)C)c(C(C)(C)C)c4)c4cc(-c5cc(-c6cc(-c7ccccc7)cc(-c7ccccc7)c6)cc(-c6cc(-c7ccccc7)cc(-c7ccccc7)c6)n5)cc2c43)cc(C(C)(C)C)c1 ; CC(C)(C)c1cc(N2c3ccc(C(C)(C)C)cc3B3c4cc(C(C)(C)C)ccc4N(c4cc(C(C)(C)C)cc(C(C)(C)C)c4)c4cc(-c5ccccn5)cc2c43)cc(C(C)(C)C)c1 ; CC(C)(C)c1cc(N2c3ccc(C(C)(C)C)cc3B3c4cc(C(C)(C)C)ccc4N(c4cc(C(C)(C)C)cc(C(C)(C)C)c4)c4cc(-c5cc(-c6ccccc6)cc(-c6ccccc6)n5)cc2c43)cc(C(C)(C)C)c1]

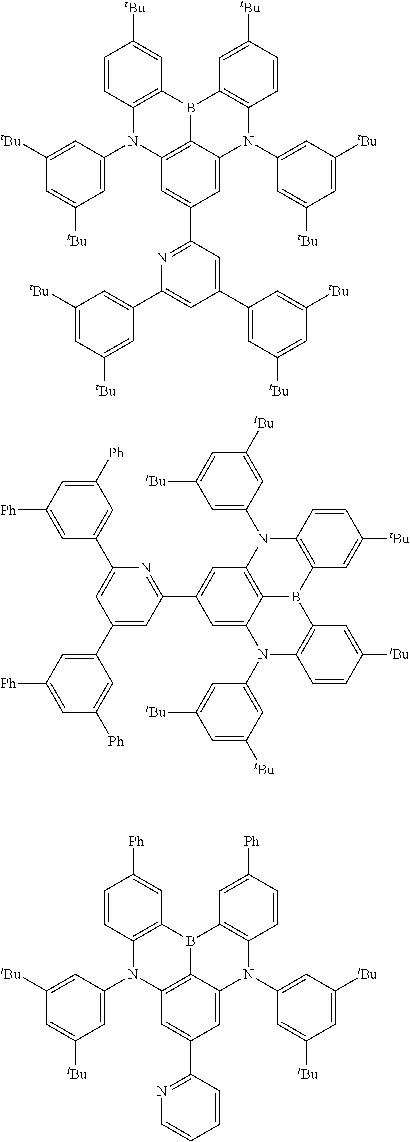

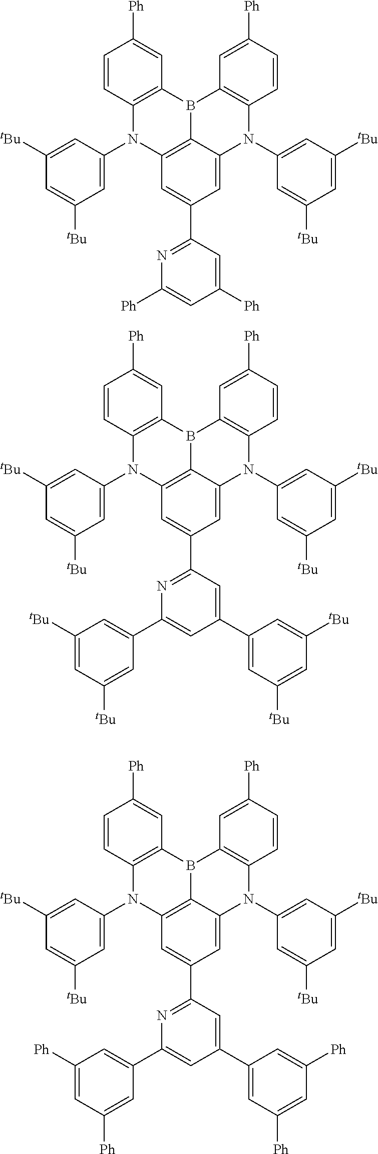

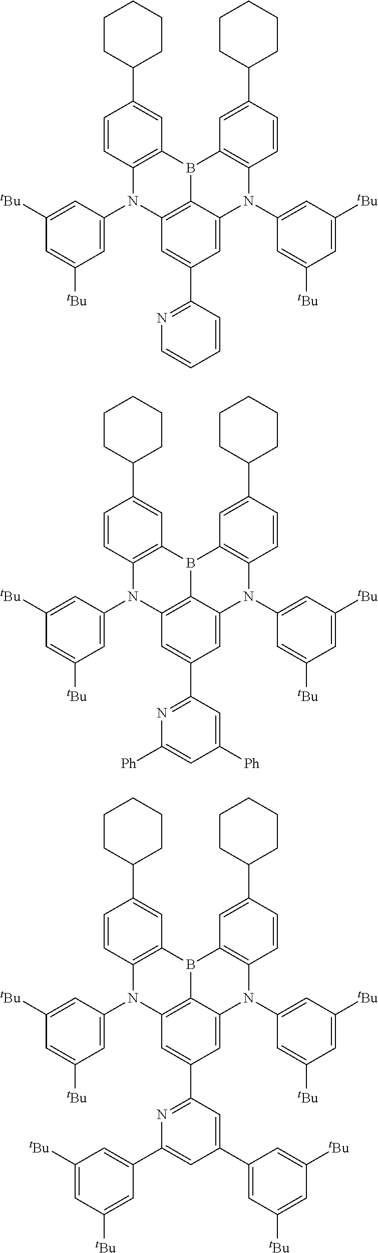

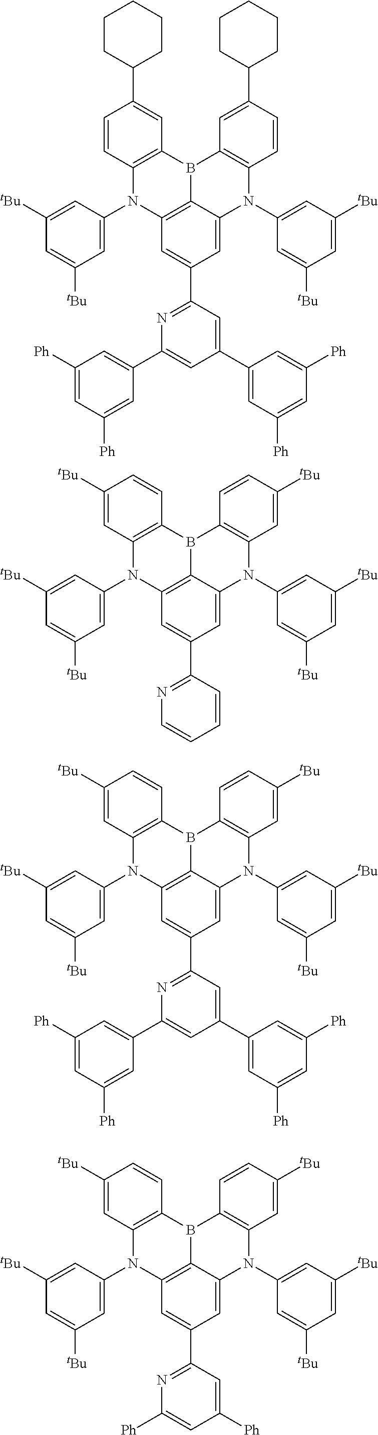

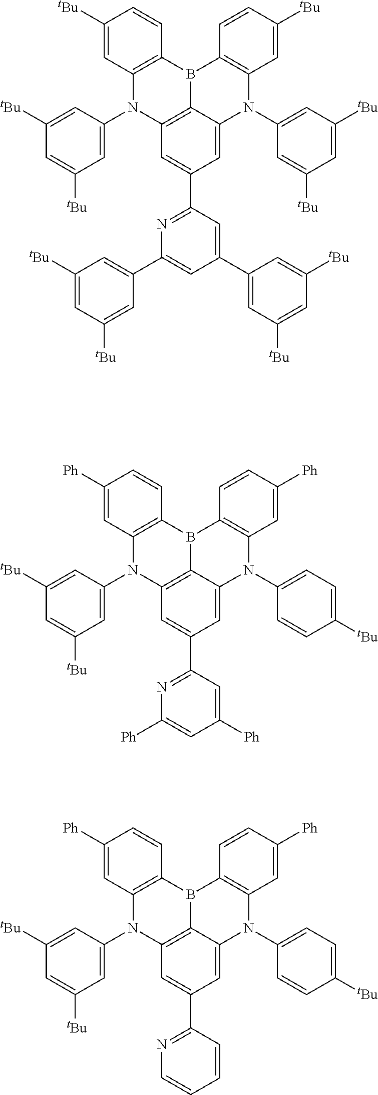 [SMILES: CC(C)(C)c1cc(-c2cc(-c3cc(C(C)(C)C)cc(C(C)(C)C)c3)nc(-c3cc4c5c(c3)N(c3cc(C(C)(C)C)cc(C(C)(C)C)c3)c3cc(C(C)(C)C)ccc3B5c3ccc(C(C)(C)C)cc3N4c3cc(C(C)(C)C)cc(C(C)(C)C)c3)c2)cc(C(C)(C)C)c1 ; CC(C)(C)c1ccc(N2c3cc(-c4ccccc4)ccc3B3c4ccc(-c5ccccc5)cc4N(c4cc(C(C)(C)C)cc(C(C)(C)C)c4)c4cc(-c5cc(-c6ccccc6)cc(-c6ccccc6)n5)cc2c43)cc1 ; CC(C)(C)c1ccc(N2c3cc(-c4ccccc4)ccc3B3c4ccc(-c5ccccc5)cc4N(c4cc(C(C)(C)C)cc(C(C)(C)C)c4)c4cc(-c5ccccn5)cc2c43)cc1]

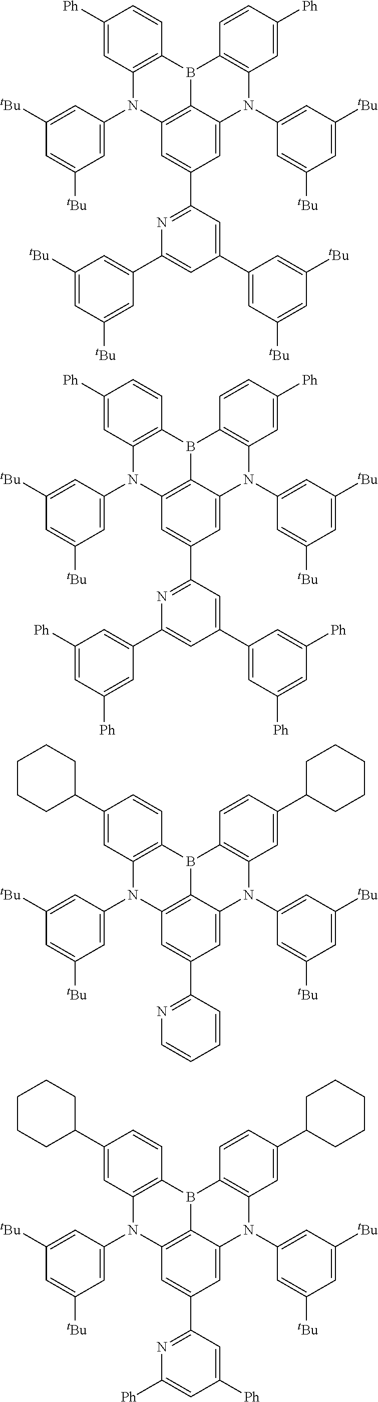

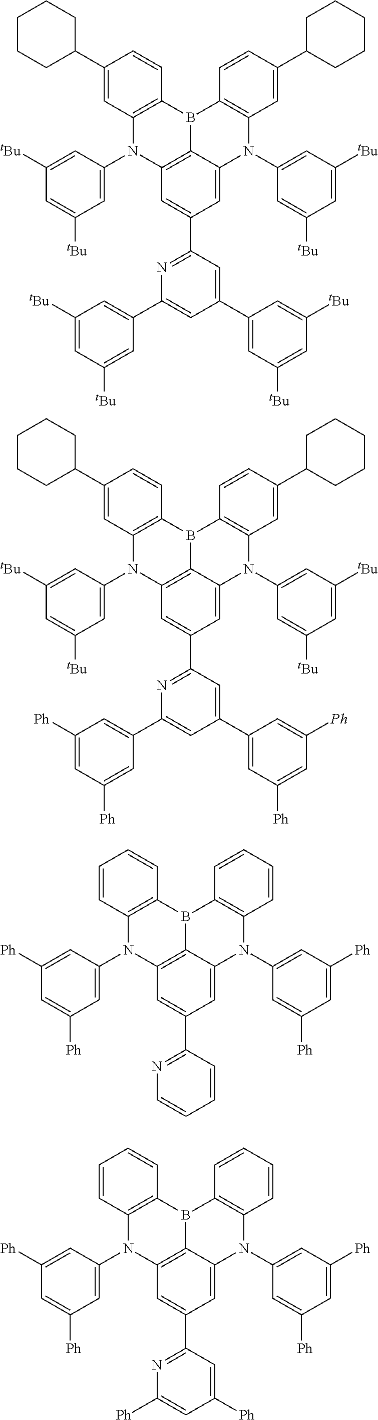

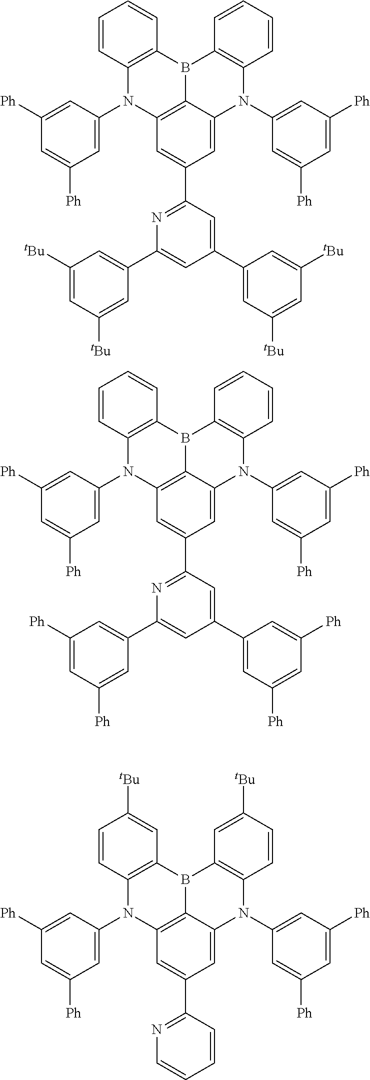

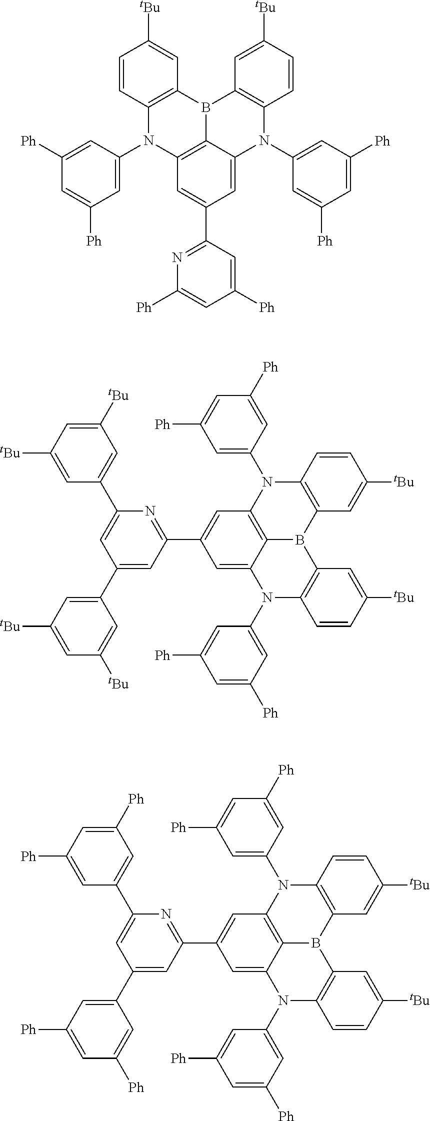

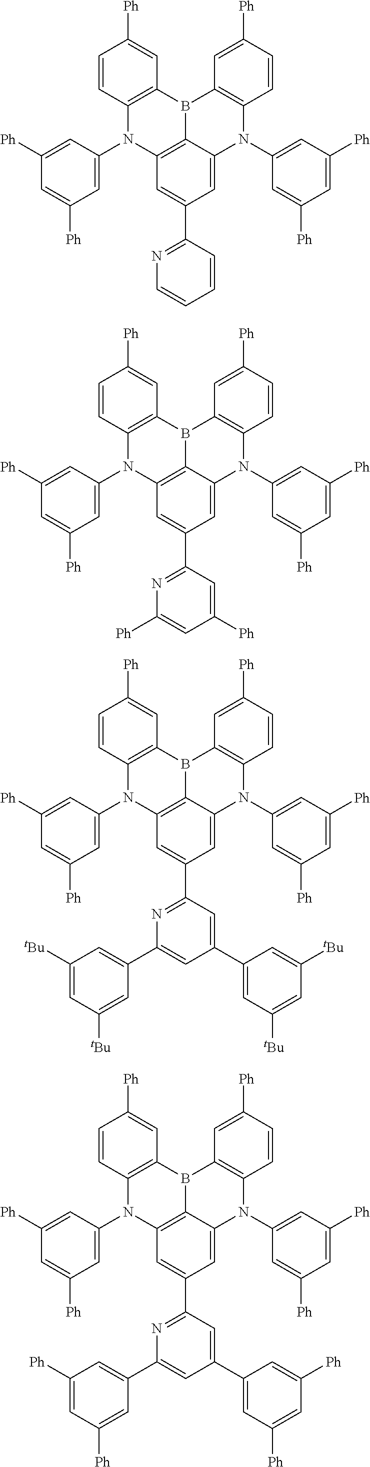

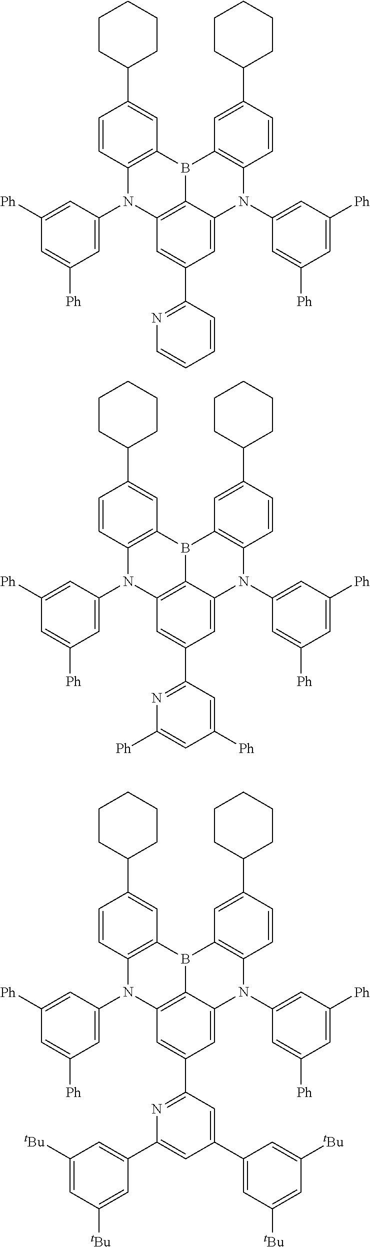

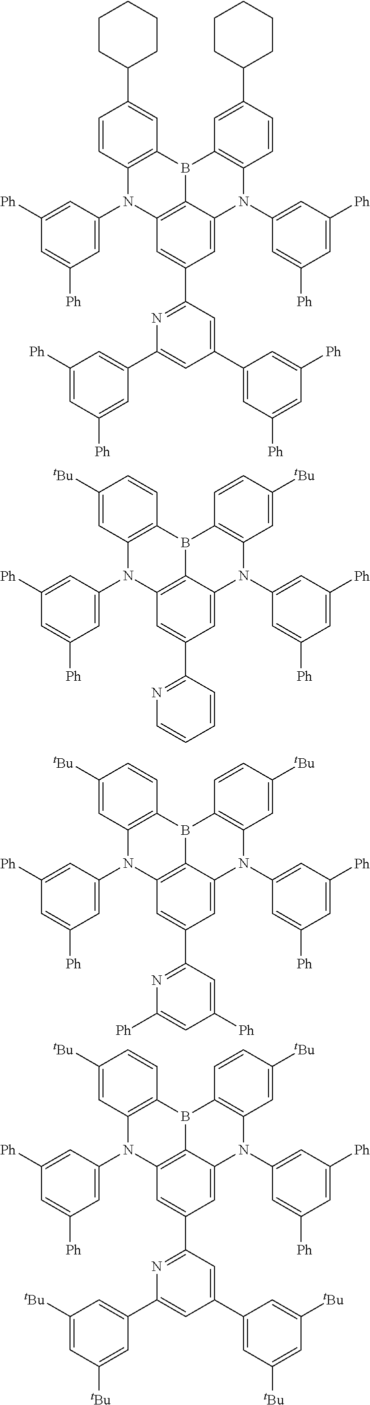

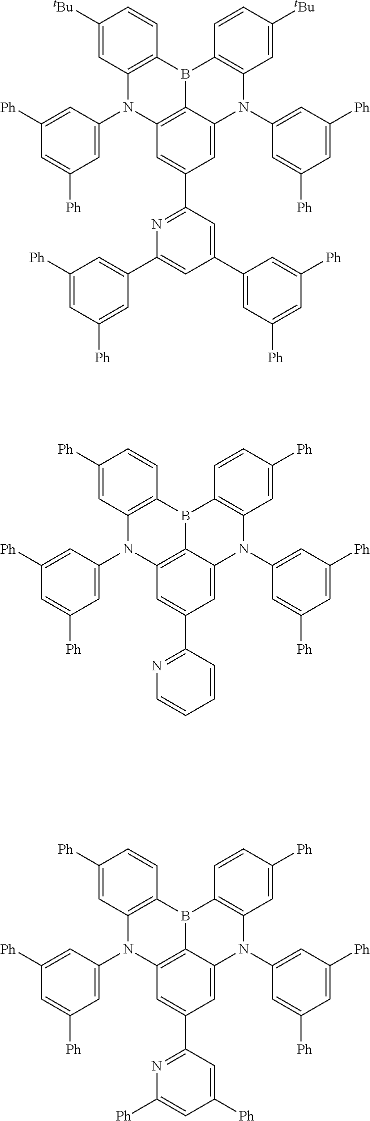 [SMILES: CC(C)(C)c1ccc2c(c1)N(c1cc(-c3ccccc3)cc(-c3ccccc3)c1)c1cc(-c3cc(-c4cc(-c5ccccc5)cc(-c5ccccc5)c4)cc(-c4cc(-c5ccccc5)cc(-c5ccccc5)c4)n3)cc3c1B2c1ccc(C(C)(C)C)cc1N3c1cc(-c2ccccc2)cc(-c2ccccc2)c1 ; c1ccc(-c2cc(-c3ccccc3)cc(N3c4cc(-c5ccccc5)ccc4B4c5ccc(-c6ccccc6)cc5N(c5cc(-c6ccccc6)cc(-c6ccccc6)c5)c5cc(-c6ccccn6)cc3c54)c2)cc1 ; c1ccc(-c2cc(-c3ccccc3)cc(N3c4cc(-c5ccccc5)ccc4B4c5ccc(-c6ccccc6)cc5N(c5cc(-c6ccccc6)cc(-c6ccccc6)c5)c5cc(-c6cc(-c7ccccc7)cc(-c7ccccc7)n6)cc3c54)c2)cc1]

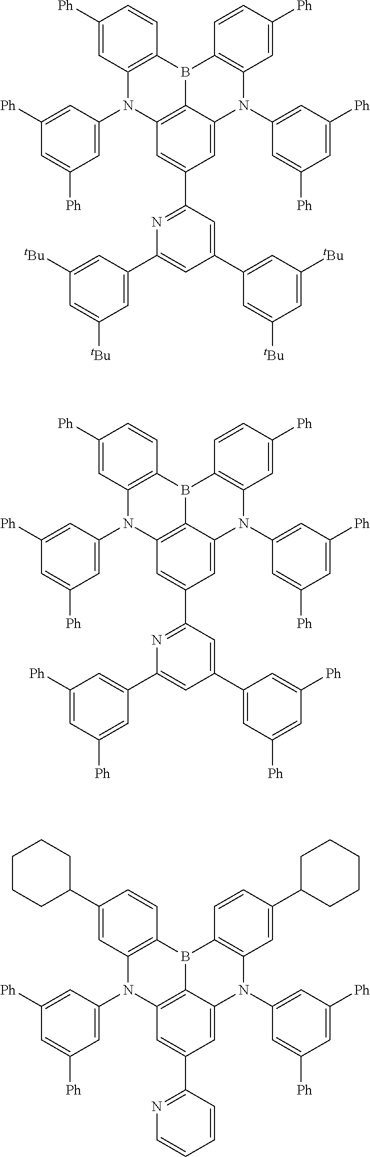

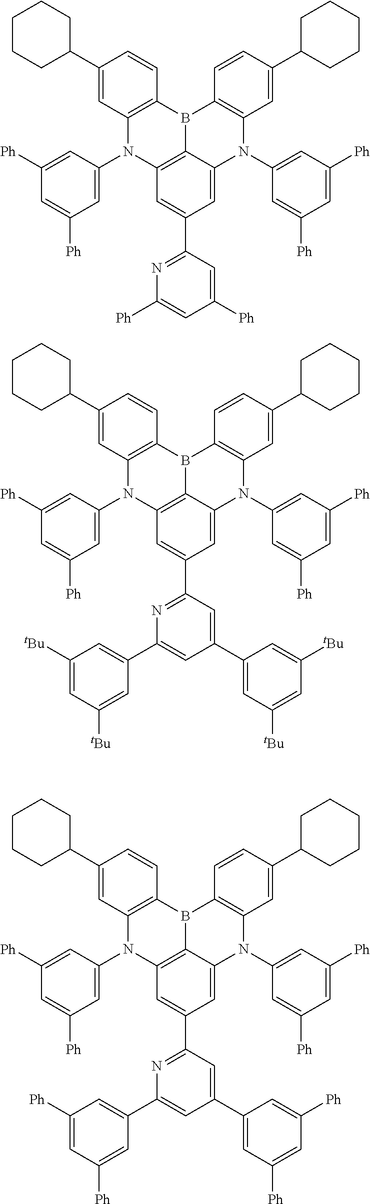

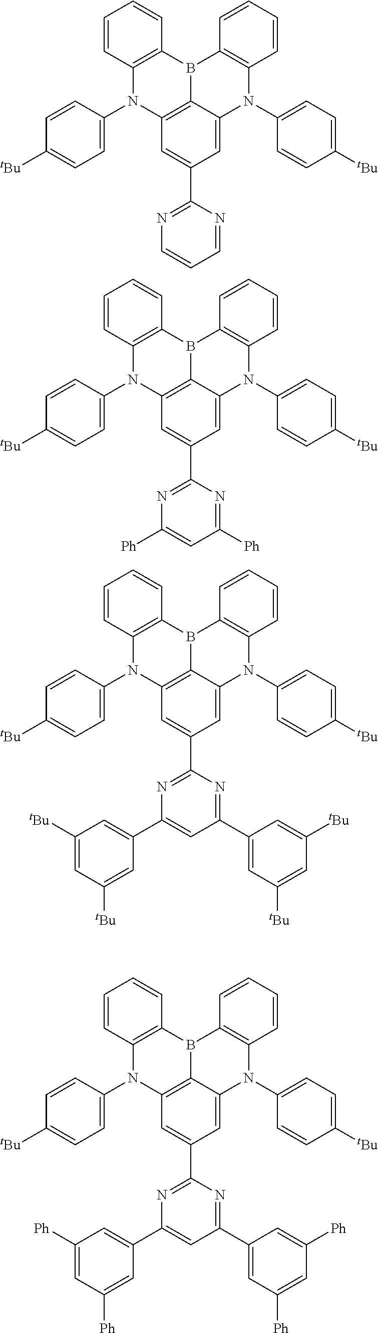

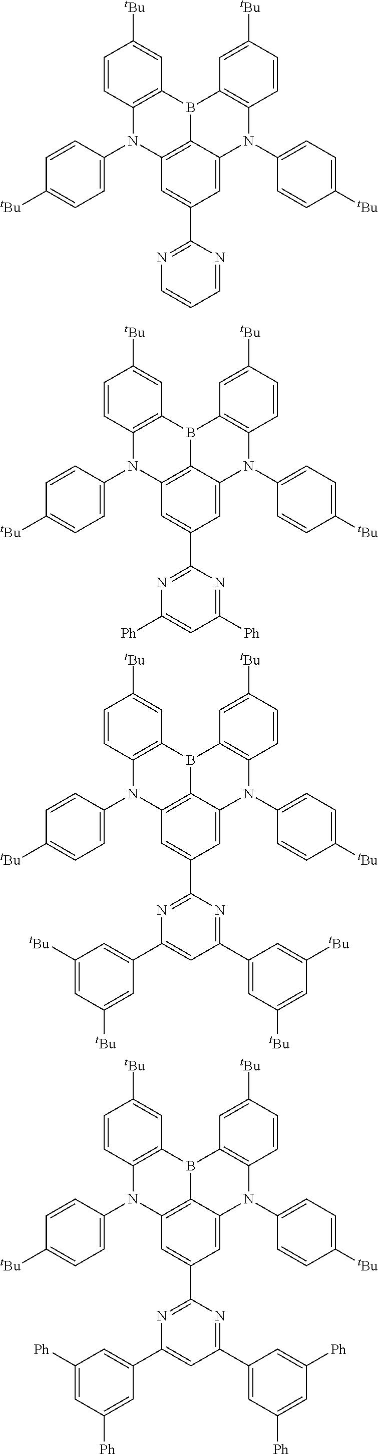

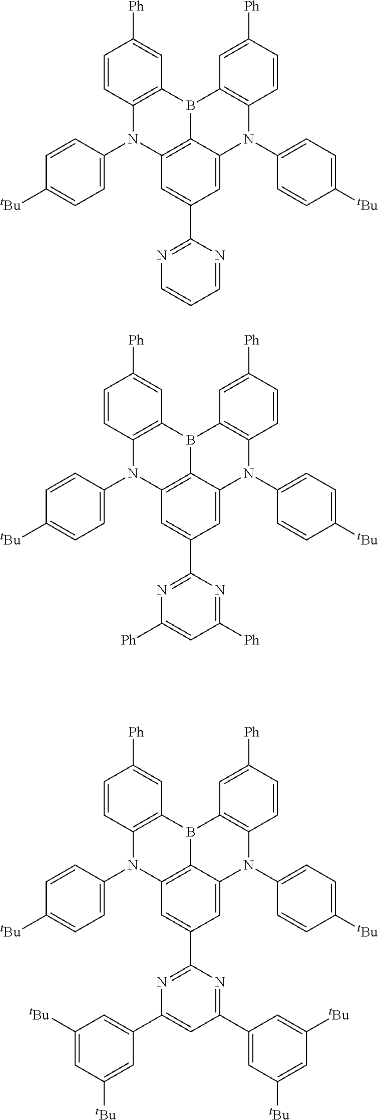 [SMILES: CC(C)(C)c1ccc(N2c3ccc(-c4ccccc4)cc3B3c4cc(-c5ccccc5)ccc4N(c4ccc(C(C)(C)C)cc4)c4cc(-c5ncccn5)cc2c43)cc1 ; CC(C)(C)c1ccc(N2c3ccc(-c4ccccc4)cc3B3c4cc(-c5ccccc5)ccc4N(c4ccc(C(C)(C)C)cc4)c4cc(-c5nc(-c6ccccc6)cc(-c6ccccc6)n5)cc2c43)cc1 ; CC(C)(C)c1ccc(N2c3ccc(-c4ccccc4)cc3B3c4cc(-c5ccccc5)ccc4N(c4ccc(C(C)(C)C)cc4)c4cc(-c5nc(-c6cc(C(C)(C)C)cc(C(C)(C)C)c6)cc(-c6cc(C(C)(C)C)cc(C(C)(C)C)c6)n5)cc2c43)cc1]

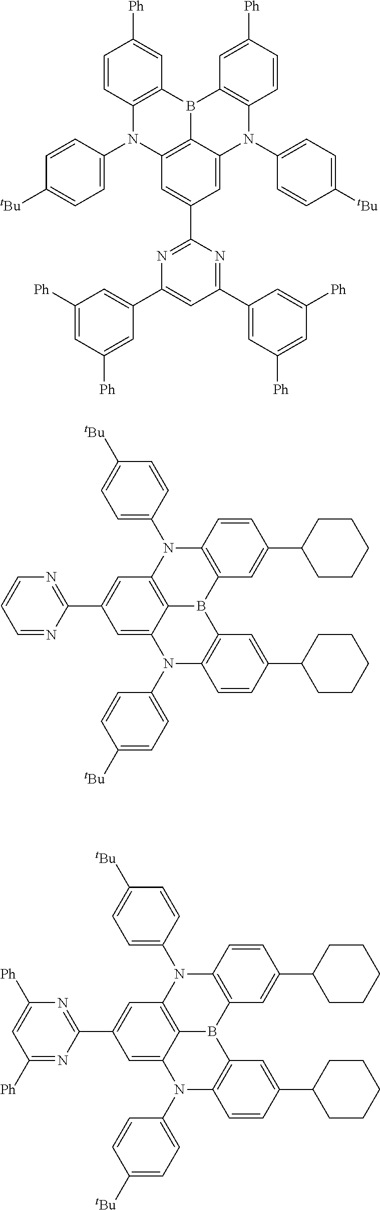

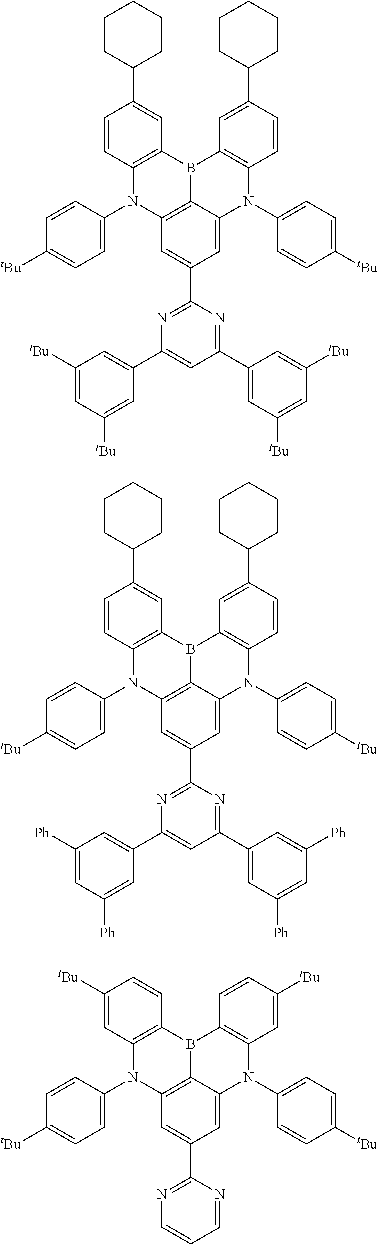

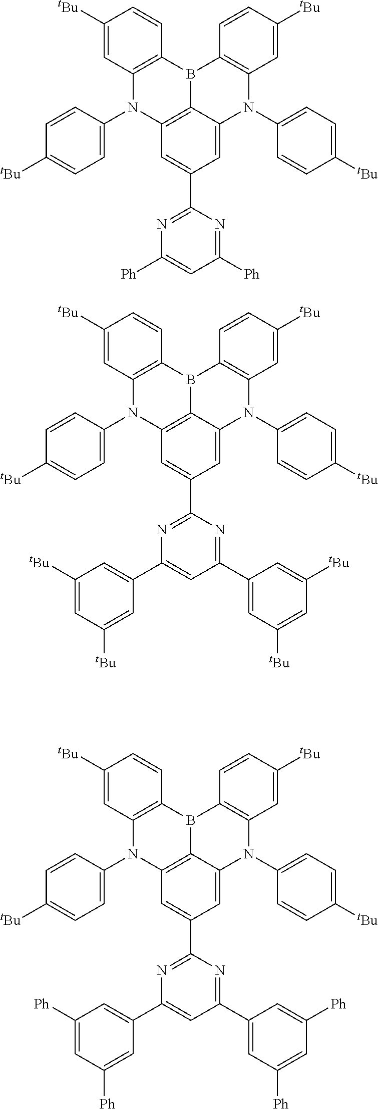

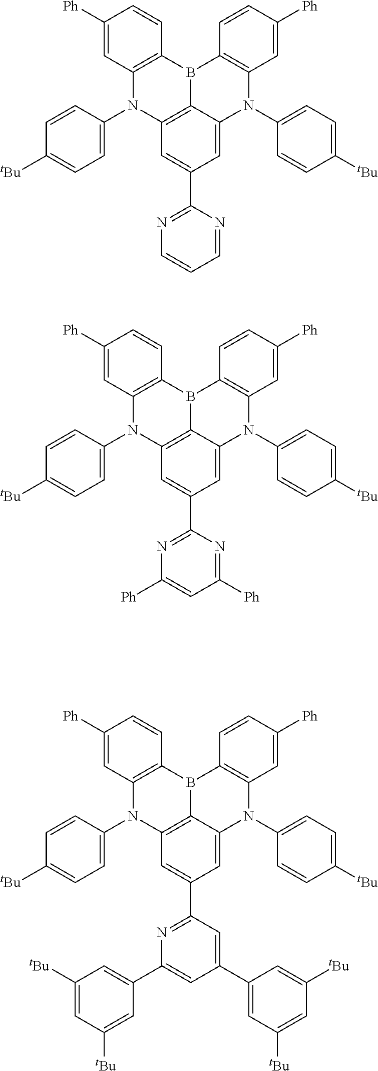 [SMILES: CC(C)(C)c1ccc(N2c3cc(-c4ccccc4)ccc3B3c4ccc(-c5ccccc5)cc4N(c4ccc(C(C)(C)C)cc4)c4cc(-c5ncccn5)cc2c43)cc1 ; CC(C)(C)c1ccc(N2c3cc(-c4ccccc4)ccc3B3c4ccc(-c5ccccc5)cc4N(c4ccc(C(C)(C)C)cc4)c4cc(-c5nc(-c6ccccc6)cc(-c6ccccc6)n5)cc2c43)cc1 ; CC(C)(C)c1ccc(N2c3cc(-c4ccccc4)ccc3B3c4ccc(-c5ccccc5)cc4N(c4ccc(C(C)(C)C)cc4)c4cc(-c5cc(-c6cc(C(C)(C)C)cc(C(C)(C)C)c6)cc(-c6cc(C(C)(C)C)cc(C(C)(C)C)c6)n5)cc2c43)cc1]

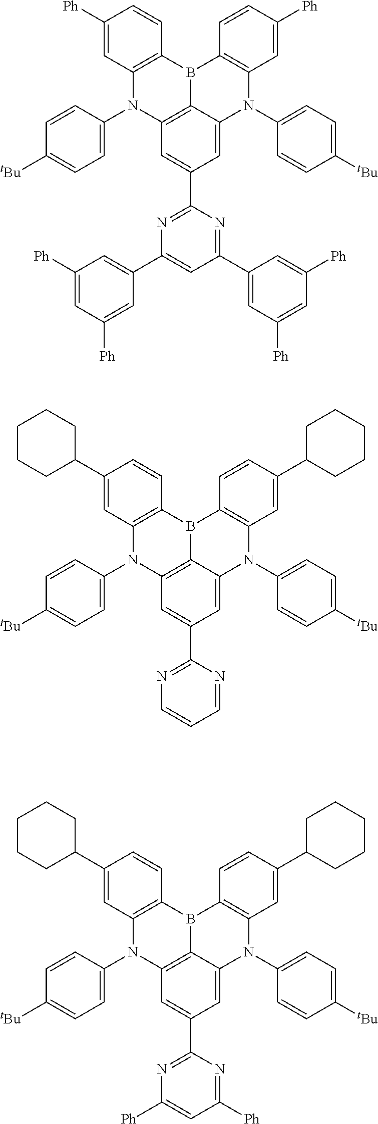 [SMILES: CC(C)(C)c1ccc(N2c3cc(-c4ccccc4)ccc3B3c4ccc(-c5ccccc5)cc4N(c4ccc(C(C)(C)C)cc4)c4cc(-c5nc(-c6cc(-c7ccccc7)cc(-c7ccccc7)c6)cc(-c6cc(-c7ccccc7)cc(-c7ccccc7)c6)n5)cc2c43)cc1 ; CC(C)(C)c1ccc(N2c3cc(C4CCCCC4)ccc3B3c4ccc(C5CCCCC5)cc4N(c4ccc(C(C)(C)C)cc4)c4cc(-c5ncccn5)cc2c43)cc1 ; CC(C)(C)c1ccc(N2c3cc(C4CCCCC4)ccc3B3c4ccc(C5CCCCC5)cc4N(c4ccc(C(C)(C)C)cc4)c4cc(-c5nc(-c6ccccc6)cc(-c6ccccc6)n5)cc2c43)cc1]

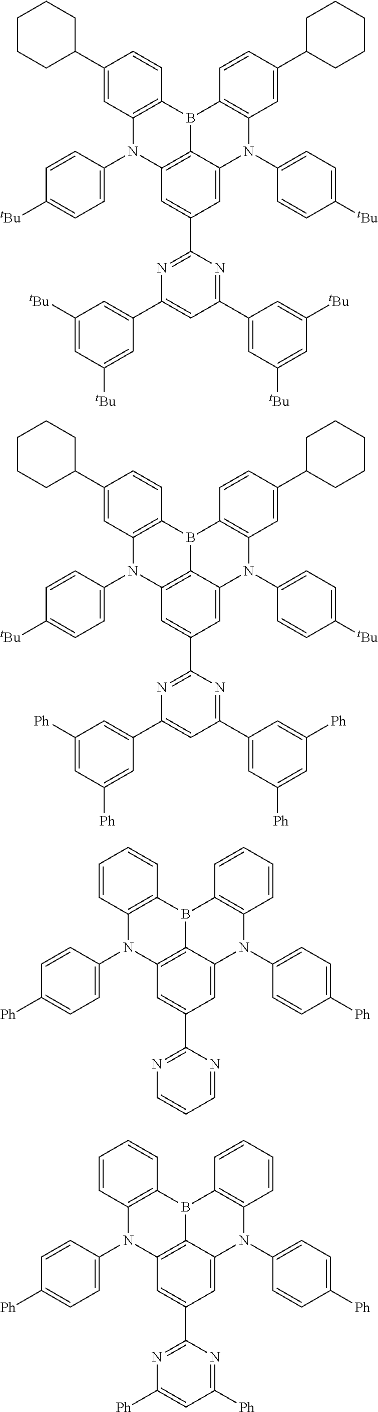

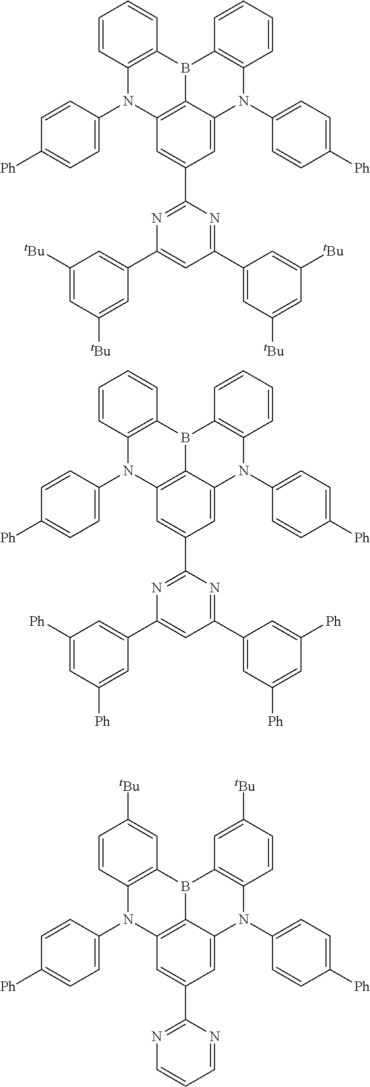

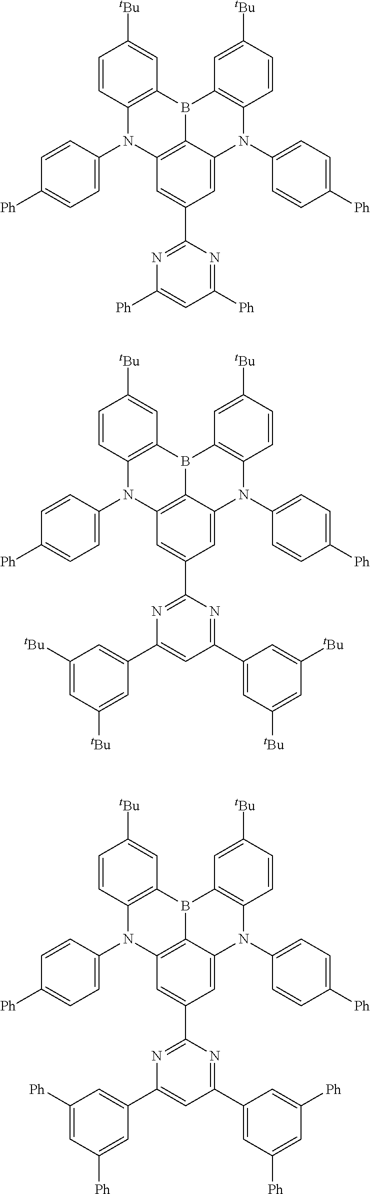 [SMILES: CC(C)(C)c1ccc2c(c1)B1c3cc(C(C)(C)C)ccc3N(c3ccc(-c4ccccc4)cc3)c3cc(-c4nc(-c5ccccc5)cc(-c5ccccc5)n4)cc(c31)N2c1ccc(-c2ccccc2)cc1 ; CC(C)(C)c1cc(-c2cc(-c3cc(C(C)(C)C)cc(C(C)(C)C)c3)nc(-c3cc4c5c(c3)N(c3ccc(-c6ccccc6)cc3)c3ccc(C(C)(C)C)cc3B5c3cc(C(C)(C)C)ccc3N4c3ccc(-c4ccccc4)cc3)n2)cc(C(C)(C)C)c1 ; CC(C)(C)c1ccc2c(c1)B1c3cc(C(C)(C)C)ccc3N(c3ccc(-c4ccccc4)cc3)c3cc(-c4nc(-c5cc(-c6ccccc6)cc(-c6ccccc6)c5)cc(-c5cc(-c6ccccc6)cc(-c6ccccc6)c5)n4)cc(c31)N2c1ccc(-c2ccccc2)cc1]

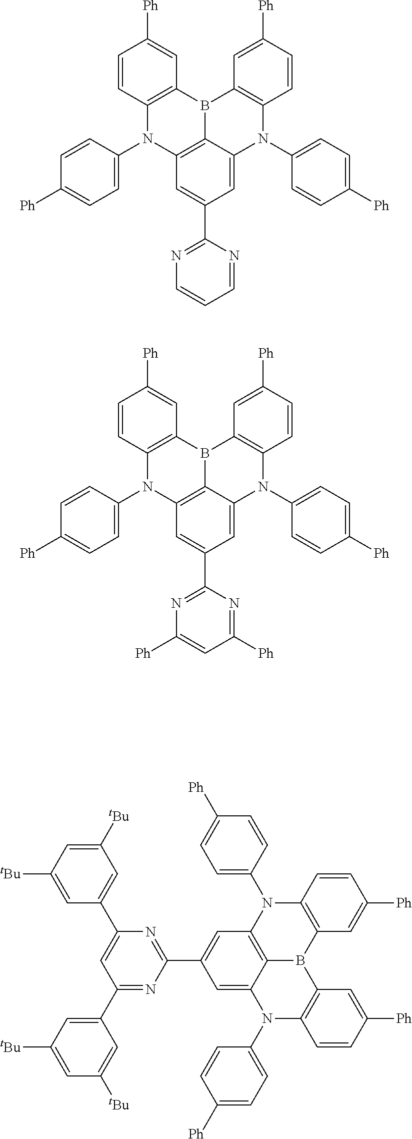 [SMILES: c1ccc(-c2ccc(N3c4ccc(-c5ccccc5)cc4B4c5cc(-c6ccccc6)ccc5N(c5ccc(-c6ccccc6)cc5)c5cc(-c6ncccn6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)cc(-c7ccccc7)n6)cc3c54)cc2)cc1 ; CC(C)(C)c1cc(-c2cc(-c3cc(C(C)(C)C)cc(C(C)(C)C)c3)nc(-c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)n2)cc(C(C)(C)C)c1]

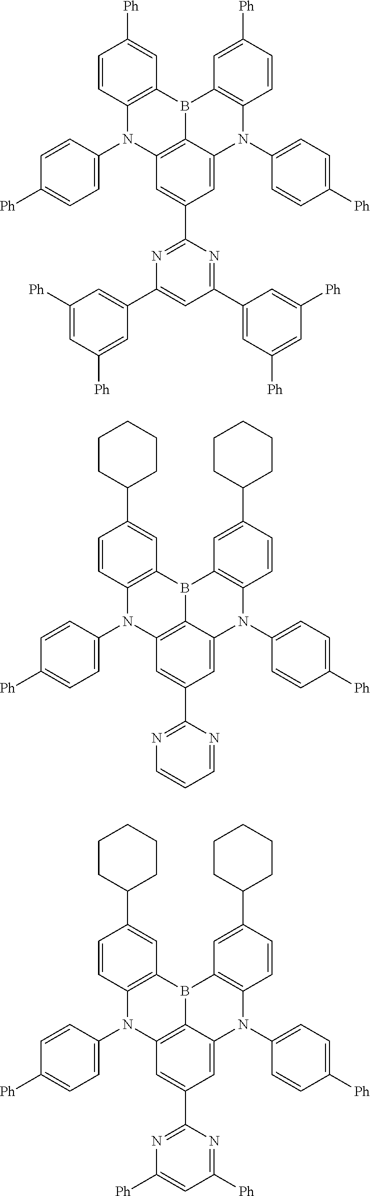

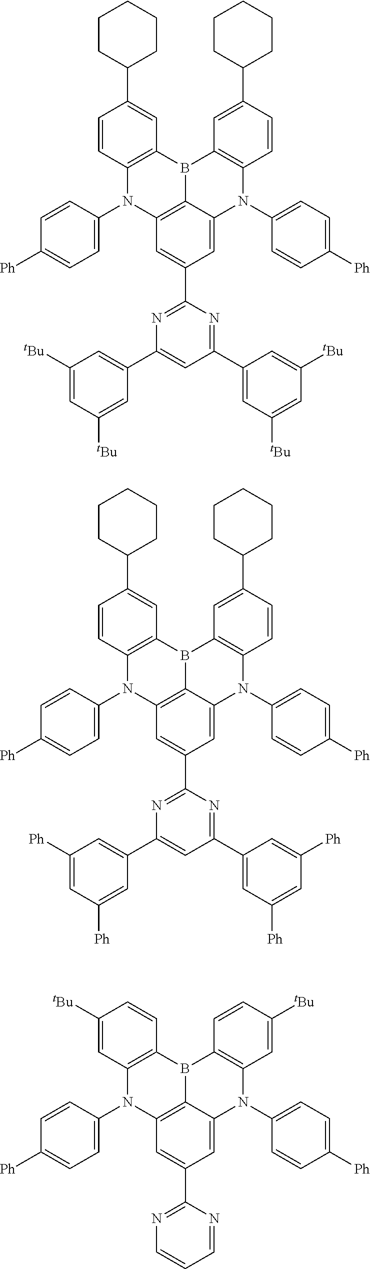 [SMILES: CC(C)(C)c1cc(-c2cc(-c3cc(C(C)(C)C)cc(C(C)(C)C)c3)nc(-c3cc4c5c(c3)N(c3ccc(-c6ccccc6)cc3)c3ccc(C6CCCCC6)cc3B5c3cc(C5CCCCC5)ccc3N4c3ccc(-c4ccccc4)cc3)n2)cc(C(C)(C)C)c1 ; c1ccc(-c2ccc(N3c4ccc(C5CCCCC5)cc4B4c5cc(C6CCCCC6)ccc5N(c5ccc(-c6ccccc6)cc5)c5cc(-c6nc(-c7cc(-c8ccccc8)cc(-c8ccccc8)c7)cc(-c7cc(-c8ccccc8)cc(-c8ccccc8)c7)n6)cc3c54)cc2)cc1 ; CC(C)(C)c1ccc2c(c1)N(c1ccc(-c3ccccc3)cc1)c1cc(-c3ncccn3)cc3c1B2c1ccc(C(C)(C)C)cc1N3c1ccc(-c2ccccc2)cc1]

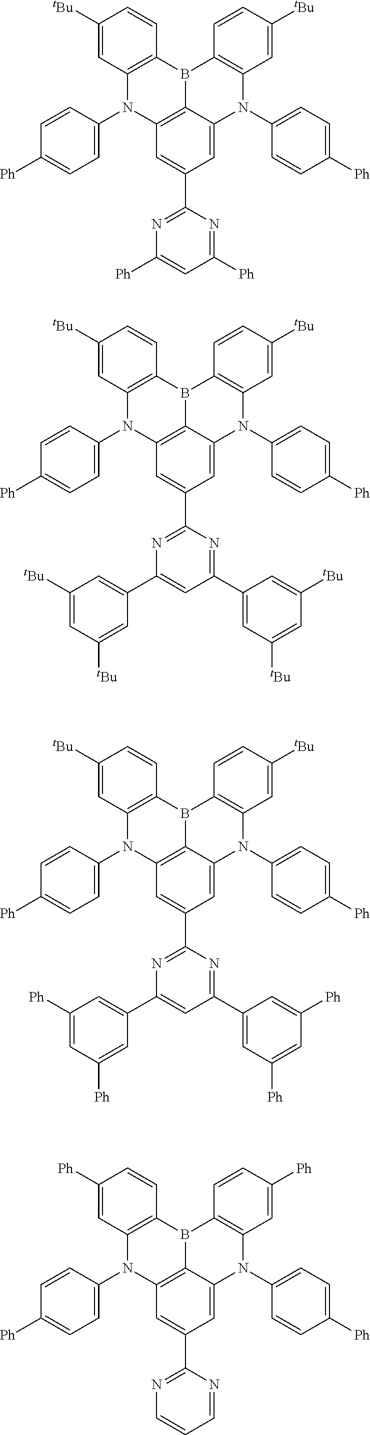

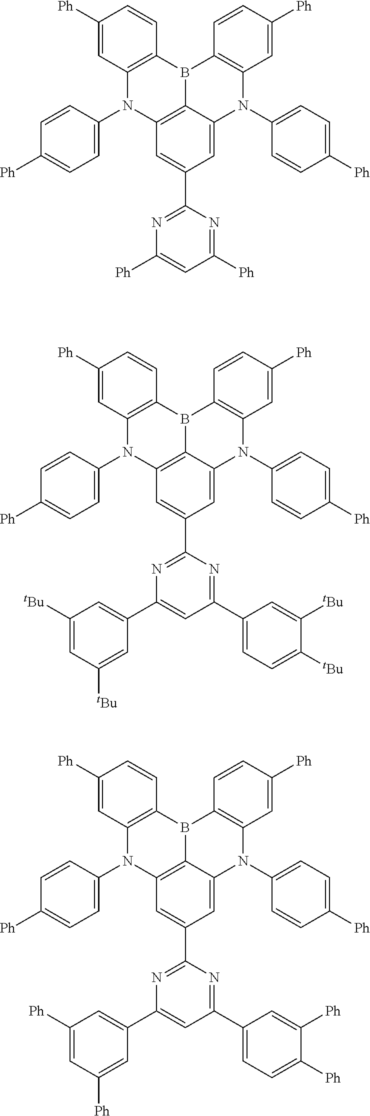

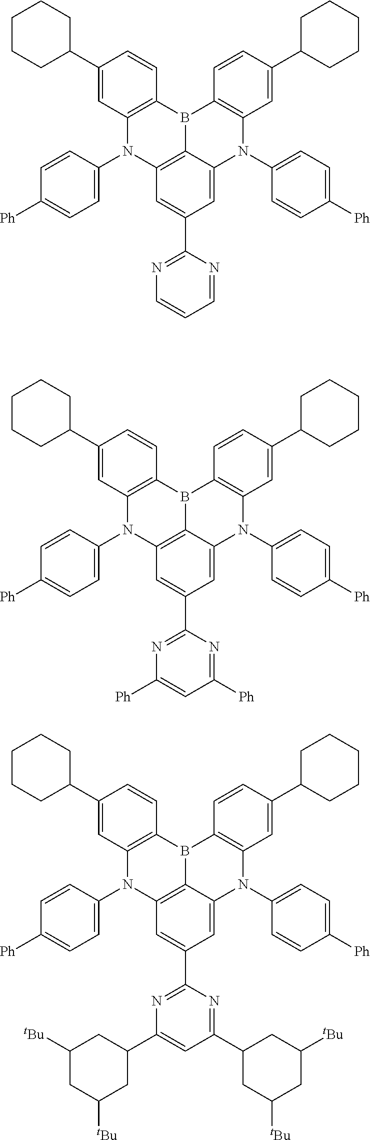 [SMILES: c1ccc(-c2ccc(N3c4cc(C5CCCCC5)ccc4B4c5ccc(C6CCCCC6)cc5N(c5ccc(-c6ccccc6)cc5)c5cc(-c6ncccn6)cc3c54)cc2)cc1 ; c1ccc(-c2ccc(N3c4cc(C5CCCCC5)ccc4B4c5ccc(C6CCCCC6)cc5N(c5ccc(-c6ccccc6)cc5)c5cc(-c6nc(-c7ccccc7)cc(-c7ccccc7)n6)cc3c54)cc2)cc1 ; CC(C)(C)C1CC(c2cc(C3CC(C(C)(C)C)CC(C(C)(C)C)C3)nc(-c3cc4c5c(c3)N(c3ccc(-c6ccccc6)cc3)c3cc(C6CCCCC6)ccc3B5c3ccc(C5CCCCC5)cc3N4c3ccc(-c4ccccc4)cc3)n2)CC(C(C)(C)C)C1]

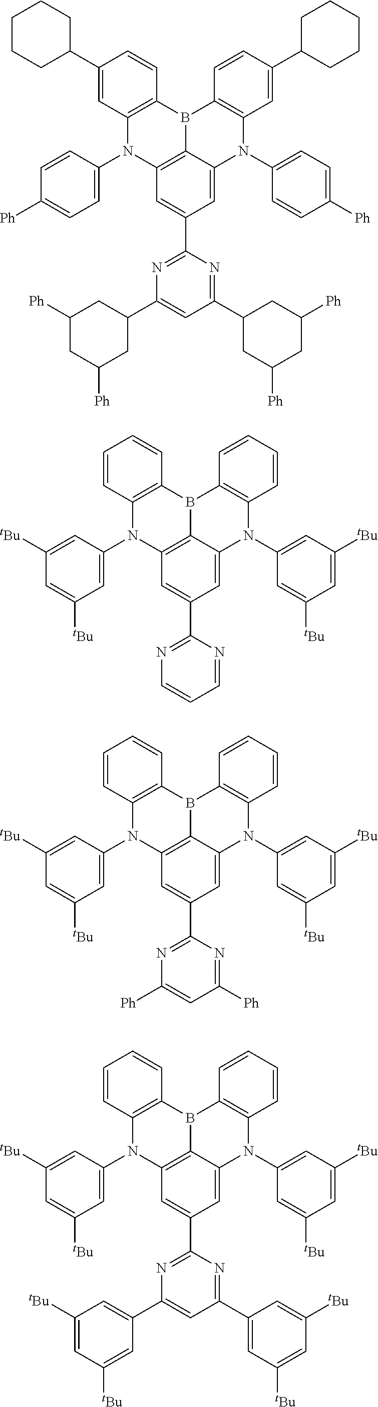

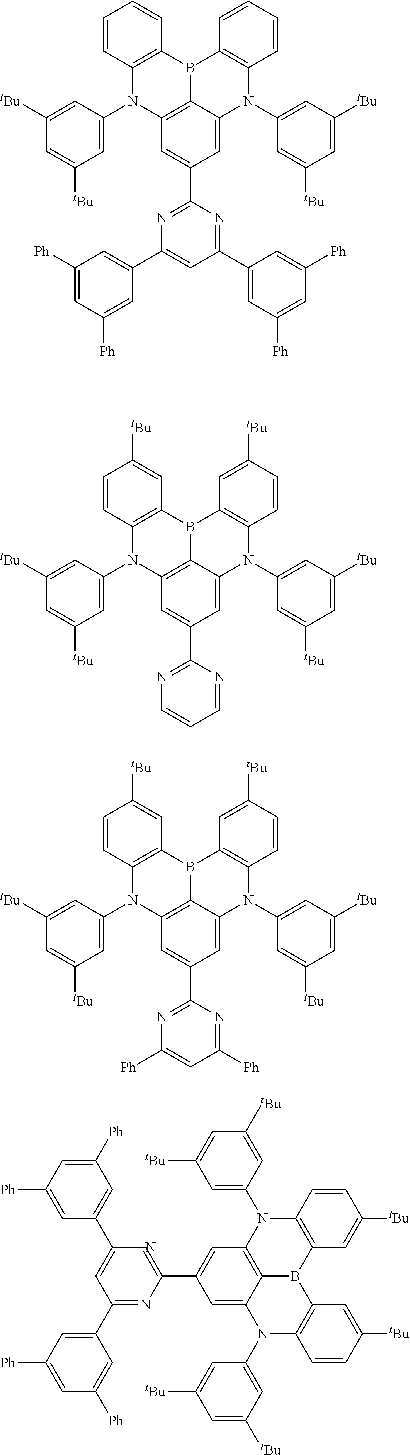

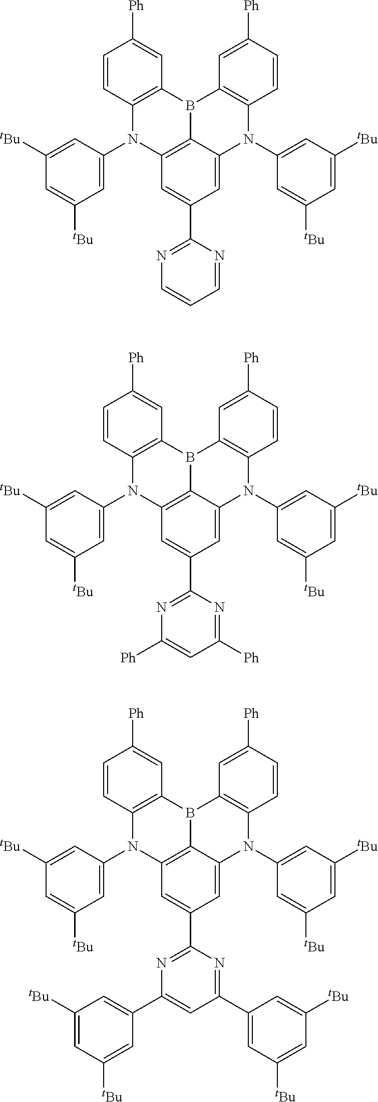 [SMILES: CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5ncccn5)cc2c43)cc(C(C)(C)C)c1 ; CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5nc(-c6ccccc6)cc(-c6ccccc6)n5)cc2c43)cc(C(C)(C)C)c1 ; CC(C)(C)c1cc(-c2cc(-c3cc(C(C)(C)C)cc(C(C)(C)C)c3)nc(-c3cc4c5c(c3)N(c3cc(C(C)(C)C)cc(C(C)(C)C)c3)c3ccc(-c6ccccc6)cc3B5c3cc(-c5ccccc5)ccc3N4c3cc(C(C)(C)C)cc(C(C)(C)C)c3)n2)cc(C(C)(C)C)c1]

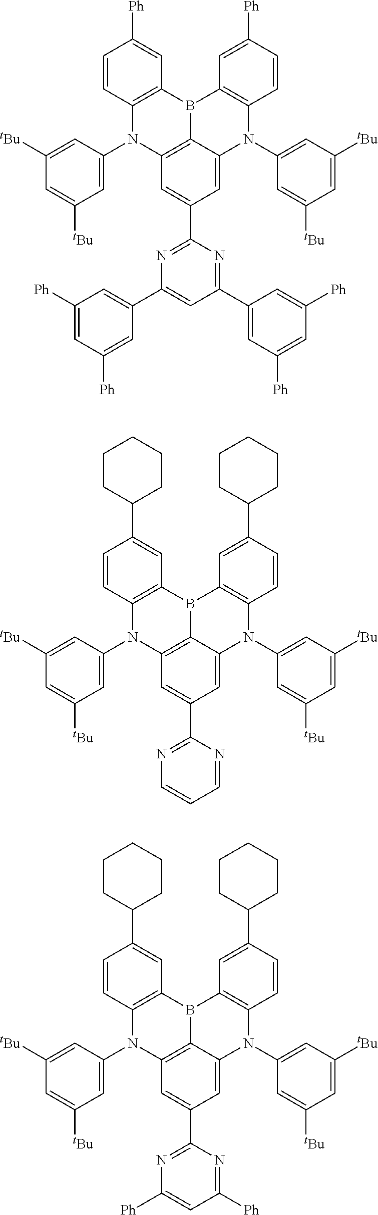 [SMILES: CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5nc(-c6cc(-c7ccccc7)cc(-c7ccccc7)c6)cc(-c6cc(-c7ccccc7)cc(-c7ccccc7)c6)n5)cc2c43)cc(C(C)(C)C)c1 ; CC(C)(C)c1cc(N2c3ccc(C4CCCCC4)cc3B3c4cc(C5CCCCC5)ccc4N(c4cc(C(C)(C)C)cc(C(C)(C)C)c4)c4cc(-c5ncccn5)cc2c43)cc(C(C)(C)C)c1 ; CC(C)(C)c1cc(N2c3ccc(C4CCCCC4)cc3B3c4cc(C5CCCCC5)ccc4N(c4cc(C(C)(C)C)cc(C(C)(C)C)c4)c4cc(-c5nc(-c6ccccc6)cc(-c6ccccc6)n5)cc2c43)cc(C(C)(C)C)c1]

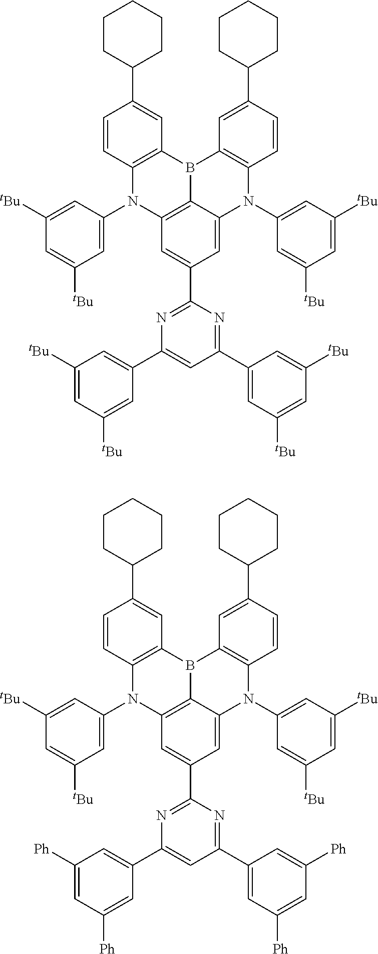 [SMILES: CC(C)(C)c1cc(-c2cc(-c3cc(C(C)(C)C)cc(C(C)(C)C)c3)nc(-c3cc4c5c(c3)N(c3cc(C(C)(C)C)cc(C(C)(C)C)c3)c3ccc(C6CCCCC6)cc3B5c3cc(C5CCCCC5)ccc3N4c3cc(C(C)(C)C)cc(C(C)(C)C)c3)n2)cc(C(C)(C)C)c1 ; CC(C)(C)c1cc(N2c3ccc(C4CCCCC4)cc3B3c4cc(C5CCCCC5)ccc4N(c4cc(C(C)(C)C)cc(C(C)(C)C)c4)c4cc(-c5nc(-c6cc(-c7ccccc7)cc(-c7ccccc7)c6)cc(-c6cc(-c7ccccc7)cc(-c7ccccc7)c6)n5)cc2c43)cc(C(C)(C)C)c1]

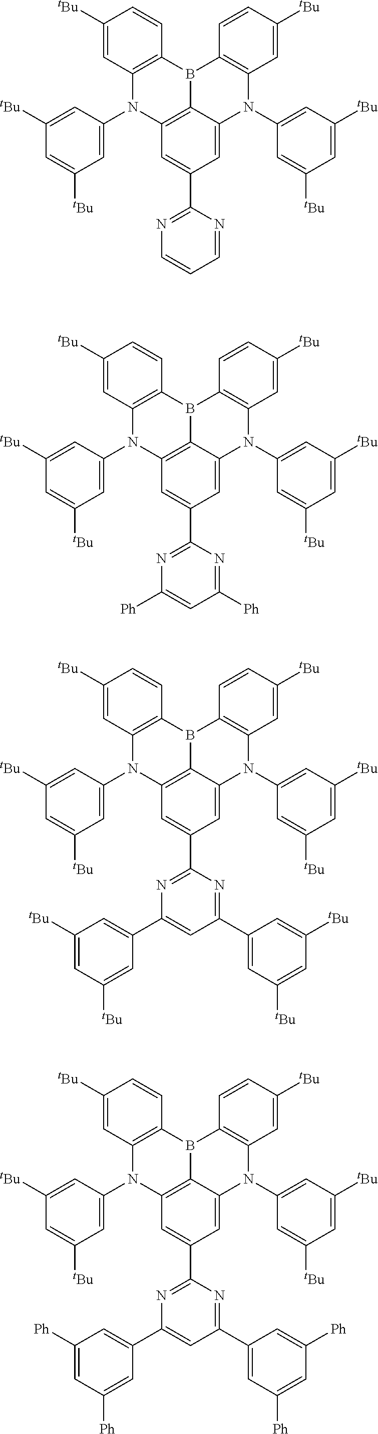

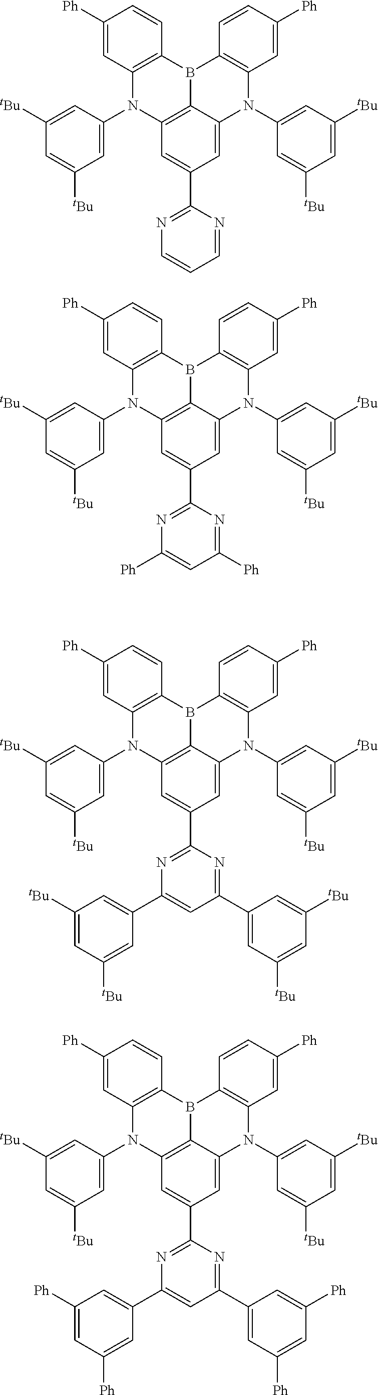

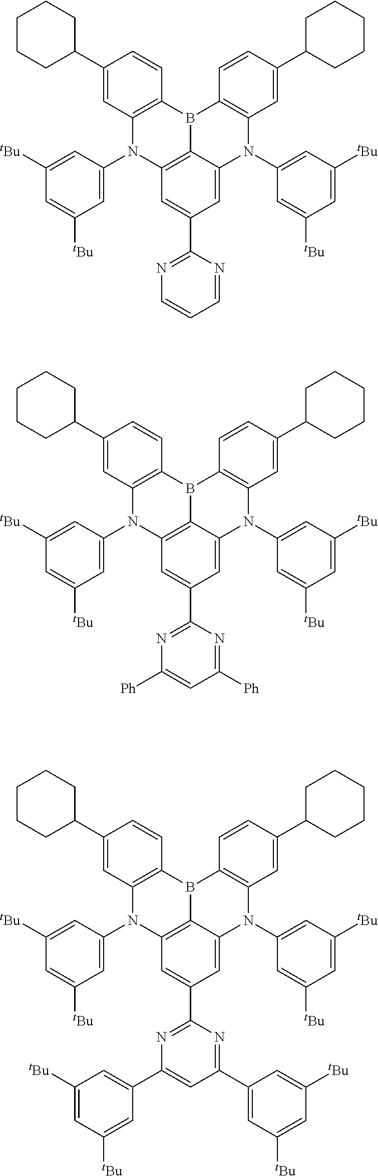 [SMILES: CC(C)(C)c1cc(N2c3cc(C4CCCCC4)ccc3B3c4ccc(C5CCCCC5)cc4N(c4cc(C(C)(C)C)cc(C(C)(C)C)c4)c4cc(-c5ncccn5)cc2c43)cc(C(C)(C)C)c1 ; CC(C)(C)c1cc(N2c3cc(C4CCCCC4)ccc3B3c4ccc(C5CCCCC5)cc4N(c4cc(C(C)(C)C)cc(C(C)(C)C)c4)c4cc(-c5nc(-c6ccccc6)cc(-c6ccccc6)n5)cc2c43)cc(C(C)(C)C)c1 ; CC(C)(C)c1cc(-c2cc(-c3cc(C(C)(C)C)cc(C(C)(C)C)c3)nc(-c3cc4c5c(c3)N(c3cc(C(C)(C)C)cc(C(C)(C)C)c3)c3cc(C6CCCCC6)ccc3B5c3ccc(C5CCCCC5)cc3N4c3cc(C(C)(C)C)cc(C(C)(C)C)c3)n2)cc(C(C)(C)C)c1]

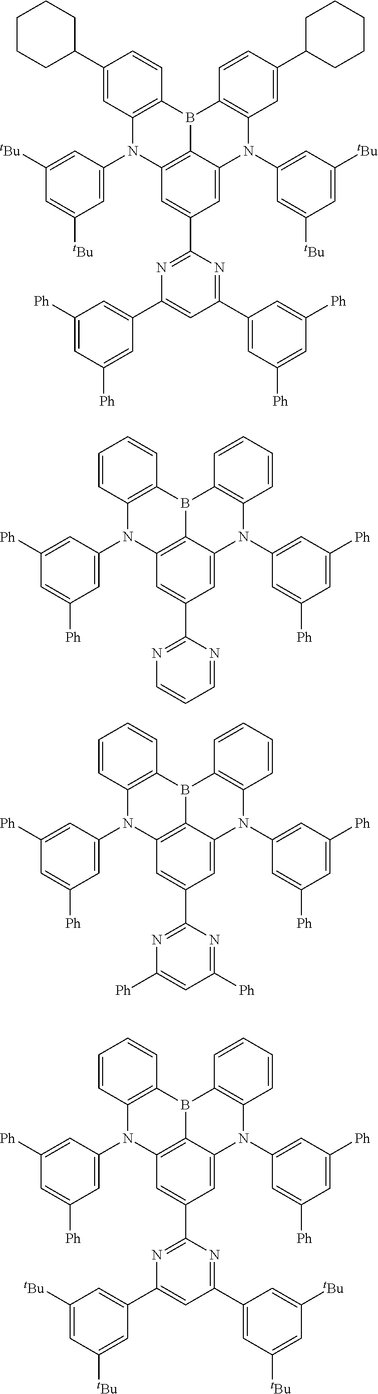

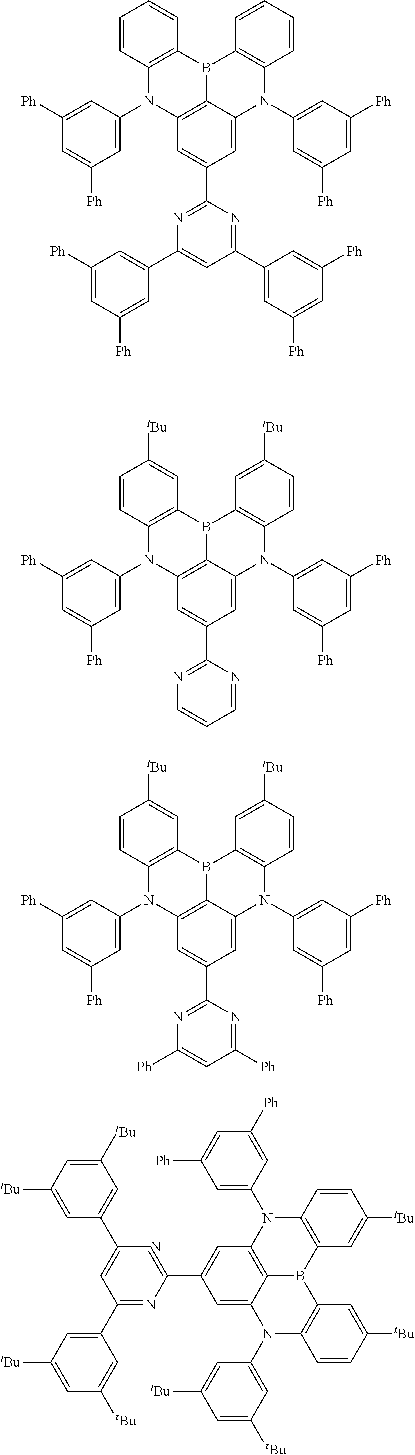

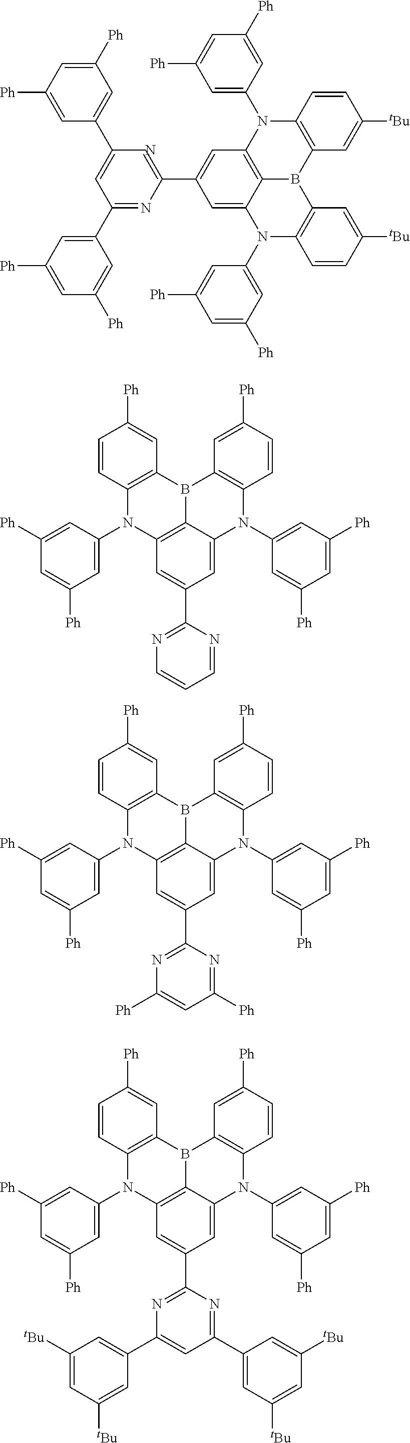

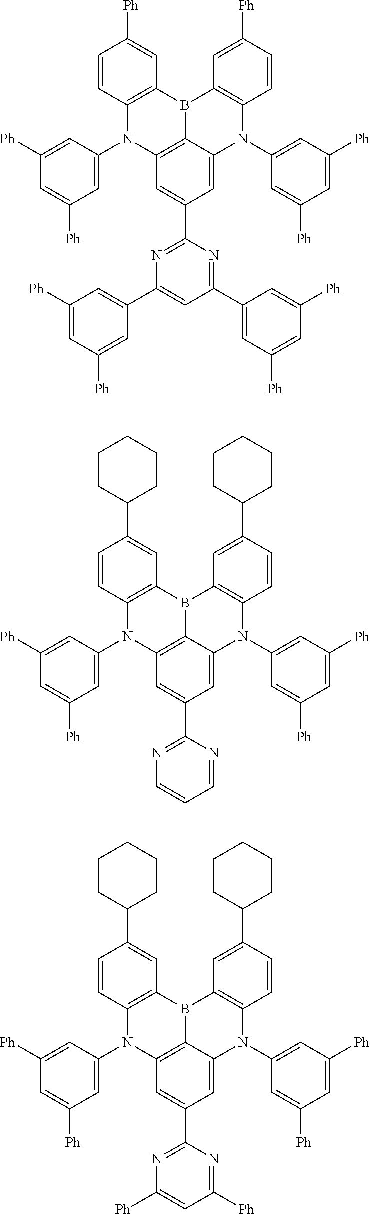 [SMILES: c1ccc(-c2cc(-c3ccccc3)cc(-c3cc(-c4cc(-c5ccccc5)cc(-c5ccccc5)c4)nc(-c4cc5c6c(c4)N(c4cc(-c7ccccc7)cc(-c7ccccc7)c4)c4ccc(-c7ccccc7)cc4B6c4cc(-c6ccccc6)ccc4N5c4cc(-c5ccccc5)cc(-c5ccccc5)c4)n3)c2)cc1 ; c1ccc(-c2cc(-c3ccccc3)cc(N3c4ccc(C5CCCCC5)cc4B4c5cc(C6CCCCC6)ccc5N(c5cc(-c6ccccc6)cc(-c6ccccc6)c5)c5cc(-c6ncccn6)cc3c54)c2)cc1 ; c1ccc(-c2cc(-c3ccccc3)cc(N3c4ccc(C5CCCCC5)cc4B4c5cc(C6CCCCC6)ccc5N(c5cc(-c6ccccc6)cc(-c6ccccc6)c5)c5cc(-c6nc(-c7ccccc7)cc(-c7ccccc7)n6)cc3c54)c2)cc1]

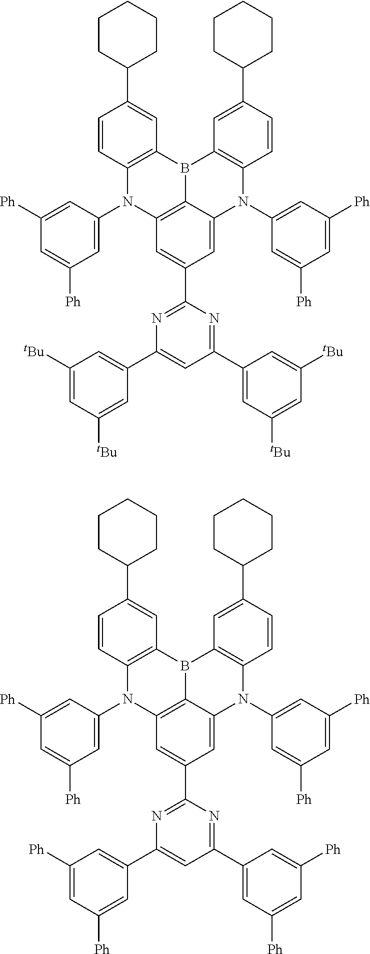 [SMILES: CC(C)(C)c1cc(-c2cc(-c3cc(C(C)(C)C)cc(C(C)(C)C)c3)nc(-c3cc4c5c(c3)N(c3cc(-c6ccccc6)cc(-c6ccccc6)c3)c3ccc(C6CCCCC6)cc3B5c3cc(C5CCCCC5)ccc3N4c3cc(-c4ccccc4)cc(-c4ccccc4)c3)n2)cc(C(C)(C)C)c1 ; c1ccc(-c2cc(-c3ccccc3)cc(-c3cc(-c4cc(-c5ccccc5)cc(-c5ccccc5)c4)nc(-c4cc5c6c(c4)N(c4cc(-c7ccccc7)cc(-c7ccccc7)c4)c4ccc(C7CCCCC7)cc4B6c4cc(C6CCCCC6)ccc4N5c4cc(-c5ccccc5)cc(-c5ccccc5)c4)n3)c2)cc1]

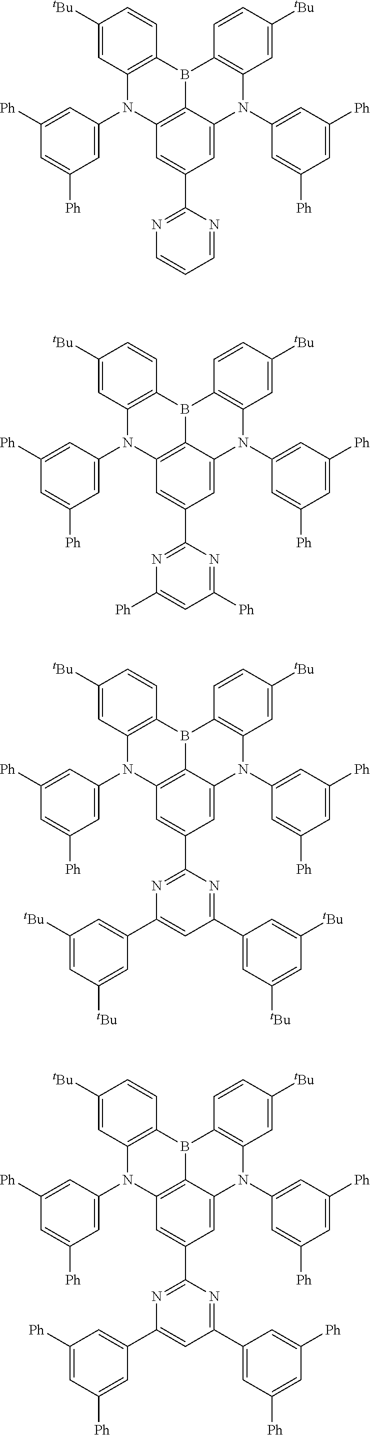

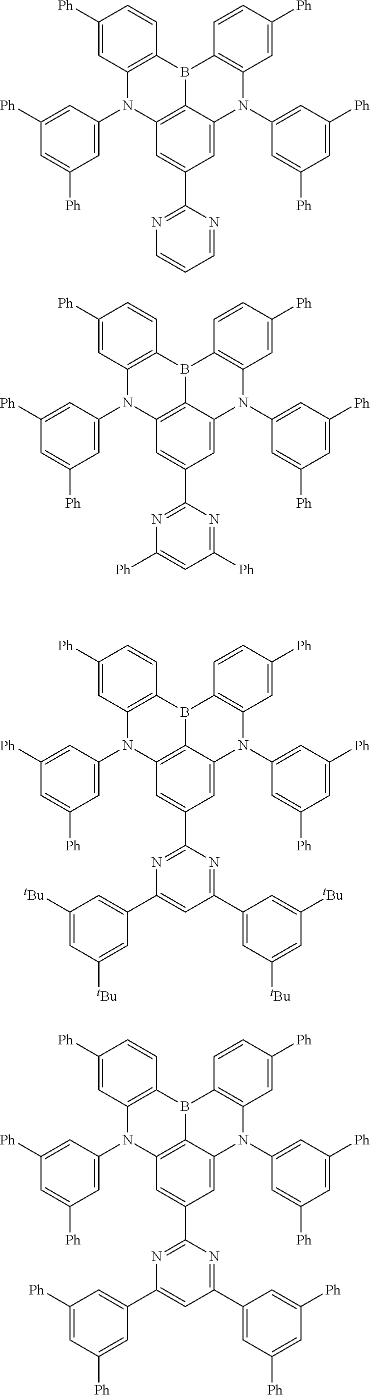

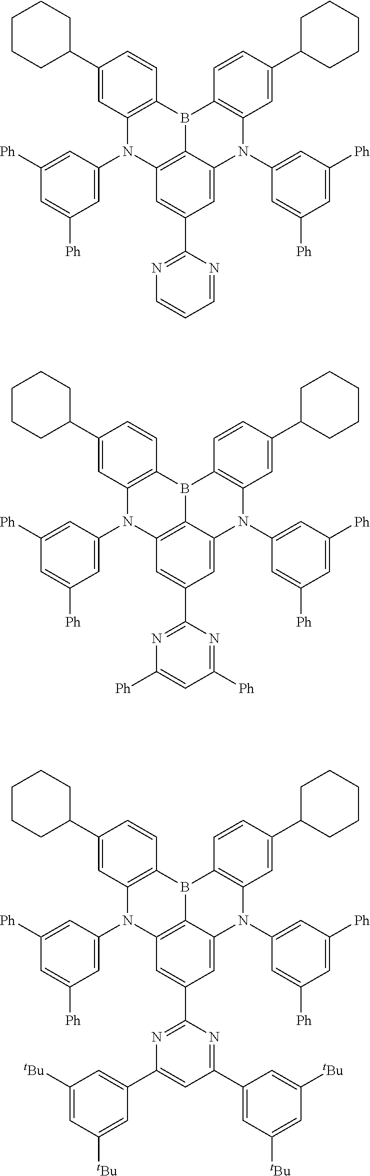 [SMILES: c1ccc(-c2cc(-c3ccccc3)cc(N3c4cc(C5CCCCC5)ccc4B4c5ccc(C6CCCCC6)cc5N(c5cc(-c6ccccc6)cc(-c6ccccc6)c5)c5cc(-c6ncccn6)cc3c54)c2)cc1 ; c1ccc(-c2cc(-c3ccccc3)cc(N3c4cc(C5CCCCC5)ccc4B4c5ccc(C6CCCCC6)cc5N(c5cc(-c6ccccc6)cc(-c6ccccc6)c5)c5cc(-c6nc(-c7ccccc7)cc(-c7ccccc7)n6)cc3c54)c2)cc1 ; CC(C)(C)c1cc(-c2cc(-c3cc(C(C)(C)C)cc(C(C)(C)C)c3)nc(-c3cc4c5c(c3)N(c3cc(-c6ccccc6)cc(-c6ccccc6)c3)c3cc(C6CCCCC6)ccc3B5c3ccc(C5CCCCC5)cc3N4c3cc(-c4ccccc4)cc(-c4ccccc4)c3)n2)cc(C(C)(C)C)c1]

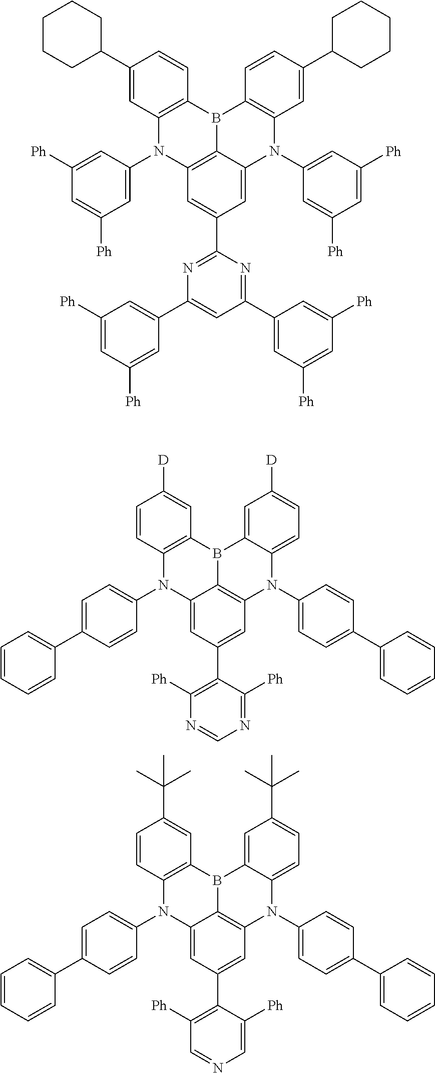

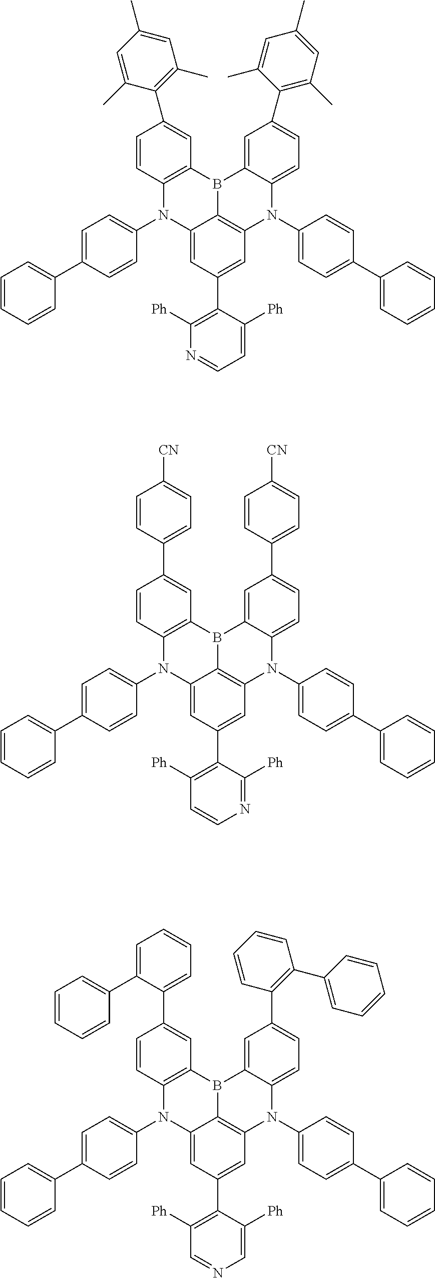 [SMILES: Cc1cc(C)c(-c2ccc3c(c2)B2c4cc(-c5c(C)cc(C)cc5C)ccc4N(c4ccc(-c5ccccc5)cc4)c4cc(-c5c(-c6ccccc6)ccnc5-c5ccccc5)cc(c42)N3c2ccc(-c3ccccc3)cc2)c(C)c1 ; N#Cc1ccc(-c2ccc3c(c2)B2c4cc(-c5ccc(C#N)cc5)ccc4N(c4ccc(-c5ccccc5)cc4)c4cc(-c5c(-c6ccccc6)ccnc5-c5ccccc5)cc(c42)N3c2ccc(-c3ccccc3)cc2)cc1 ; c1ccc(-c2ccc(N3c4ccc(-c5ccccc5-c5ccccc5)cc4B4c5cc(-c6ccccc6-c6ccccc6)ccc5N(c5ccc(-c6ccccc6)cc5)c5cc(-c6c(-c7ccccc7)cncc6-c6ccccc6)cc3c54)cc2)cc1]

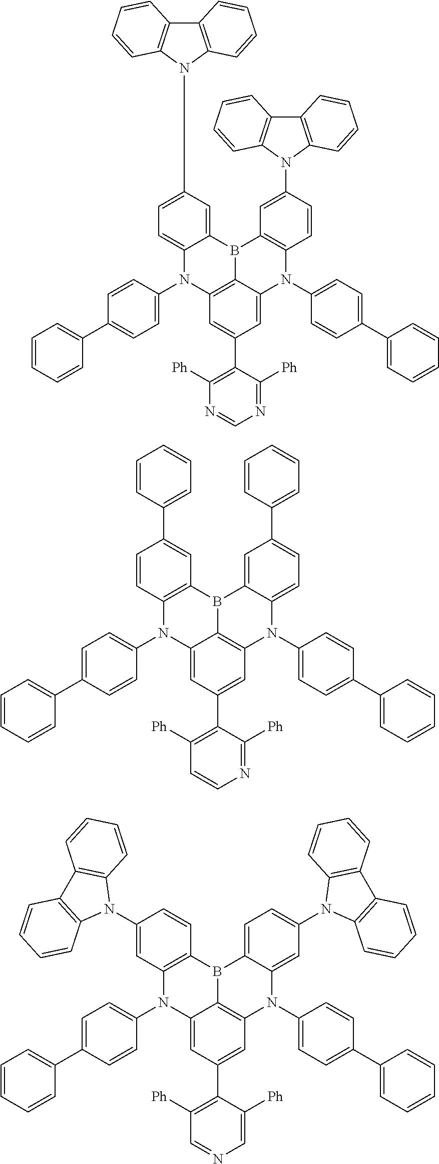

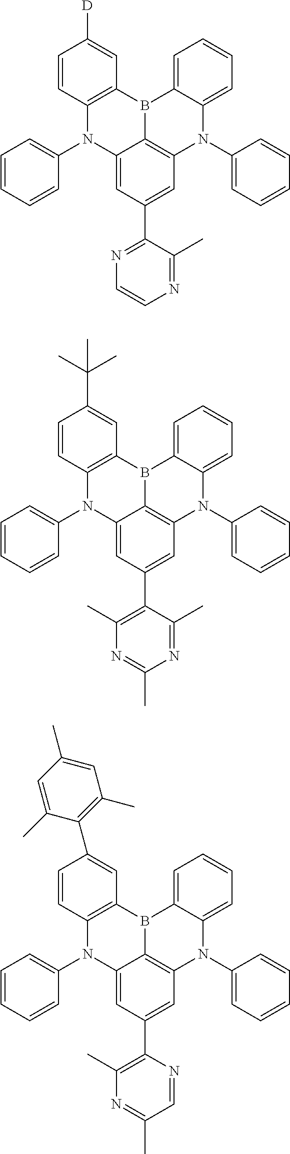 [SMILES: [2H]c1ccc2c(c1)B1c3ccccc3N(c3ccccc3)c3cc(-c4nccnc4C)cc(c31)N2c1ccccc1 ; Cc1nc(C)c(-c2cc3c4c(c2)N(c2ccccc2)c2ccc(C(C)(C)C)cc2B4c2ccccc2N3c2ccccc2)c(C)n1 ; Cc1cc(C)c(-c2ccc3c(c2)B2c4ccccc4N(c4ccccc4)c4cc(-c5ncc(C)nc5C)cc(c42)N3c2ccccc2)c(C)c1]

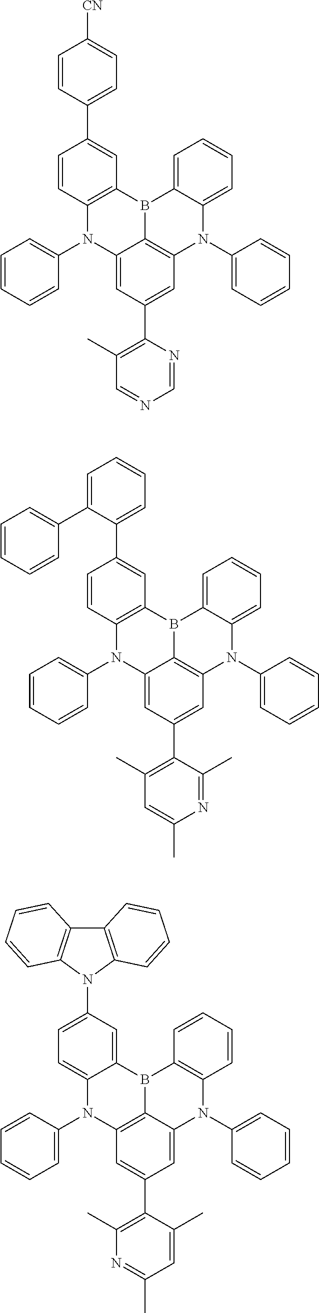 [SMILES: Cc1cncnc1-c1cc2c3c(c1)N(c1ccccc1)c1ccc(-c4ccc(C#N)cc4)cc1B3c1ccccc1N2c1ccccc1 ; Cc1cc(C)c(-c2cc3c4c(c2)N(c2ccccc2)c2ccc(-c5ccccc5-c5ccccc5)cc2B4c2ccccc2N3c2ccccc2)c(C)n1 ; Cc1cc(C)c(-c2cc3c4c(c2)N(c2ccccc2)c2ccc(-n5c6ccccc6c6ccccc65)cc2B4c2ccccc2N3c2ccccc2)c(C)n1]

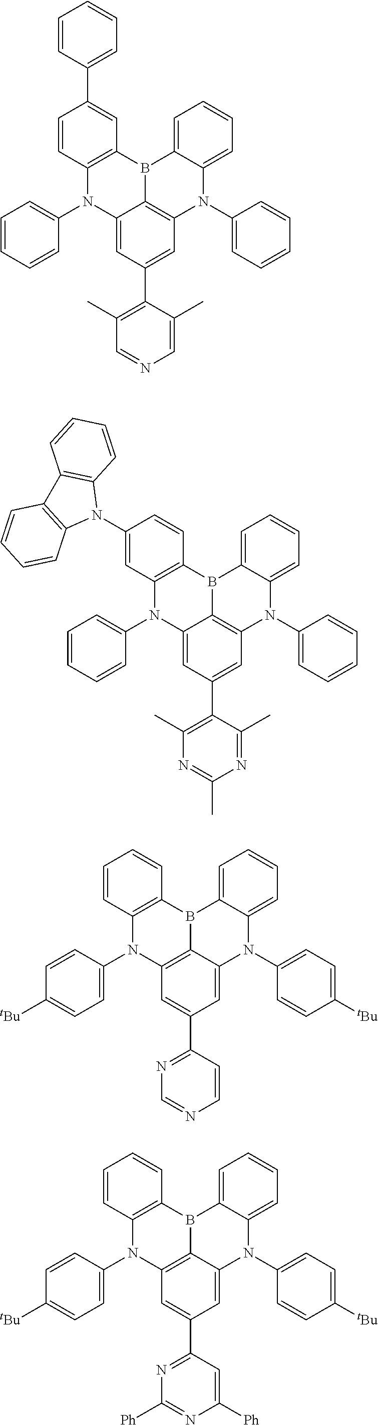 [SMILES: Cc1cncc(C)c1-c1cc2c3c(c1)N(c1ccccc1)c1ccc(-c4ccccc4)cc1B3c1ccccc1N2c1ccccc1 ; Cc1nc(C)c(-c2cc3c4c(c2)N(c2ccccc2)c2cc(-n5c6ccccc6c6ccccc65)ccc2B4c2ccccc2N3c2ccccc2)c(C)n1 ; CC(C)(C)c1ccc(N2c3ccccc3B3c4ccccc4N(c4ccc(C(C)(C)C)cc4)c4cc(-c5ccncn5)cc2c43)cc1 ; CC(C)(C)c1ccc(N2c3ccccc3B3c4ccccc4N(c4ccc(C(C)(C)C)cc4)c4cc(-c5cc(-c6ccccc6)nc(-c6ccccc6)n5)cc2c43)cc1]

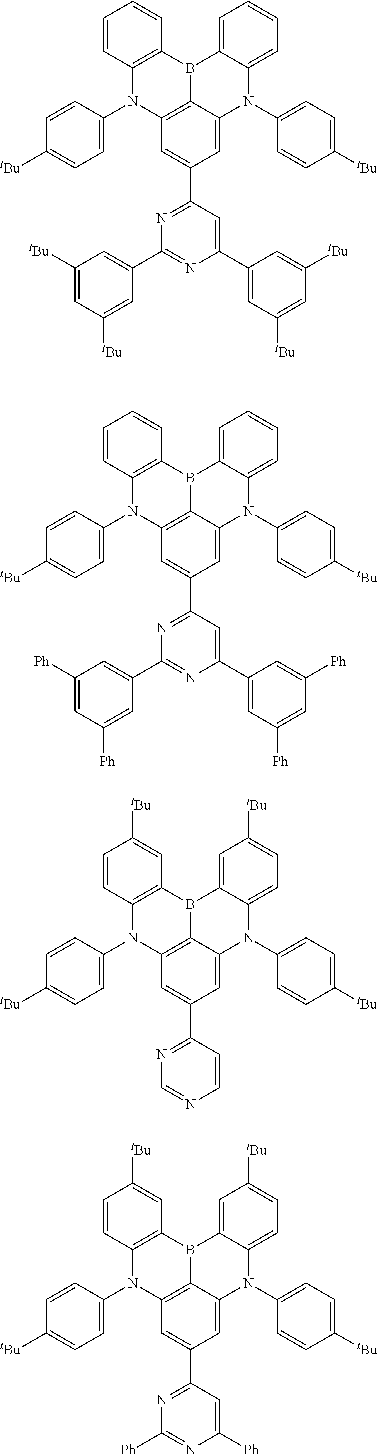

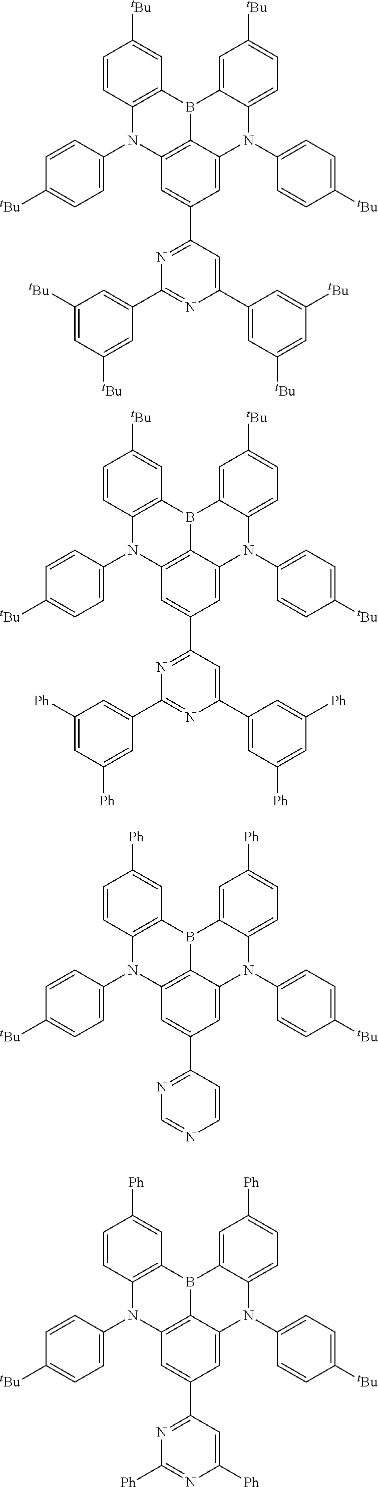

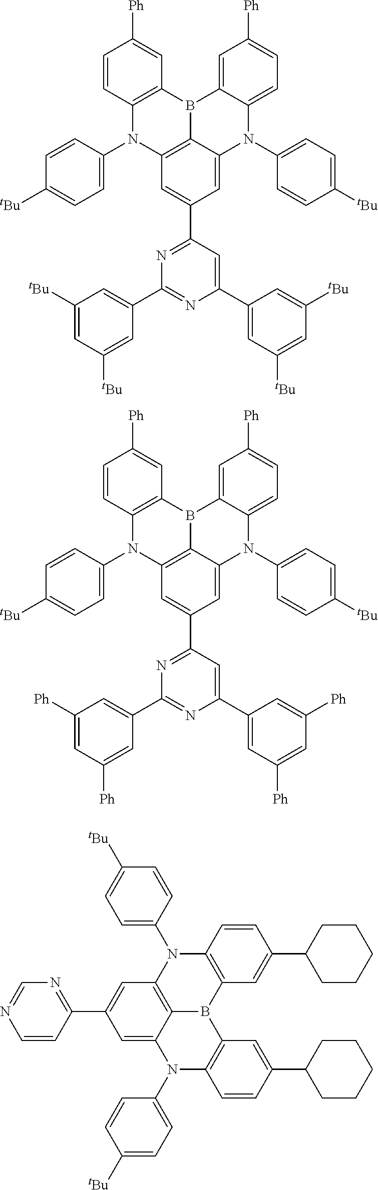

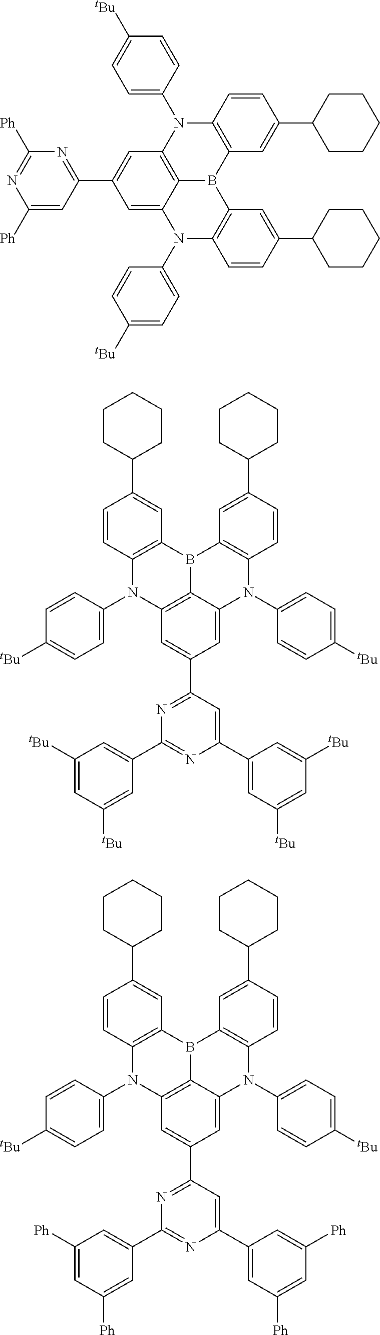

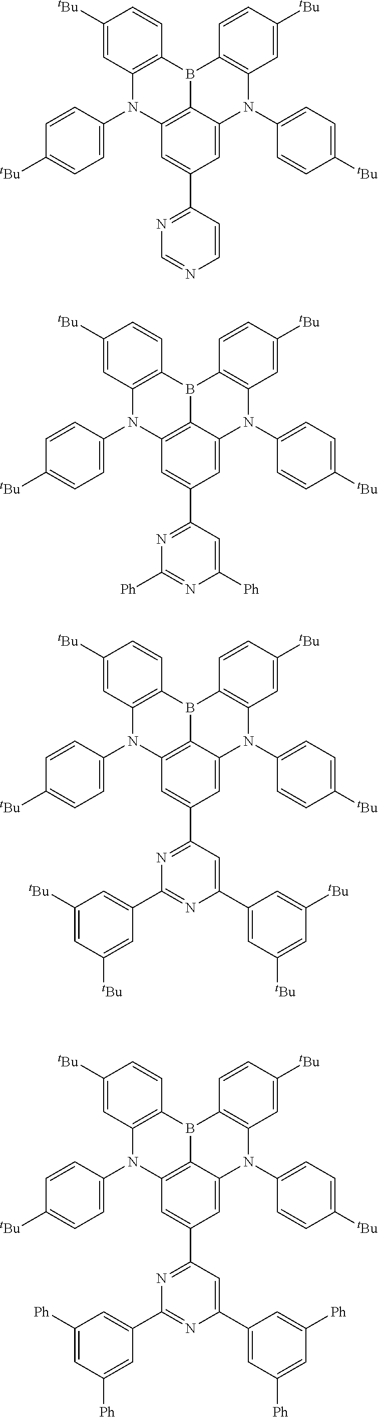

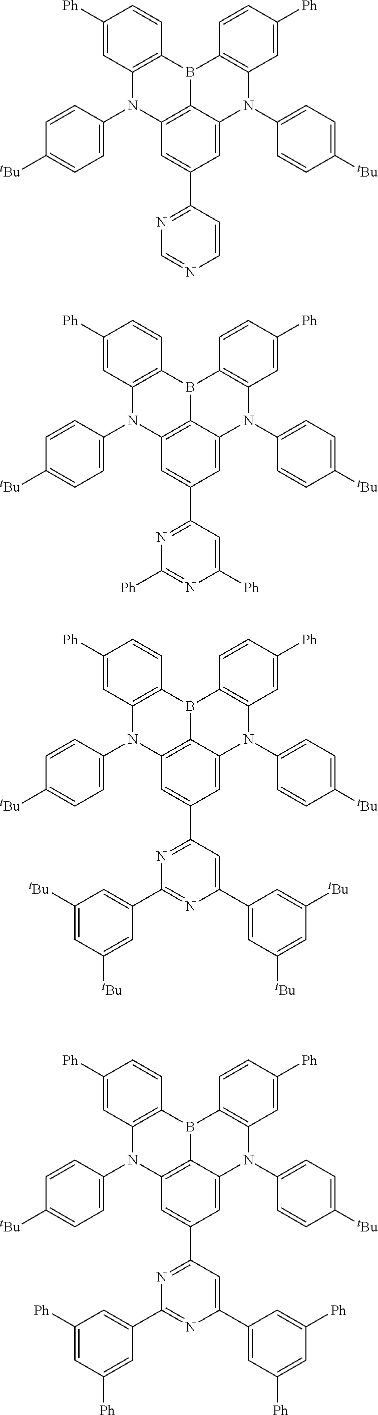

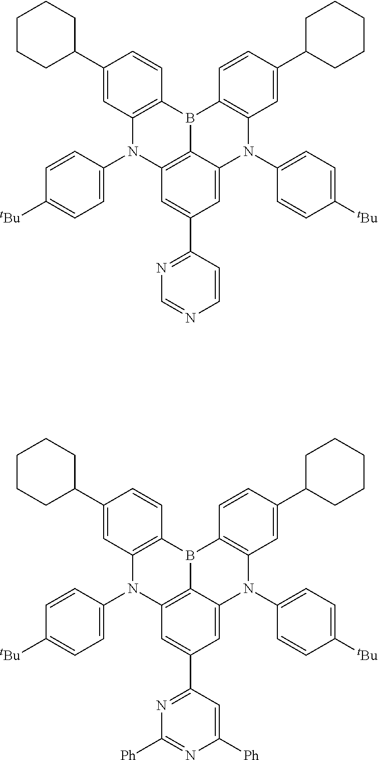 [SMILES: CC(C)(C)c1ccc(N2c3cc(C4CCCCC4)ccc3B3c4ccc(C5CCCCC5)cc4N(c4ccc(C(C)(C)C)cc4)c4cc(-c5ccncn5)cc2c43)cc1 ; CC(C)(C)c1ccc(N2c3cc(C4CCCCC4)ccc3B3c4ccc(C5CCCCC5)cc4N(c4ccc(C(C)(C)C)cc4)c4cc(-c5cc(-c6ccccc6)nc(-c6ccccc6)n5)cc2c43)cc1]

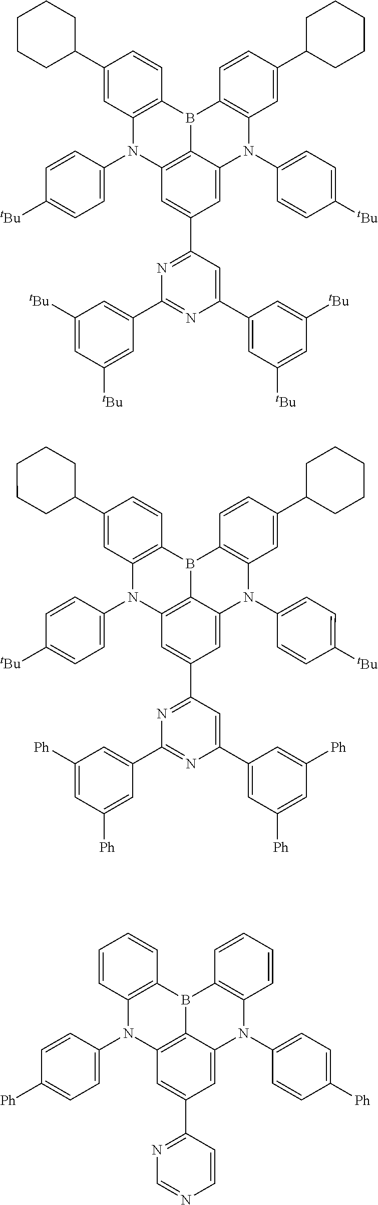 [SMILES: CC(C)(C)c1ccc(N2c3cc(C4CCCCC4)ccc3B3c4ccc(C5CCCCC5)cc4N(c4ccc(C(C)(C)C)cc4)c4cc(-c5cc(-c6cc(C(C)(C)C)cc(C(C)(C)C)c6)nc(-c6cc(C(C)(C)C)cc(C(C)(C)C)c6)n5)cc2c43)cc1 ; CC(C)(C)c1ccc(N2c3cc(C4CCCCC4)ccc3B3c4ccc(C5CCCCC5)cc4N(c4ccc(C(C)(C)C)cc4)c4cc(-c5cc(-c6cc(-c7ccccc7)cc(-c7ccccc7)c6)nc(-c6cc(-c7ccccc7)cc(-c7ccccc7)c6)n5)cc2c43)cc1 ; c1ccc(-c2ccc(N3c4ccccc4B4c5ccccc5N(c5ccc(-c6ccccc6)cc5)c5cc(-c6ccncn6)cc3c54)cc2)cc1]

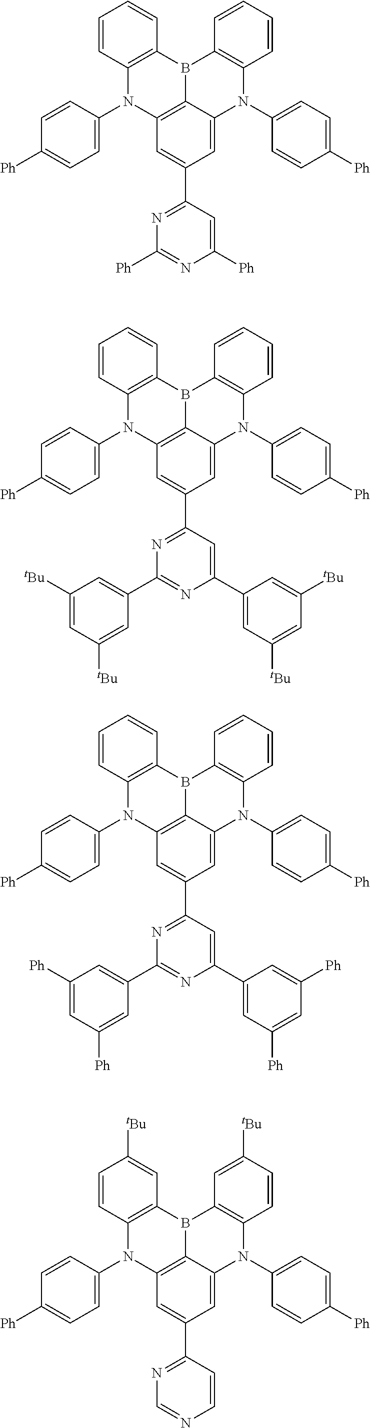

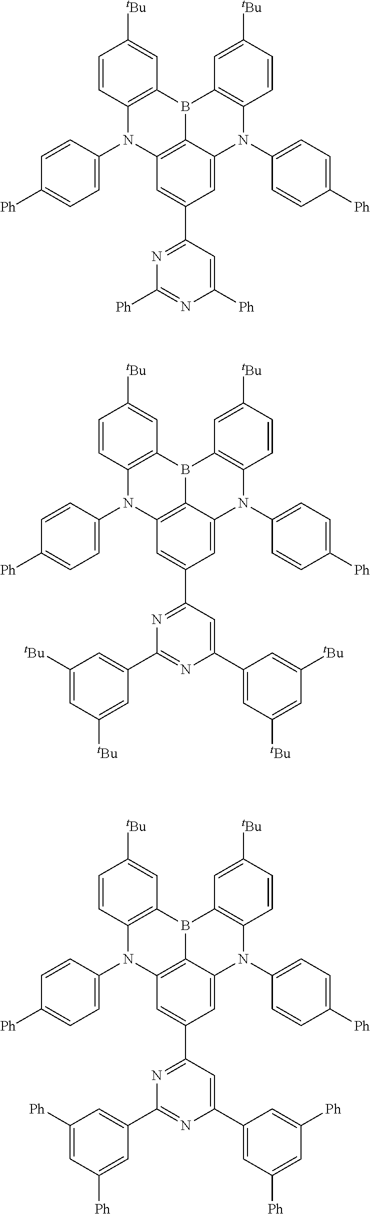 [SMILES: CC(C)(C)c1ccc2c(c1)B1c3cc(C(C)(C)C)ccc3N(c3ccc(-c4ccccc4)cc3)c3cc(-c4cc(-c5ccccc5)nc(-c5ccccc5)n4)cc(c31)N2c1ccc(-c2ccccc2)cc1 ; CC(C)(C)c1cc(-c2cc(-c3cc4c5c(c3)N(c3ccc(-c6ccccc6)cc3)c3ccc(C(C)(C)C)cc3B5c3cc(C(C)(C)C)ccc3N4c3ccc(-c4ccccc4)cc3)nc(-c3cc(C(C)(C)C)cc(C(C)(C)C)c3)n2)cc(C(C)(C)C)c1 ; CC(C)(C)c1ccc2c(c1)B1c3cc(C(C)(C)C)ccc3N(c3ccc(-c4ccccc4)cc3)c3cc(-c4cc(-c5cc(-c6ccccc6)cc(-c6ccccc6)c5)nc(-c5cc(-c6ccccc6)cc(-c6ccccc6)c5)n4)cc(c31)N2c1ccc(-c2ccccc2)cc1]

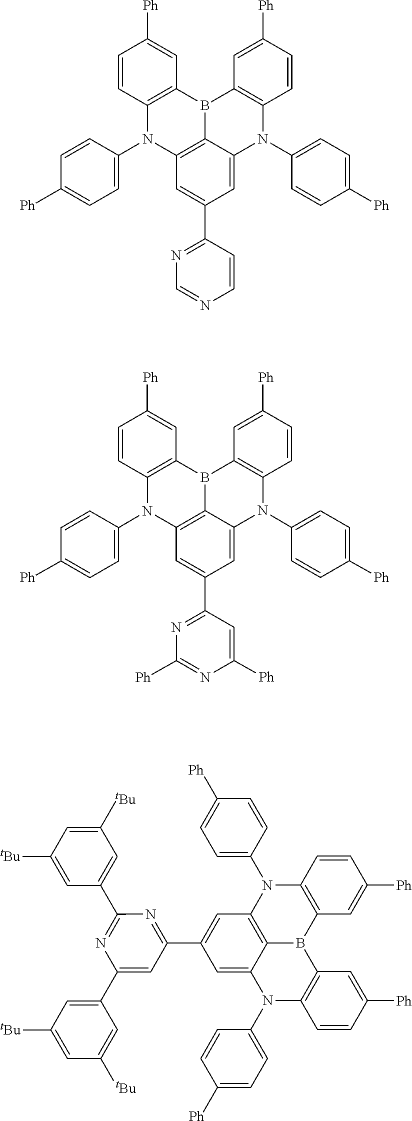 [SMILES: c1ccc(-c2ccc(N3c4ccc(-c5ccccc5)cc4B4c5cc(-c6ccccc6)ccc5N(c5ccc(-c6ccccc6)cc5)c5cc(-c6ccncn6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6cc(-c7ccccc7)nc(-c7ccccc7)n6)cc3c54)cc2)cc1 ; CC(C)(C)c1cc(-c2cc(-c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)nc(-c3cc(C(C)(C)C)cc(C(C)(C)C)c3)n2)cc(C(C)(C)C)c1]

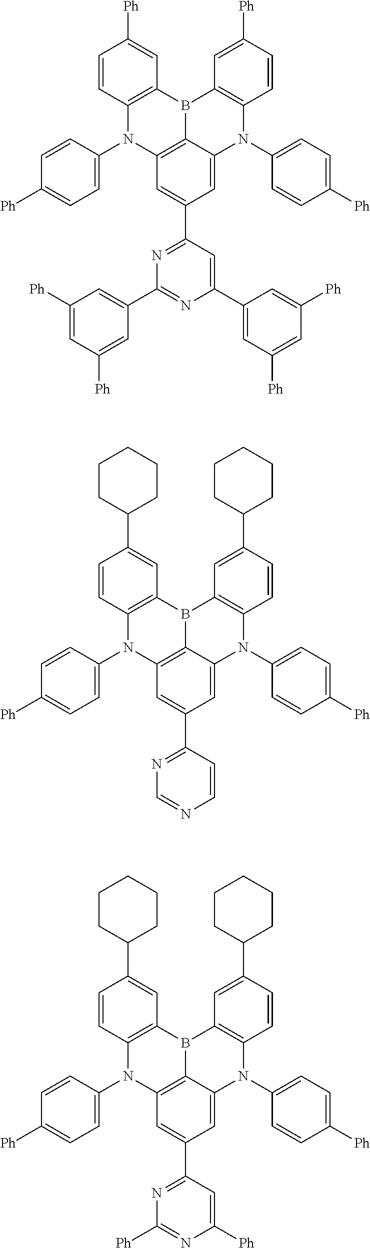 [SMILES: c1ccc(-c2ccc(N3c4ccc(-c5ccccc5)cc4B4c5cc(-c6ccccc6)ccc5N(c5ccc(-c6ccccc6)cc5)c5cc(-c6cc(-c7cc(-c8ccccc8)cc(-c8ccccc8)c7)nc(-c7cc(-c8ccccc8)cc(-c8ccccc8)c7)n6)cc3c54)cc2)cc1 ; c1ccc(-c2ccc(N3c4ccc(C5CCCCC5)cc4B4c5cc(C6CCCCC6)ccc5N(c5ccc(-c6ccccc6)cc5)c5cc(-c6ccncn6)cc3c54)cc2)cc1 ; c1ccc(-c2ccc(N3c4ccc(C5CCCCC5)cc4B4c5cc(C6CCCCC6)ccc5N(c5ccc(-c6ccccc6)cc5)c5cc(-c6cc(-c7ccccc7)nc(-c7ccccc7)n6)cc3c54)cc2)cc1]

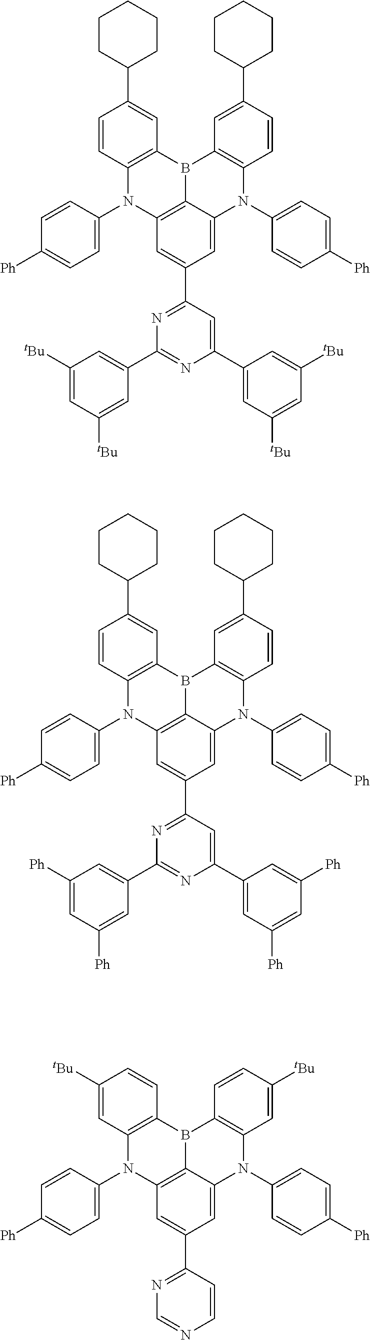 [SMILES: CC(C)(C)c1cc(-c2cc(-c3cc4c5c(c3)N(c3ccc(-c6ccccc6)cc3)c3ccc(C6CCCCC6)cc3B5c3cc(C5CCCCC5)ccc3N4c3ccc(-c4ccccc4)cc3)nc(-c3cc(C(C)(C)C)cc(C(C)(C)C)c3)n2)cc(C(C)(C)C)c1 ; c1ccc(-c2ccc(N3c4ccc(C5CCCCC5)cc4B4c5cc(C6CCCCC6)ccc5N(c5ccc(-c6ccccc6)cc5)c5cc(-c6cc(-c7cc(-c8ccccc8)cc(-c8ccccc8)c7)nc(-c7cc(-c8ccccc8)cc(-c8ccccc8)c7)n6)cc3c54)cc2)cc1 ; CC(C)(C)c1ccc2c(c1)N(c1ccc(-c3ccccc3)cc1)c1cc(-c3ccncn3)cc3c1B2c1ccc(C(C)(C)C)cc1N3c1ccc(-c2ccccc2)cc1]

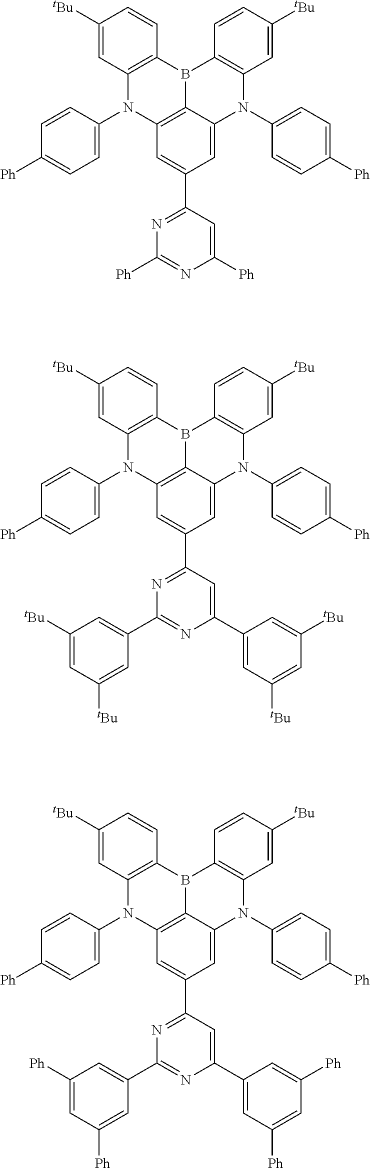 [SMILES: CC(C)(C)c1ccc2c(c1)N(c1ccc(-c3ccccc3)cc1)c1cc(-c3cc(-c4ccccc4)nc(-c4ccccc4)n3)cc3c1B2c1ccc(C(C)(C)C)cc1N3c1ccc(-c2ccccc2)cc1 ; CC(C)(C)c1cc(-c2cc(-c3cc4c5c(c3)N(c3ccc(-c6ccccc6)cc3)c3cc(C(C)(C)C)ccc3B5c3ccc(C(C)(C)C)cc3N4c3ccc(-c4ccccc4)cc3)nc(-c3cc(C(C)(C)C)cc(C(C)(C)C)c3)n2)cc(C(C)(C)C)c1 ; CC(C)(C)c1ccc2c(c1)N(c1ccc(-c3ccccc3)cc1)c1cc(-c3cc(-c4cc(-c5ccccc5)cc(-c5ccccc5)c4)nc(-c4cc(-c5ccccc5)cc(-c5ccccc5)c4)n3)cc3c1B2c1ccc(C(C)(C)C)cc1N3c1ccc(-c2ccccc2)cc1]

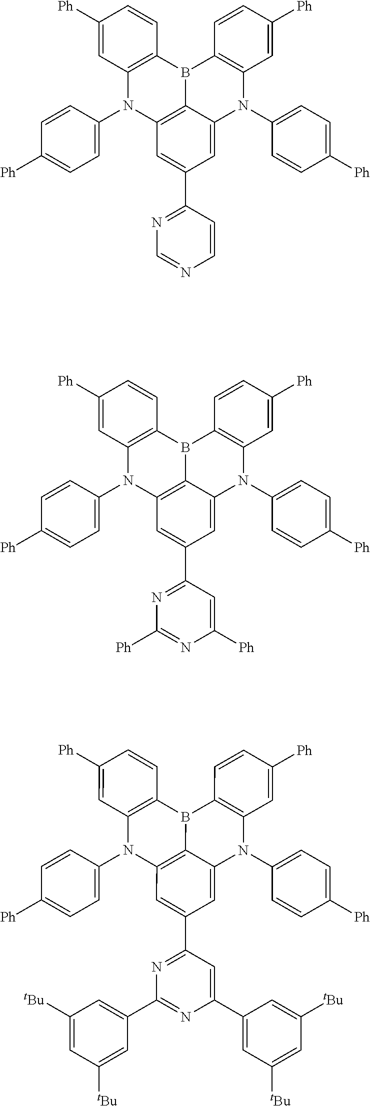 [SMILES: c1ccc(-c2ccc(N3c4cc(-c5ccccc5)ccc4B4c5ccc(-c6ccccc6)cc5N(c5ccc(-c6ccccc6)cc5)c5cc(-c6ccncn6)cc3c54)cc2)cc1 ; c1ccc(-c2ccc(N3c4cc(-c5ccccc5)ccc4B4c5ccc(-c6ccccc6)cc5N(c5ccc(-c6ccccc6)cc5)c5cc(-c6cc(-c7ccccc7)nc(-c7ccccc7)n6)cc3c54)cc2)cc1 ; CC(C)(C)c1cc(-c2cc(-c3cc4c5c(c3)N(c3ccc(-c6ccccc6)cc3)c3cc(-c6ccccc6)ccc3B5c3ccc(-c5ccccc5)cc3N4c3ccc(-c4ccccc4)cc3)nc(-c3cc(C(C)(C)C)cc(C(C)(C)C)c3)n2)cc(C(C)(C)C)c1]

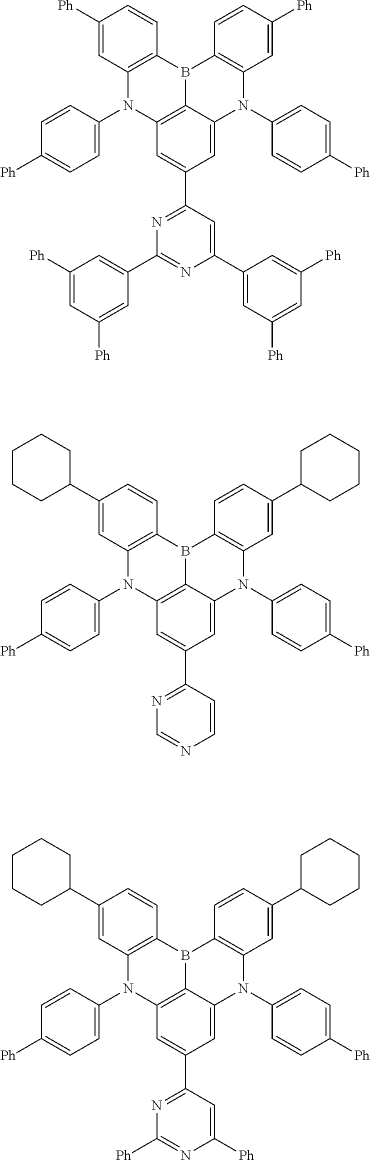 [SMILES: c1ccc(-c2ccc(N3c4cc(-c5ccccc5)ccc4B4c5ccc(-c6ccccc6)cc5N(c5ccc(-c6ccccc6)cc5)c5cc(-c6cc(-c7cc(-c8ccccc8)cc(-c8ccccc8)c7)nc(-c7cc(-c8ccccc8)cc(-c8ccccc8)c7)n6)cc3c54)cc2)cc1 ; c1ccc(-c2ccc(N3c4cc(C5CCCCC5)ccc4B4c5ccc(C6CCCCC6)cc5N(c5ccc(-c6ccccc6)cc5)c5cc(-c6ccncn6)cc3c54)cc2)cc1 ; c1ccc(-c2ccc(N3c4cc(C5CCCCC5)ccc4B4c5ccc(C6CCCCC6)cc5N(c5ccc(-c6ccccc6)cc5)c5cc(-c6cc(-c7ccccc7)nc(-c7ccccc7)n6)cc3c54)cc2)cc1]

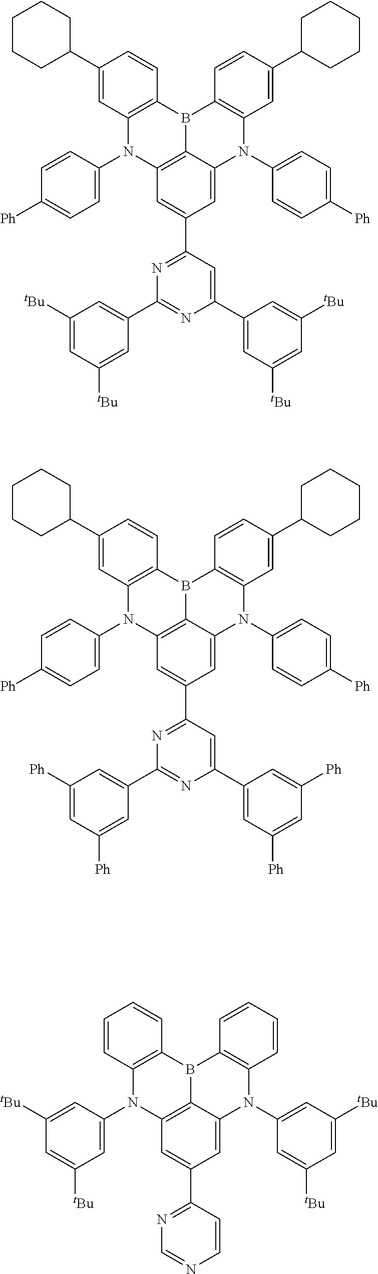 [SMILES: CC(C)(C)c1cc(-c2cc(-c3cc4c5c(c3)N(c3ccc(-c6ccccc6)cc3)c3cc(C6CCCCC6)ccc3B5c3ccc(C5CCCCC5)cc3N4c3ccc(-c4ccccc4)cc3)nc(-c3cc(C(C)(C)C)cc(C(C)(C)C)c3)n2)cc(C(C)(C)C)c1 ; c1ccc(-c2ccc(N3c4cc(C5CCCCC5)ccc4B4c5ccc(C6CCCCC6)cc5N(c5ccc(-c6ccccc6)cc5)c5cc(-c6cc(-c7cc(-c8ccccc8)cc(-c8ccccc8)c7)nc(-c7cc(-c8ccccc8)cc(-c8ccccc8)c7)n6)cc3c54)cc2)cc1 ; CC(C)(C)c1cc(N2c3ccccc3B3c4ccccc4N(c4cc(C(C)(C)C)cc(C(C)(C)C)c4)c4cc(-c5ccncn5)cc2c43)cc(C(C)(C)C)c1]

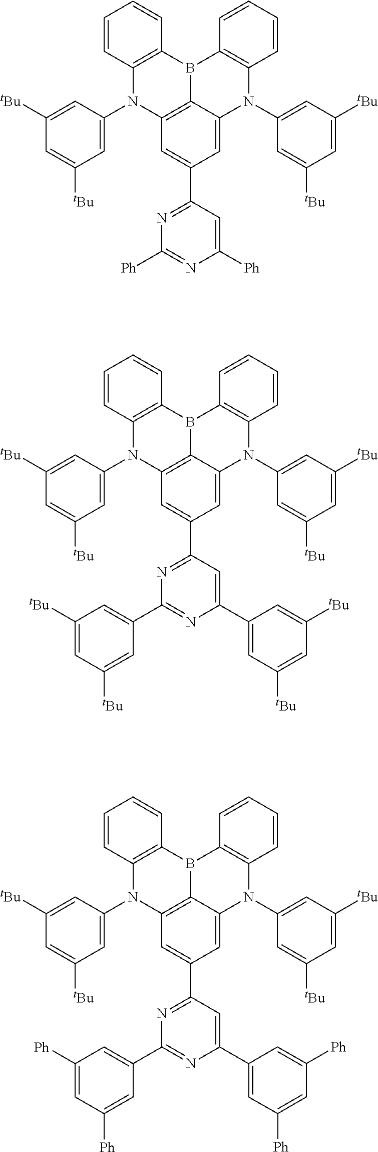 [SMILES: CC(C)(C)c1cc(N2c3ccccc3B3c4ccccc4N(c4cc(C(C)(C)C)cc(C(C)(C)C)c4)c4cc(-c5cc(-c6ccccc6)nc(-c6ccccc6)n5)cc2c43)cc(C(C)(C)C)c1 ; CC(C)(C)c1cc(-c2cc(-c3cc4c5c(c3)N(c3cc(C(C)(C)C)cc(C(C)(C)C)c3)c3ccccc3B5c3ccccc3N4c3cc(C(C)(C)C)cc(C(C)(C)C)c3)nc(-c3cc(C(C)(C)C)cc(C(C)(C)C)c3)n2)cc(C(C)(C)C)c1 ; CC(C)(C)c1cc(N2c3ccccc3B3c4ccccc4N(c4cc(C(C)(C)C)cc(C(C)(C)C)c4)c4cc(-c5cc(-c6cc(-c7ccccc7)cc(-c7ccccc7)c6)nc(-c6cc(-c7ccccc7)cc(-c7ccccc7)c6)n5)cc2c43)cc(C(C)(C)C)c1]

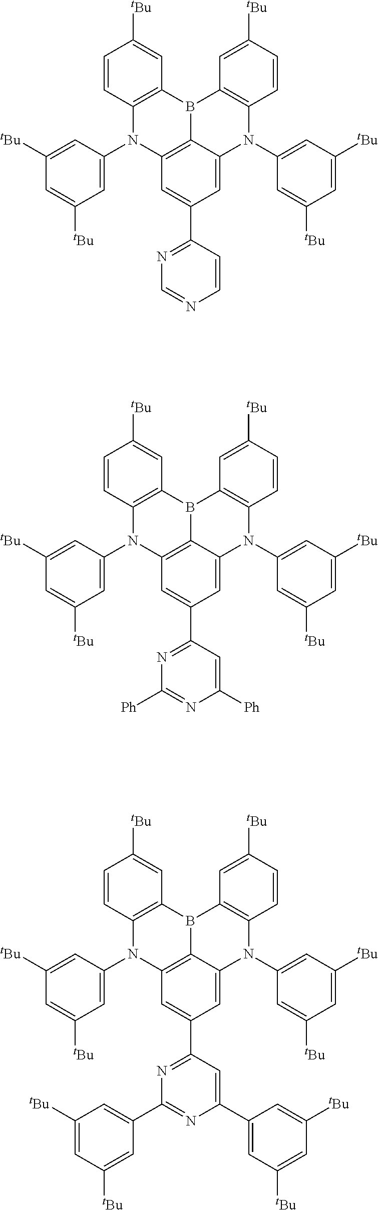 [SMILES: CC(C)(C)c1cc(N2c3ccc(C(C)(C)C)cc3B3c4cc(C(C)(C)C)ccc4N(c4cc(C(C)(C)C)cc(C(C)(C)C)c4)c4cc(-c5ccncn5)cc2c43)cc(C(C)(C)C)c1 ; CC(C)(C)c1cc(N2c3ccc(C(C)(C)C)cc3B3c4cc(C(C)(C)C)ccc4N(c4cc(C(C)(C)C)cc(C(C)(C)C)c4)c4cc(-c5cc(-c6ccccc6)nc(-c6ccccc6)n5)cc2c43)cc(C(C)(C)C)c1 ; CC(C)(C)c1cc(-c2cc(-c3cc4c5c(c3)N(c3cc(C(C)(C)C)cc(C(C)(C)C)c3)c3ccc(C(C)(C)C)cc3B5c3cc(C(C)(C)C)ccc3N4c3cc(C(C)(C)C)cc(C(C)(C)C)c3)nc(-c3cc(C(C)(C)C)cc(C(C)(C)C)c3)n2)cc(C(C)(C)C)c1]

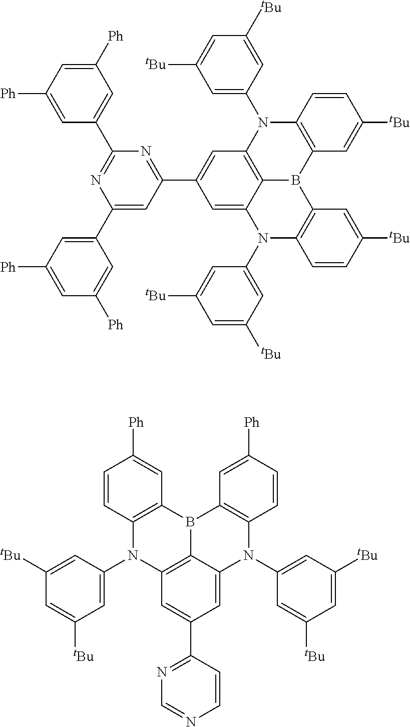 [SMILES: CC(C)(C)c1cc(N2c3ccc(C(C)(C)C)cc3B3c4cc(C(C)(C)C)ccc4N(c4cc(C(C)(C)C)cc(C(C)(C)C)c4)c4cc(-c5cc(-c6cc(-c7ccccc7)cc(-c7ccccc7)c6)nc(-c6cc(-c7ccccc7)cc(-c7ccccc7)c6)n5)cc2c43)cc(C(C)(C)C)c1 ; CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5ccncn5)cc2c43)cc(C(C)(C)C)c1]

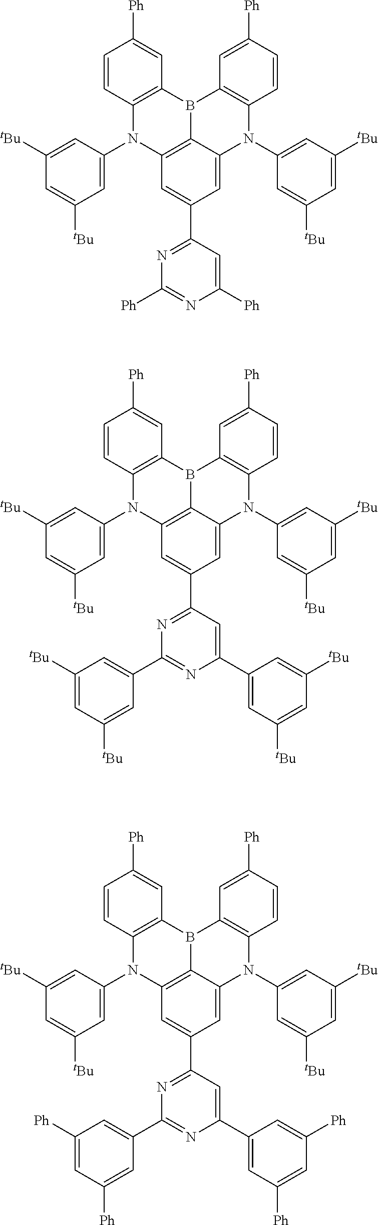

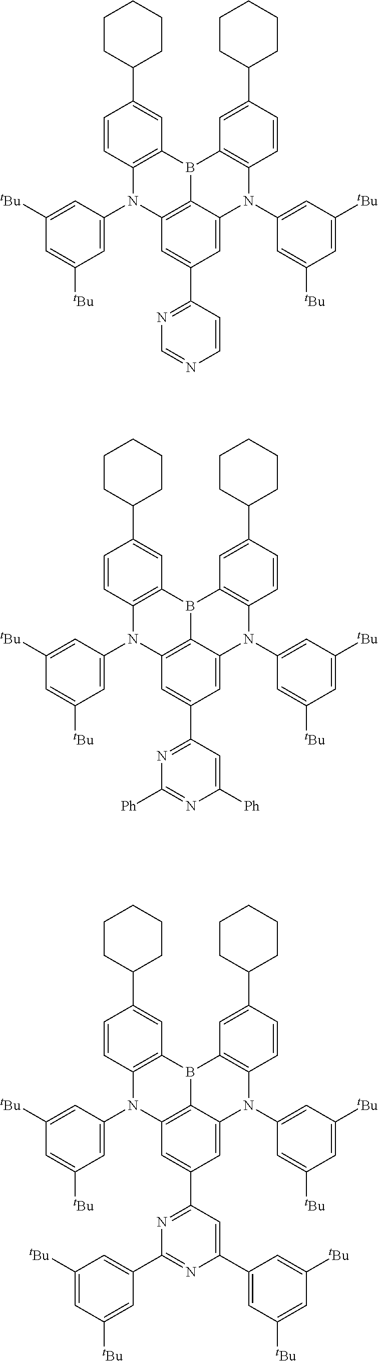 [SMILES: CC(C)(C)c1cc(N2c3ccc(C4CCCCC4)cc3B3c4cc(C5CCCCC5)ccc4N(c4cc(C(C)(C)C)cc(C(C)(C)C)c4)c4cc(-c5ccncn5)cc2c43)cc(C(C)(C)C)c1 ; CC(C)(C)c1cc(N2c3ccc(C4CCCCC4)cc3B3c4cc(C5CCCCC5)ccc4N(c4cc(C(C)(C)C)cc(C(C)(C)C)c4)c4cc(-c5cc(-c6ccccc6)nc(-c6ccccc6)n5)cc2c43)cc(C(C)(C)C)c1 ; CC(C)(C)c1cc(-c2cc(-c3cc4c5c(c3)N(c3cc(C(C)(C)C)cc(C(C)(C)C)c3)c3ccc(C6CCCCC6)cc3B5c3cc(C5CCCCC5)ccc3N4c3cc(C(C)(C)C)cc(C(C)(C)C)c3)nc(-c3cc(C(C)(C)C)cc(C(C)(C)C)c3)n2)cc(C(C)(C)C)c1]

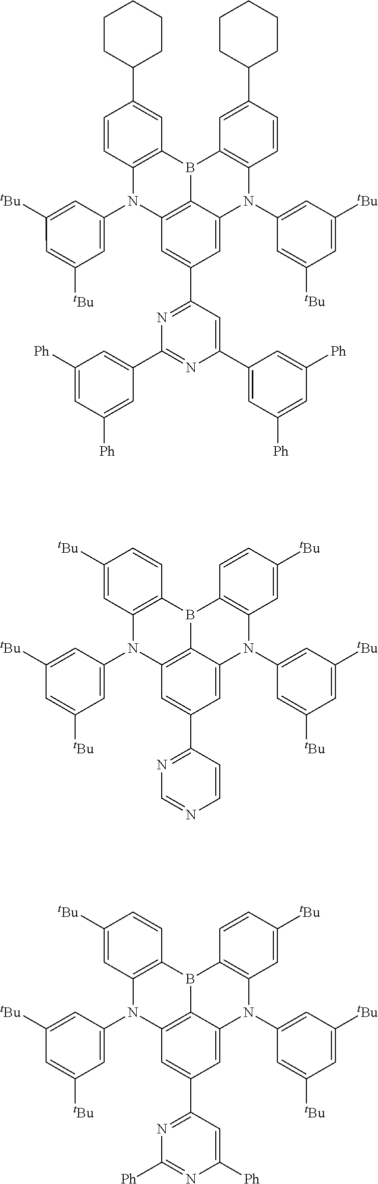 [SMILES: CC(C)(C)c1cc(N2c3ccc(C4CCCCC4)cc3B3c4cc(C5CCCCC5)ccc4N(c4cc(C(C)(C)C)cc(C(C)(C)C)c4)c4cc(-c5cc(-c6cc(-c7ccccc7)cc(-c7ccccc7)c6)nc(-c6cc(-c7ccccc7)cc(-c7ccccc7)c6)n5)cc2c43)cc(C(C)(C)C)c1 ; CC(C)(C)c1cc(N2c3cc(C(C)(C)C)ccc3B3c4ccc(C(C)(C)C)cc4N(c4cc(C(C)(C)C)cc(C(C)(C)C)c4)c4cc(-c5ccncn5)cc2c43)cc(C(C)(C)C)c1 ; CC(C)(C)c1cc(N2c3cc(C(C)(C)C)ccc3B3c4ccc(C(C)(C)C)cc4N(c4cc(C(C)(C)C)cc(C(C)(C)C)c4)c4cc(-c5cc(-c6ccccc6)nc(-c6ccccc6)n5)cc2c43)cc(C(C)(C)C)c1]

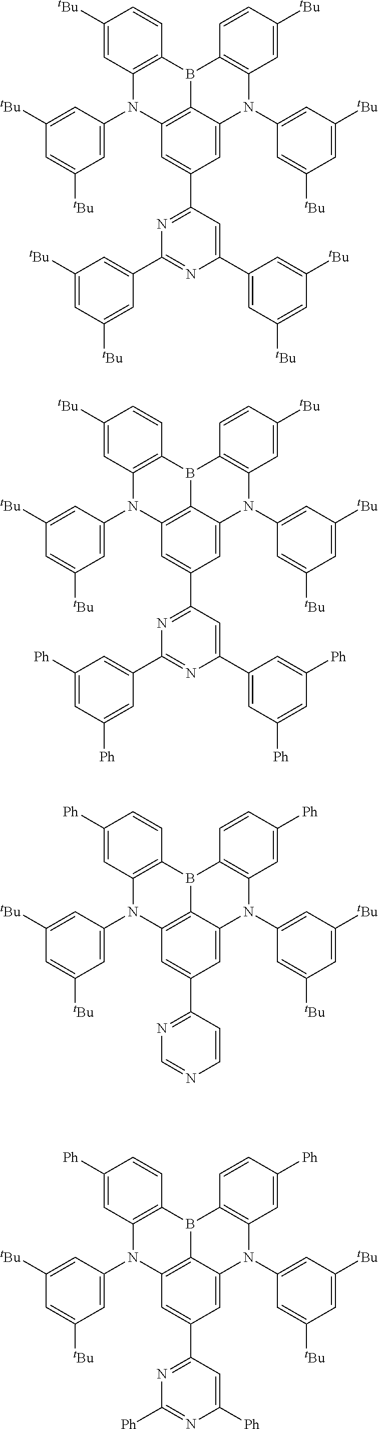

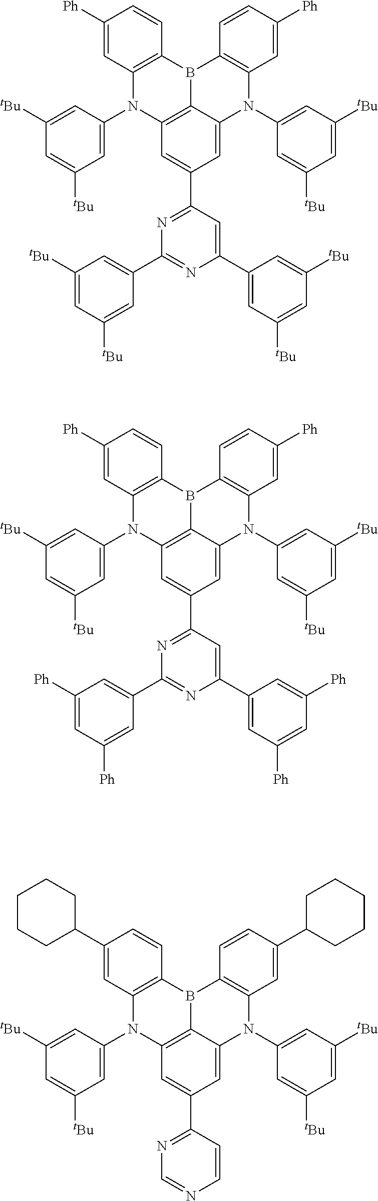 [SMILES: CC(C)(C)c1cc(-c2cc(-c3cc4c5c(c3)N(c3cc(C(C)(C)C)cc(C(C)(C)C)c3)c3cc(-c6ccccc6)ccc3B5c3ccc(-c5ccccc5)cc3N4c3cc(C(C)(C)C)cc(C(C)(C)C)c3)nc(-c3cc(C(C)(C)C)cc(C(C)(C)C)c3)n2)cc(C(C)(C)C)c1 ; CC(C)(C)c1cc(N2c3cc(-c4ccccc4)ccc3B3c4ccc(-c5ccccc5)cc4N(c4cc(C(C)(C)C)cc(C(C)(C)C)c4)c4cc(-c5cc(-c6cc(-c7ccccc7)cc(-c7ccccc7)c6)nc(-c6cc(-c7ccccc7)cc(-c7ccccc7)c6)n5)cc2c43)cc(C(C)(C)C)c1 ; CC(C)(C)c1cc(N2c3cc(C4CCCCC4)ccc3B3c4ccc(C5CCCCC5)cc4N(c4cc(C(C)(C)C)cc(C(C)(C)C)c4)c4cc(-c5ccncn5)cc2c43)cc(C(C)(C)C)c1]

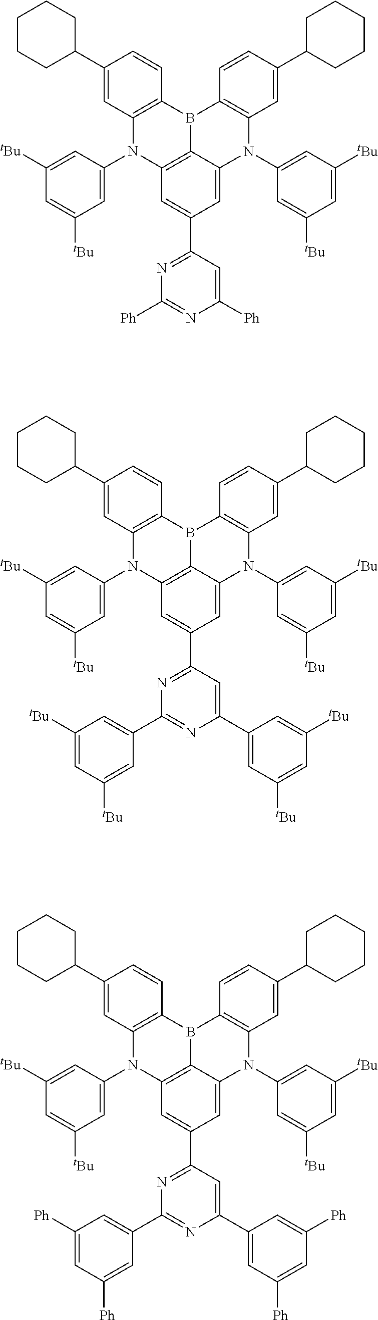

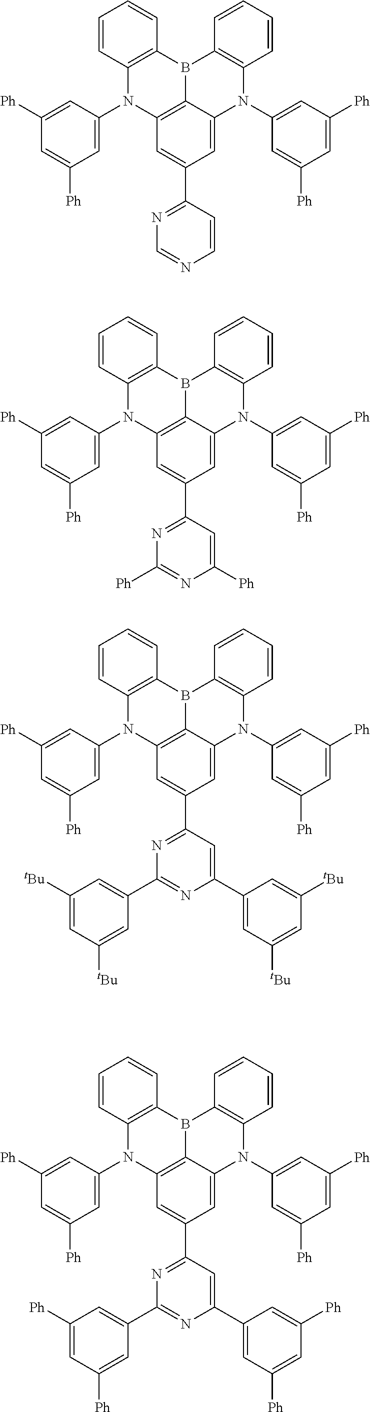

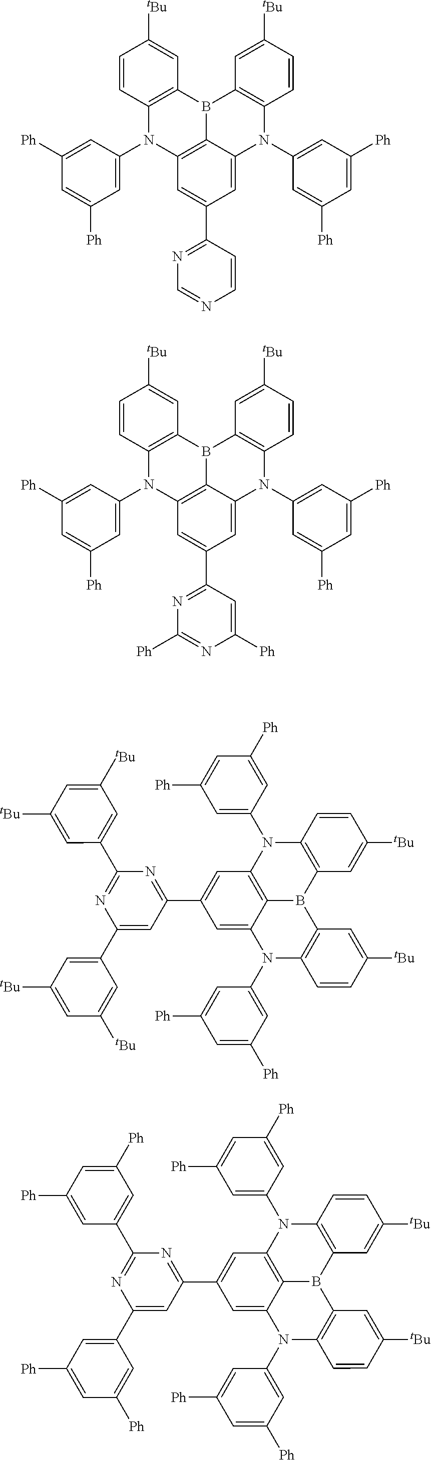

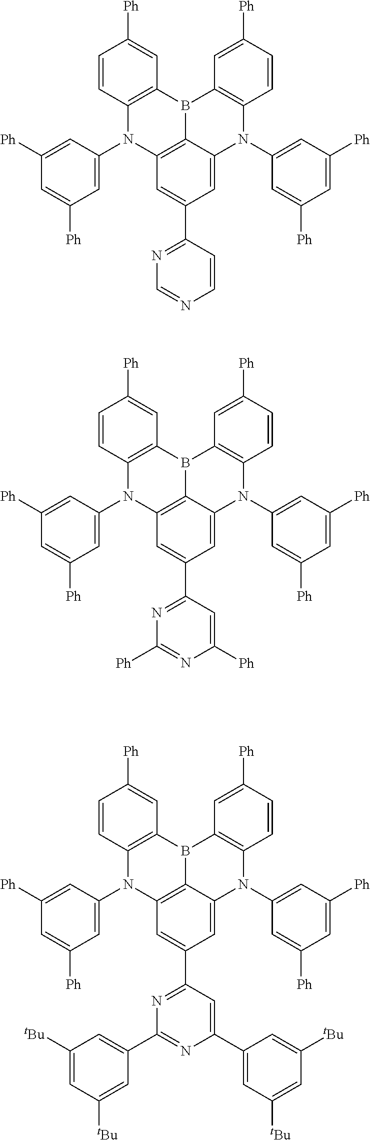 [SMILES: c1ccc(-c2cc(-c3ccccc3)cc(N3c4ccc(-c5ccccc5)cc4B4c5cc(-c6ccccc6)ccc5N(c5cc(-c6ccccc6)cc(-c6ccccc6)c5)c5cc(-c6ccncn6)cc3c54)c2)cc1 ; c1ccc(-c2cc(-c3ccccc3)cc(N3c4ccc(-c5ccccc5)cc4B4c5cc(-c6ccccc6)ccc5N(c5cc(-c6ccccc6)cc(-c6ccccc6)c5)c5cc(-c6cc(-c7ccccc7)nc(-c7ccccc7)n6)cc3c54)c2)cc1 ; CC(C)(C)c1cc(-c2cc(-c3cc4c5c(c3)N(c3cc(-c6ccccc6)cc(-c6ccccc6)c3)c3ccc(-c6ccccc6)cc3B5c3cc(-c5ccccc5)ccc3N4c3cc(-c4ccccc4)cc(-c4ccccc4)c3)nc(-c3cc(C(C)(C)C)cc(C(C)(C)C)c3)n2)cc(C(C)(C)C)c1]

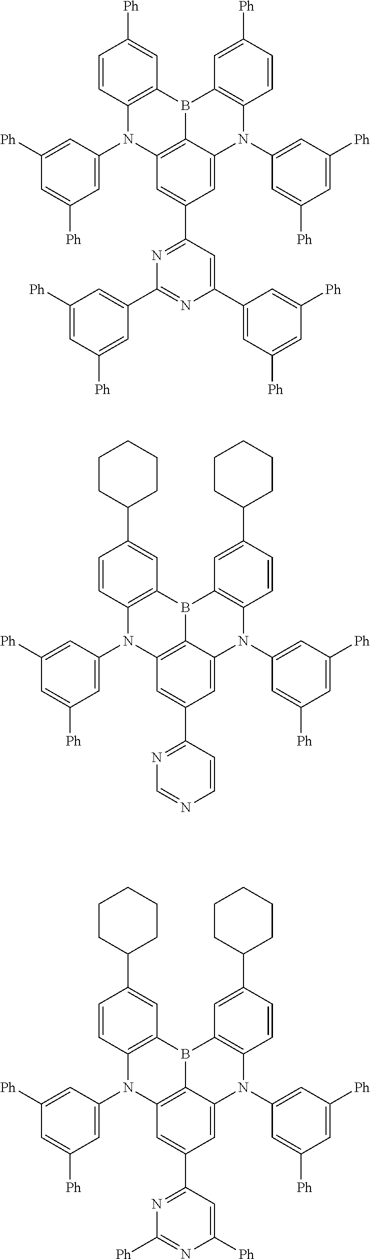 [SMILES: c1ccc(-c2cc(-c3ccccc3)cc(-c3cc(-c4cc5c6c(c4)N(c4cc(-c7ccccc7)cc(-c7ccccc7)c4)c4ccc(-c7ccccc7)cc4B6c4cc(-c6ccccc6)ccc4N5c4cc(-c5ccccc5)cc(-c5ccccc5)c4)nc(-c4cc(-c5ccccc5)cc(-c5ccccc5)c4)n3)c2)cc1 ; c1ccc(-c2cc(-c3ccccc3)cc(N3c4ccc(C5CCCCC5)cc4B4c5cc(C6CCCCC6)ccc5N(c5cc(-c6ccccc6)cc(-c6ccccc6)c5)c5cc(-c6ccncn6)cc3c54)c2)cc1 ; c1ccc(-c2cc(-c3ccccc3)cc(N3c4ccc(C5CCCCC5)cc4B4c5cc(C6CCCCC6)ccc5N(c5cc(-c6ccccc6)cc(-c6ccccc6)c5)c5cc(-c6cc(-c7ccccc7)nc(-c7ccccc7)n6)cc3c54)c2)cc1]

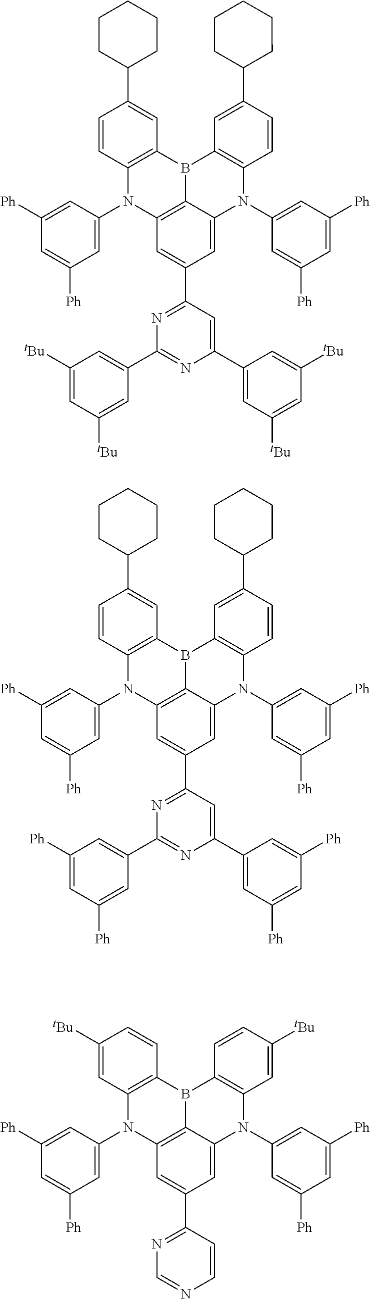

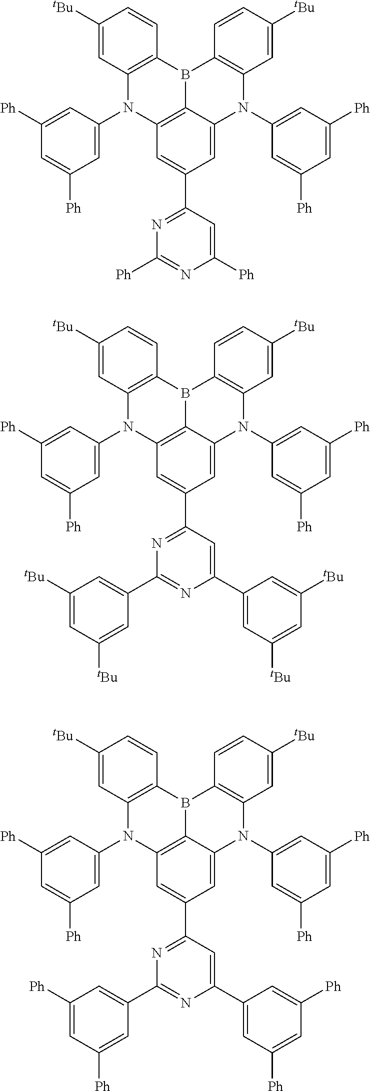

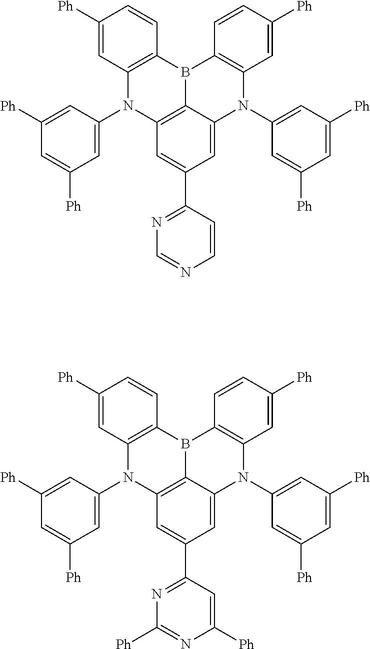 [SMILES: c1ccc(-c2cc(-c3ccccc3)cc(N3c4cc(-c5ccccc5)ccc4B4c5ccc(-c6ccccc6)cc5N(c5cc(-c6ccccc6)cc(-c6ccccc6)c5)c5cc(-c6ccncn6)cc3c54)c2)cc1 ; c1ccc(-c2cc(-c3ccccc3)cc(N3c4cc(-c5ccccc5)ccc4B4c5ccc(-c6ccccc6)cc5N(c5cc(-c6ccccc6)cc(-c6ccccc6)c5)c5cc(-c6cc(-c7ccccc7)nc(-c7ccccc7)n6)cc3c54)c2)cc1]

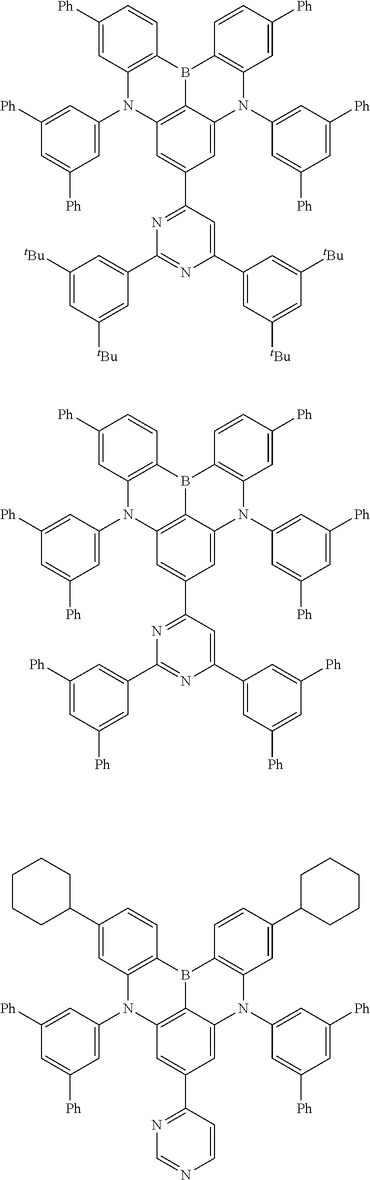

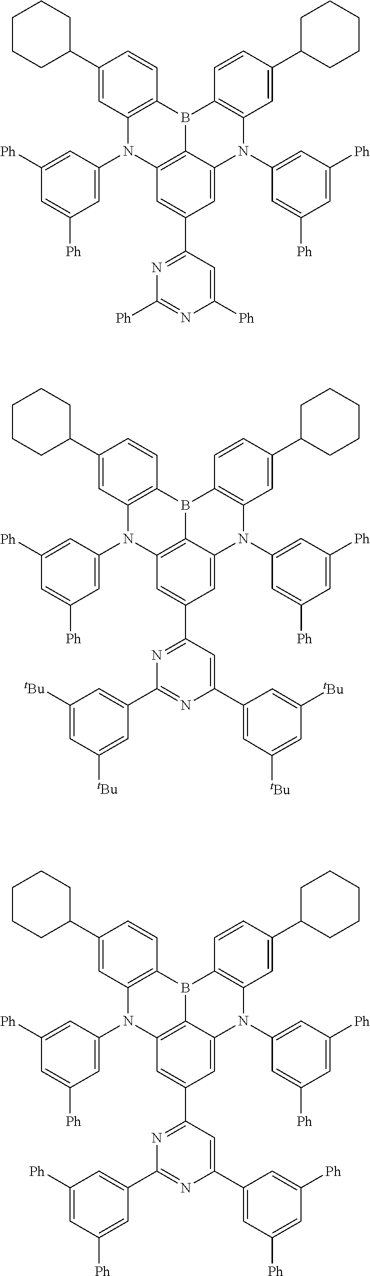

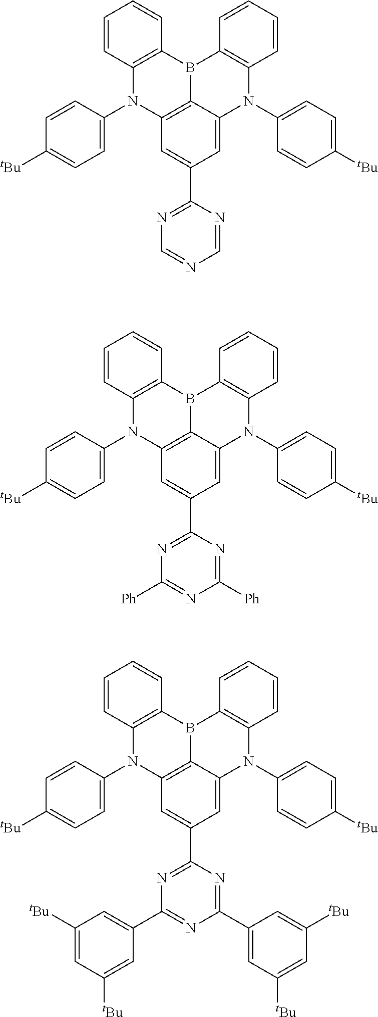 [SMILES: CC(C)(C)c1ccc(N2c3ccccc3B3c4ccccc4N(c4ccc(C(C)(C)C)cc4)c4cc(-c5ncncn5)cc2c43)cc1 ; CC(C)(C)c1ccc(N2c3ccccc3B3c4ccccc4N(c4ccc(C(C)(C)C)cc4)c4cc(-c5nc(-c6ccccc6)nc(-c6ccccc6)n5)cc2c43)cc1 ; CC(C)(C)c1ccc(N2c3ccccc3B3c4ccccc4N(c4ccc(C(C)(C)C)cc4)c4cc(-c5nc(-c6cc(C(C)(C)C)cc(C(C)(C)C)c6)nc(-c6cc(C(C)(C)C)cc(C(C)(C)C)c6)n5)cc2c43)cc1]

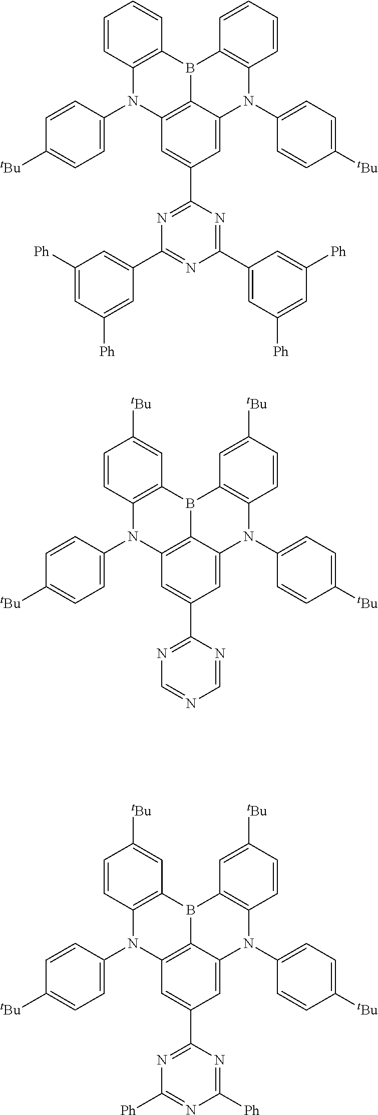 [SMILES: CC(C)(C)c1ccc(N2c3ccccc3B3c4ccccc4N(c4ccc(C(C)(C)C)cc4)c4cc(-c5nc(-c6cc(-c7ccccc7)cc(-c7ccccc7)c6)nc(-c6cc(-c7ccccc7)cc(-c7ccccc7)c6)n5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ncncn5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5nc(-c6ccccc6)nc(-c6ccccc6)n5)cc2c43)cc1]

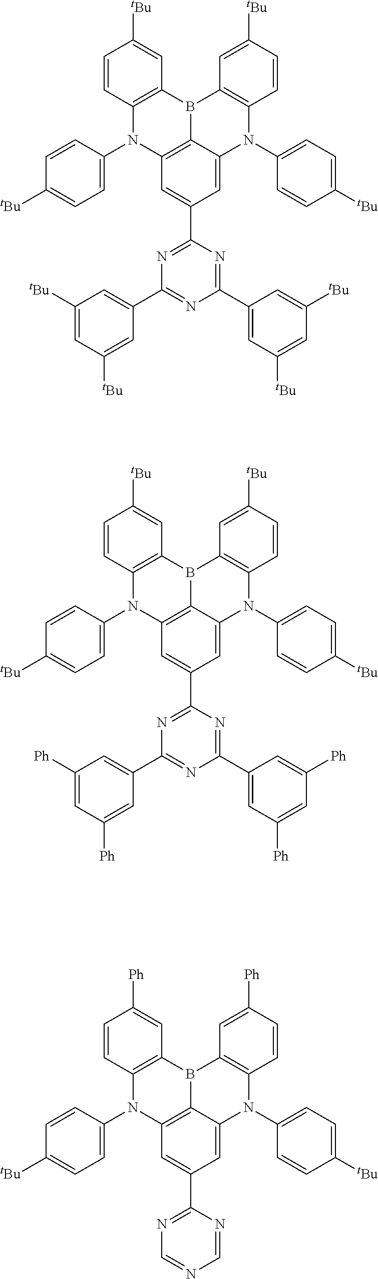 [SMILES: CC(C)(C)c1ccc(N2c3ccc(C(C)(C)C)cc3B3c4cc(C(C)(C)C)ccc4N(c4ccc(C(C)(C)C)cc4)c4cc(-c5nc(-c6cc(C(C)(C)C)cc(C(C)(C)C)c6)nc(-c6cc(C(C)(C)C)cc(C(C)(C)C)c6)n5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5nc(-c6cc(-c7ccccc7)cc(-c7ccccc7)c6)nc(-c6cc(-c7ccccc7)cc(-c7ccccc7)c6)n5)cc2c43)cc1 ; CC(C)(C)c1ccc(N2c3ccc(-c4ccccc4)cc3B3c4cc(-c5ccccc5)ccc4N(c4ccc(C(C)(C)C)cc4)c4cc(-c5ncncn5)cc2c43)cc1]

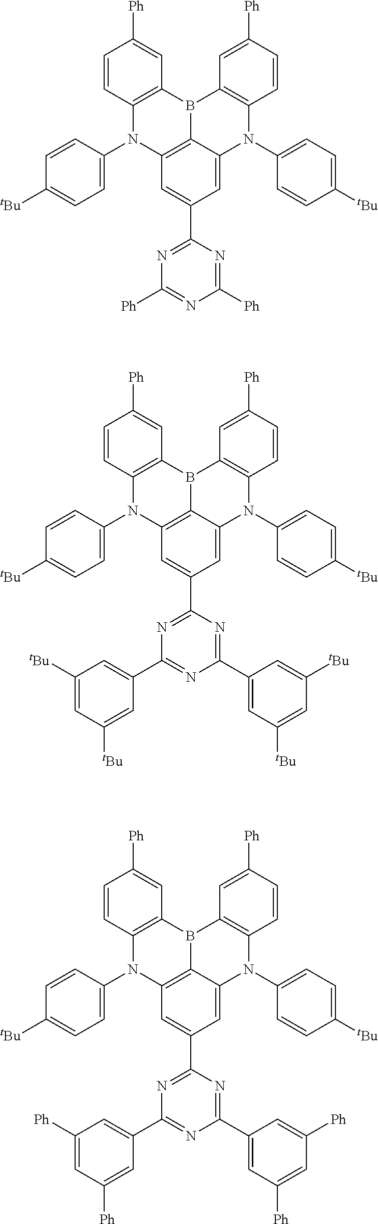 [SMILES: CC(C)(C)c1ccc(N2c3ccc(-c4ccccc4)cc3B3c4cc(-c5ccccc5)ccc4N(c4ccc(C(C)(C)C)cc4)c4cc(-c5nc(-c6ccccc6)nc(-c6ccccc6)n5)cc2c43)cc1 ; CC(C)(C)c1ccc(N2c3ccc(-c4ccccc4)cc3B3c4cc(-c5ccccc5)ccc4N(c4ccc(C(C)(C)C)cc4)c4cc(-c5nc(-c6cc(C(C)(C)C)cc(C(C)(C)C)c6)nc(-c6cc(C(C)(C)C)cc(C(C)(C)C)c6)n5)cc2c43)cc1 ; CC(C)(C)c1ccc(N2c3ccc(-c4ccccc4)cc3B3c4cc(-c5ccccc5)ccc4N(c4ccc(C(C)(C)C)cc4)c4cc(-c5nc(-c6cc(-c7ccccc7)cc(-c7ccccc7)c6)nc(-c6cc(-c7ccccc7)cc(-c7ccccc7)c6)n5)cc2c43)cc1]

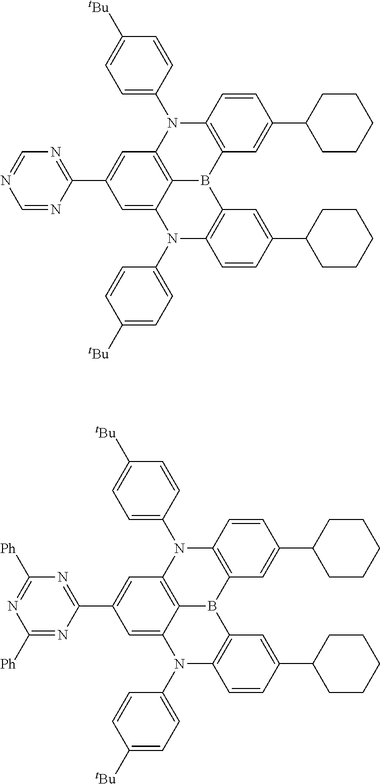 [SMILES: CC(C)(C)c1ccc(N2c3ccc(C4CCCCC4)cc3B3c4cc(C5CCCCC5)ccc4N(c4ccc(C(C)(C)C)cc4)c4cc(-c5ncncn5)cc2c43)cc1 ; CC(C)(C)c1ccc(N2c3ccc(C4CCCCC4)cc3B3c4cc(C5CCCCC5)ccc4N(c4ccc(C(C)(C)C)cc4)c4cc(-c5nc(-c6ccccc6)nc(-c6ccccc6)n5)cc2c43)cc1]

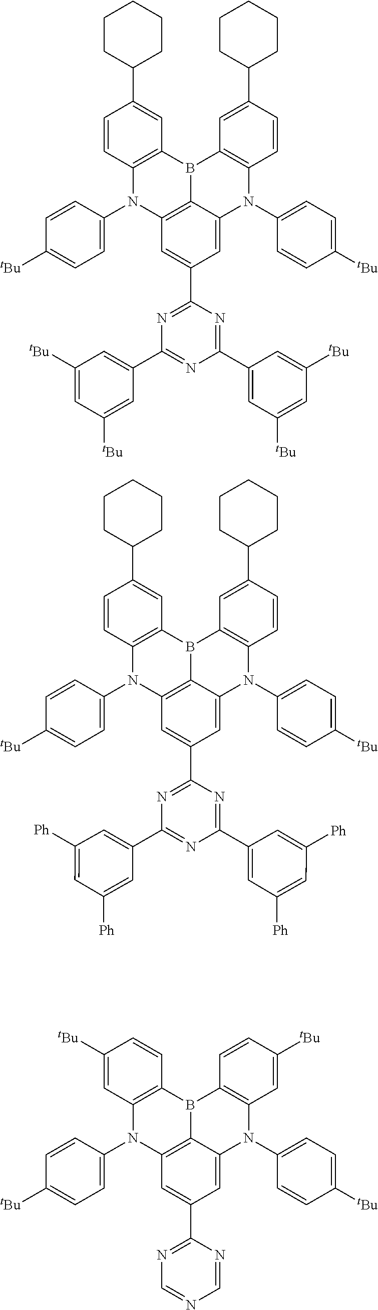

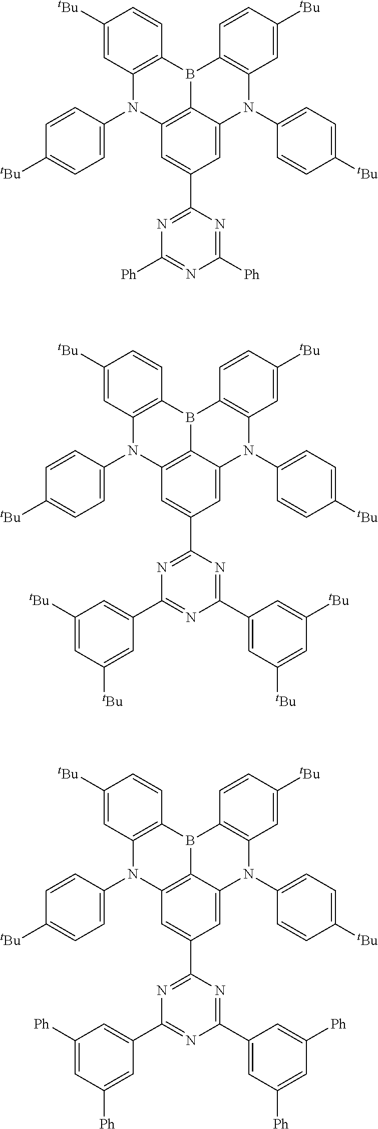 [SMILES: CC(C)(C)c1ccc(N2c3cc(C(C)(C)C)ccc3B3c4ccc(C(C)(C)C)cc4N(c4ccc(C(C)(C)C)cc4)c4cc(-c5nc(-c6ccccc6)nc(-c6ccccc6)n5)cc2c43)cc1 ; CC(C)(C)c1ccc(N2c3cc(C(C)(C)C)ccc3B3c4ccc(C(C)(C)C)cc4N(c4ccc(C(C)(C)C)cc4)c4cc(-c5nc(-c6cc(C(C)(C)C)cc(C(C)(C)C)c6)nc(-c6cc(C(C)(C)C)cc(C(C)(C)C)c6)n5)cc2c43)cc1 ; CC(C)(C)c1ccc(N2c3cc(C(C)(C)C)ccc3B3c4ccc(C(C)(C)C)cc4N(c4ccc(C(C)(C)C)cc4)c4cc(-c5nc(-c6cc(-c7ccccc7)cc(-c7ccccc7)c6)nc(-c6cc(-c7ccccc7)cc(-c7ccccc7)c6)n5)cc2c43)cc1]

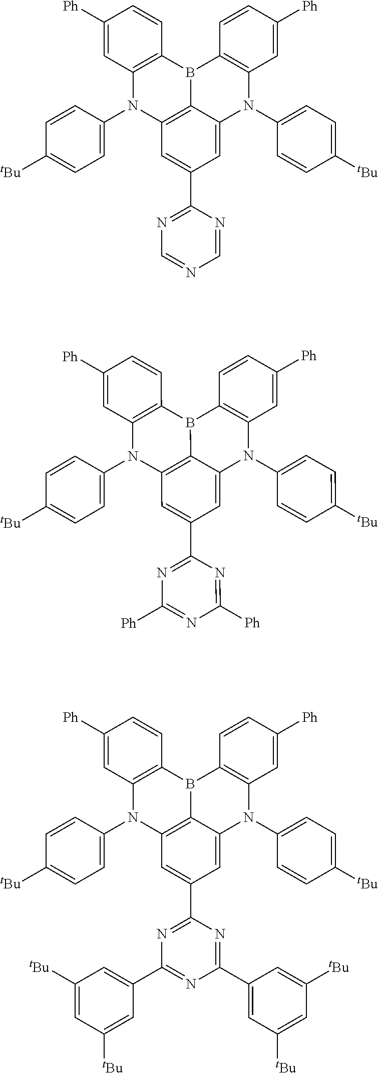 [SMILES: CC(C)(C)c1ccc(N2c3cc(-c4ccccc4)ccc3B3c4ccc(-c5ccccc5)cc4N(c4ccc(C(C)(C)C)cc4)c4cc(-c5ncncn5)cc2c43)cc1 ; CC(C)(C)c1ccc(N2c3cc(-c4ccccc4)ccc3B3c4ccc(-c5ccccc5)cc4N(c4ccc(C(C)(C)C)cc4)c4cc(-c5nc(-c6ccccc6)nc(-c6ccccc6)n5)cc2c43)cc1 ; CC(C)(C)c1ccc(N2c3cc(-c4ccccc4)ccc3B3c4ccc(-c5ccccc5)cc4N(c4ccc(C(C)(C)C)cc4)c4cc(-c5nc(-c6cc(C(C)(C)C)cc(C(C)(C)C)c6)nc(-c6cc(C(C)(C)C)cc(C(C)(C)C)c6)n5)cc2c43)cc1]

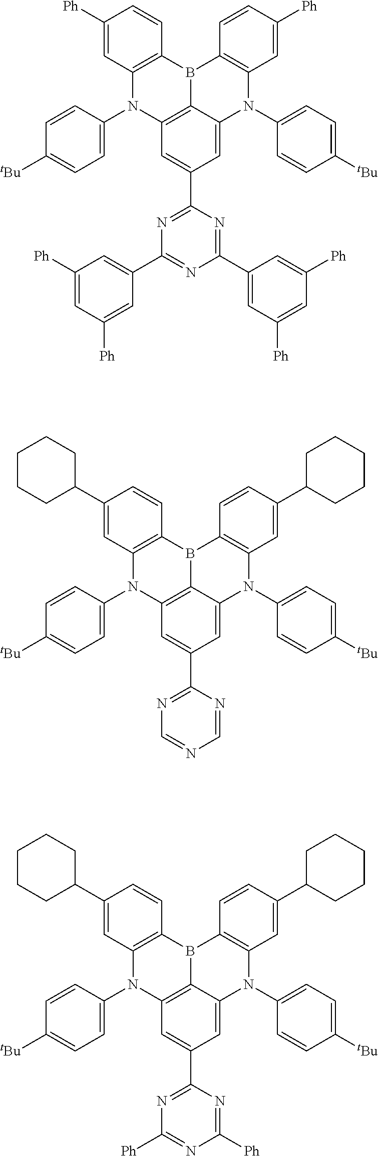 [SMILES: CC(C)(C)c1ccc(N2c3cc(-c4ccccc4)ccc3B3c4ccc(-c5ccccc5)cc4N(c4ccc(C(C)(C)C)cc4)c4cc(-c5nc(-c6cc(-c7ccccc7)cc(-c7ccccc7)c6)nc(-c6cc(-c7ccccc7)cc(-c7ccccc7)c6)n5)cc2c43)cc1 ; CC(C)(C)c1ccc(N2c3cc(C4CCCCC4)ccc3B3c4ccc(C5CCCCC5)cc4N(c4ccc(C(C)(C)C)cc4)c4cc(-c5ncncn5)cc2c43)cc1 ; CC(C)(C)c1ccc(N2c3cc(C4CCCCC4)ccc3B3c4ccc(C5CCCCC5)cc4N(c4ccc(C(C)(C)C)cc4)c4cc(-c5nc(-c6ccccc6)nc(-c6ccccc6)n5)cc2c43)cc1]

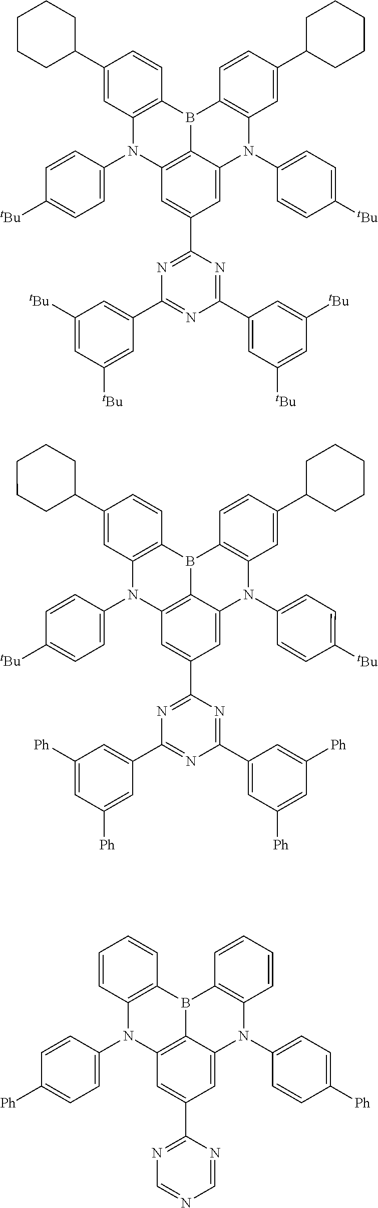 [SMILES: CC(C)(C)c1ccc(N2c3cc(C4CCCCC4)ccc3B3c4ccc(C5CCCCC5)cc4N(c4ccc(C(C)(C)C)cc4)c4cc(-c5nc(-c6cc(C(C)(C)C)cc(C(C)(C)C)c6)nc(-c6cc(C(C)(C)C)cc(C(C)(C)C)c6)n5)cc2c43)cc1 ; CC(C)(C)c1ccc(N2c3cc(C4CCCCC4)ccc3B3c4ccc(C5CCCCC5)cc4N(c4ccc(C(C)(C)C)cc4)c4cc(-c5nc(-c6cc(-c7ccccc7)cc(-c7ccccc7)c6)nc(-c6cc(-c7ccccc7)cc(-c7ccccc7)c6)n5)cc2c43)cc1 ; c1ccc(-c2ccc(N3c4ccccc4B4c5ccccc5N(c5ccc(-c6ccccc6)cc5)c5cc(-c6ncncn6)cc3c54)cc2)cc1]

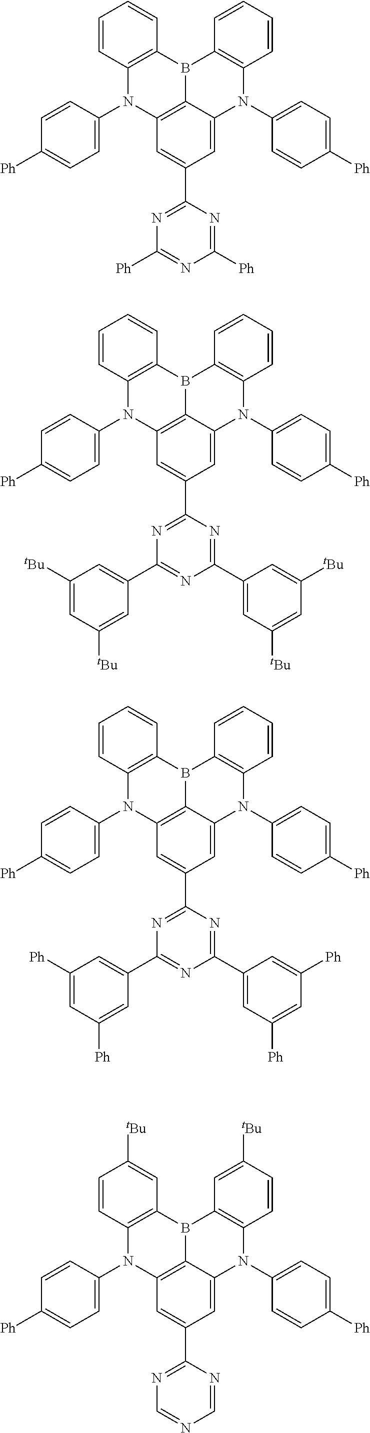

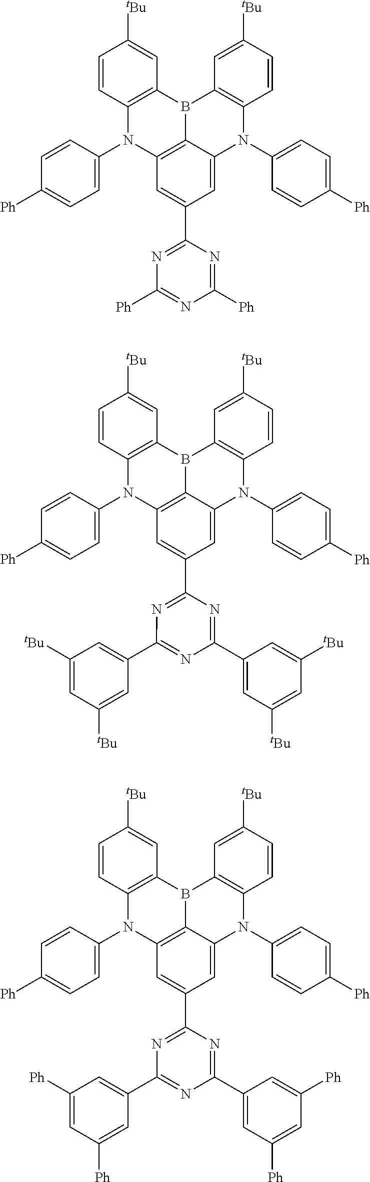 [SMILES: CC(C)(C)c1ccc2c(c1)B1c3cc(C(C)(C)C)ccc3N(c3ccc(-c4ccccc4)cc3)c3cc(-c4nc(-c5ccccc5)nc(-c5ccccc5)n4)cc(c31)N2c1ccc(-c2ccccc2)cc1 ; CC(C)(C)c1cc(-c2nc(-c3cc(C(C)(C)C)cc(C(C)(C)C)c3)nc(-c3cc4c5c(c3)N(c3ccc(-c6ccccc6)cc3)c3ccc(C(C)(C)C)cc3B5c3cc(C(C)(C)C)ccc3N4c3ccc(-c4ccccc4)cc3)n2)cc(C(C)(C)C)c1 ; CC(C)(C)c1ccc2c(c1)B1c3cc(C(C)(C)C)ccc3N(c3ccc(-c4ccccc4)cc3)c3cc(-c4nc(-c5cc(-c6ccccc6)cc(-c6ccccc6)c5)nc(-c5cc(-c6ccccc6)cc(-c6ccccc6)c5)n4)cc(c31)N2c1ccc(-c2ccccc2)cc1]

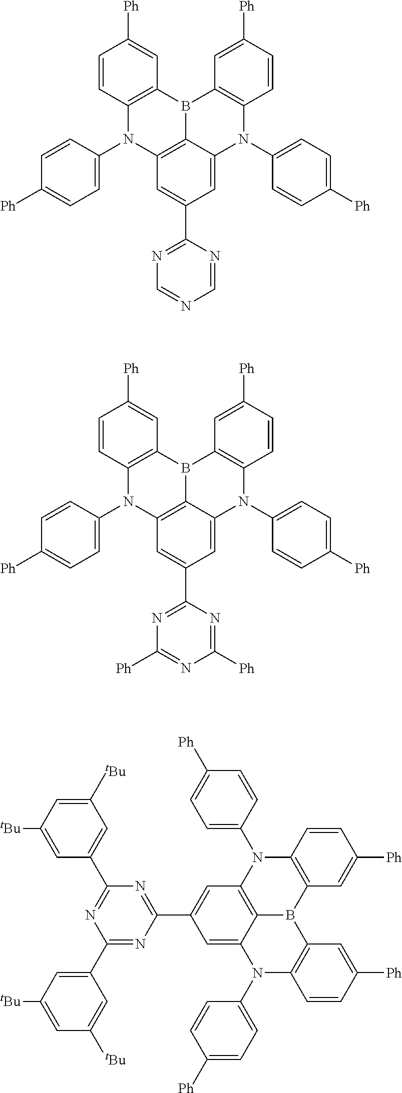 [SMILES: c1ccc(-c2ccc(N3c4ccc(-c5ccccc5)cc4B4c5cc(-c6ccccc6)ccc5N(c5ccc(-c6ccccc6)cc5)c5cc(-c6ncncn6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)nc(-c7ccccc7)n6)cc3c54)cc2)cc1 ; CC(C)(C)c1cc(-c2nc(-c3cc(C(C)(C)C)cc(C(C)(C)C)c3)nc(-c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)n2)cc(C(C)(C)C)c1]

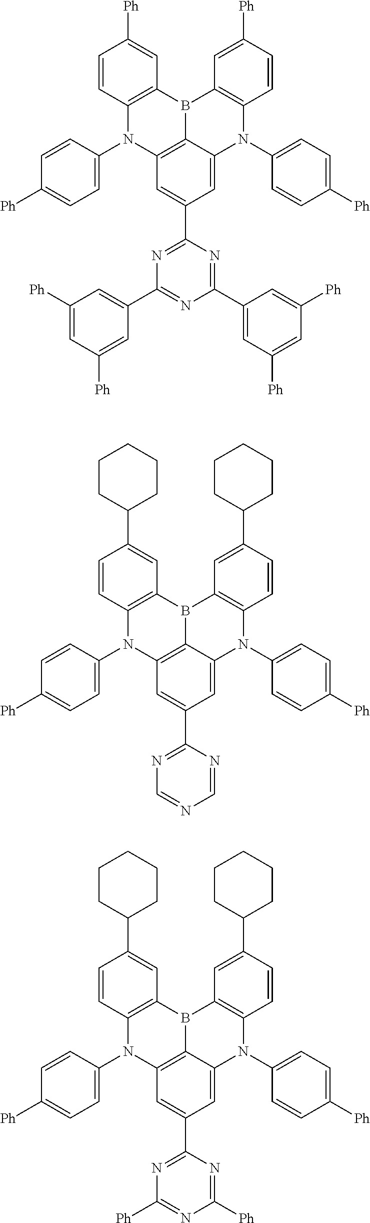 [SMILES: c1ccc(-c2ccc(N3c4ccc(-c5ccccc5)cc4B4c5cc(-c6ccccc6)ccc5N(c5ccc(-c6ccccc6)cc5)c5cc(-c6nc(-c7cc(-c8ccccc8)cc(-c8ccccc8)c7)nc(-c7cc(-c8ccccc8)cc(-c8ccccc8)c7)n6)cc3c54)cc2)cc1 ; c1ccc(-c2ccc(N3c4ccc(C5CCCCC5)cc4B4c5cc(C6CCCCC6)ccc5N(c5ccc(-c6ccccc6)cc5)c5cc(-c6ncncn6)cc3c54)cc2)cc1 ; c1ccc(-c2ccc(N3c4ccc(C5CCCCC5)cc4B4c5cc(C6CCCCC6)ccc5N(c5ccc(-c6ccccc6)cc5)c5cc(-c6nc(-c7ccccc7)nc(-c7ccccc7)n6)cc3c54)cc2)cc1]

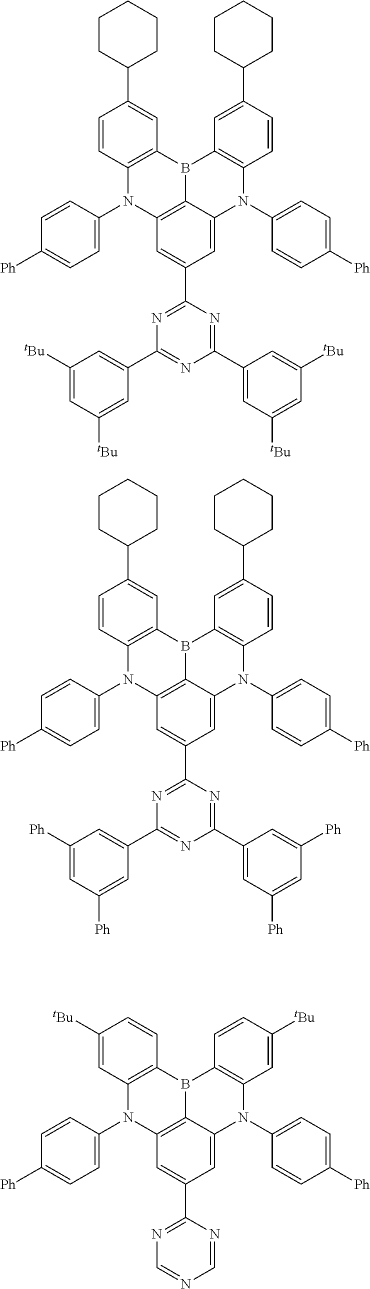

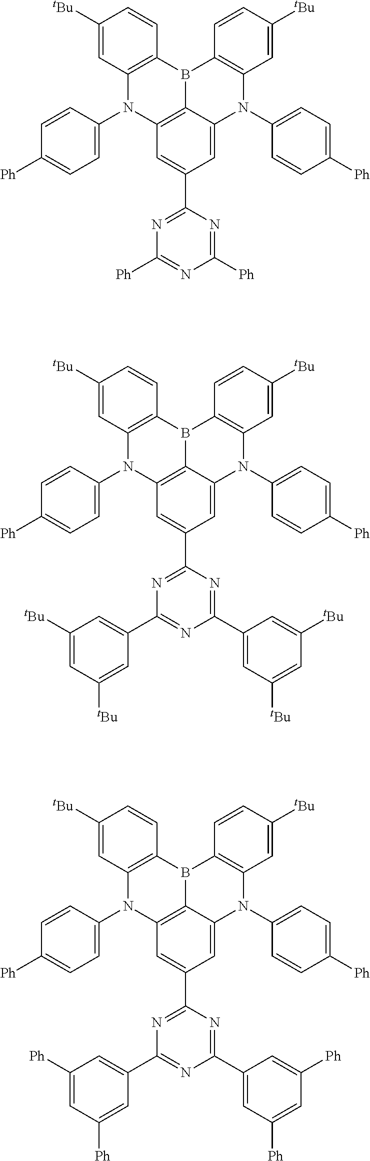 [SMILES: CC(C)(C)c1ccc2c(c1)N(c1ccc(-c3ccccc3)cc1)c1cc(-c3nc(-c4ccccc4)nc(-c4ccccc4)n3)cc3c1B2c1ccc(C(C)(C)C)cc1N3c1ccc(-c2ccccc2)cc1 ; CC(C)(C)c1cc(-c2nc(-c3cc(C(C)(C)C)cc(C(C)(C)C)c3)nc(-c3cc4c5c(c3)N(c3ccc(-c6ccccc6)cc3)c3cc(C(C)(C)C)ccc3B5c3ccc(C(C)(C)C)cc3N4c3ccc(-c4ccccc4)cc3)n2)cc(C(C)(C)C)c1 ; CC(C)(C)c1ccc2c(c1)N(c1ccc(-c3ccccc3)cc1)c1cc(-c3nc(-c4cc(-c5ccccc5)cc(-c5ccccc5)c4)nc(-c4cc(-c5ccccc5)cc(-c5ccccc5)c4)n3)cc3c1B2c1ccc(C(C)(C)C)cc1N3c1ccc(-c2ccccc2)cc1]

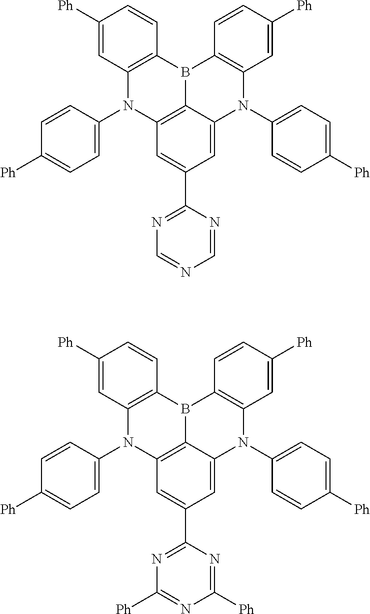 [SMILES: c1ccc(-c2ccc(N3c4cc(-c5ccccc5)ccc4B4c5ccc(-c6ccccc6)cc5N(c5ccc(-c6ccccc6)cc5)c5cc(-c6ncncn6)cc3c54)cc2)cc1 ; c1ccc(-c2ccc(N3c4cc(-c5ccccc5)ccc4B4c5ccc(-c6ccccc6)cc5N(c5ccc(-c6ccccc6)cc5)c5cc(-c6nc(-c7ccccc7)nc(-c7ccccc7)n6)cc3c54)cc2)cc1]

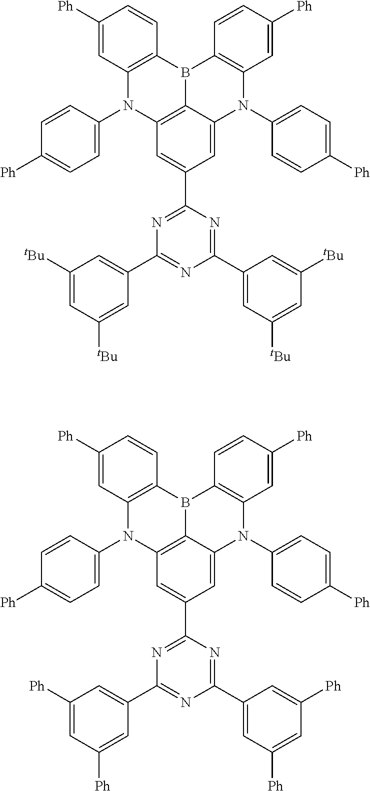 [SMILES: CC(C)(C)c1cc(-c2nc(-c3cc(C(C)(C)C)cc(C(C)(C)C)c3)nc(-c3cc4c5c(c3)N(c3ccc(-c6ccccc6)cc3)c3cc(-c6ccccc6)ccc3B5c3ccc(-c5ccccc5)cc3N4c3ccc(-c4ccccc4)cc3)n2)cc(C(C)(C)C)c1 ; c1ccc(-c2ccc(N3c4cc(-c5ccccc5)ccc4B4c5ccc(-c6ccccc6)cc5N(c5ccc(-c6ccccc6)cc5)c5cc(-c6nc(-c7cc(-c8ccccc8)cc(-c8ccccc8)c7)nc(-c7cc(-c8ccccc8)cc(-c8ccccc8)c7)n6)cc3c54)cc2)cc1]

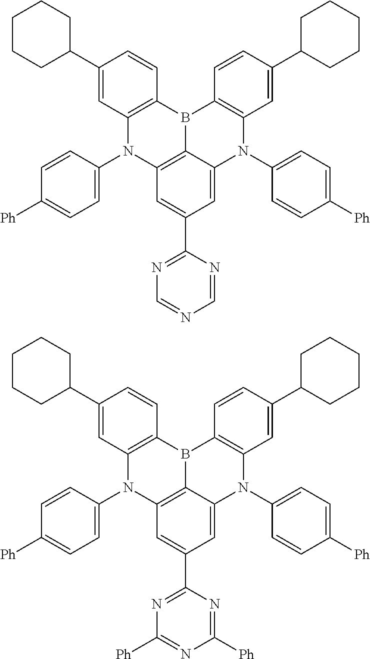 [SMILES: c1ccc(-c2ccc(N3c4cc(C5CCCCC5)ccc4B4c5ccc(C6CCCCC6)cc5N(c5ccc(-c6ccccc6)cc5)c5cc(-c6ncncn6)cc3c54)cc2)cc1 ; c1ccc(-c2ccc(N3c4cc(C5CCCCC5)ccc4B4c5ccc(C6CCCCC6)cc5N(c5ccc(-c6ccccc6)cc5)c5cc(-c6nc(-c7ccccc7)nc(-c7ccccc7)n6)cc3c54)cc2)cc1]

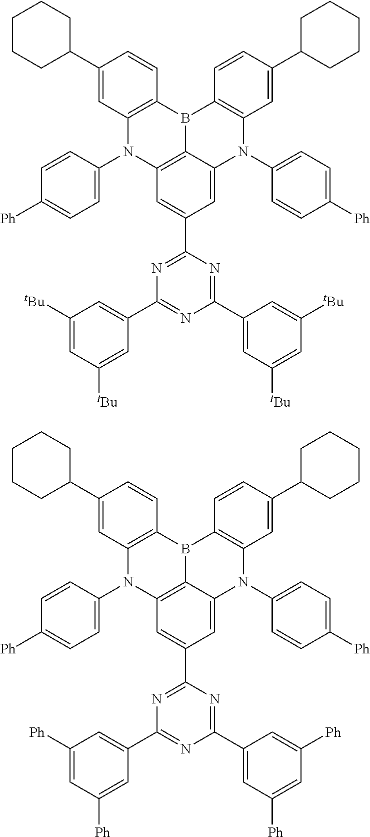 [SMILES: CC(C)(C)c1cc(-c2nc(-c3cc(C(C)(C)C)cc(C(C)(C)C)c3)nc(-c3cc4c5c(c3)N(c3ccc(-c6ccccc6)cc3)c3cc(C6CCCCC6)ccc3B5c3ccc(C5CCCCC5)cc3N4c3ccc(-c4ccccc4)cc3)n2)cc(C(C)(C)C)c1 ; c1ccc(-c2ccc(N3c4cc(C5CCCCC5)ccc4B4c5ccc(C6CCCCC6)cc5N(c5ccc(-c6ccccc6)cc5)c5cc(-c6nc(-c7cc(-c8ccccc8)cc(-c8ccccc8)c7)nc(-c7cc(-c8ccccc8)cc(-c8ccccc8)c7)n6)cc3c54)cc2)cc1]

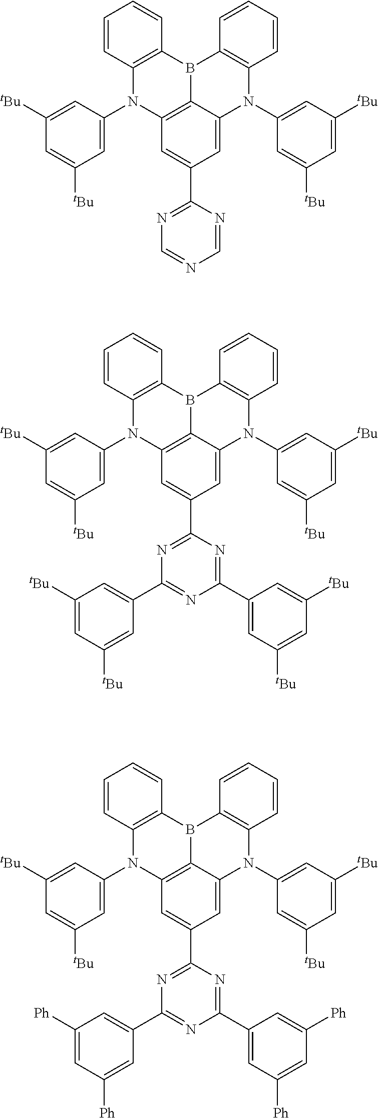 [SMILES: CC(C)(C)c1cc(N2c3ccccc3B3c4ccccc4N(c4cc(C(C)(C)C)cc(C(C)(C)C)c4)c4cc(-c5ncncn5)cc2c43)cc(C(C)(C)C)c1 ; CC(C)(C)c1cc(-c2nc(-c3cc(C(C)(C)C)cc(C(C)(C)C)c3)nc(-c3cc4c5c(c3)N(c3cc(C(C)(C)C)cc(C(C)(C)C)c3)c3ccccc3B5c3ccccc3N4c3cc(C(C)(C)C)cc(C(C)(C)C)c3)n2)cc(C(C)(C)C)c1 ; CC(C)(C)c1cc(N2c3ccccc3B3c4ccccc4N(c4cc(C(C)(C)C)cc(C(C)(C)C)c4)c4cc(-c5nc(-c6cc(-c7ccccc7)cc(-c7ccccc7)c6)nc(-c6cc(-c7ccccc7)cc(-c7ccccc7)c6)n5)cc2c43)cc(C(C)(C)C)c1]

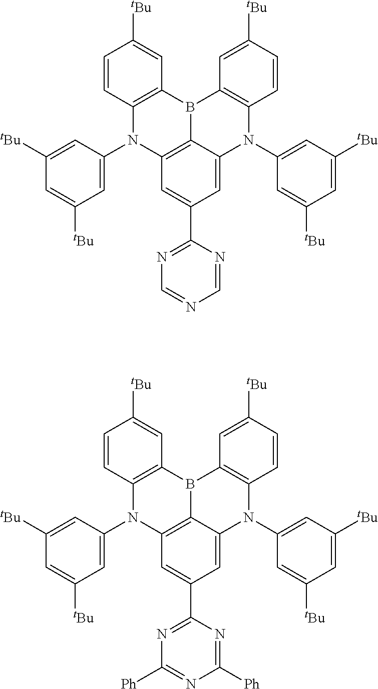 [SMILES: CC(C)(C)c1cc(N2c3ccc(C(C)(C)C)cc3B3c4cc(C(C)(C)C)ccc4N(c4cc(C(C)(C)C)cc(C(C)(C)C)c4)c4cc(-c5ncncn5)cc2c43)cc(C(C)(C)C)c1 ; CC(C)(C)c1cc(N2c3ccc(C(C)(C)C)cc3B3c4cc(C(C)(C)C)ccc4N(c4cc(C(C)(C)C)cc(C(C)(C)C)c4)c4cc(-c5nc(-c6ccccc6)nc(-c6ccccc6)n5)cc2c43)cc(C(C)(C)C)c1]

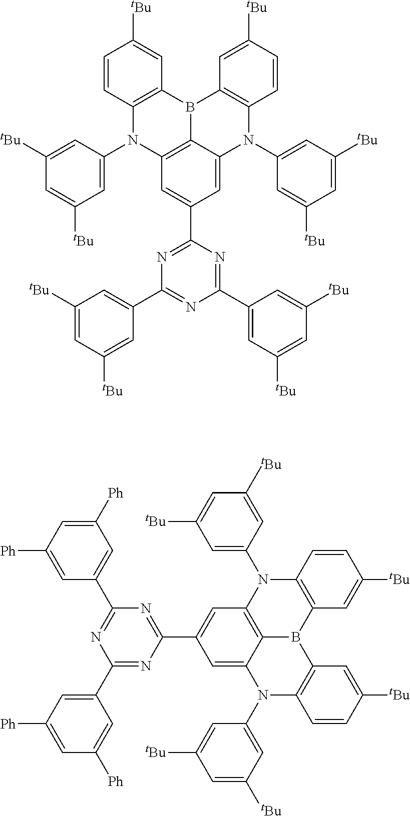 [SMILES: CC(C)(C)c1cc(-c2nc(-c3cc(C(C)(C)C)cc(C(C)(C)C)c3)nc(-c3cc4c5c(c3)N(c3cc(C(C)(C)C)cc(C(C)(C)C)c3)c3ccc(C(C)(C)C)cc3B5c3cc(C(C)(C)C)ccc3N4c3cc(C(C)(C)C)cc(C(C)(C)C)c3)n2)cc(C(C)(C)C)c1 ; CC(C)(C)c1cc(N2c3ccc(C(C)(C)C)cc3B3c4cc(C(C)(C)C)ccc4N(c4cc(C(C)(C)C)cc(C(C)(C)C)c4)c4cc(-c5nc(-c6cc(-c7ccccc7)cc(-c7ccccc7)c6)nc(-c6cc(-c7ccccc7)cc(-c7ccccc7)c6)n5)cc2c43)cc(C(C)(C)C)c1]

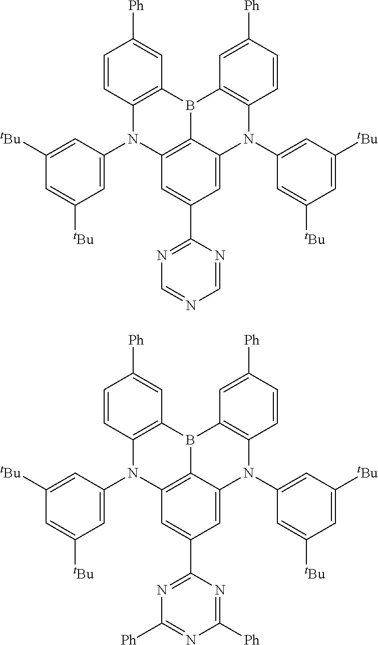 [SMILES: CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5ncncn5)cc2c43)cc(C(C)(C)C)c1 ; CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5nc(-c6ccccc6)nc(-c6ccccc6)n5)cc2c43)cc(C(C)(C)C)c1]

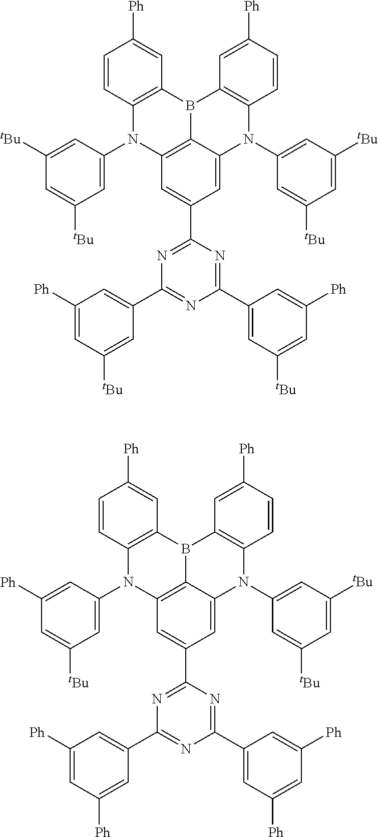 [SMILES: CC(C)(C)c1cc(-c2ccccc2)cc(-c2nc(-c3cc(-c4ccccc4)cc(C(C)(C)C)c3)nc(-c3cc4c5c(c3)N(c3cc(C(C)(C)C)cc(C(C)(C)C)c3)c3ccc(-c6ccccc6)cc3B5c3cc(-c5ccccc5)ccc3N4c3cc(C(C)(C)C)cc(C(C)(C)C)c3)n2)c1 ; CC(C)(C)c1cc(-c2ccccc2)cc(N2c3ccc(-c4ccccc4)cc3B3c4cc(-c5ccccc5)ccc4N(c4cc(C(C)(C)C)cc(C(C)(C)C)c4)c4cc(-c5nc(-c6cc(-c7ccccc7)cc(-c7ccccc7)c6)nc(-c6cc(-c7ccccc7)cc(-c7ccccc7)c6)n5)cc2c43)c1]

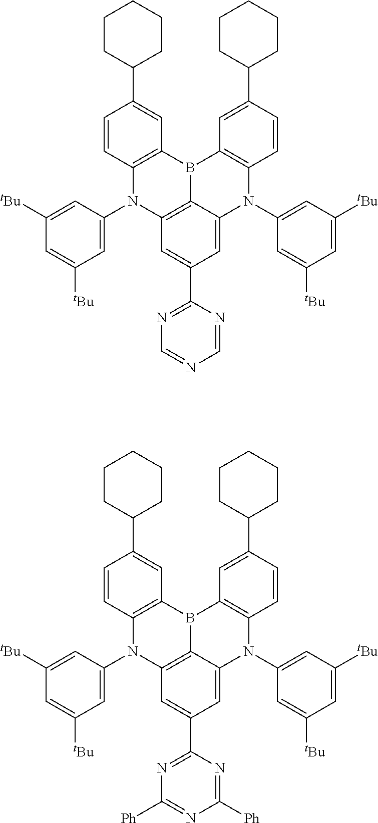 [SMILES: CC(C)(C)c1cc(N2c3ccc(C4CCCCC4)cc3B3c4cc(C5CCCCC5)ccc4N(c4cc(C(C)(C)C)cc(C(C)(C)C)c4)c4cc(-c5ncncn5)cc2c43)cc(C(C)(C)C)c1 ; CC(C)(C)c1cc(N2c3ccc(C4CCCCC4)cc3B3c4cc(C5CCCCC5)ccc4N(c4cc(C(C)(C)C)cc(C(C)(C)C)c4)c4cc(-c5nc(-c6ccccc6)nc(-c6ccccc6)n5)cc2c43)cc(C(C)(C)C)c1]

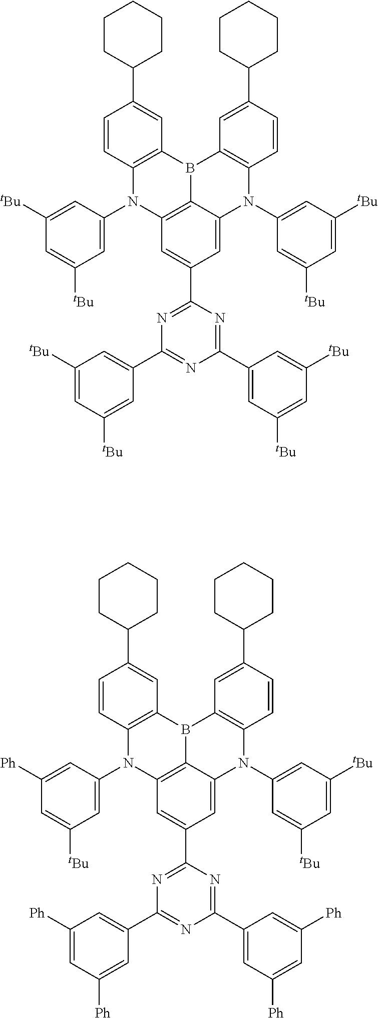 [SMILES: CC(C)(C)c1cc(-c2nc(-c3cc(C(C)(C)C)cc(C(C)(C)C)c3)nc(-c3cc4c5c(c3)N(c3cc(C(C)(C)C)cc(C(C)(C)C)c3)c3ccc(C6CCCCC6)cc3B5c3cc(C5CCCCC5)ccc3N4c3cc(C(C)(C)C)cc(C(C)(C)C)c3)n2)cc(C(C)(C)C)c1 ; CC(C)(C)c1cc(-c2ccccc2)cc(N2c3ccc(C4CCCCC4)cc3B3c4cc(C5CCCCC5)ccc4N(c4cc(C(C)(C)C)cc(C(C)(C)C)c4)c4cc(-c5nc(-c6cc(-c7ccccc7)cc(-c7ccccc7)c6)nc(-c6cc(-c7ccccc7)cc(-c7ccccc7)c6)n5)cc2c43)c1]

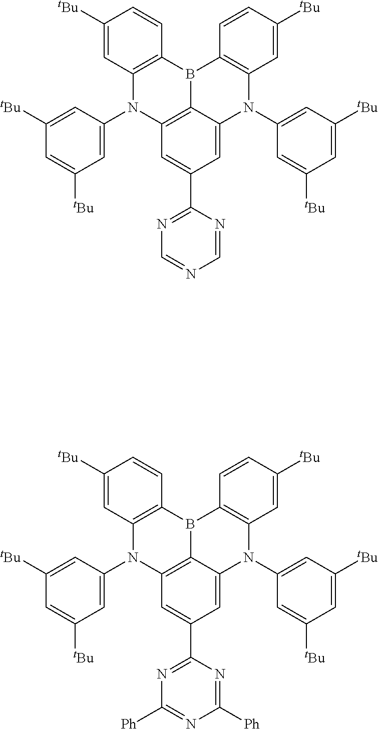 [SMILES: CC(C)(C)c1cc(N2c3cc(C(C)(C)C)ccc3B3c4ccc(C(C)(C)C)cc4N(c4cc(C(C)(C)C)cc(C(C)(C)C)c4)c4cc(-c5ncncn5)cc2c43)cc(C(C)(C)C)c1 ; CC(C)(C)c1cc(N2c3cc(C(C)(C)C)ccc3B3c4ccc(C(C)(C)C)cc4N(c4cc(C(C)(C)C)cc(C(C)(C)C)c4)c4cc(-c5nc(-c6ccccc6)nc(-c6ccccc6)n5)cc2c43)cc(C(C)(C)C)c1]

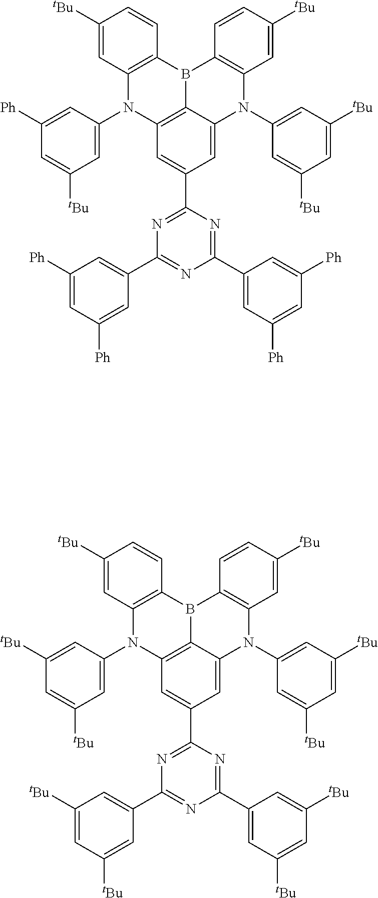 [SMILES: CC(C)(C)c1cc(-c2ccccc2)cc(N2c3cc(C(C)(C)C)ccc3B3c4ccc(C(C)(C)C)cc4N(c4cc(C(C)(C)C)cc(C(C)(C)C)c4)c4cc(-c5nc(-c6cc(-c7ccccc7)cc(-c7ccccc7)c6)nc(-c6cc(-c7ccccc7)cc(-c7ccccc7)c6)n5)cc2c43)c1 ; CC(C)(C)c1cc(-c2nc(-c3cc(C(C)(C)C)cc(C(C)(C)C)c3)nc(-c3cc4c5c(c3)N(c3cc(C(C)(C)C)cc(C(C)(C)C)c3)c3cc(C(C)(C)C)ccc3B5c3ccc(C(C)(C)C)cc3N4c3cc(C(C)(C)C)cc(C(C)(C)C)c3)n2)cc(C(C)(C)C)c1]

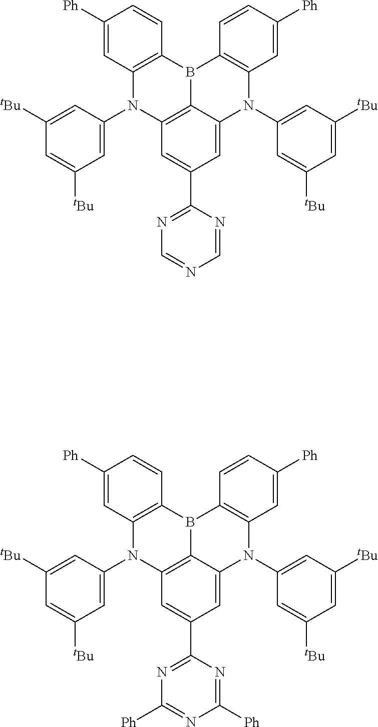 [SMILES: CC(C)(C)c1cc(N2c3cc(-c4ccccc4)ccc3B3c4ccc(-c5ccccc5)cc4N(c4cc(C(C)(C)C)cc(C(C)(C)C)c4)c4cc(-c5ncncn5)cc2c43)cc(C(C)(C)C)c1 ; CC(C)(C)c1cc(N2c3cc(-c4ccccc4)ccc3B3c4ccc(-c5ccccc5)cc4N(c4cc(C(C)(C)C)cc(C(C)(C)C)c4)c4cc(-c5nc(-c6ccccc6)nc(-c6ccccc6)n5)cc2c43)cc(C(C)(C)C)c1]

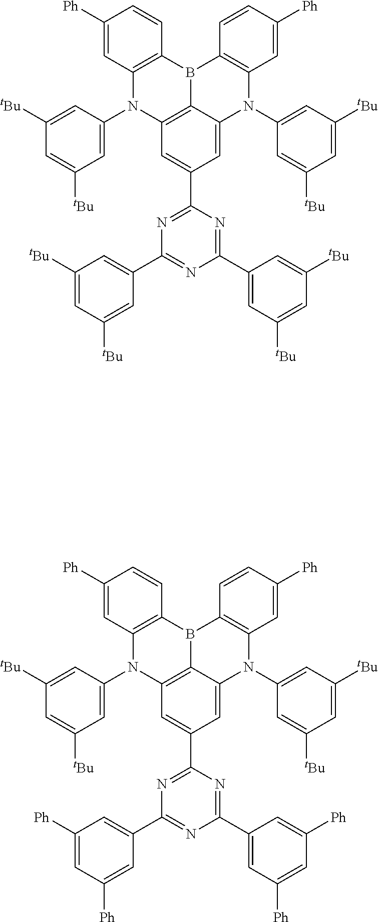 [SMILES: CC(C)(C)c1cc(-c2nc(-c3cc(C(C)(C)C)cc(C(C)(C)C)c3)nc(-c3cc4c5c(c3)N(c3cc(C(C)(C)C)cc(C(C)(C)C)c3)c3cc(-c6ccccc6)ccc3B5c3ccc(-c5ccccc5)cc3N4c3cc(C(C)(C)C)cc(C(C)(C)C)c3)n2)cc(C(C)(C)C)c1 ; CC(C)(C)c1cc(N2c3cc(-c4ccccc4)ccc3B3c4ccc(-c5ccccc5)cc4N(c4cc(C(C)(C)C)cc(C(C)(C)C)c4)c4cc(-c5nc(-c6cc(-c7ccccc7)cc(-c7ccccc7)c6)nc(-c6cc(-c7ccccc7)cc(-c7ccccc7)c6)n5)cc2c43)cc(C(C)(C)C)c1]

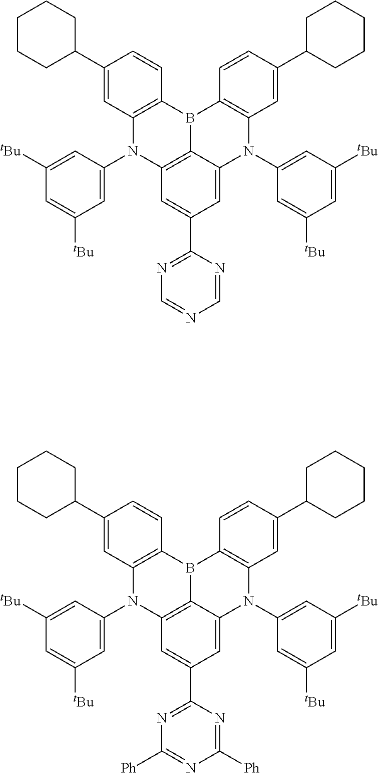 [SMILES: CC(C)(C)c1cc(N2c3cc(C4CCCCC4)ccc3B3c4ccc(C5CCCCC5)cc4N(c4cc(C(C)(C)C)cc(C(C)(C)C)c4)c4cc(-c5ncncn5)cc2c43)cc(C(C)(C)C)c1 ; CC(C)(C)c1cc(N2c3cc(C4CCCCC4)ccc3B3c4ccc(C5CCCCC5)cc4N(c4cc(C(C)(C)C)cc(C(C)(C)C)c4)c4cc(-c5nc(-c6ccccc6)nc(-c6ccccc6)n5)cc2c43)cc(C(C)(C)C)c1]

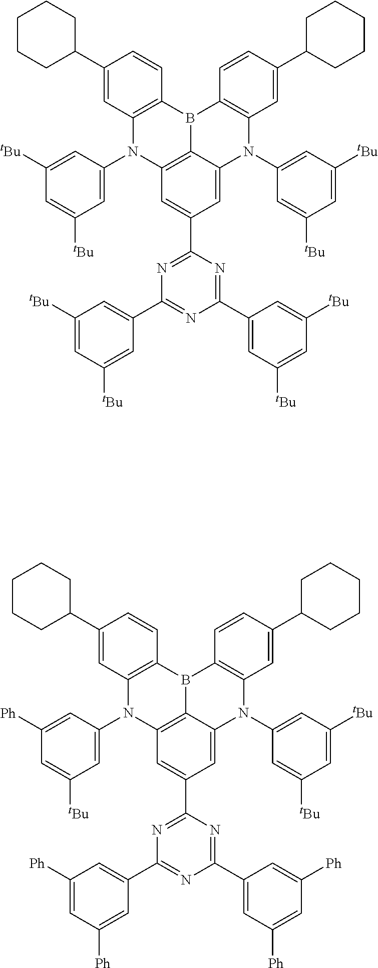 [SMILES: CC(C)(C)c1cc(-c2nc(-c3cc(C(C)(C)C)cc(C(C)(C)C)c3)nc(-c3cc4c5c(c3)N(c3cc(C(C)(C)C)cc(C(C)(C)C)c3)c3cc(C6CCCCC6)ccc3B5c3ccc(C5CCCCC5)cc3N4c3cc(C(C)(C)C)cc(C(C)(C)C)c3)n2)cc(C(C)(C)C)c1 ; CC(C)(C)c1cc(-c2ccccc2)cc(N2c3cc(C4CCCCC4)ccc3B3c4ccc(C5CCCCC5)cc4N(c4cc(C(C)(C)C)cc(C(C)(C)C)c4)c4cc(-c5nc(-c6cc(-c7ccccc7)cc(-c7ccccc7)c6)nc(-c6cc(-c7ccccc7)cc(-c7ccccc7)c6)n5)cc2c43)c1]

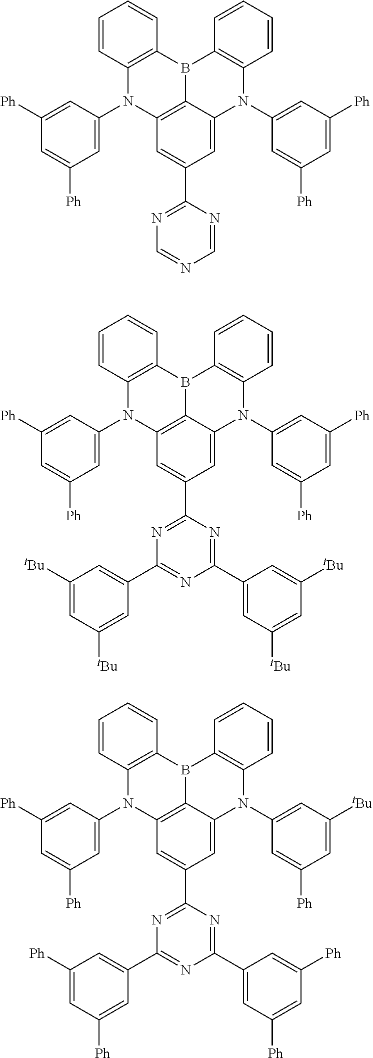 [SMILES: c1ccc(-c2cc(-c3ccccc3)cc(N3c4ccccc4B4c5ccccc5N(c5cc(-c6ccccc6)cc(-c6ccccc6)c5)c5cc(-c6ncncn6)cc3c54)c2)cc1 ; CC(C)(C)c1cc(-c2nc(-c3cc(C(C)(C)C)cc(C(C)(C)C)c3)nc(-c3cc4c5c(c3)N(c3cc(-c6ccccc6)cc(-c6ccccc6)c3)c3ccccc3B5c3ccccc3N4c3cc(-c4ccccc4)cc(-c4ccccc4)c3)n2)cc(C(C)(C)C)c1 ; CC(C)(C)c1cc(-c2ccccc2)cc(N2c3ccccc3B3c4ccccc4N(c4cc(-c5ccccc5)cc(-c5ccccc5)c4)c4cc(-c5nc(-c6cc(-c7ccccc7)cc(-c7ccccc7)c6)nc(-c6cc(-c7ccccc7)cc(-c7ccccc7)c6)n5)cc2c43)c1]

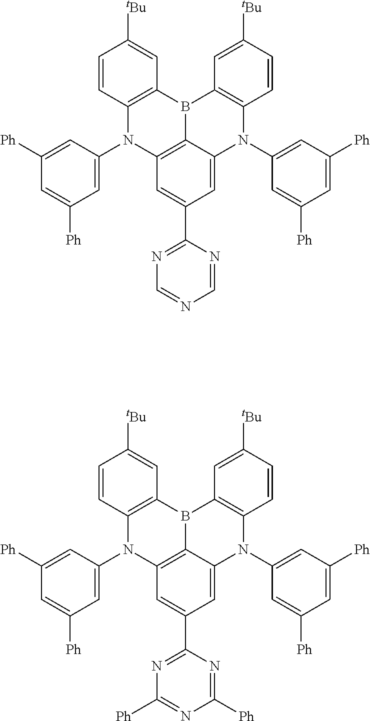 [SMILES: CC(C)(C)c1ccc2c(c1)B1c3cc(C(C)(C)C)ccc3N(c3cc(-c4ccccc4)cc(-c4ccccc4)c3)c3cc(-c4ncncn4)cc(c31)N2c1cc(-c2ccccc2)cc(-c2ccccc2)c1 ; CC(C)(C)c1ccc2c(c1)B1c3cc(C(C)(C)C)ccc3N(c3cc(-c4ccccc4)cc(-c4ccccc4)c3)c3cc(-c4nc(-c5ccccc5)nc(-c5ccccc5)n4)cc(c31)N2c1cc(-c2ccccc2)cc(-c2ccccc2)c1]

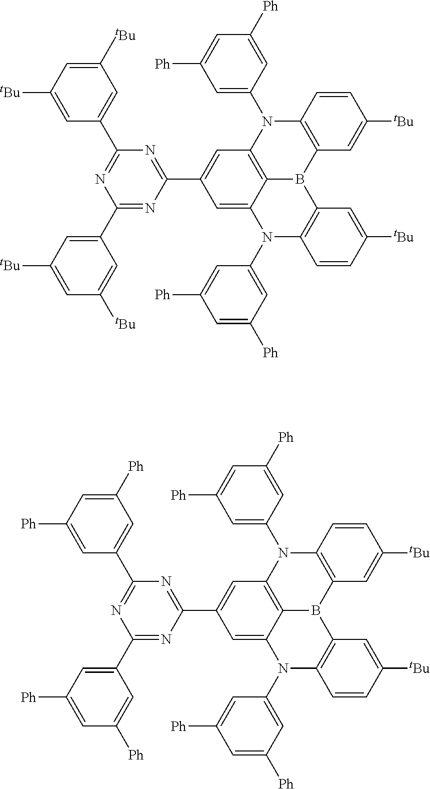 [SMILES: CC(C)(C)c1cc(-c2nc(-c3cc(C(C)(C)C)cc(C(C)(C)C)c3)nc(-c3cc4c5c(c3)N(c3cc(-c6ccccc6)cc(-c6ccccc6)c3)c3ccc(C(C)(C)C)cc3B5c3cc(C(C)(C)C)ccc3N4c3cc(-c4ccccc4)cc(-c4ccccc4)c3)n2)cc(C(C)(C)C)c1 ; CC(C)(C)c1ccc2c(c1)B1c3cc(C(C)(C)C)ccc3N(c3cc(-c4ccccc4)cc(-c4ccccc4)c3)c3cc(-c4nc(-c5cc(-c6ccccc6)cc(-c6ccccc6)c5)nc(-c5cc(-c6ccccc6)cc(-c6ccccc6)c5)n4)cc(c31)N2c1cc(-c2ccccc2)cc(-c2ccccc2)c1]

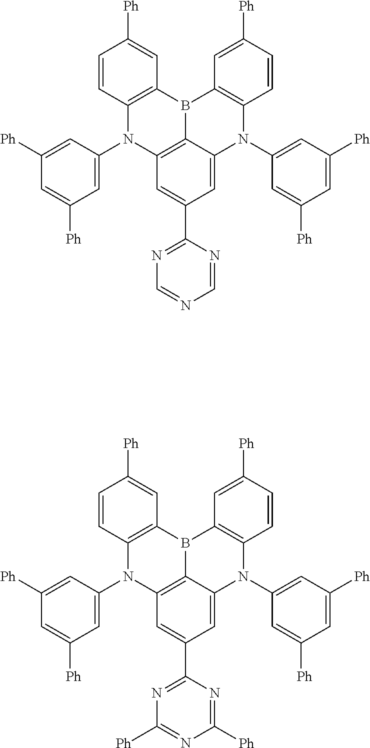 [SMILES: c1ccc(-c2cc(-c3ccccc3)cc(N3c4ccc(-c5ccccc5)cc4B4c5cc(-c6ccccc6)ccc5N(c5cc(-c6ccccc6)cc(-c6ccccc6)c5)c5cc(-c6ncncn6)cc3c54)c2)cc1 ; c1ccc(-c2cc(-c3ccccc3)cc(N3c4ccc(-c5ccccc5)cc4B4c5cc(-c6ccccc6)ccc5N(c5cc(-c6ccccc6)cc(-c6ccccc6)c5)c5cc(-c6nc(-c7ccccc7)nc(-c7ccccc7)n6)cc3c54)c2)cc1]

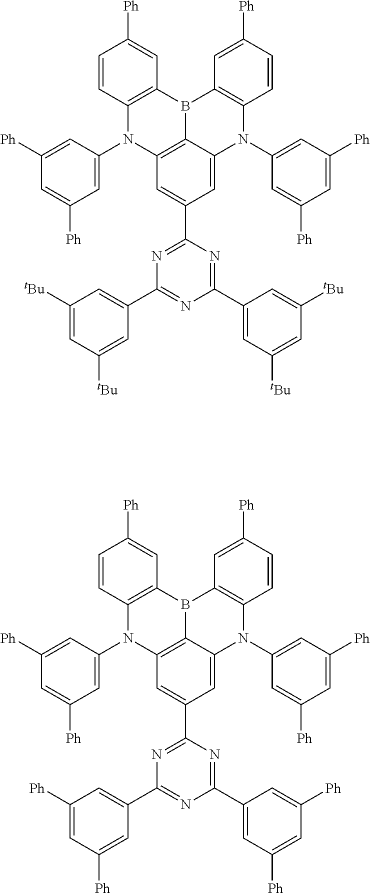 [SMILES: CC(C)(C)c1cc(-c2nc(-c3cc(C(C)(C)C)cc(C(C)(C)C)c3)nc(-c3cc4c5c(c3)N(c3cc(-c6ccccc6)cc(-c6ccccc6)c3)c3ccc(-c6ccccc6)cc3B5c3cc(-c5ccccc5)ccc3N4c3cc(-c4ccccc4)cc(-c4ccccc4)c3)n2)cc(C(C)(C)C)c1 ; c1ccc(-c2cc(-c3ccccc3)cc(-c3nc(-c4cc(-c5ccccc5)cc(-c5ccccc5)c4)nc(-c4cc5c6c(c4)N(c4cc(-c7ccccc7)cc(-c7ccccc7)c4)c4ccc(-c7ccccc7)cc4B6c4cc(-c6ccccc6)ccc4N5c4cc(-c5ccccc5)cc(-c5ccccc5)c4)n3)c2)cc1]

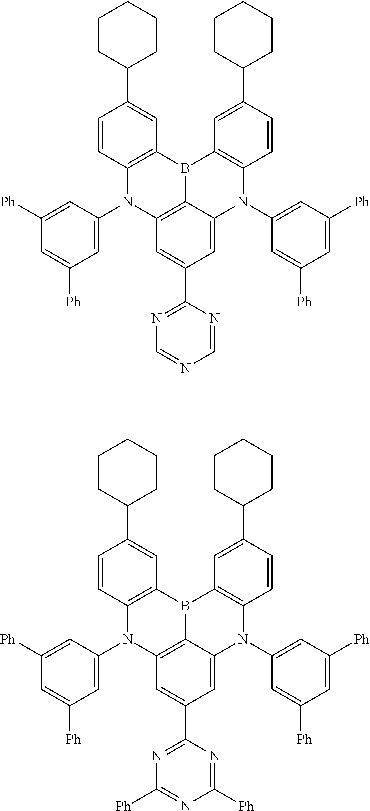 [SMILES: c1ccc(-c2cc(-c3ccccc3)cc(N3c4ccc(C5CCCCC5)cc4B4c5cc(C6CCCCC6)ccc5N(c5cc(-c6ccccc6)cc(-c6ccccc6)c5)c5cc(-c6ncncn6)cc3c54)c2)cc1 ; c1ccc(-c2cc(-c3ccccc3)cc(N3c4ccc(C5CCCCC5)cc4B4c5cc(C6CCCCC6)ccc5N(c5cc(-c6ccccc6)cc(-c6ccccc6)c5)c5cc(-c6nc(-c7ccccc7)nc(-c7ccccc7)n6)cc3c54)c2)cc1]

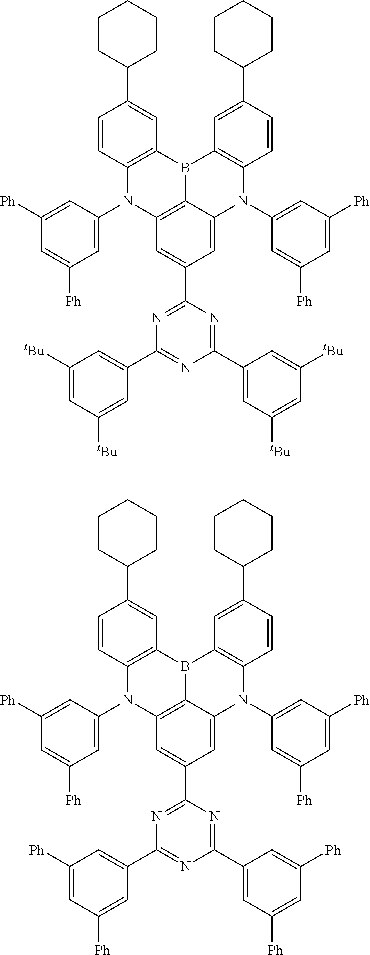 [SMILES: CC(C)(C)c1cc(-c2nc(-c3cc(C(C)(C)C)cc(C(C)(C)C)c3)nc(-c3cc4c5c(c3)N(c3cc(-c6ccccc6)cc(-c6ccccc6)c3)c3ccc(C6CCCCC6)cc3B5c3cc(C5CCCCC5)ccc3N4c3cc(-c4ccccc4)cc(-c4ccccc4)c3)n2)cc(C(C)(C)C)c1 ; c1ccc(-c2cc(-c3ccccc3)cc(-c3nc(-c4cc(-c5ccccc5)cc(-c5ccccc5)c4)nc(-c4cc5c6c(c4)N(c4cc(-c7ccccc7)cc(-c7ccccc7)c4)c4ccc(C7CCCCC7)cc4B6c4cc(C6CCCCC6)ccc4N5c4cc(-c5ccccc5)cc(-c5ccccc5)c4)n3)c2)cc1]

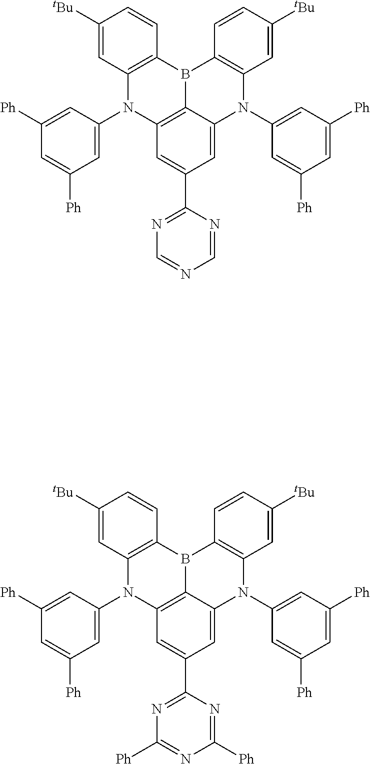 [SMILES: CC(C)(C)c1ccc2c(c1)N(c1cc(-c3ccccc3)cc(-c3ccccc3)c1)c1cc(-c3ncncn3)cc3c1B2c1ccc(C(C)(C)C)cc1N3c1cc(-c2ccccc2)cc(-c2ccccc2)c1 ; CC(C)(C)c1ccc2c(c1)N(c1cc(-c3ccccc3)cc(-c3ccccc3)c1)c1cc(-c3nc(-c4ccccc4)nc(-c4ccccc4)n3)cc3c1B2c1ccc(C(C)(C)C)cc1N3c1cc(-c2ccccc2)cc(-c2ccccc2)c1]

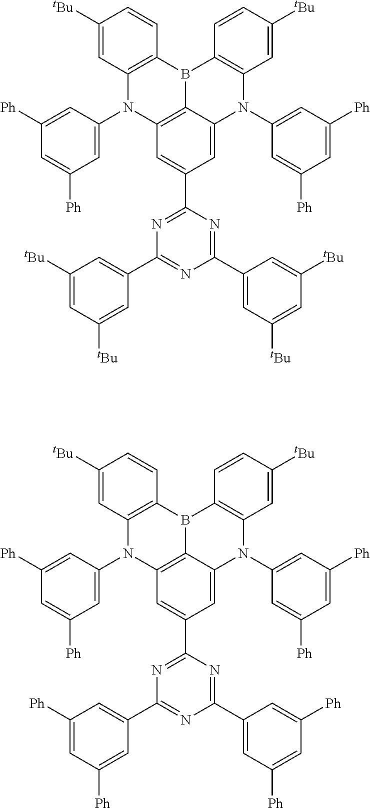 [SMILES: CC(C)(C)c1cc(-c2nc(-c3cc(C(C)(C)C)cc(C(C)(C)C)c3)nc(-c3cc4c5c(c3)N(c3cc(-c6ccccc6)cc(-c6ccccc6)c3)c3cc(C(C)(C)C)ccc3B5c3ccc(C(C)(C)C)cc3N4c3cc(-c4ccccc4)cc(-c4ccccc4)c3)n2)cc(C(C)(C)C)c1 ; CC(C)(C)c1ccc2c(c1)N(c1cc(-c3ccccc3)cc(-c3ccccc3)c1)c1cc(-c3nc(-c4cc(-c5ccccc5)cc(-c5ccccc5)c4)nc(-c4cc(-c5ccccc5)cc(-c5ccccc5)c4)n3)cc3c1B2c1ccc(C(C)(C)C)cc1N3c1cc(-c2ccccc2)cc(-c2ccccc2)c1]

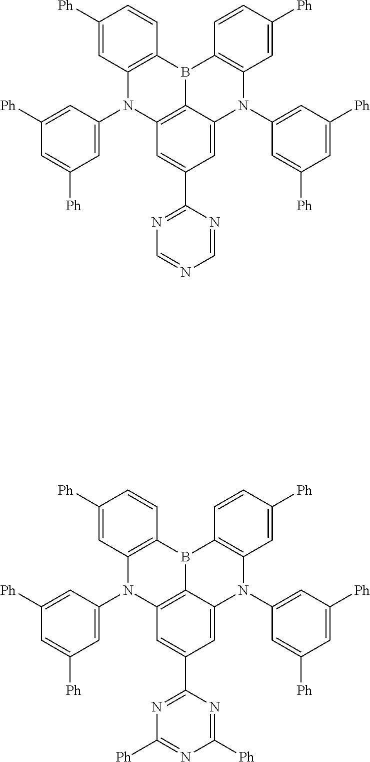 [SMILES: c1ccc(-c2cc(-c3ccccc3)cc(N3c4cc(-c5ccccc5)ccc4B4c5ccc(-c6ccccc6)cc5N(c5cc(-c6ccccc6)cc(-c6ccccc6)c5)c5cc(-c6ncncn6)cc3c54)c2)cc1 ; c1ccc(-c2cc(-c3ccccc3)cc(N3c4cc(-c5ccccc5)ccc4B4c5ccc(-c6ccccc6)cc5N(c5cc(-c6ccccc6)cc(-c6ccccc6)c5)c5cc(-c6nc(-c7ccccc7)nc(-c7ccccc7)n6)cc3c54)c2)cc1]

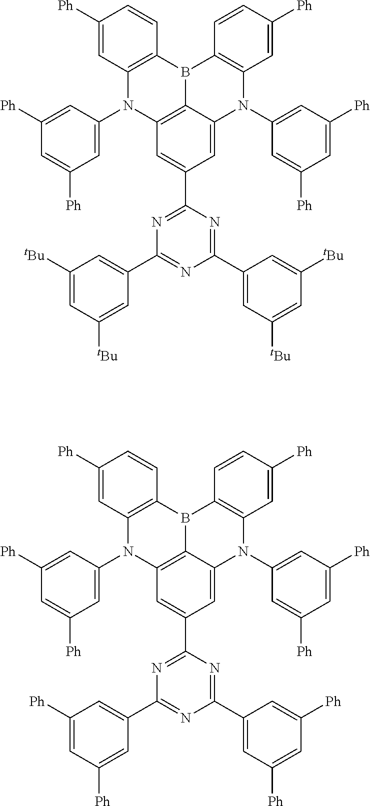 [SMILES: CC(C)(C)c1cc(-c2nc(-c3cc(C(C)(C)C)cc(C(C)(C)C)c3)nc(-c3cc4c5c(c3)N(c3cc(-c6ccccc6)cc(-c6ccccc6)c3)c3cc(-c6ccccc6)ccc3B5c3ccc(-c5ccccc5)cc3N4c3cc(-c4ccccc4)cc(-c4ccccc4)c3)n2)cc(C(C)(C)C)c1 ; c1ccc(-c2cc(-c3ccccc3)cc(-c3nc(-c4cc(-c5ccccc5)cc(-c5ccccc5)c4)nc(-c4cc5c6c(c4)N(c4cc(-c7ccccc7)cc(-c7ccccc7)c4)c4cc(-c7ccccc7)ccc4B6c4ccc(-c6ccccc6)cc4N5c4cc(-c5ccccc5)cc(-c5ccccc5)c4)n3)c2)cc1]

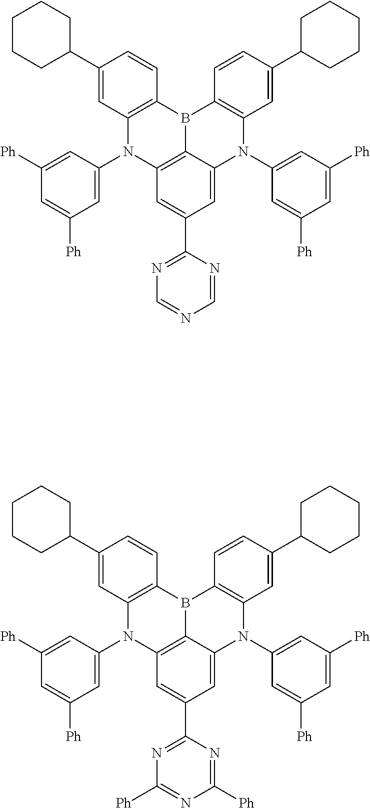 [SMILES: c1ccc(-c2cc(-c3ccccc3)cc(N3c4cc(C5CCCCC5)ccc4B4c5ccc(C6CCCCC6)cc5N(c5cc(-c6ccccc6)cc(-c6ccccc6)c5)c5cc(-c6ncncn6)cc3c54)c2)cc1 ; c1ccc(-c2cc(-c3ccccc3)cc(N3c4cc(C5CCCCC5)ccc4B4c5ccc(C6CCCCC6)cc5N(c5cc(-c6ccccc6)cc(-c6ccccc6)c5)c5cc(-c6nc(-c7ccccc7)nc(-c7ccccc7)n6)cc3c54)c2)cc1]

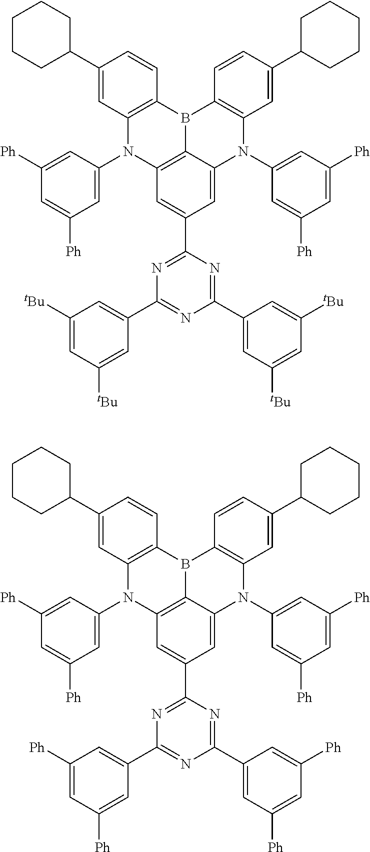 [SMILES: CC(C)(C)c1cc(-c2nc(-c3cc(C(C)(C)C)cc(C(C)(C)C)c3)nc(-c3cc4c5c(c3)N(c3cc(-c6ccccc6)cc(-c6ccccc6)c3)c3cc(C6CCCCC6)ccc3B5c3ccc(C5CCCCC5)cc3N4c3cc(-c4ccccc4)cc(-c4ccccc4)c3)n2)cc(C(C)(C)C)c1 ; c1ccc(-c2cc(-c3ccccc3)cc(-c3nc(-c4cc(-c5ccccc5)cc(-c5ccccc5)c4)nc(-c4cc5c6c(c4)N(c4cc(-c7ccccc7)cc(-c7ccccc7)c4)c4cc(C7CCCCC7)ccc4B6c4ccc(C6CCCCC6)cc4N5c4cc(-c5ccccc5)cc(-c5ccccc5)c4)n3)c2)cc1]

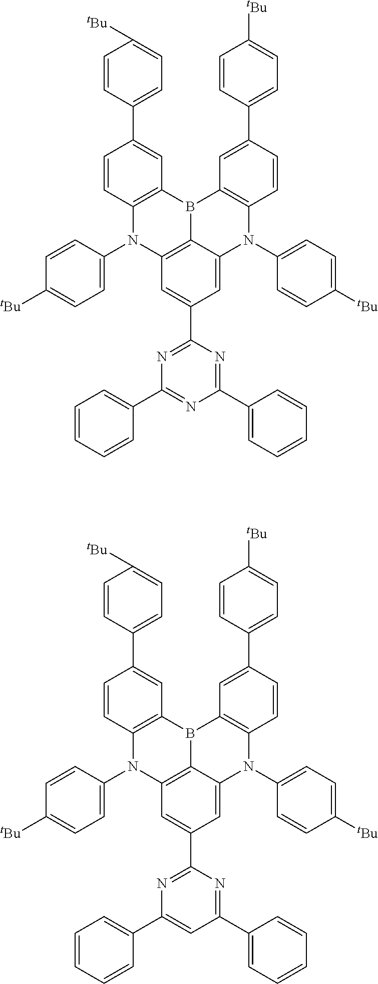 [SMILES: CC(C)(C)c1ccc(-c2ccc3c(c2)B2c4cc(-c5ccc(C(C)(C)C)cc5)ccc4N(c4ccc(C(C)(C)C)cc4)c4cc(-c5nc(-c6ccccc6)nc(-c6ccccc6)n5)cc(c42)N3c2ccc(C(C)(C)C)cc2)cc1 ; CC(C)(C)c1ccc(-c2ccc3c(c2)B2c4cc(-c5ccc(C(C)(C)C)cc5)ccc4N(c4ccc(C(C)(C)C)cc4)c4cc(-c5nc(-c6ccccc6)cc(-c6ccccc6)n5)cc(c42)N3c2ccc(C(C)(C)C)cc2)cc1]

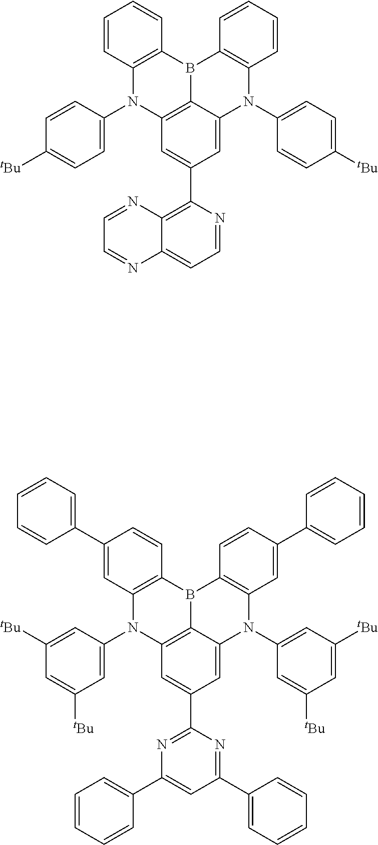 [SMILES: CC(C)(C)c1ccc(N2c3ccccc3B3c4ccccc4N(c4ccc(C(C)(C)C)cc4)c4cc(-c5nccc6nccnc56)cc2c43)cc1 ; CC(C)(C)c1cc(N2c3cc(-c4ccccc4)ccc3B3c4ccc(-c5ccccc5)cc4N(c4cc(C(C)(C)C)cc(C(C)(C)C)c4)c4cc(-c5nc(-c6ccccc6)cc(-c6ccccc6)n5)cc2c43)cc(C(C)(C)C)c1]

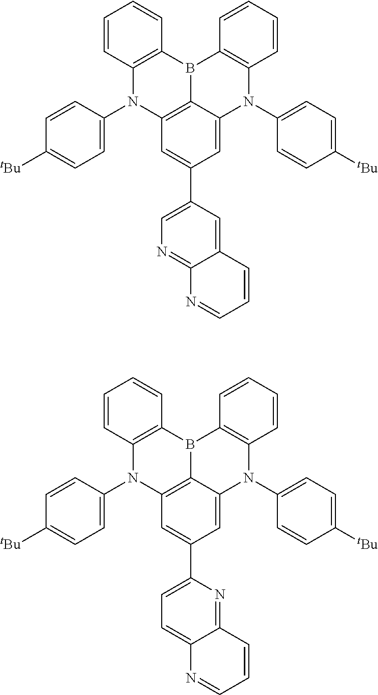 [SMILES: CC(C)(C)c1ccc(N2c3ccccc3B3c4ccccc4N(c4ccc(C(C)(C)C)cc4)c4cc(-c5cnc6ncccc6c5)cc2c43)cc1 ; CC(C)(C)c1ccc(N2c3ccccc3B3c4ccccc4N(c4ccc(C(C)(C)C)cc4)c4cc(-c5ccc6ncccc6n5)cc2c43)cc1]

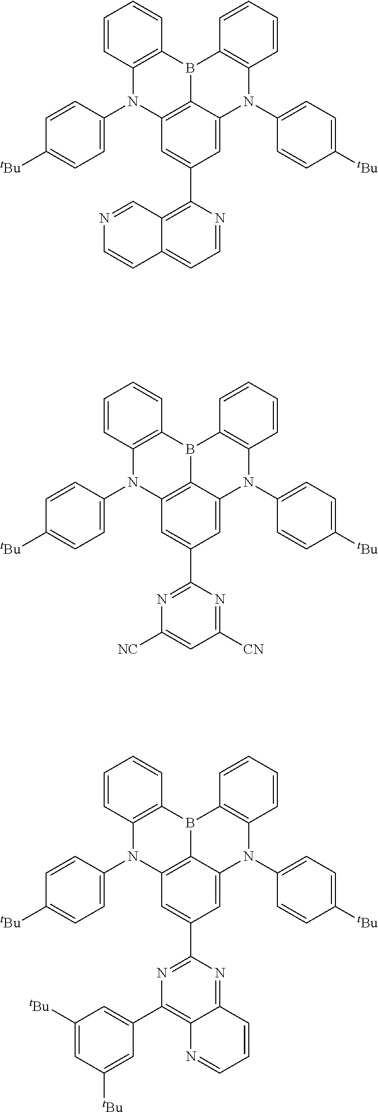 [SMILES: CC(C)(C)c1ccc(N2c3ccccc3B3c4ccccc4N(c4ccc(C(C)(C)C)cc4)c4cc(-c5nccc6ccncc56)cc2c43)cc1 ; CC(C)(C)c1ccc(N2c3ccccc3B3c4ccccc4N(c4ccc(C(C)(C)C)cc4)c4cc(-c5nc(C#N)cc(C#N)n5)cc2c43)cc1 ; CC(C)(C)c1ccc(N2c3ccccc3B3c4ccccc4N(c4ccc(C(C)(C)C)cc4)c4cc(-c5nc(-c6cc(C(C)(C)C)cc(C(C)(C)C)c6)c6ncccc6n5)cc2c43)cc1]

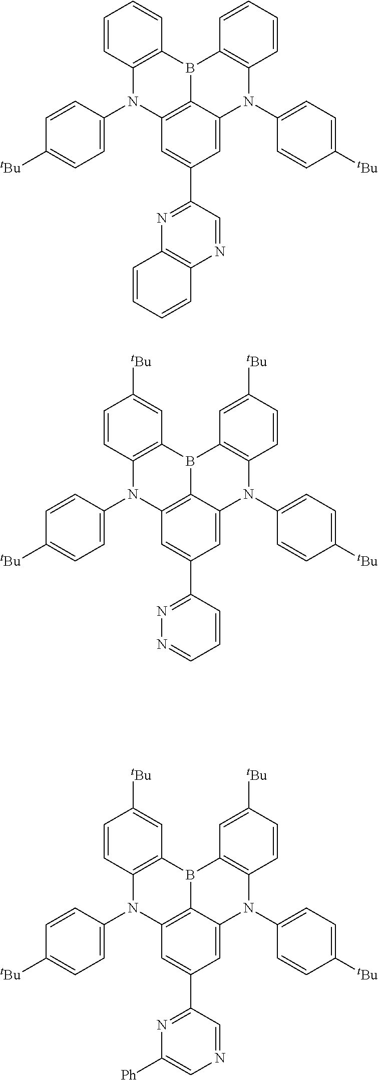 [SMILES: CC(C)(C)c1ccc(N2c3ccccc3B3c4ccccc4N(c4ccc(C(C)(C)C)cc4)c4cc(-c5cnc6ccccc6n5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5cccnn5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5cncc(-c6ccccc6)n5)cc2c43)cc1]

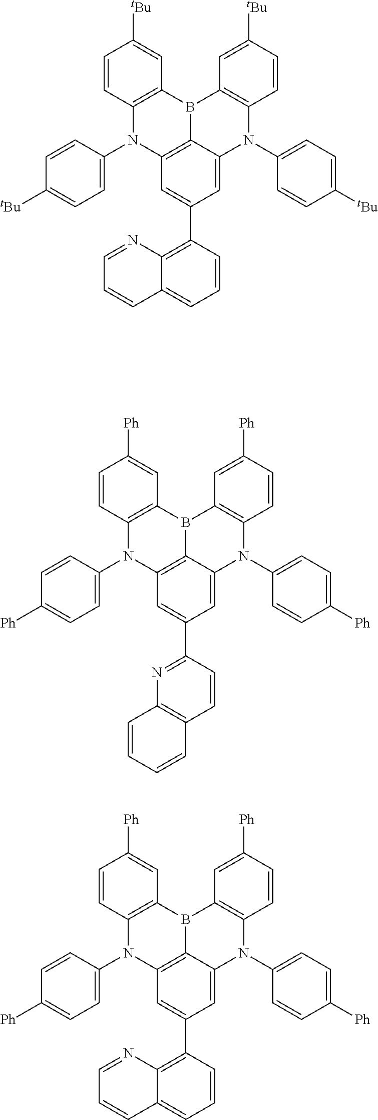 [SMILES: CC(C)(C)c1ccc(N2c3ccc(C(C)(C)C)cc3B3c4cc(C(C)(C)C)ccc4N(c4ccc(C(C)(C)C)cc4)c4cc(-c5cccc6cccnc56)cc2c43)cc1 ; c1ccc(-c2ccc(N3c4ccc(-c5ccccc5)cc4B4c5cc(-c6ccccc6)ccc5N(c5ccc(-c6ccccc6)cc5)c5cc(-c6ccc7ccccc7n6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6cccc7cccnc67)cc3c54)cc2)cc1]

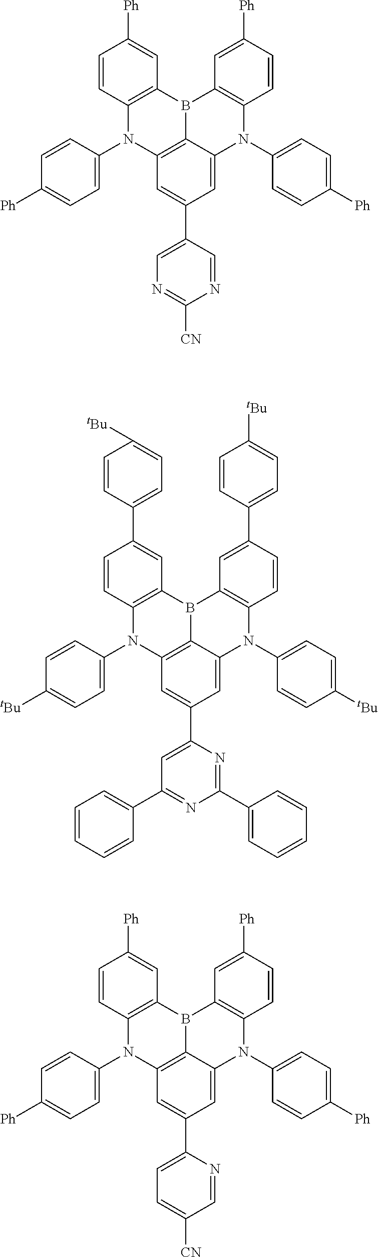 [SMILES: N#Cc1ncc(-c2cc3c4c(c2)N(c2ccc(-c5ccccc5)cc2)c2ccc(-c5ccccc5)cc2B4c2cc(-c4ccccc4)ccc2N3c2ccc(-c3ccccc3)cc2)cn1 ; CC(C)(C)c1ccc(-c2ccc3c(c2)B2c4cc(-c5ccc(C(C)(C)C)cc5)ccc4N(c4ccc(C(C)(C)C)cc4)c4cc(-c5cc(-c6ccccc6)nc(-c6ccccc6)n5)cc(c42)N3c2ccc(C(C)(C)C)cc2)cc1 ; N#Cc1ccc(-c2cc3c4c(c2)N(c2ccc(-c5ccccc5)cc2)c2ccc(-c5ccccc5)cc2B4c2cc(-c4ccccc4)ccc2N3c2ccc(-c3ccccc3)cc2)nc1]

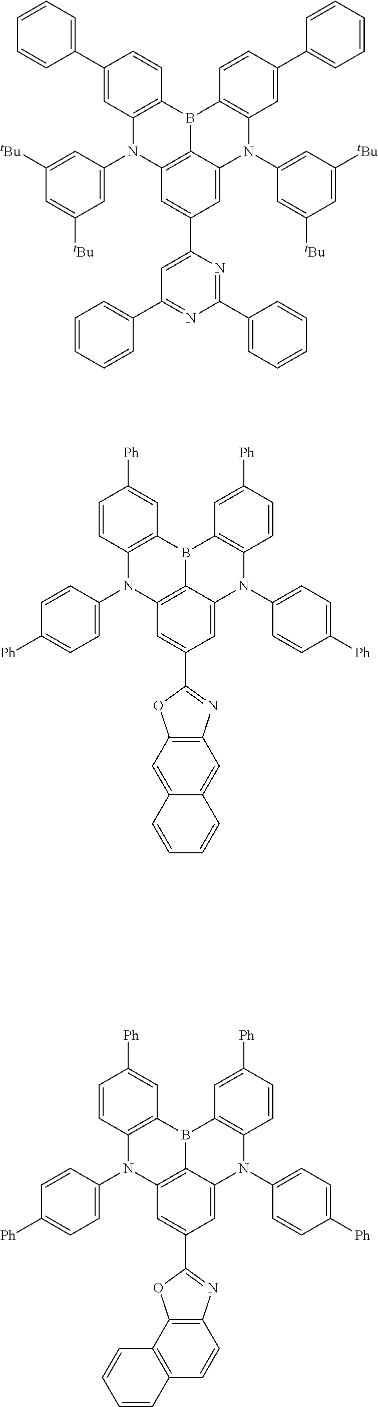 [SMILES: CC(C)(C)c1cc(N2c3cc(-c4ccccc4)ccc3B3c4ccc(-c5ccccc5)cc4N(c4cc(C(C)(C)C)cc(C(C)(C)C)c4)c4cc(-c5cc(-c6ccccc6)nc(-c6ccccc6)n5)cc2c43)cc(C(C)(C)C)c1 ; c1ccc(-c2ccc(N3c4ccc(-c5ccccc5)cc4B4c5cc(-c6ccccc6)ccc5N(c5ccc(-c6ccccc6)cc5)c5cc(-c6nc7cc8ccccc8cc7o6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6nc7ccc8ccccc8c7o6)cc3c54)cc2)cc1]

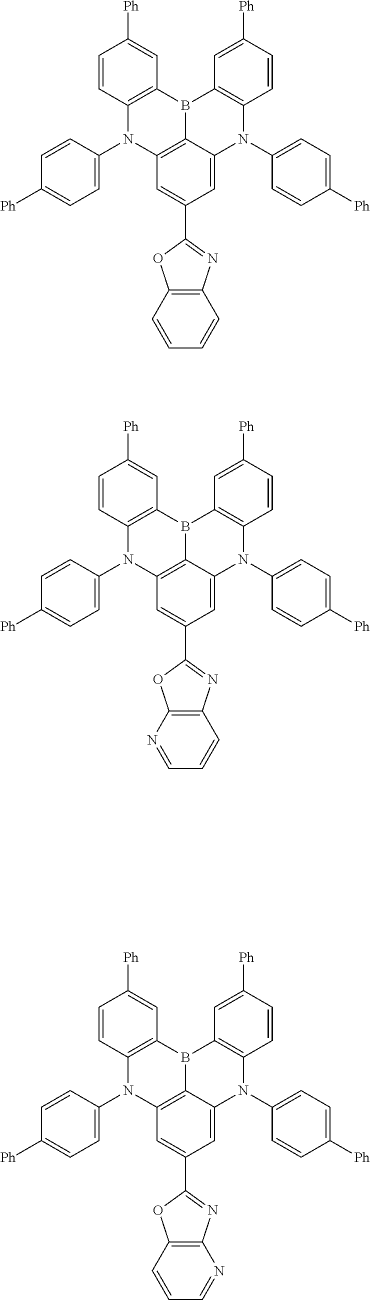 [SMILES: c1ccc(-c2ccc(N3c4ccc(-c5ccccc5)cc4B4c5cc(-c6ccccc6)ccc5N(c5ccc(-c6ccccc6)cc5)c5cc(-c6nc7ccccc7o6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6nc7cccnc7o6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6nc7ncccc7o6)cc3c54)cc2)cc1]

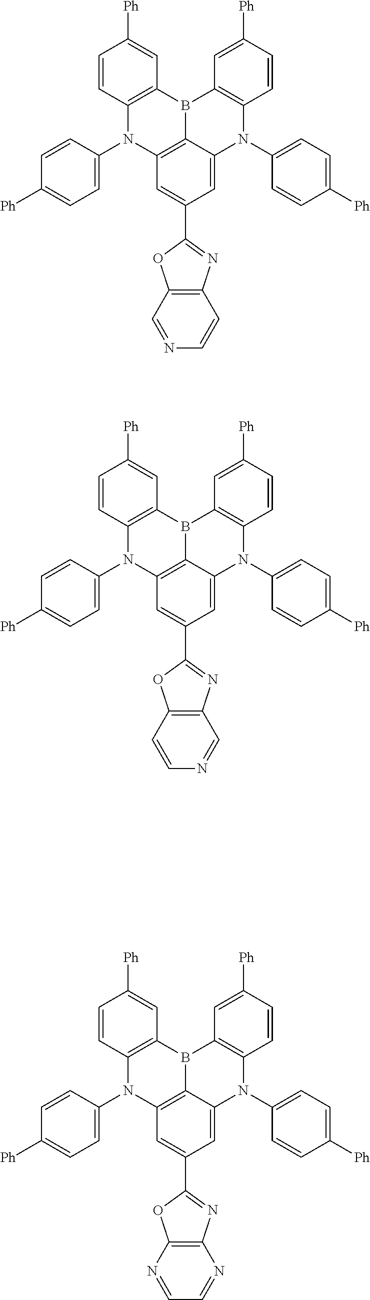 [SMILES: c1ccc(-c2ccc(N3c4ccc(-c5ccccc5)cc4B4c5cc(-c6ccccc6)ccc5N(c5ccc(-c6ccccc6)cc5)c5cc(-c6nc7ccncc7o6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6nc7cnccc7o6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6nc7nccnc7o6)cc3c54)cc2)cc1]

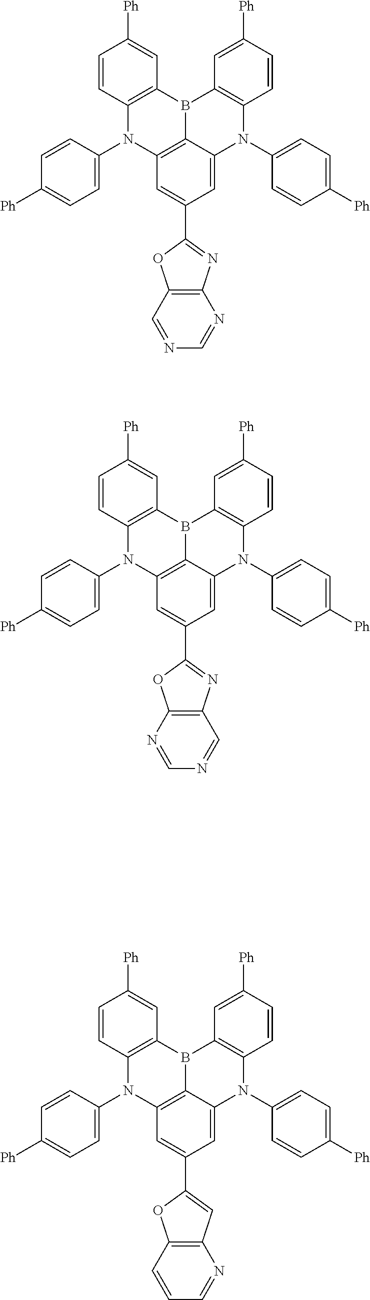 [SMILES: c1ccc(-c2ccc(N3c4ccc(-c5ccccc5)cc4B4c5cc(-c6ccccc6)ccc5N(c5ccc(-c6ccccc6)cc5)c5cc(-c6nc7ncncc7o6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6nc7cncnc7o6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6cc7ncccc7o6)cc3c54)cc2)cc1]

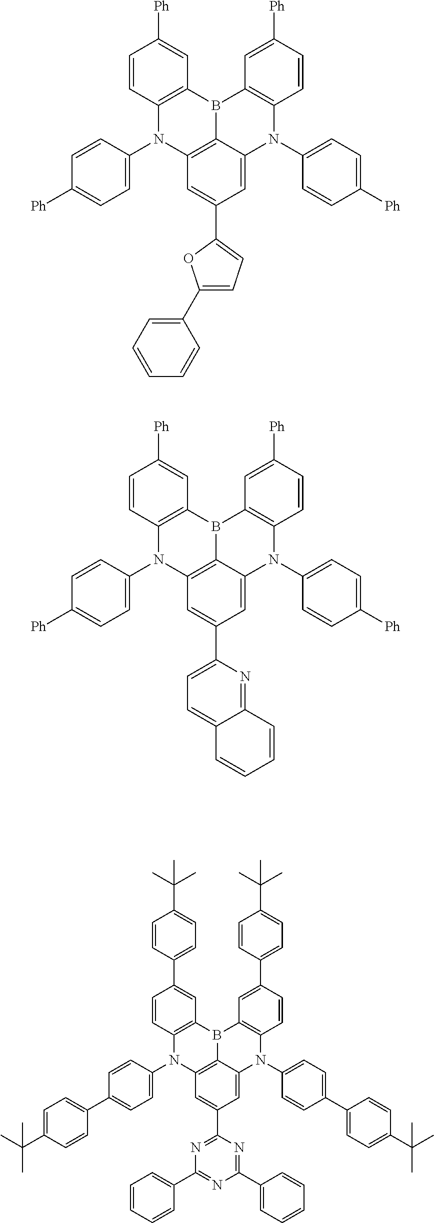 [SMILES: c1ccc(-c2ccc(N3c4ccc(-c5ccccc5)cc4B4c5cc(-c6ccccc6)ccc5N(c5ccc(-c6ccccc6)cc5)c5cc(-c6ccc(-c7ccccc7)o6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6ccc7ccccc7n6)cc3c54)cc2)cc1 ; CC(C)(C)c1ccc(-c2ccc(N3c4ccc(-c5ccc(C(C)(C)C)cc5)cc4B4c5cc(-c6ccc(C(C)(C)C)cc6)ccc5N(c5ccc(-c6ccc(C(C)(C)C)cc6)cc5)c5cc(-c6nc(-c7ccccc7)nc(-c7ccccc7)n6)cc3c54)cc2)cc1]

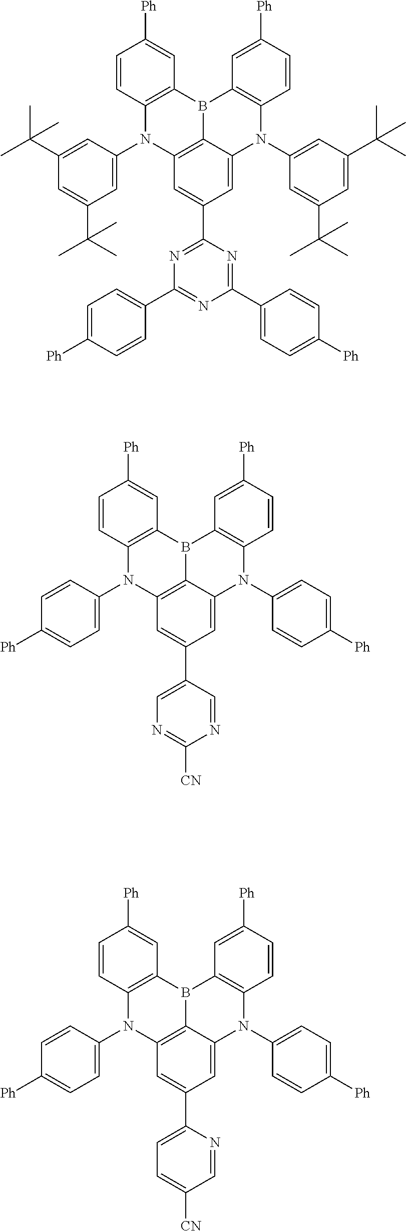 [SMILES: CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5nc(-c6ccc(-c7ccccc7)cc6)nc(-c6ccc(-c7ccccc7)cc6)n5)cc2c43)cc(C(C)(C)C)c1 ; N#Cc1ncc(-c2cc3c4c(c2)N(c2ccc(-c5ccccc5)cc2)c2ccc(-c5ccccc5)cc2B4c2cc(-c4ccccc4)ccc2N3c2ccc(-c3ccccc3)cc2)cn1 ; N#Cc1ccc(-c2cc3c4c(c2)N(c2ccc(-c5ccccc5)cc2)c2ccc(-c5ccccc5)cc2B4c2cc(-c4ccccc4)ccc2N3c2ccc(-c3ccccc3)cc2)nc1]

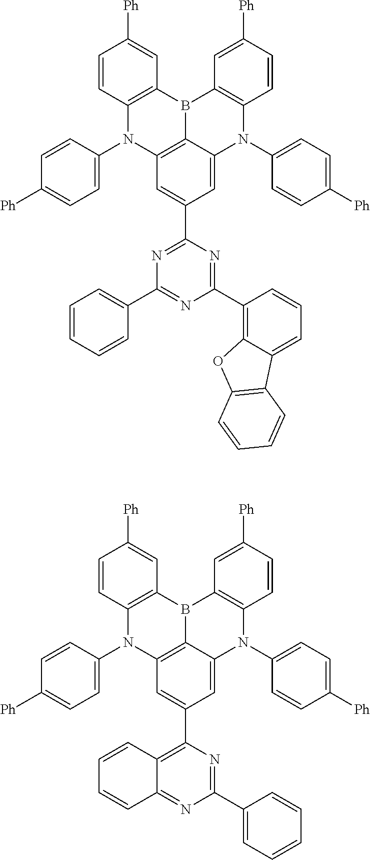 [SMILES: c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)nc(-c7cccc8c7oc7ccccc78)n6)cc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)nc7ccccc67)cc3c54)cc2)cc1]

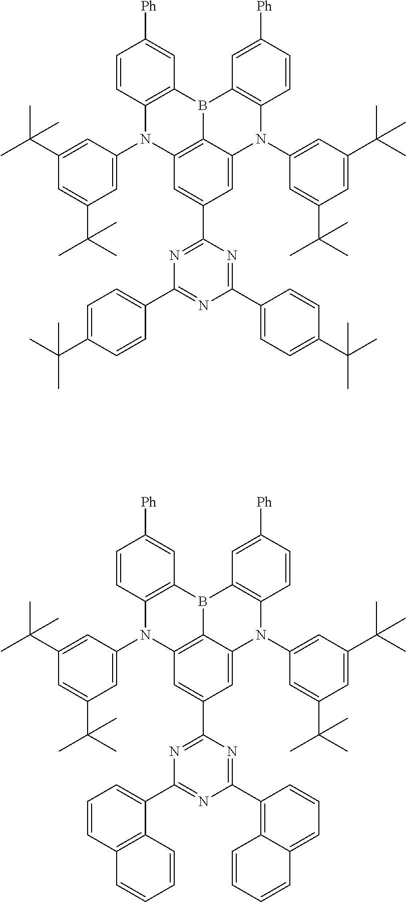

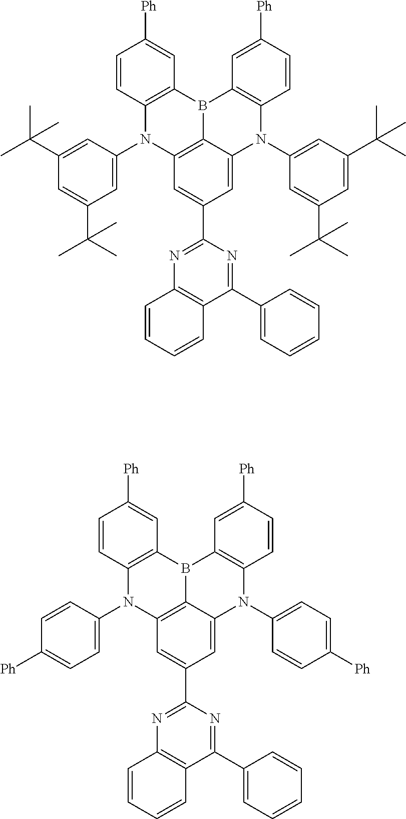 [SMILES: CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5nc(-c6ccccc6)c6ccccc6n5)cc2c43)cc(C(C)(C)C)c1 ; c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)c7ccccc7n6)cc3c54)cc2)cc1]

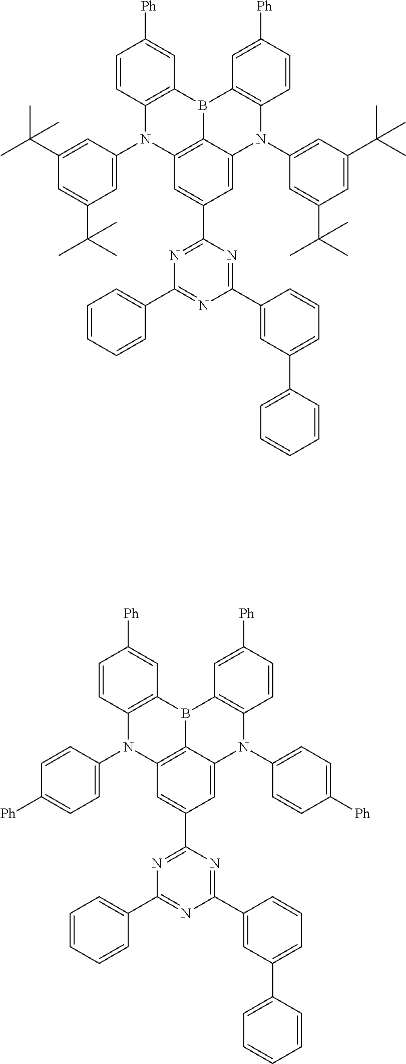 [SMILES: CC(C)(C)c1cc(N2c3ccc(-c4ccccc4)cc3B3c4cc(-c5ccccc5)ccc4N(c4cc(C(C)(C)C)cc(C(C)(C)C)c4)c4cc(-c5nc(-c6ccccc6)nc(-c6cccc(-c7ccccc7)c6)n5)cc2c43)cc(C(C)(C)C)c1 ; c1ccc(-c2ccc(N3c4ccc(-c5ccccc5)cc4B4c5cc(-c6ccccc6)ccc5N(c5ccc(-c6ccccc6)cc5)c5cc(-c6nc(-c7ccccc7)nc(-c7cccc(-c8ccccc8)c7)n6)cc3c54)cc2)cc1]

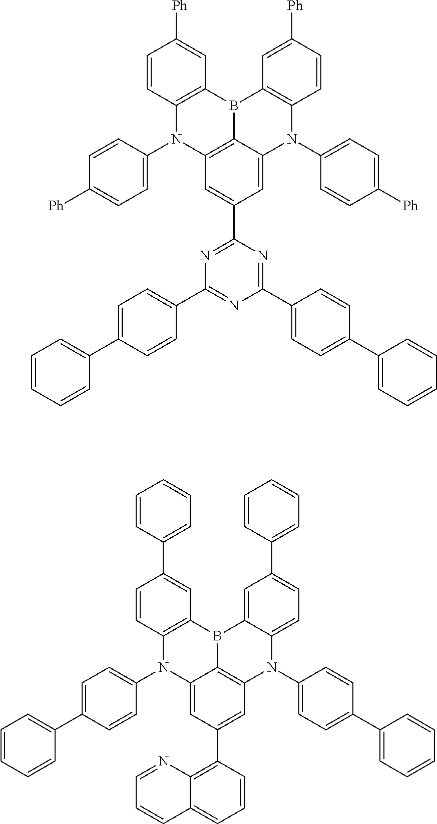 [SMILES: c1ccc(-c2ccc(-c3nc(-c4ccc(-c5ccccc5)cc4)nc(-c4cc5c6c(c4)N(c4ccc(-c7ccccc7)cc4)c4ccc(-c7ccccc7)cc4B6c4cc(-c6ccccc6)ccc4N5c4ccc(-c5ccccc5)cc4)n3)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-c6cccc7cccnc67)cc3c54)cc2)cc1]

### FIGURES

FIG. 1 Emission spectrum of example 1 (5% by weight) in PMMA.

FIG. 2 Emission spectrum of example 2 (1% by weight) in PMMA.

FIG. 3 Emission spectrum of example 3 (2% by weight) in PMMA.

FIG. 4 Emission spectrum of example 4 (1% by weight) in PMMA.

FIG. 5 Emission spectrum of example 5 (2% by weight) in PMMA.

FIG. 6 Emission spectrum of example 6 (2% by weight) in PMMA.

FIG. 7 Emission spectrum of example 7 (2% by weight) in PMMA.

FIG. 8 Emission spectrum of example 8 (2% by weight) in PMMA.

FIG. 9 Emission spectrum of example 9 (2% by weight) in PMMA.

FIG. 10 Emission spectrum of example 10 (2% by weight) in PMMA.

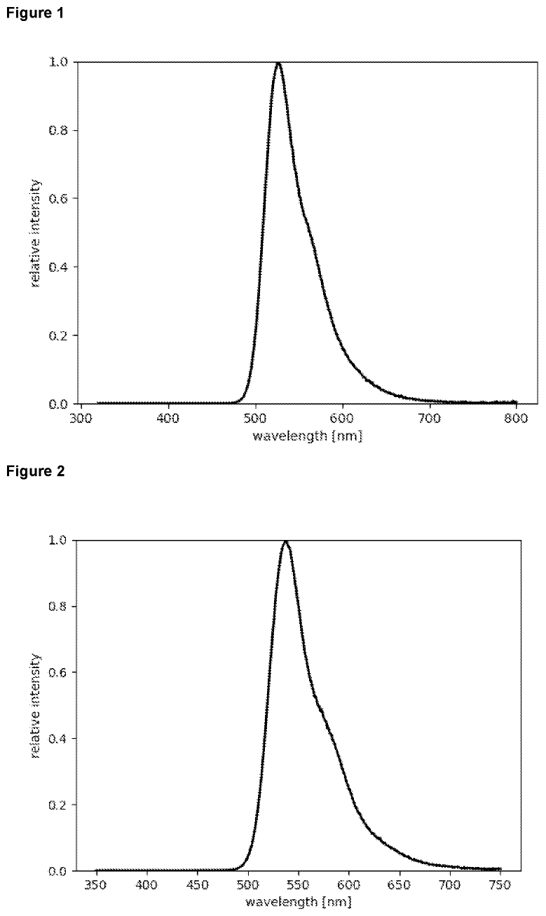

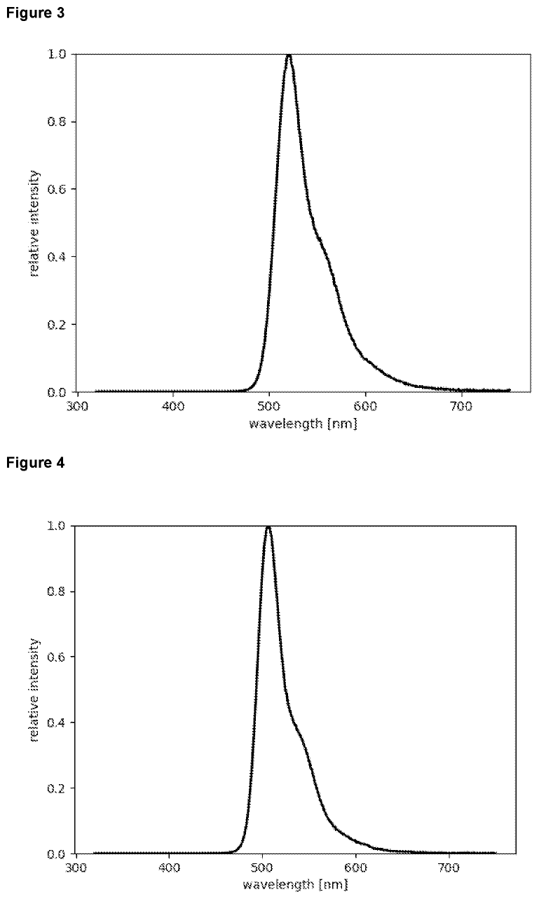

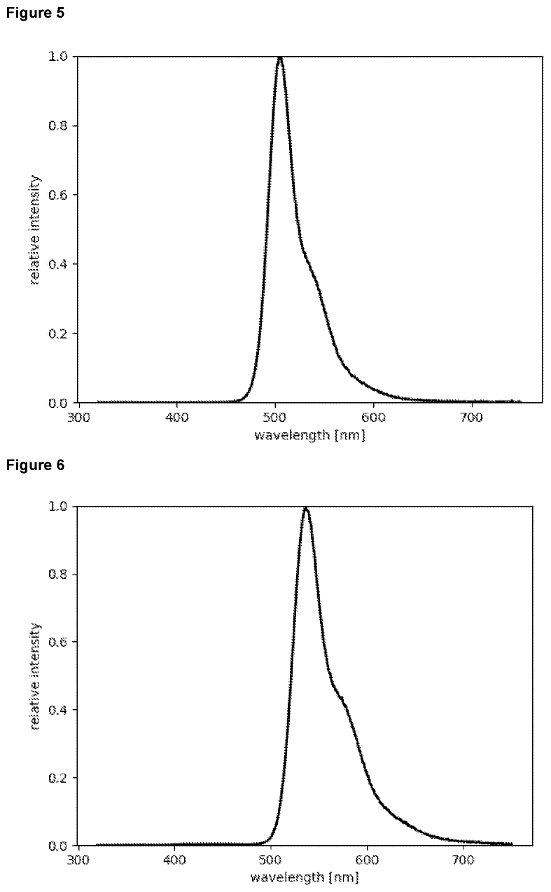

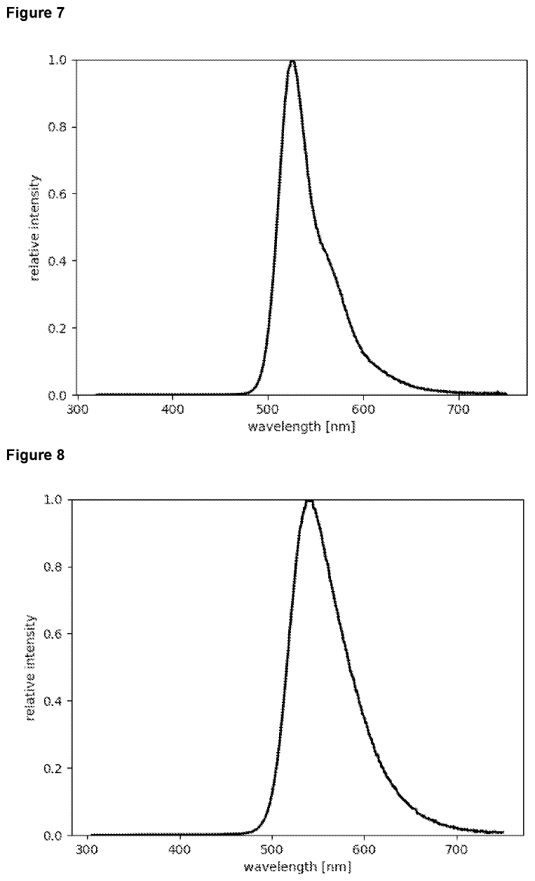

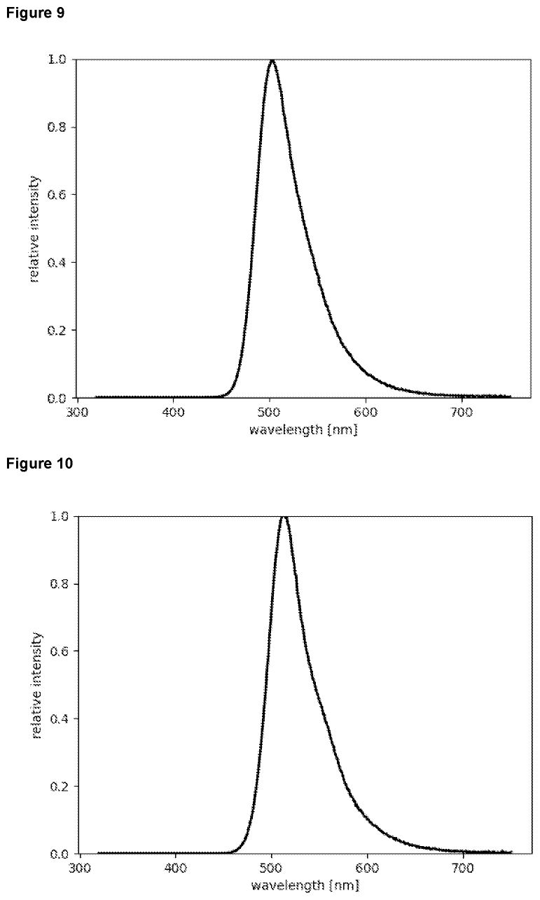

1. An organic molecule, comprising a structure of Formula I: 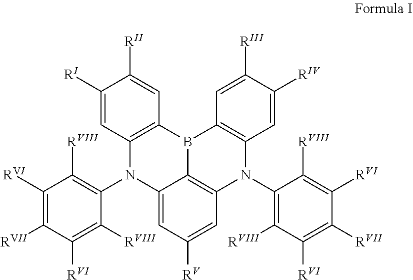
wherein
R^{I}, R^{II}, R^{III} and R^{IV} are independently from another selected from the group consisting of:
hydrogen,
deuterium,
N(R⁵)₂,
OR⁵,
SR⁵,
Si(R⁵)₃,
B(OR⁵)₂,
OSO₂R⁵,
CF₃,
CN,
halogen,
C₁-C₄₀-alkyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-alkoxy,
which is optionally substituted with one or more substituents R⁵ and
wherein one or more non-adjacent CH₂-groups are optionally substituted by R⁵C═CR⁵, C—C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-thioalkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkenyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkynyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₆-C₆₀-aryl,
which is optionally substituted with one or more substituents R⁵; and
C₃-C₅₇-heteroaryl,
which is optionally substituted with one or more substituents R⁵;
R⁵ is at each occurrence independently from another selected from the group consisting of:
hydrogen, deuterium, OPh, SPh, CF₃, CN, F, Si(C₁-C₅-alkyl)₃, Si(Ph)₃,
C₁-C₅-alkyl,
wherein optionally one or more hydrogen atoms are independently from each other substituted by deuterium, CN, CF₃, or F;
C₁-C₅-alkoxy,
wherein optionally one or more hydrogen atoms are independently from each other substituted by deuterium, CN, CF₃, or F;
C₁-C₅-thioalkoxy,
wherein optionally one or more hydrogen atoms are independently from each other substituted by deuterium, CN, CF₃, or F;
C₂-C₅-alkenyl,
wherein optionally one or more hydrogen atoms are independently from each other substituted by deuterium, CN, CF₃, or F;
C₂-C₅-alkynyl,
wherein optionally one or more hydrogen atoms are independently from each other substituted by deuterium, CN, CF₃, or F;
C₆-C₁₈-aryl,
which is optionally substituted with one or more C₁-C₅-alkyl substituents;
C₃-C₁₇-heteroaryl,
which is optionally substituted with one or more C₁-C₅-alkyl substituents;
N(C₆-C₁₈-aryl)₂,
N(C₃-C₁₇-heteroaryl)₂; and
N(C₃-C₁₇-heteroaryl)(C₆-C₁₈-aryl);
R^{V} is at each occurrence independently from another selected from the group consisting of:
a C₃-C₁₅-heteroaryl,
wherein optionally one or more hydrogen atoms are independently from each other substituted by deuterium, halogen, C₁-C₅-alkyl, CN, CF₃, SiMe₃, SiPh₃,
C₃-C₁₅-heteroaryl, and
C₆-C₁₈-aryl, wherein optionally one or more hydrogen atoms are independently from each other substituted by C₁-C₅-alkyl, CN, CF₃ and Ph;
R^{VI}, R^{VII} and R^{VIII} are at each occurrence independently from another selected from the group consisting of: hydrogen, deuterium,
C₁-C₅-alkyl,
wherein one or more hydrogen atoms are optionally substituted by deuterium;
C₆-C₁₈-aryl,
wherein optionally one or more hydrogen atoms are independently from each other
substituted by C₁-C₅-alkyl, C₆-Cis aryl or C₃-C₁₇-heteroaryl;
C₃-C₁₅-heteroaryl,
wherein optionally one or more hydrogen atoms are independently from each other substituted by C₁-C₅-alkyl, C₆-Cis aryl or C₃-C₁₇-heteroaryl.

2. The organic molecule according to claim 1, wherein R^{V} comprises a structure of Formula I-0: 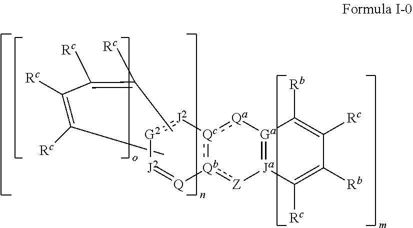
wherein
m is 0 or 1;
n is 0 or 1;
is 0 or 1;
if n=0, then o=0;
G^{a} is C if m=1; G^{a} is CR^{a} or N if m=0;
J^{a} is C if m=1; J^{a} is CR^{a} or N if m=0;
Q^{b} is C if n=1; Q^{b} is CR¹ or N if n=0;
Q^{c} is C if n=1; Q^{c} is CR² or N if n=0;
G² is C if o=1; G² is CR^{b} or N if o=0;
if o=1 exactly one J² is C and the other J² is N or CR^{b};
if o=0 J² is at each occurrence independently from another selected from the group consisting of N and CR^{b};
Z is Q or a direct single bond;
Q is at each occurrence independently from each other selected from the group consisting
of N and C—R²;
if Z is a direct bond:
Q^{a} is selected from a group consisting of NR^{c}, O;
Q^{b} and Q^{c} are connected via a double bond and Q^{c} and Q^{a} are connected via a single bond;
if Z is Q:
Q^{a} is selected from a group consisting N, O, C—R^{a};
Z and Q^{b} are connected via a double bond, while Q^{b} and Q^{c} are connected via a single bond and Q^{c} and Q^{a} are connected via a double bond;
R^{a} is at each occurrence independently from another selected from the group consisting of the binding site or R²
R¹ and R² are at each occurrence independently from another selected from the group consisting of:
hydrogen, deuterium, halogen, C₁-C₅-alkyl, CN, CF₃, SiMe₃, SiPh₃ (Ph=phenyl), C₃-C₁₅-heteroaryl, and
C₆-C₁₈-aryl, wherein optionally one or more hydrogen atoms are independently from each other substituted by C₁-C₅-alkyl, CN, CF₃ and Ph;
R^{b} is at each occurrence independently from another selected from the group consisting of the binding site, hydrogen, deuterium, C₁-C₅-alkyl, C₆-Cis aryl or C₃-C₁₇-heteroaryl;
R^{c} is at each occurrence independently from another selected from the group consisting of hydrogen, deuterium, C₁-C₅-alkyl, C₆-Cis aryl or C₃-C₁₇-heteroaryl;
exactly one of R^{a} and R^{b} is the binding site of R^{V} to the structure shown in formula I; and
at least one atom of Formula I-0 is N or O.

3. The organic molecule according to claim 1, wherein
R^{I}, R^{II}, R^{III} and R^{IV} are independently from another selected from the group consisting of:
hydrogen, deuterium, halogen, Me, ⁱPr, ^{t}Bu, CN, CF₃, SiMe₃, SiPh₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph,
carbazolyl, which is optionally substituted with one or more substituents independently from each other selected from the group consisting of Me, ⁱPr, ^{t}Bu, CN, CF₃, and Ph,
triazinyl, which is optionally substituted with one or more substituents independently from each other selected from the group consisting of Me, ⁱPr, ^{t}Bu, CN, CF₃, and Ph,
and N(Ph)₂.

4. The organic molecule according to claim 1, wherein the organic molecule comprises a structure of Formula Ia:

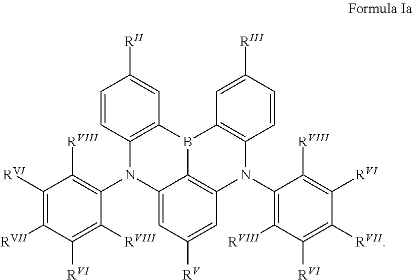

5. The organic molecule according to claim 1, wherein R^{II} is equal to R^{III}.

6. The organic molecule according to claim 1, wherein R^{V} is selected from the group consisting of Formulas 2a, 2b, 2c, 2d, 2e, 2f, 2g, 2h, 2i, 2j, 2k and 2l: 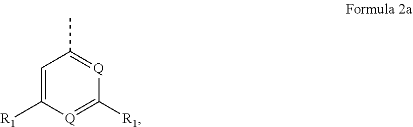
wherein
Q is at each occurrence independently from each other selected from the group consisting of N and C—R².
R¹ and R² are at each occurrence independently from another selected from the group consisting of:
hydrogen, deuterium, halogen, Me, ⁱPr, ^{t}Bu, CN, CF₃, SiMe₃, SiPh₃ (Ph=phenyl), and C₆-C₁₈-aryl,
wherein optionally one or more hydrogen atoms are independently from each other substituted by C₁-C₅-alkyl, CN, CF₃ and Ph 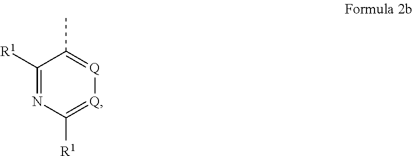
wherein
Q is at each occurrence independently from each other selected from the group consisting of N and C—R²;
R¹ and R² are at each occurrence independently from another selected from the group consisting of:
hydrogen, deuterium, halogen, Me, ⁱPr, ^{t}Bu, CN, CF₃, SiMe₃, SiPh₃ (Ph=phenyl), and C₆-C₁₈-aryl,
wherein optionally one or more hydrogen atoms are independently from each other substituted by C₁-C₅-alkyl, CN, CF₃ and Ph 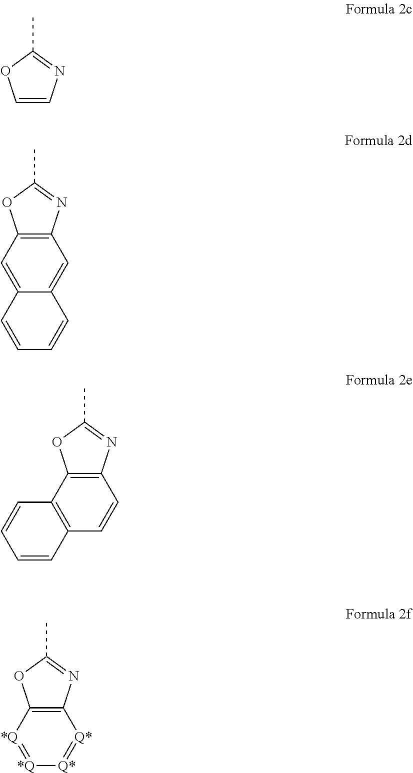
Q* is at each occurrence independently from each other selected from the group consisting of N and C—H, wherein optionally one or more hydrogen atoms are independently from each other substituted by R^{c}; 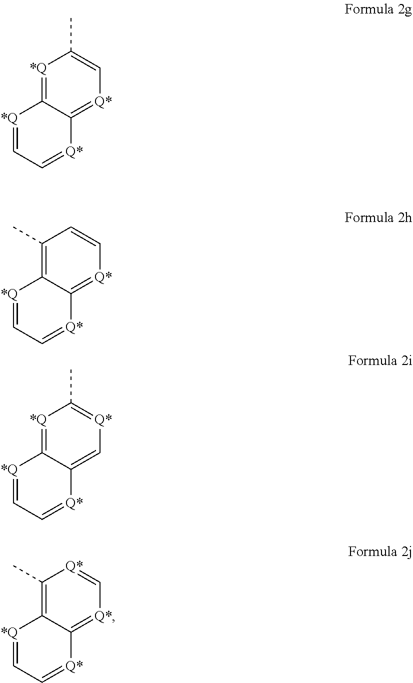
wherein
Q* is at each occurrence independently from each other selected from the group consisting of N and C—H, wherein optionally one or more hydrogen atoms are independently from each other substituted by R^{c}, wherein at least one Q*=N;
and 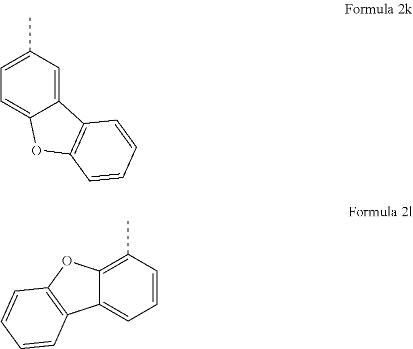
wherein optionally one or more hydrogen atoms are independently from each other substituted by R^{c};
which is bonded via the position marked by a dotted line: “ [symbol] ”.

7. The organic molecule according to claim 1, comprising a structure of Formula 3:

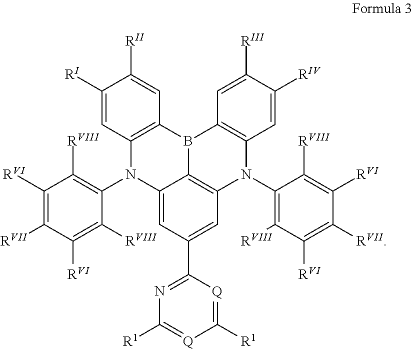

8. The organic molecule according to claim 1, comprising a structure of Formula Ib:

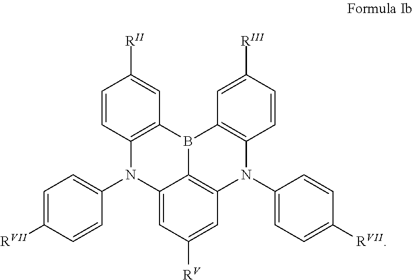

9. The organic molecule according to claim 1, comprising a structure of Formula Ig:

10. (canceled)

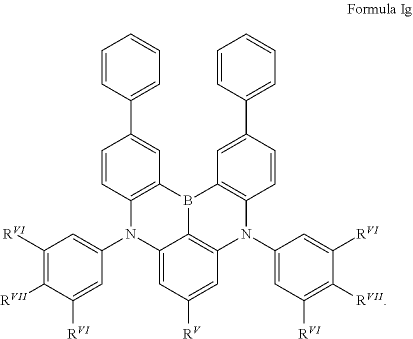

11. (canceled)

12. A composition, comprising:
(a) an organic molecule according to claim 1, as an emitter and/or a host;
(b) an emitter and/or a host material different from the organic molecule according to claim 1; and
(c) optionally, a dye and/or a solvent.

13. (canceled)

14. (canceled)

15. (canceled)

16. An optoelectronic device comprising the organic molecule according to claim 1.

17. The optoelectronic device according to claim 16, wherein the optoelectronic device is an organic light-emitting diode, a light-emitting electrochemical cell, an organic light-emitting sensor, an organic diode, an organic solar cell, an organic transistor, an organic field-effect transistor, an organic laser or a down-conversion element.

18. The optoelectronic device according to claim 16, comprising:
a substrate;
an anode;
a cathode, wherein the anode or the cathode is disposed on the substrate; and
at least one light-emitting layer disposed between the anode and the cathode and which comprises the organic molecule.

19. An optoelectronic device comprising the organic molecule according to claim 1, wherein the organic molecule is one of a luminescent emitter, an electron transport material, a hole injection material or a hole blocking material in the optoelectronic device.

20. An optoelectronic device comprising the organic molecule according to claim 2, wherein the optoelectronic device is an organic light-emitting diode, a light-emitting electrochemical cell, an organic light-emitting sensor, an organic diode, an organic solar cell, an organic transistor, an organic field-effect transistor, an organic laser or a down-conversion element.

21. The optoelectronic device according to claim 20, comprising:
a substrate;
an anode;
a cathode, wherein the anode or the cathode is applied to the substrate; and
at least one light-emitting layer disposed between the anode and the cathode and which comprises the organic molecule.

22. An optoelectronic device comprising the composition according to claim 12.

23. The optoelectronic device according to claim 22, wherein the optoelectronic device is an organic light-emitting diode, a light-emitting electrochemical cell, an organic light-emitting sensor, an organic diode, an organic solar cell, an organic transistor, an organic field-effect transistor, an organic laser or a down-conversion element.

24. The optoelectronic device according to claim 22, comprising:
a substrate;
an anode;
a cathode, wherein the anode or the cathode is disposed on the substrate; and
at least one light-emitting layer disposed between the anode and the cathode and which comprises the composition.

25. A method for producing an optoelectronic device, comprising processing of the organic molecule according to claim 1 by a vacuum evaporation method or from a solution.
